SEQUENCE LISTING (6 sequences)

SEQ ID NO: 1 - DNA, 18 bp, Artificial Sequence
  Primers for Neomycin gene
ccggctacctgcccattc

SEQ ID NO: 2 - DNA, 22 bp, Artificial Sequence
  Primers for Neomycin gene
ccagatcatcctgatcgacaag

SEQ ID NO: 3 - DNA, 24 bp, Artificial Sequence
  Primers for Neomycin gene
acatcgcatcgagcgagcacgtac

SEQ ID NO: 4 - DNA, 36 bp, Artificial Sequence
  Primers for Neomycin gene
acatgcgcggcatctagaccggctacctgcccattc

SEQ ID NO: 5 - DNA, 18 bp, Artificial Sequence
  Primers for Neomycin gene
acatgcgcggcatctaga

SEQ ID NO: 6 - DNA, 22 bp, Artificial Sequence
  Primers for Neomycin gene
ccagatcatcctgatcgacaag

(19) United States
(12) Patent Application Publication
(10) Pub. No.: US 2004/0147739 A1
(43) Pub. Date: Jul. 29, 2004

(54) Novel anti-infectives

(76) Inventors: Deping Chai, Collegeville, PA (US); Michael G. Darey, King of Prussia, PA (US); Dashyant Dhanak, King of Prussia, PA (US); Kevin J. Duffy, Collegeville, PA (US); Greg A. Erickson, Research Triangle Park, NC (US); Duke M. Fitch, Collegeville, PA (US); Adam T. Gates, Royersford, PA (US); Victor K. Johnston, Pennsburg, PA (US); Robert T. Sarisky, Lansdale, PA (US); Matthew J. Sharp, Research Triangle Park, NC (US); Antony N. Shaw, Collegeville, PA (US); Rosanna Tedesco, Collegeville, PA (US); Kenneth J. Wiggall, Collegeville, PA (US); Michael N. Zimmerman, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/479,358

(22) PCT Filed: Jun. 7, 2002
(86) PCT No.: PCT/US02/18491

(51) Int. Cl. C07D 285/22; A61K 31/675; A61K 31/549

(52) U.S. Cl. 544/12; 514/80; 514/223.2

(57) ABSTRACT

Compounds useful as HCV anti-infectives having the formula: wherein the formula variables are as defined herein, are disclosed. Also disclosed are methods of making and using the same.

### FIELD OF THE INVENTION

The present invention relates to compounds that inhibit an RNA-containing virus and methods of making and using the same. Specifically, the present invention relates to inhibitors of hepatitis C virus (HCV).

### BACKGROUND OF THE INVENTION

In the U.S., an estimated 4.5 million Americans are chronically infected with HCV. Although only 30% of acute infections are symptomatic, greater than 85% of infected individuals develop chronic, persistent infection. Treatment costs for HCV infection have been estimated at $5.46 billion for the U.S. in 1997. Worldwide, over 200 million people are estimated to be infected chronically. HCV infection is responsible for 40-60% of all chronic liver disease and 30% of all liver transplants. The CDC estimates that the number of deaths due to HCV will minimally increase to ³8,000/yr. by the year 2010.

Due to the high degree of variability in the viral surface antigens, existence of multiple viral genotypes, and demonstrated specificity of immunity, the development of a successful vaccine in the near future is unlikely. Alpha-interferon (alone or in combination with ribavirin) has been widely used since its approval for treatment of chronic HCV infection. However, adverse side effects are commonly associated with this treatment: flu-like symptoms, leukopenia, thrombocytopenia, and depression from interferon, as well as hemolytic anemia induced by ribavirin (Lindsay, K. L. (1997) Hepatology 26 (Suppl. 1):71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of those treated, 50-70% relapse within 6 months of cessation of treatment. Recently with the introduction of pegylated interferon (Peg-IFN), both initial and sustained response rates have improved substantially, and combination treatment of Peg-IFN with ribavirin constitutes the gold standard for therapy. However, the side effects associated with combination therapy and the impaired response in patients with genotype 1 present opportunities for improvement in the management of this disease.

First identified by molecular cloning in 1989 (Choo, Q-L. et al., (1989) Science 244:359-362), HCV is now widely accepted as the most common causative agent of post-transfusion non A, non-B hepatitis (NANBH) (Kuo, G. et al., (1989) Science 244:362-364). Due to its genome structure and sequence homology, this virus was assigned as a new genus in the Flaviviridae family. Like the other members of the Flaviviridae (such as flaviviruses (e.g., yellow fever virus and Dengue virus types 14) and pestiviruses (e.g., bovine viral diarrhea virus, border disease virus, and classic swine fever virus (Choo et al., 1989; Miller, R. H. and R. H. Purcell (1990) Proc. Natl. Acad. Sci. USA 87:2057-2061)), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang, C. Y., Le, S. Y., Ali, N., Siddiqui, A., Rna-A Publication of the Rna Society. 1(5): 526-537, 1995 July). This element is followed by a region which encodes a single long open reading frame (ORF) encoding a polypeptide of ˜3000 amino acids comprising both the structural and nonstructural viral proteins.

Upon entry into the cytoplasm of the cell, the HCV-RNA is directly translated into a polypeptide of ˜3000 amino acids comprising both the structural and nonstructural viral proteins. This large polypeptide is subsequently processed into the individual structural and nonstructural proteins by a combination of host and virally-encoded proteinases (Rice, C. M. (1996) in B. N. Fields, D. M. Knipe and P. M. Howley (Eds.) Virology, 2nd Edition, p931-960, Raven Press, NY). Following the termination codon at the end of the long ORF, there is a 3′ NTR which roughly consists of three regions: an ˜40 base region which is poorly conserved among various genotypes, a variable length poly(U)/polypyrimidine tract, and a highly conserved 98 base element also called the “3′ X-tail” (Kolykhalov, A. et al., (1996) J. Virology 70:3363-3371; Tanaka, T. et al., (1995) Biochem Biophys. Res. Commun. 215:744-749; Tanaka, T. et al., (1996) J. Virology 70:3307-3312; Yamada, N. et al., (1996) Virology 223:255-261). The 3′NTR is predicted to form a stable secondary structure that is essential for HCV growth in chimps and is believed to function in the initiation and regulation of viral RNA replication.

The NS5B protein (591 amino acids, 65 kDa) of HCV (Behrens, S. E., et al., (1996) EMBO J. 15:12-22), encodes an RNA-dependent RNA polymerase (RdRp) activity and contains canonical motifs present in other RNA viral polymerases. The NS5B protein is fairly well conserved both intra-typically (˜95-98% amino acid (aa) identity across 1b isolates) and inter-typically (˜85% aa identity between genotype 1a and 1b isolates). The essentiality of the HCV NS5B RdRp activity for the generation of infectious progeny virions has been formally proven in chimpanzees (Kolykhalov, A. A., et al., (2000) J. Virology 74:2046-2051). Thus, inhibition of NS5B RdRp activity (inhibition of RNA replication) is predicted to cure HCV infection.

Positive strand hepatitis C viral RNA is the nucleic acid strand that is translated and initially copied upon entry of the HCV-RNA into the cell. Once in the cell, positive strand viral RNA generates a negative strand replicative intermediate. Negative strand RNA is the template used to generate the positive strand message that is generally packaged into productive virions. Presently, HCV inhibitor compounds are only evaluated for their ability to inhibit positive strand HCV-RNA. However, it would be desirable to develop inhibitor compounds having the ability to inhibit both positive and negative strand replication to obtain complete clearance of the HCV virus.

Accordingly, there exists a significant need to identify synthetic or biological compounds for their ability to inhibit HCV. Preferably, such synthetic or biological compounds inhibit both positive and negative strand replication of the hepatitis C virus.

### SUMMARY OF THE INVENTION

This invention is directed to compounds having Formula I, as follows:

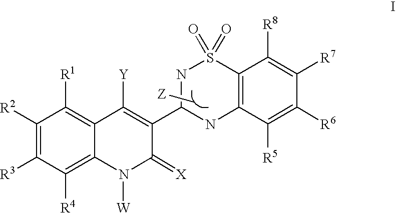

wherein:

R¹ is hydrogen, halogen, C₁-C₄ alkyl, —OR¹¹, —SR¹¹, —NR¹⁰R¹¹, aryl, —C(O)OH, —C(O)NHR¹¹, cyano or nitro;

R² is hydrogen, C₁-C₈ alkyl, C₂-C₈ alkenyl, C₂-C₈ alkynyl, C₃-C₆ cycloalkyl, heterocycloalkyl, aryl, heteroaryl, nitro, cyano, halogen, —C(O)OR⁹, —C(O)R⁹, —C(O)NR⁹R¹⁰, —OR⁹, —SR⁹, —S(O)R², —S(O)₂R¹², —NR⁹R¹⁰, protected —OH, —N(R¹⁰)C(O)R⁹, —OC(O)NR⁹R¹⁰, —N(R¹⁰)C(O)NR⁹R¹⁰, —P(O)(OR⁹)₂, —SO₂NR⁹R¹⁰, —SO₃H, or —N(R¹⁰)SO₂R¹²,

where said C₁-C₈ alkyl, C₂-C₈ alkenyl or C₂-C₈ alkyl is unsubstituted or substituted with one or more substituents independently selected from halogen, —OH, —SH, —OC₁-C₄ alkyl, —SC₁-C₄ alkyl, —NR¹⁰R¹¹, cyano, nitro, —CO₂R¹⁰, —C(O)OC₁-C₄ alkyl, —CONR¹⁰R¹¹, —CONH₂, aryl, and heteroaryl,

and where said cycloalkyl, heterocycloalkyl, aryl or heteroaryl is unsubstituted or substituted with one or more substituents independently selected from C₁-C₆ alkyl, C₁-C₆ haloalkyl, halogen, —OH, —SH, —NH₂, —OC₁-C₄ alkyl, —SC₁-C₄ alkyl, —N(C₁-C₄ alkyl)(C₁-C₄ alkyl), —NH(C₁-C₄ alkyl), cyano, nitro, —CO₂H, —C(O)OC₁-C₄ alkyl, —CON(C₁-C₄ alkyl)(C₁-C₄ alkyl), —CONH(C₁-C₄ alkyl) and —CONH₂;

R³ is hydrogen, halogen, cyano, C₁-C₆ alkyl, —OH, or —CO₂H;

R⁴, R⁵ and R⁶ are each independently selected from the group consisting of hydrogen, halogen, cyano, C₁-C₆ alkyl, —OH, and —OC₁-C₄ alkyl;

R⁷ is hydrogen, C₁-C₈ alkyl, C₂-C₈ alkenyl, C₂-C₈ alkynyl, C₃-C₆ cycloalkyl, heterocycloalkyl, aryl, heteroaryl, nitro, cyano, halogen, —C(O)OR⁹, —C(O)R⁹, —C(O)NR⁹R¹⁰, —OR⁹, —SR⁹, —S(O)R¹², —S(O)₂R¹², —NR⁹R¹⁰, protected —OH, —N(R¹⁰)C(O)R⁹, —OC(O)NR⁹R¹⁰, —N(R¹⁰)C(O)NR⁹R¹⁰, —P(O)(OR⁹)₂, —SO₂NR⁹R¹⁰, —SO₃H, or —N(R¹⁰)SO₂R¹²,

where said C₁-C₈ alkyl, C₂-C₈ alkenyl or C₂-C₈ alkyl is unsubstituted or substituted with one or more substituents independently selected from halogen, —OH, —SH, —OC₁-C₄ alkyl, —SC₁-C₄ alkyl, —NR¹⁰R¹¹, cyano, nitro, —CO₂H, —C(O)OC₁-C₄ alkyl, —CONR¹⁰R¹¹, —CONH₂, aryl, heteroaryl, heterocycloalkyl, —C(O)aryl, —C(O)heterocycloalkyl, and —C(O)heteroaryl, where said aryl, heteroaryl, heterocycloalkyl, aryl, —C(O)aryl, —C(O)heterocycloalkyl, or —C(O)heteroaryl is unsubstituted or substituted with one or more substituents independently selected from C₁-C₄ alkyl, C₁-C₄ haloalkyl, halogen, —OH, —SH, —NH₂, —OC₁-C₄ alkyl, —SC₁-C₄ alkyl, —N(C₁-C₄ alkyl)(C₁-C₄ alkyl), —NH(C₁-C₄ alkyl), cyano and nitro,

and where said cycloalkyl, heterocycloalkyl, aryl or heteroaryl is unsubstituted or substituted with one or more substituents independently selected from C₁-C₆ alkyl, C₁-C₆ haloalkyl, halogen, —OH, —SH, —NH₂, —OC₁-C₄ alkyl, —SC₁-C₄ alkyl, —N(C₁-C₄ alkyl)(C₁-C₄ alkyl), —NH(C₁-C₄ alkyl), cyano, nitro, —CO₂H, —C(O)OC₁-C₄ alkyl, —CON(C₁-C₄ alkyl)(C₁-C₄ alkyl), —CONH(C₁-C₄ alkyl) and —CONH₂;

R⁸ is hydrogen, halogen, hydroxyl or C₁-C₄ alkyl;

or R¹ and R² or R⁵ and R⁶ or R⁶ and R⁷ or R⁷ and R⁸ taken together are alkylenedioxy;

W is hydrogen, —C(O)OR¹¹, C₁-C₁₀ alkyl, C₂-C₁₀ alkenyl, C₂-C₁₀ alkynyl, C₃-C₆ cycloalkyl, —(C₁-C₆ alkyl)-(C₃-C₆ cycloalkyl), —(C₂-C₆ alkenyl)-(C₃-C₆ cycloalkyl), —(C₂-C₆ alkynyl)-(C₃-C₆ cycloalkyl), —(C₁-C₆ alkyl)-heterocycloalkyl, —(C₂-C₆ alkenyl)-heterocycloalkyl, —(C₂-C₆ alkynyl)-heterocycloalkyl, —(C₁-C₆ alkyl)-aryl, (C₂-C₆ alkenyl)-aryl, (C₂-C₆ alkynyl)-aryl, —(C₁-C₆ alkyl)-heteroaryl, —(C₂-C₆ alkenyl)-heteroaryl, or —C₂-C₆ alkynyl)-heteroaryl,

where said C₁-C₁₀ alkyl, C₂-C₁₀ alkenyl, C₂-C₁₀ alkynyl is unsubstituted or substituted with one or more substituents independently selected from halogen, cyano, —OH, —OC₁-C₄ alkyl, —SH, —SC₁-C₄ alkyl, —(O)(C₁-C₄ alkyl), —SO₃H, and —S(O)₂(C₁-C₄ alkyl),

said C₃-C₆ cycloalkyl is unsubstituted or substituted with one or more substituents independently selected from halogen, cyano, C₁-C₄ alkyl, —OH, —OC₁-C₄ alkyl, —SH, —SC₁-C₄ alkyl, —S(O)(C₁-C₄ alkyl), —SO₃H, and —S(O)₂(C₁-C₄ alkyl),

and where the cycloalkyl, heterocycloalkyl, aryl or heteroaryl moiety of said —(C₁-C₆ allyl)-(C₃-C₆ cycloalkyl), —C₂-C₆ alkenyl)-(C₃-C₆ cycloalkyl), —(C₂-C₆ alkynyl)-(C₃-C₆ cycloalkyl), —(C₁-C₆ alkyl)-heterocycloalkyl, —(C₂-C₆ alkenyl)-heterocycloalkyl, —(C₂-C₆ alkynyl)-heterocycloalkyl, —(C₁-C₆ alkyl)-aryl, (C₂-C₆ alkenyl)-aryl, —(C₂-C₆ alkynyl)-aryl, —(C₁-C₆ alkyl)-heteroaryl, —(C₂-C₆ alkenyl)-heteroaryl, or —(C₂-C₆ alkynyl)-heteroaryl is unsubstituted or substituted with one or more substituents independently selected from C₁-C₆ alkyl, C₁-C₆ haloalkyl, halogen, cyano, nitro, —OH, —NH₂, —OC₁-C₄ alkyl, —N(C₁-C₄ alkyl)(C₁-C₄ alkyl), and —NH(C₁-C₄ alkyl);

X is O or S;

Y is —OH or —SH;

Z is hydrogen or C₁-C₄ alkyl;

wherein each R⁹ is independently selected from the group consisting of hydrogen, C₁-C₈ alkyl, C₂-C₈ alkenyl, C₂-C₈ alkynyl, C₃-C₈ cycloalkyl, heterocycloalkyl, aryl, heteroaryl, —C₁-C₆ alkyl-C₃-C₈ cycloalkyl, —C₁-C₆ alkyl-heterocycloalkyl, —C₁-C₆ alkyl-aryl, and —C₁-C₆ alkyl-heteroaryl, —C₂-C₆ alkenyl-C₃-C₈ cycloalkyl, —C₂-C₆ alkenyl-heterocycloalkyl, —C₂-C₆ alkenyl-aryl, —C₂C₆ alkenyl-heteroaryl, —C₂-C₆ alkynyl-C₃-C₈ cycloalkyl, —C₂-C₆ alkynyl-heterocycloalkyl, —C₂-C₆ alkynyl-aryl, and —C₂-C₆ alkynyl-heteroaryl,

where said C₁-C₈ alkyl, C₂-C₈ alkenyl, or C₂-C₈ alkynyl is unsubstituted or substituted with one or more substituents independently selected from halogen, —OR¹¹, —NR¹⁰R¹¹, cyano, nitro, —CO₂R¹¹, —CONR¹⁰R¹¹, —NR¹⁰CONR¹⁰R¹¹, —OCONR¹⁰R¹¹, —SO₂NR¹⁰R¹¹, and —COR¹¹,

and where any of said cycloalkyl, heterocycloalkyl, aryl or heteroaryl (including the cycloalkyl, heterocycloalkyl, aryl or heteroaryl moieties of said —C₁-C₆ alkyl-C₃-C₈ cycloalkyl, —C₁-C₆ alkyl-heterocycloalkyl, —C₁-C₆ alkyl-aryl, or —C₁-C₆ alkyl-heteroaryl) is unsubstituted or substituted with one or more substituents independently selected from C₁-C₄ alkyl, C₁-C₄ haloalkyl, halogen, —OR¹¹, —NR¹⁰R¹¹, cyano, nitro, —CO₂R′″, —CONR¹⁰R¹¹, —NR¹⁰CONR¹⁰R¹¹, —OCONR¹⁰R¹¹, —SO₂NR¹⁰R¹¹, and —COR¹¹;

each R¹⁰ is independently selected from hydrogen and C₁-C₆ alkyl;

each R¹¹ is independently selected from the group consisting of hydrogen, C₁-C₆ alkyl, C₃-C₆ cycloalkyl, heterocycloalkyl, aryl, heteroaryl, —C₁-C₄ alkyl-C₃-C₈ cycloalkyl, —C₁-C₄ alkyl-heterocycloalkyl, —C₁-C₄ alkyl-aryl, or —C₁-C₄ alkyl-heteroaryl

where said cycloalkyl, heterocycloalkyl, aryl, heteroaryl, -alkylcycloalkyl, -alkylheterocycloalkyl, -alkylaryl or -alkylheteroaryl is unsubstituted or substituted with one or more substituents independently selected from C₁-C₆ alkyl, C₁-C₆ haloalkyl, halogen —OC₁-C₆ alkyl, —OC₁-C₆ haloalkyl, cyano, —N(C₁-C₆ alkyl)(C₁-C₆ alkyl), —NH(C₁-C₆ alkyl), —NH₂, —CO₂C₁-C₆ alkyl, —CO₂H, —CON(C₁-C₆ alkyl)(C₁-C₆ alkyl), —CONH(C₁-C₆ alkyl), and —CONH₂;

or, when present in any NR⁹R¹⁰ or NR¹⁰R¹¹, each R⁹ and R¹⁰ or each R¹⁰ and R¹, independently, taken together with the nitrogen to which they are attached represent a 3-6-membered saturated ring optionally containing one other heteroatom selected from oxygen and nitrogen, where said 3-6-membered ring is unsubstituted or substituted with one or more substituents independently selected from hydrogen, C₁-C₆ alkyl, halogen, cyano, —OC₁-C₆ alkyl, —OH, —N(C₁-C₆ alkyl)(C₁-C₆ alkyl), —NH(C₁-C₆ alkyl), —NH₂, —CO₂H, —C(O)OC₁-C₆ alkyl, —C(O)C₁-C₆ alkyl, —CON(C₁-C₆ alkyl)(C₁-C₆ alkyl), —CONH(C₁-C₆ alkyl), —CONH₂, C₃-C₆ cycloalkyl, heterocycloalkyl, aryl, heteroaryl, C₃-C₆ cycloalkyl-C₁-C₆ alkyl-, heterocycloalkyl-C₁-C₆ alkyl-, aryl-C₁-C₆ alkyl- and heteroaryl-C₁-C₆ alkyl-, and where said cycloalkyl, heterocycloalkyl, aryl, heteroaryl, cycloalkylalkyl-, heterocycloalkylalkyl-, arylalkyl- or heteroarylalkyl- is unsubstituted or substituted with one or more substituents independently selected from C₁-C₆ alkyl, C₁-C₆ haloalkyl, halogen —OC₁-C₆ alkyl, —OC₁-C₆ haloalkyl, cyano, —N(C₁-C₆ alkyl)(C₁-C₆ alkyl), —NH(C₁-C₆ alkyl), —NH₂, —CO₂C₁-C₆ alkyl, —CO₂H, —CON(C₁-C₆ alkyl)(C₁-C₆ alkyl), —CONH(C₁-C₆ alkyl), and —CONH₂;

each R¹² is independently selected from the group consisting of C₁-C₈ alkyl, C₂-C₈ alkenyl, C₂-C₈ alkynyl, C₃-C₈ cycloalkyl, heterocycloalkyl, aryl, heteroaryl, —C₁-C₆ alkyl-C₃-C₈ cycloalkyl, —C₁-C₆ alkyl-heterocycloalkyl, —C₁-C₆ alkyl-aryl, and —C₁-C₆ alkyl-heteroaryl, —C₂-C₆ alkenyl-C₃-C₈ cycloalkyl, —C₂-C₆ alkenyl-heterocycloalkyl, —C₂-C₆ alkenyl-aryl, —C₂-C₆ alkenyl-heteroaryl, —C₂-C₆ alkynyl-C₃-C₈ cycloalkyl, —C₂-C₆ alkynyl-heterocycloalkyl, —C₂-C₆ alkynyl-aryl, and —C₂-C₆ alkynyl-heteroaryl,

where said C₁-C₈ alkyl, C₂-C₈ alkenyl, or C₂-C₈ alkynyl is unsubstituted or substituted with one or more substituents independently selected from halogen, —OR¹³, —NR¹⁰R¹³, cyano, nitro, —CO₂R¹³, —CONR¹⁰R¹³, —NR¹¹CONR¹⁰R¹³, —OCONR¹⁰R¹³, —SO₂NR¹⁰R¹³, and —COR¹³,

and where any of said cycloalkyl, heterocycloalkyl, aryl or heteroaryl (including the cycloalkyl, heterocycloalkyl, aryl or heteroaryl moieties of said —C₁-C₆ alkyl-C₃-C₈ cycloalkyl, —C₁-C₆ alkyl-heterocycloalkyl, —C₁-C₆ alkyl-aryl, or —C₁-C₆ alkyl-heteroaryl) is unsubstituted or substituted with one or more substituents independently selected from C₁-C₄ alkyl, C₁-C₄ haloalkyl, halogen, —OR¹³, —NR¹⁰R¹³, cyano, nitro, —CO₂R¹³, —CONR¹⁰R¹³, —NR¹⁰CONR¹¹R¹³, —OCONR¹⁰R¹³, —SO₂NR¹⁰R¹³, and —COR¹³;

each R¹³ is independently selected from the group consisting of hydrogen, C₁-C₈ alkyl, C₂-C₈ alkenyl, C₂-C₈ alkynyl, C₃-C₈ cycloalkyl, heterocycloalkyl, aryl, heteroaryl, —C₁-C₆ alkyl-C₃-C₈ cycloalkyl, —C₁-C₆ alkyl-heterocycloalkyl, —C₁-C₆ alkyl-aryl, and —C₁-C₆ alkyl-heteroaryl; provided that when X is O, Y is OH, and Z, R¹, R², R³, R⁴, R⁵, R⁶, R⁷ and R⁸ are hydrogen: W is not hydrogen, —CH₃, —C₂H₅, -nC₃H₇, -nC₄H₉, -n₅H₁₁, -nC₆H₁₃, —C₇H₁₅, —CH₂)CH(CH₃)₂, —(CH₂)₂CH(CH₃)₂, —CH₂CH═CH₂, —CH₂CH═CH(CH₃), —(CH₂)₃CN, —(CH₂)₄CN, —(CH₂)phenyl, —(CH₂)pyridin-2-yl, or —(CH₂)₂OCH₃,

or a tautomer thereof, or a pharmaceutically acceptable salt or solvate thereof.

This invention is also directed to a prodrug of a compound according to Formula I, or a tautomer thereof, or a pharmaceutically acceptable salt or solvate thereof. In addition, this invention is directed to pharmaceutical compositions comprising a compound according to Formula I, or a tautomer thereof, or a prodrug thereof, or salts or solvates thereof.

In another embodiment, this invention is directed to a method of inhibiting an RNA-containing virus comprising contacting the virus with an effective amount of a compound of Formula I. In yet another embodiment, this invention is directed to a method of treating infection or disease caused by an RNA-containing virus which comprises administering to a subject in need thereof, an effective amount of a compound according to Formula I. This invention is particularly directed to methods of inhibiting hepatitis C virus. This invention is also directed to a method for inhibiting replication of hepatitis C virus which comprises inhibiting replication of both positive and negative strand HCV-RNA.

### DETAILED DESCRIPTION OF THE INVENTION

It will be appreciated by those skilled in the art that the compounds of this invention, represented by generic Formula I, above, exist in tautomeric forms having Formula I-A and Formula I-B, as follows:

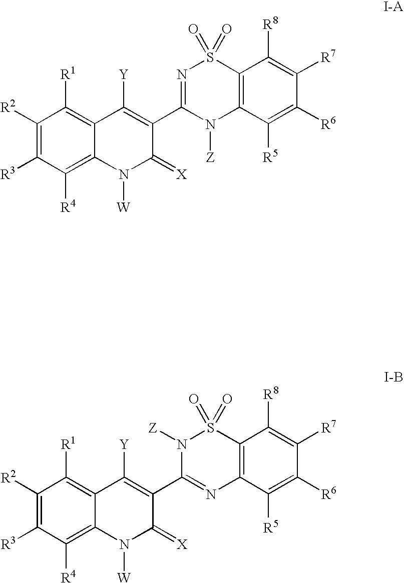

In addition, it will be appreciated by those skilled in the art, that the compounds of this invention may exist in several other tautomeric forms. All tautomeric forms of the compounds described herein are intended to be encompassed within the scope of the present invention. Examples of some of the other possible tautomeric forms of the compounds of this invention include, but are not limited to:

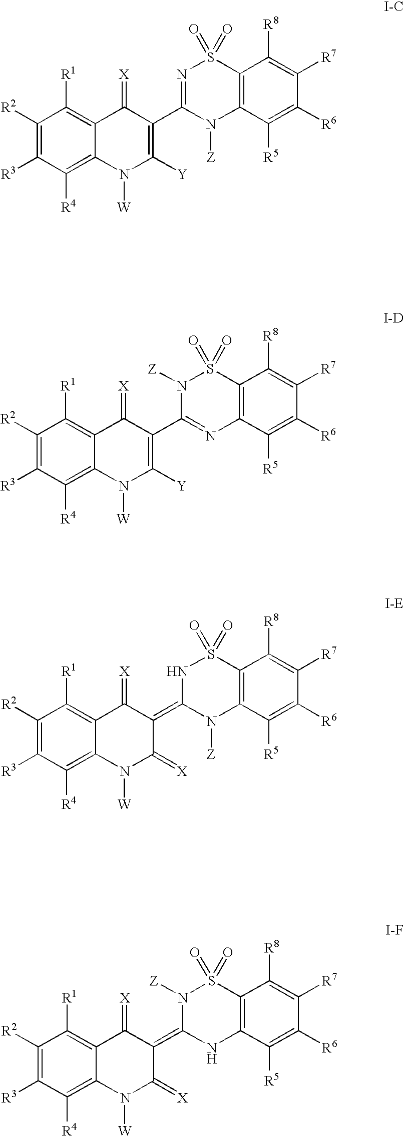

As a convention, the compounds exemplified herein have been assigned names based on the structure of the tautomer of Formula I-A. It is to be understood that any reference to named compounds of this invention is intended to encompass all tautomers of the named compounds and any mixtures of tautomers of the named compounds.

As used herein, the term “alkyl” represents a straight-or branched-chain saturated hydrocarbon, which may be unsubstituted or substituted by one, or more of the substituents defined herein. Exemplary alkyls include, but are not limited to methyl (Me), ethyl (Et), propyl, isopropyl, butyl, isobutyl, t-butyl and pentyl. The term “lower alkyl” refers to an alkyl containing from 1 to 4 carbon atoms.

When the term “alkyl” (or alkenyl or alkynyl) is used in combination with other substituent groups, such as “haloalkyl” or “arylalkyl”, the term “alkyl” is intended to encompass a divalent straight or branched-chain hydrocarbon radical. For example, “cycloalkylalkyl” is intended to mean the radical -alkyl-cycloalkyl, wherein the alkyl moiety thereof is a divalent straight or branched-chain hydrocarbon radical and the cycloalkyl moiety thereof is as defined herein, and is represented by the bonding arrangement present in the groups —CH₂-cyclopropyl, —CH₂-cyclohexyl, or —CH₂(CH₃)CHCH₂-cyclopentenyl. “Arylalkyl” is intended to mean the radical -alkylaryl, wherein the alkyl moiety thereof is a divalent straight or branched-chain carbon radical and the aryl moiety thereof is as defined herein, and is represented by the bonding arrangement present in a benzyl group (—CH₂-phenyl).

As used herein, the term “alkenyl” represents a straight-or branched-chain hydrocarbon containing one or more carbon-carbon double bonds. An alkenyl may be unsubstituted or substituted by one or more of the substituents defined herein. Exemplary alkenyls include, but are not limited ethenyl, propenyl, butenyl, isobutenyl and pentenyl.

As used herein, the term “alkynyl” represents a straight-or branched-chain hydrocarbon containing one or more carbon-carbon triple bonds and, optionally, one or more carbon-carbon double bonds. An alkynyl may be unsubstituted or substituted by one or more of the substituents defined herein. Exemplary alkynyls include, but are not limited ethynyl, butynyl, propynyl (propargyl, isopropynyl), pentynyl and hexynyl.

“Cycloalkyl” represents a group or moiety comprising a non-aromatic monocyclic, bicyclic, or tricyclic hydrocarbon containing from 3 to 14 carbon atoms which may be unsubstituted or substituted by one or more of the substituents defined herein and may be saturated or partially unsaturated. Exemplary cycloalkyls include monocyclic rings having from 3-7, preferably 3-6, carbon atoms, such as cyclopropyl, cyclobutyl, cyclopentyl, cyclopentenyl, cyclopentadienyl, cyclohexyl, cyclohexenyl and cycloheptyl.

“Heterocycloalkyl” represents a group or moiety comprising a non-aromatic, monovalent monocyclic, bicyclic, or tricyclic radical, which is saturated or partially unsaturated, containing 3 to 18 ring atoms, which includes 1 to 5 heteroatoms selected from nitrogen, oxygen and sulfur, and which may be unsubstituted or substituted by one or more of the substituents defined herein. Illustrative examples of heterocycloalkyls include, but are not limited to, azetidinyl, pyrrolidyl (or pyrrolidinyl), piperidinyl, piperazinyl, morpholinyl, tetrahydro-2H-1,4-thiazinyl, tetrahydrofuryl (or tetrahydrofuranyl), dihydrofuryl, oxazolinyl, thiazolinyl, pyrazolinyl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, oxabicylo[2.2.1]heptyl and 1,5,9-triazacyclododecyl. Generally, in the compounds of this invention, heterocycloalkyl is a monocyclic heterocycloalkyl, such as azetidinyl, pyrrolidyl (or pyrrolidinyl), piperidyl (or piperidinyl), piperazinyl, morpholinyl, tetrahydro-2H-1,4-thiazinyl, tetrahydrofuryl (or tetrahydrofuranyl), tetrahydrothienyl, dihydrofuryl, tetrahydropyranyl, dihydropyranyl, 1,3-dioxolanyl, 1,3-ioxanyl, 1,4-dioxanyl, 1,3-oxathianyl, 1,3-dithianyl, oxazolinyl, thiazolinyl and pyrazolinyl.

“Aryl” represents a group or moiety comprising an aromatic, monovalent monocyclic or bicyclic hydrocarbon radical containing from 6 to 10 carbon ring atoms, which may be unsubstituted or substituted by one or more of the substituents defined herein, and to which may be fused one or more cycloalkyl rings, which may be unsubstituted or substituted by one or more substituents defined herein. Generally, in the compounds of this invention, aryl is phenyl.

“Heteroaryl” represents a group or moiety comprising an aromatic monovalent monocyclic, bicyclic, or tricyclic radical, containing 5 to 18 ring atoms, including 1 to 5 heteroatoms selected from nitrogen, oxygen and sulfur, which may be unsubstituted or substituted by one or more of the substituents defined herein. This term also encompasses bicyclic or tricyclic heterocyclic-aryl compounds containing an aryl ring moiety fused to a heterocycloalkyl ring moiety, containing 5 to 16 ring atoms, including 1 to 5 heteroatoms selected from nitrogen, oxygen and sulfur, which may be unsubstituted or substituted by one or more of the substituents defined herein. Illustrative examples of heteroaryls include, but are not limited to, thienyl, pyrrolyl, imidazolyl, pyrazolyl, furyl (or furanyl), isothiazolyl, furazanyl, isoxazolyl, oxazolyl, oxadiazolyl, thiazolyl, pyridyl (or pyridinyl), pyrazinyl, pyrimidinyl, pyridazinyl, triazinyl, tetrazinyl, triazolyl, tetrazolyl, benzo[b]thienyl, naphtho[2,3-b]thianthrenyl, isobenzofuryl, 2,3-dihydrobenzofuryl, chromenyl, chromanyl, xanthenyl, phenoxathienyl, indolizinyl, isoindolyl, indolyl, indazolyl, purinyl, isoquinolyl, quinolyl, phthalazinyl, naphthridinyl, quinzolinyl, benzothiazolyl, benzimidazolyl, tetrahydroquinolinyl, cinnolinyl, pteridinyl, carbozolyl, beta-carbolinyl, phenanthridinyl, acridinyl, perimidinyl, phenanthrolinyl, phenazinyl, isothiazolyl, phenathiazinyl, and phenoxazinyl. Generally, in the compounds of this invention, heteroaryl is a monocyclic heteroaryl, such as thienyl, pyrrolyl, imidazolyl, pyrazolyl, furyl, isothiazolyl, furazanyl, isoxazolyl, oxazolyl, oxadiazolyl, thiazolyl, pyridyl, pyrazinyl, pyrimidinyl, pyridazinyl, triazinyl, tetrazinyl, triazolyl and tetrazolyl.

The terms “halogen” and “halo” represent chloro, fluoro, bromo or iodo substituents. “Hydroxy” is intended to mean the radical —OH. “Alkoxy” is intended to mean the radical —ORₐ, where Rₐis an optionally substituted alkyl group. Exemplary alkoxy include methoxy, ethoxy, propoxy, and the like. “Lower alkoxy” groups have optionally substituted alkyl moieties from 1 to 4 carbons. “Alkylenedioxy” is intended to mean the divalent radical —ORₐO— which is bonded to adjacent atoms (e.g., adjacent atoms on a phenyl or naphthyl ring), wherein Rₐis a C₁-C₂ alkyl group. Exemplary alkylenedioxy-substituted phenyls include benzo[1,3]dioxyl and 2,3-dihydro-benzo[1,4]dioxyl.

In the following embodiments, the following definitions apply:

each R^{a}is independently H or C₁-C₄ alkyl;

each R^{b} is independently H or C₁-C₄ alkyl, where the alkyl is optionally unsubstituted or substituted by one or more substituents independently selected from the group consisting of halogen, cyano, —OC₁-C₄ alkyl, —OH, —N(C₁-C₄ aklyl)(C₁-C₄ alkyl), —NH(C₁-C₄ alkyl), —NH₂, —CO₂H, —C(O)OC₁-C₄ alkyl, —CON(C₁-C₄ alkyl)(C₁-C₄ alkyl), —CONH(C₁-C₄ alkyl), —CONH₂, aryl, heteroaryl, heterocycloalkyl, —C(O)aryl, —C(O)heterocycloalkyl, and —C(O)heteroaryl, where said aryl, heteroaryl, heterocycloalkyl, —C(O)aryl, —C(O)heterocycloalkyl, or —C(O)heteroaryl is unsubstituted or substituted with one or more substituents independently selected from C₁-C₄ alkyl, C₁-C₄ haloalkyl, halogen, —OR^{a}, —SR^{a}, —NR^{a}R^{a}, cyano and nitro;

each R^{c} is independently C₁-C₄ alkyl, optionally unsubstituted or substituted by one or more substituents independently selected from the group consisting of halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —CON(C₁-C₄ alkyl)(C₁-C₄ alkyl), —CONH(C₁-C₄ allyl), —CONH₂, aryl and heteroaryl, and where said aryl or heteroaryl is unsubstituted or substituted with one or more substituents independently selected from C₁-C₄ alkyl, C₁-C₄ haloalkyl, halogen, —OR^{a}, —SR^{a}, —NR^{a}R^{a}, cyano and nitro;

each R^{d} is independently H or C₁-C₄ alkyl, where the alkyl is optionally substituted by one or more substituents independently selected from the group consisting of halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —CON(C₁-C₄ alkyl)(C₁-C₄ alkyl), —CONH(C₁-C₄ alkyl), —CONH₂, —C(O)C₁-C₄ alkyl, —C(O)aryl, —C(O)heteroaryl, cycloalkyl, heterocycloalkyl, aryl and heteroaryl, and where said aryl or heteroaryl is unsubstituted or substituted with one or more substituents independently selected from C₁-C₄ alkyl, C₁-C₄ haloalkyl, halogen, —OR^{a}, —SR^{a}, —NR^{a}R^{a}, cyano and nitro;

or, when present in any NR^{a}R^{b} or NR^{a}R^{d}, each R^{a} and R^{b} or each R^{a} and R^{d}, independently, taken together with the nitrogen atom to which they are attached form a 5- or 6-membered heterocycloalkyl ring, which optionally contains one or more heteroatoms selected from oxygen or nitrogen and which is unsubstituted or substituted with one or more substituents selected from the group halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —C(O)C₁-C₄ alkyl, —CON(C₁-C₄ alkyl)(C₁-C₄ alkyl), —CONH(C₁-C₄ alkyl), —CONH₂, —C(O)C₁-C₄ alkyl;

In one embodiment of the compounds of Formula I of this invention, R¹ is hydrogen, halogen, C₁-C₄ alkyl, aryl, —OR^{a}, —C(O)OR^{a}, —C(O)NR^{a}R^{a} or cyano. More specifically, R¹ is H, phenyl, —CH₃, F, Cl, Br, —OH, —C(O)OH, or —C(O)NHCH₃. Preferably, R¹ is H or halogen; specifically R¹ is H or F.

In another embodiment, R² is hydrogen, C₁-C₆ alkyl, C₁-C₆ haloalkyl, aryl, heteroaryl, nitro, cyano, halogen, —C(O)OR^{a}, —C(O)C₁-C₆ alkyl, —C(O)NR^{a}R^{a}, —OR^{b}, protected —OH, —SR^{b}, —S(O)R^{c}, —S(O)₂R^{b}, —NR^{a}R^{c}, —NR^{a}C(O)C₁-C₆ alkyl, —NR^{a}COaryl, —NR⁴CO(C₁-C₄ alkyl)aryl, —NR^{a}C(O)heteroaryl, —NR^{a}C(O)(C₁-C₄ alkyl)heteroaryl, —NR^{a}C(O)cycloalkyl, —NR^{a}C(O)(C₁-C₄ alkyl)cycloalkyl, —NR^{c}(O)heterocycloalkyl, —NR^{a}C(O)(C₁-C₄ alkyl)heterocycloalkyl, where each of said C₁-C₆ alkyl is optionally unsubstituted or substituted by one or more substituents independently selected from the group consisting of cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —CON(C₁-C₄ alkyl)(C₁-C₄ alkyl), —CONH(C₁-C₄ alkyl), and —CONH₂, and where each of said aryl, heteroaryl, cycloalkyl, or heterocycloalkyl is optionally unsubstituted or substituted with one or more substituents independently selected from C₁-C₄ alkyl, C₁-C₄ haloalkyl, halogen, —OR^{a}, —R^{a}, —NR^{a}R^{a}, —CON(C₁-C₄ aklyl)(C₁-C₄ alkyl), —CONH(C₁-C₄ alkyl), —CONH₂, nitro and cyano. Preferably, R² is hydrogen, halogen, —OR^{b′}; —NHR^{b′}, NO₂, where R^{b′} is H or C₁-C₂ alkyl, where the C₁-C₂ alkyl is optionally unsubstituted or substituted by a substituent selected from the group consisting of cyano, —OH, —CO₂H, —CONH₂, —C(O)OC₁-C₂ alkyl, —CONH(C₁-C₂ alkyl), and unsubstituted monocyclic heteroaryl.

In specific embodiments of this invention, R² is H, F, Cl, Br, —OH, —OCH₃, —CH₃, —CH₂(40CH₃-phenyl), —CH═CHC(O)NH₂, —NO₂, —NH₂, —NHCH₃, —N(CH₃)₂, —CONHCH₃, —CON(CH₃)₂, —CO₂H, —CO₂CH₂CH₃, —O(CH₂)₂CH(CH₃)₂, —O(CH₂)₃CN, —OCH₂CN, —O(CH₂)₂OCH₃, —O(CH₂)₂OH, —OCH₂CH(OH)CH₂CH₃, —O(CH₂)₂N(CH₃)₂, —OCH₂phenyl, —OCH₂CONH₂, —O(6-Br-pyridin-2-yl), —O(6-OCH₃-pyridin-2-yl), —OSi(CH₃)₂(tBu), —NHCH₂CO₂H, —NHCH₂CO₂CH₂CH₃, —NHCH₂-2-furyl, —NH(CH₂)₂OH, —NHCH₂CN, —NHCH₂C(O)NH₂, —NHC(O)CH₃, —NHC(O)CH₂CH(CH₃)₂, —NHC(O)CH₂N(CH₃)₂, —NHC(O)phenyl, —NHC(O)(3-CH₃O-phenyl), —NHC(O)(4NO₂-phenyl), —NHC(O)(3-CN-phenyl), —NHC(O)(3-CF₃-phenyl), —NHC(O)(3-F-phenyl), —NHC(O)(3-pyridyl), —NHC(O)(2-furyl), —NHC(O)(2-thienyl), —NHC(O)(4OCH₃-phenyl), —NHC(O)(cyclopentyl). Preferably, R² is H F, Cl, —OH, —NH₂, NO₂, —OCH₃, —NHCH₃, —O(CH₂)₂OH, —NH(CH₂)₂OH, —OCH₂CN, —NHCH₂CN, —OCH₂CONH₂, —NHCH₂CO₂H, —NHCH₂CO₂Et, or —NHCH₂(2-furyl).

In yet another embodiment of the compounds of this invention, R³ is H, halogen or —C(O)OH. In specific embodiments, R³ is H, F, Cl, Br, or CO₂H. Preferably, R³ is H or halogen; specifically, R³ is H or F.

In a further embodiment, R⁴ is H, halogen, or C₁-C₄ alkyl. In specific embodiments, R⁴ is H, Br or —(CH₂)₂CH(CH₃)₂. Preferably, R⁴ is H.

In another embodiment, R⁴ is H, halogen, C₁-C₄ alkyl, or —OR^{a}. In specific embodiments, R⁵ is H, —CH₃, —OCH₃ or —OH. Preferably, R⁵ is H or —OH.

In one other embodiment, R⁶ is H, halogen, or —OR^{a}. In specific embodiments, R⁶ is H, Br, —OH, or —OCH₃. Preferably, R⁶ is H.

In one embodiment of the compounds of Formula I of this invention, R⁷ is hydrogen, C₁-C₆ alkyl, C₂-C₆ alkenyl, C₂-C₆ alkynyl, aryl, heteroaryl, nitro, cyano, halogen, —C(O)OR^{a}, —C(O)C₁-C₆ alkyl, —C(O)NR^{a}R^{d}, —OR^{b}, —NR^{a}R^{d}, —N(R^{a})C(O)R^{d}, —OC(O)NR^{a}R^{d}, or —N(R^{a})C(O)NR^{a}R^{d}, where said alkyl, alkenyl or alkynyl is unsubstituted or substituted with one or more substituents independently selected from halogen, —OR^{a}, —SR^{a}, —NR^{a}R^{a}, cyano, nitro, —CO₂H, —C(O)OC₁-C₄ alkyl, —CON(C₁-C₄ alkyl)(C₁-C₄ alkyl), —CONH(C₁-C₄ alkyl), —CONH₂, aryl, and heteroaryl, and where said aryl or heteroaryl is unsubstituted or substituted with one or more substituents independently selected from C₁-C₆ alkyl, C₁-C₆ haloalkyl, halogen, —OR^{a}, —SR^{a}, —NR^{a}R^{a}, cyano and nitro.

Preferably, R⁷ is hydrogen, halogen, C₁-C₂ alkyl, C₂ alkenyl, —C(O)OR^{a′}; —C(O)R^{a′}, —OR^{b″}; —NR^{a′}R^{d′}, —C(O)NR^{a′}R^{d′}, where said alkyl or alkenyl is unsubstituted or substituted with a substituent selected from —NH₂ and —CONH₂, R^{a′} is H or methyl, R^{b″} is H or C₁-C₄ alkyl, where the C₁-C₄ alkyl is optionally unsubstituted or substituted by a substituent selected from the group consisting of cyano, —NH₂, —CO₂H, —CONH₂, —C(O)OC₁-C₂ alkyl, —CON(C₁-C₄ alkyl)(C₁-C₄ alkyl), —CONH(C₁-C₄ alkyl), monocyclic heteroaryl, —C(O)monocyclic heterocycloalkyl, and —C(O)monocyclic heteroaryl, where said heteroaryl, —C(O)heterocycloalkyl, or —C(O)heteroaryl are unsubstituted or substituted one or more of C₁-C₄ alkyl, halogen, cyano, —OH, —NH₂, and —CONH₂, R^{d′} is H or C₁-C₂ alkyl, where the C₁-C₂ alkyl is unsubstituted or substituted by a substituent selected from the group consisting of cyano and unsubstituted aryl, or R^{a′} and R^{d′} taken together with the nitrogen atom to which they are attached form a 5- or 6-membered heterocycloalkyl ring, which optionally contains an additional nitrogen heteroatom and which is unsubstituted or substituted with —C(O)C₁-C₂ alkyl.

In specific embodiments, R⁷ is H, —CH₃, —OH, —OCH₃, phenyl, F, Cl, Br, I, NO₂, —NH₂, —N(CH₃)₂, —NHCH₂CN, —CN, —CH₂NH₂, —CH₂CH₂C(O)NH₂, —CH═CHC(O)NH₂, —(CH₂)₂CH(CH₃)OCH₃, —CHO, —C(O)CH₃, —CO₂CH₃, —CO₂H, —C(O)NH₂, —C(O)NHCH₃, —C(O)N(CH₃)₂, —OCH₂CO₂CH₃, —OCH₂CO₂H, —OCH₂CH(NH₂)CH₂CH₃, —O(CH₂)₂N(CH₃)₂, —OCH₂CN, —O(CH₂)₂NH₂, —OCH₂C(O)NH₂, —OCH₂CONHCH₃, —OCH₂CON(CH₃)₂, —OCH(CH₃)C(O)NH₂, —OCH₂-tetrazol-5-yl, —OCH₂C(O)(3-pyridyl), —OCH₂C(O)(N-pyrrolidinyl), —OCH₂C(O)(N-piperazinyl), —OCH₂C(O)(N-morpholinyl), —OCH₂(5-methyl-1,3,4-oxadiazol-2-yl), —C(O)NH(CH₂)₃(N-imidazoyl), —C(O)NHCH₂CH(OCH₃)₂, —C(O)(4-acetylpiperizin-1-yl), —C(O)NHCH₂(2-tetrahydrofuryl), —C(O)NHCH₂phenyl, —C(O)NH(CH₂)₃N(CH₂CH₃)₂, —C(O)(N-pyrrolidinyl), —C(O)NH(CH₂)₂(4-OCH₃phenyl), or —NHCH₂phenyl. Preferably, R⁷ is H, F, Cl, —CH₃, —CH₂NH₂, —CH₂CH₂CONH₂, —CH═CHC(O)NH₂, —OH, —OCH₃, —O(CH₂)₂NH₂, —OCH₂CH((R)—NH₂)CH₂CH₃, —OCH₂CN, —OC(CH₃)₂CONH₂, —OCH₂CO₂CH₃, —OCH₂CONH₂, —OCH₂CONHCH₃, —OCH₂CON(CH₃)₂, —OCH₂CO₂H, —OCH₂C(O)(3-pyridinyl), —OCH₂C(O)(N-pyrrolidinyl), —OCH₂C(O)(N-morpholinyl), —OCH₂(5-methyl-1,3,4-oxadiazol-2-yl), —OCH(CH₃)CONH₂, —OCH₂-tetrazol-5-yl, —NH₂, —N(CH₃)₂, —NHCH₂CN, —NHCH₂Ph, —CO₂CH₃, —CO₂H, —C(O)NH₂, —C(O)NHCH₃, —C(O)N(CH₃)₂, —CHO, —C(O)(N-acetylpiperizinyl), or —C(O)(N-pyrrolidinyl).

In another embodiment, R⁸ is hydrogen or halogen. In specific embodiments, R⁸ is H.

In yet another embodiment, R¹ and R² or R⁵ and R⁶ or R⁶ and R⁷ or R⁷ and R⁸ taken together are alkylenedioxy. Preferably, R¹ and R² taken together are alkylenedioxy. In a specific embodiment, R¹ and R² taken together are methylenedioxy.

In another embodiment, W is hydrogen, —C(O)OR^{a}, C₃-C₈ alkyl, C₃-C₆alkenyl, C₃-C₆ alkynyl, —(C₁-C₄ alkyl)-C₃-C₆ cycloalkyl), —(C₁-C₄ alkyl)-heterocycloalkyl, —(C₁-C₄ alkyl)-aryl, or —(C₁-C₄ alkyl)-heteroaryl, where the C₁-C₈ alkyl, C₂-C₆ alkenyl or C₂-C₆ alkynyl is unsubstituted or substituted with one or more substituents independently selected from halogen, cyano, —OR^{a}, —SR, —S(O)C₁-C₄ alkyl, —S(O)₂C₁-C₄ alkyl, and where the cycloalkyl, heterocycloalkyl, aryl or heteroaryl moiety of the C₁-C₄ alkyl)-(C₃-C₆ cycloalkyl), —(C₁-C₄ alkyl)-heterocycloalkyl, —(C₁-C₄ alkyl)-aryl, or —C₁-C₄ alkyl)-heteroaryl is unsubstituted or substituted with one or more substituents independently selected from C₁-C₄ allyl, C₁-C₄ haloalkyl, halogen, nitro, cyano, —OR^{a}, —NR^{a}R^{a}. Preferably, W is C₄-C₆ alkyl, C₄ alkenyl, C₄ alkynyl, —(C₁-C₂ alkyl)-(C₃-C₆ cycloalkyl), —(C₁ alkyl)-heterocycloalkyl, —(C₁ alkyl)-aryl, or —(C₁-C₄ alkyl)-heteroaryl, where the C₄-C₆ alkyl, C₄ alkenyl or C₄ alkynyl is unsubstituted or substituted with one or more substituents independently selected from halogen, —OH, —OCH₃, —SCH₃, and where the cycloalkyl, heterocycloalkyl, aryl or heteroaryl moiety of the —(C₁-C₄ alkyl)-(C₃-C₆ cycloalkyl), —C₁-C₄ alkyl)-heterocycloalkyl, —(C₁-C₄ alkyl)-aryl, or —(C₁-C₄ alkyl)-heteroaryl is unsubstituted or substituted with one or more substituents independently selected from —CH₃, halogen, nitro, cyano, —OR^{a}, —NR^{a}R^{a}.

In specific embodiments, W is selected from the group consisting of H, —(CH₂)₁₋₃-phenyl, —CH₂—(2-CN-phenyl), —(CH₂)₁₋₂-cyclopropyl, —CH₂(2-CH₃-cycloprop-1-yl), —(CH₂)-cyclobutyl, —(CH₂)-cyclopentyl, —(CH₂)-cyclohexyl, —CH₂-(2-tetrahydrofuryl), —CH₂-(3-tetrahydrofuryl), —CH₂-(3-pyridyl), —CH₂-(6NH₂-3-pyridyl), —CH₂-(4-pyridyl), —CH₂-(2-NH₂-4-pyridyl), —CH₂-(2-CH₃-4-pyridyl), —CH₂-(4-bromophenyl), —CH₂-(3-bromophenyl), —CH₂-(3-NO₂-phenyl), —CH₂-(3-furyl), —(CH₂)-(2-thienyl), —(CH₂)₂-(3-thienyl), —(CH₂)₂CH(CH₃)₂, —(CH₂)₂C(CH₃)₃, —(CH₂)₂CH(CH₃)CH₂CH₃, —(CH₂)₂CH(CH₃)(CF₃), {CH₂)₂CH═CH₂, —CH₂CH═CH₂, —(CH₂)₂CHBr(CH₃), —(CH₂)CH═C(CH₃)₂, —(CH₂)₃CF₃, —CH₂)₃CN, —(CH₂)3-4OH, —(CH₂)₂CH(CH₃)OCH₃, —CH₂)₂C≡CH, —CH₂)₃C≡CH, —CO₂CH₂CH₃, —(CH₂)₂CH(CH₃)CH₂CH₃, —(CH₂)₂SCH₃, (CH₂)₃SCH₃, —(CH₂)₂S(O)CH₃, —(CH₂)₂S(O)₂CH₃. Preferably, W is —(CH₂)₂CH(CH₃)₂, —(CH₂)₂C(CH₃)₃, —(CH₂)₂CH(CH₃)CH₂CH₃, —(CH₂)₂CH═CH₂, —(CH₂)₂C—CH, —(CH₂)₃CF₃, —(CH₂)₂CH(CH₃)(CF₃), —(CH₂)₂CHBrCH₃, —(CH₂)₄OH, —(CH₂)₂CH(CH₃)OCH₃, —(CH₂)₂SCH₃, —CH₂(cyclopropyl), —(CH₂)₂(cyclopropyl), —CH₂(2-CH₃-cycloprop-1-yl), —CH₂(cyclobutyl), —CH₂(cyclopentyl), —CH₂(cyclohexyl), —CH₂(3-Br-phenyl), —(CH₂(3-NO₂-phenyl), —CH₂(4-Br-phenyl), —CH₂(3-furyl), —CH₂)₂(3-thienyl), —CH₂(4-pyridyl), or —CH₂(2-CH₃₄-pyridinyl).

Preferably, in the compounds of this invention, X is O, Y is OH.

In another embodiment of the compounds of this invention, Z is H or methyl. Preferably, Z is H.

It is to be understood that the present invention covers all combinations of particular and preferred groups described hereinabove.

Accordingly, one embodiment of this invention comprises compounds wherein: R¹ is hydrogen, halogen, C₁-C₄ alkyl, aryl, —OR^{a}, —C(O)OR^{a}, —C(O)NR^{a}R^{a} or cyano; R² is hydrogen, C₁-C₆ alkyl, C₁-C₆ haloalkyl, aryl, heteroaryl, nitro, cyano, halogen, —C(O)OR^{a}, —C(O)C₁-C₆ alkyl, —C(O)NR^{a}R^{a}, —OR^{b}, protected —OH, OR^{b}, —S(O)R^{c}, —S(O)₂R^{b}, —NR^{a}R^{c}, —NR^{a}C(O)C₁-C₆ alkyl, —NR^{a}COaryl, —NR^{a}CO(C₁-C₄ alkyl)aryl, —NR^{a}C(O)heteroaryl, —NR^{a}C(O)(C₁-C₄ alkyl)heteroaryl, —NR^{a}C(O)cycloalkyl, —NR^{a}C(O)(C₁-C₄ alkyl)cycloalkyl, —NR^{a}C(O)heterocycloalkyl, —NR^{a}C(O)(C₁-C₄ alkyl)heterocycloalkyl, where each of said C₁-C₆ alkyl is optionally unsubstituted or substituted by one or more substituents independently selected from the group consisting of cyano, —OC₁-C₄ alkyl, —OH, —N(C₁-C₄ alkyl)(C₁-C₄ alkyl), —NH(C₁-C₄ allyl), —NH₂, —CO₂H, —C(O)OC₁-C₄ alkyl, —CON(C₁-C₄ alkyl)(C₁-C₄ alkyl), —CONH(C₁-C₄ alkyl), and —CONH₂, and where each of said aryl, heteroaryl, cycloalkyl, or heterocycloalkyl is optionally unsubstituted or substituted with one or more substituents independently selected from C₁-C₄ alkyl, C₁-C₄ haloalkyl, halogen, —OR^{a}, —SR^{a}, —NR^{a}R^{a}, —CON(C_{I}-C₄ alkyl)(C₁-C₄ alkyl), —CONH(C₁-C₄ alkyl), —CONH₂, nitro and cyano; R³ is H, halogen, or —C(O)OH; R⁴ is H, halogen, or C₁-C₄ alkyl; R⁵ is H, halogen, C₁-C₄ alkyl, or —OR^{a}; R⁶ is H, halogen, or —OR^{a}; R⁷ is hydrogen, C₁-C₆ alkyl, C₂-C₆ alkenyl, C₂-C₆ alkynyl, aryl, heteroaryl, nitro, cyano, halogen, —C(O)OR^{a}, —C(O)C₁-C₆ alkyl, —C(O)NR^{a}R^{d}, —OR^{b}, —NR^{a}R^{d}, —N(R^{a})C(O)R^{d}, —OC(O)NR^{a}R^{d}, or —N(R^{a})C(O)NR^{a}R^{d}, where said alkyl, alkenyl or alkynyl is unsubstituted or substituted with one or more substituents independently selected from halogen, —OR^{a}, —SR^{a}, —NR^{a}R^{a}, cyano, nitro, —CO₂H, —C(O)OC₁-C₄ alkyl, —CON(C_{I}-C₄ alkyl)(C₁-C₄ alkyl), —CONH(C₁-C₄ alkyl), —CONH₂, aryl, and heteroaryl, and where said aryl or heteroaryl is unsubstituted or substituted with one or more substituents independently selected from C₁-C₆ alkyl, C₁-C₆ haloalkyl, halogen, —OR^{a}, —SR^{a}, —NR^{a}R^{a}, cyano and nitro; R⁸ is hydrogen or halogen; or R¹ and R² or R⁵ and R⁶ or R⁶ and R⁷ or R⁷ and R⁸ taken together are alkylenedioxy; W is hydrogen, —C(O)OR^{a}, C₁-C₈ alkyl, C₂-C₆ alkenyl, C₂-C₆ alkynyl, —(C₁-C₄ alkyl)-(C₃-C₆ cycloalkyl), —(C₁-C₄ alkyl)-heterocycloalkyl, —(C₁-C₄ alkyl)-aryl, or —(C₁-C₄ alkyl)-heteroaryl, where the C₁-C₈ alkyl, C₂-C₆ alkenyl or C₂-C₆ alkynyl is unsubstituted or substituted with one or more substituents independently selected from halogen, cyano, —OR^{a}, —SR^{a}, —S(O)C₁-C₄ alkyl, —S(O)₂C₁-C₄ alkyl, and where the cycloalkyl, heterocycloalkyl, aryl or heteroaryl moiety of the —C₁-C₄ alkyl)-(C₃-C₆ cycloalkyl), —(C₁-C₄ alkyl)-heterocycloalkyl, —(C₁-C₄ alkyl)-aryl, or —(C₁-C₄ alkyl)-heteroaryl is unsubstituted or substituted with one or more substituents independently selected from C₁-C₄ alkyl, C₁-C₄ haloalkyl, halogen, nitro, cyano, —OR^{a}, —NR^{a}R^{a}; X is O; Y is OH; and Z is hydrogen or methyl; where each R^{a}is independently H or C₁-C₄ alkyl; each R^{b} is independently H or C₁-C₄ alkyl, where the alkyl is optionally unsubstituted or substituted by one or more substituents independently selected from the group consisting of halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —CON(C₁-C₄ alkyl)(C₁-C₄ alkyl), —CONH(C₁-C₄ alkyl), —CONH₂, aryl, heteroaryl, heterocycloalkyl, —C(O)aryl, —C(O)heterocycloalkyl, and —C(O)heteroaryl, where said aryl, heteroaryl, heterocycloalkyl, —C(O)aryl, —C(O)heterocycloalkyl, or —C(O)heteroaryl is unsubstituted or substituted with one or more substituents independently selected from C₁-C₄ alkyl, C₁-C₄ haloalkyl, halogen, —OR^{a}, —SR^{a}, —NR^{a}R^{a}, cyano and nitro; each R^{c} is independently C₁-C₄ alkyl, optionally unsubstituted or substituted by one or more substituents independently selected from the group consisting of halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —CON(C₁-C₄ alkyl)(C₁-C₄ alkyl), —CONH(C₁-C₄ alkyl), —CONH₂, aryl and heteroaryl, and where said aryl or heteroaryl is unsubstituted or substituted with one or more substituents independently selected from C₁-C₄ alkyl, C₁-C₄ haloalkyl, halogen, —OR^{a}, —SR^{a}, —NR^{a}R^{a}, cyano and nitro; each R^{d} is independently H or C₁-C₄ alkyl, where the alkyl is optionally substituted by one or more substituents independently selected from the group consisting of halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —CON(C₁-C₄ alkyl)(C₁-C₄ alkyl), —CONH(C₁-C₄ alkyl), —CONH₂, —C(O)C₁-C₄ alkyl, —C(O)aryl, —C(O)heteroaryl, cycloalkyl, heterocycloalkyl, aryl and heteroaryl, and where said aryl or heteroaryl is unsubstituted or substituted with one or more substituents independently selected from C₁-C₄ alkyl, C₁-C₄ haloalkyl, halogen, —OR^{a}, SR^{a}, —NR^{a}R^{a}, cyano and nitro; or, when present in any NR^{a}R^{b} or NR^{a}R^{d}, each R^{a} and R^{b} or each R^{a} and R^{d}, independently, taken together with the nitrogen atom to which they are attached form a 5- or 6-membered heterocycloalkyl ring, which optionally contains one or more heteroatoms selected from oxygen or nitrogen and which is unsubstituted or substituted with one or more substituents selected from the group halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —C(O)C₁-C₄ alkyl, —CON(C₁-C₄ alkyl)(C₁-C₄ alkyl), —CONH(C₁-C₄ alkyl), —CONH₂, —C(O)C₁-C₄ alkyl, provided that when Z, R¹, R², R³, R⁴, R⁵, R⁶, R⁷ and R⁸ are hydrogen: W is not hydrogen, —CH₃, —C₂HS, -nC₃H₇, -nC₄H₉, —nC₅H₁₁, -nC₆H₁₃, -nC₇H₁₅, —(CH₂)CH(CH₃)₂, —(CH₂)₂CH(CH₃)₂, —CH₂CH═CH₂, —CH₂CH═CH(CH₃), —(CH₂)₃CN, —(CH₂)₄CN, —(CH₂)phenyl, —(CH₂)pyridin-2-yl, or —(CH₂)₂OH₃; or a tautomer thereof, or a pharmaceutically acceptable salt or solvate thereof.

Another embodiment of this invention comprises compounds of Formula I, wherein R¹ is H, phenyl, —CH₃, F, Cl, Br, —OH, —C(O)OH, or —C(O)NHCH₃; R² is H, F, Cl, Br, I, —OH, —OCH₃, —CH₃, —CH₂(40CH₃-phenyl), —CH═CHC(O)NH₂, —NO₂, —NH₂, —NHCH₃, —N(CH₃)₂, —CONHCH₃, —CON(CH₃)₂, —CO₂H, —CO₂CH₂CH₃, —O(CH₂)₂CH(CH₃)₂, —O(CH₂)₃CN, —OCH₂CN, —O(CH₂)₂OCH₃, —O(CH₂)₂OH, —OCH₂CH(OH)CH₂CH₃, —O(CH₂)₂N(CH₃)₂, —OCH₂phenyl, —OCH₂CONH₂, —O(6-Br-pyridin-2-yl), —O(6-OCH₃-pyridin-2-yl), —OSi(CH₃)₂(tBu), —NHCH₂CO₂H, —NHCH₂CO₂CH₂CH₃, —NHCH₂-2-furyl, —NH(CH₂)₂OH, —NHCH₂CN, —NHCH₂C(O)NH₂, —NHC(O)CH₃, —NHC(O)CH₂CH(CH₃)₂, —NHC(O)CH₂N(CH₃)₂, —NHC(O)phenyl, —NHC(O)(3-CH₃O-phenyl), —NHC(O)(4NO₂-phenyl), —NHC(O)(3-CN-phenyl), —NHC(O)(3-CF₃-phenyl), —NHC(O)(3-F-phenyl), —NHC(O)(3-pyridyl), —NHC(O)(2-furyl), —NHC(O)(2-thienyl), —NHC(O)(4-OCH₃-phenyl), —NHC(O)(cyclopentyl); R³ is H, F, Cl, Br, or —CO₂H; R⁴ is H, Br or —(CH₂)₂CH(CH₃)₂; R⁵ is H, —CH₃, —OCH₃ or —OH; R⁶ is H, Br, —OH, or —OCH₃; R⁷ is H, —CH₃, —OH, —OCH₃, phenyl, F, Cl, Br, I, NO₂, —NH₂, —N(CH₃)₂, —NHCH₂CN, —CN, —CH₂NH₂, —CH₂CH₂C(O)NH₂, —CH═CHC(O)NH₂, —(CH₂)₂CH(CH₃)OCH₃, —CHO, —C(O)CH₃, —CO₂CH₃, —CO₂H, —C(O)NH₂, —C(O)NHCH₃, —C(O)N(CH₃)₂, —OCH₂CO₂CH₃, —OCH₂CO₂H, —OCH₂CH(NH₂)CH₂CH₃, —O(CH₂)₂N(CH₃)₂, —OCH₂CN, —O(CH₂)₂NH₂, —OCH₂C(O)NH₂, —OCH₂CONHCH₃, —OCH₂CON(CH₃)₂, —OCH(CH₃)C(O)NH₂, —OCH₂-tetrazol-5-yl, —OCH₂C(O)(3-pyridyl), —OCH₂C(O)(N-pyrrolidinyl), —OCH₂C(O)(N-piperazinyl), —OCH₂C(O)(N-morpholinyl), —OCH₂(5-methyl-1,3,4-oxadiazol-2-yl), —C(O)NH(CH₂)₃(N-imidazoyl), —C(O)NHCH₂CH(OCH₃)₂, —C(O)(4acetylpiperizin-1-yl), —C(O)NHCH₂(2-tetrahydrofuryl), —C(O)NHCH₂phenyl, —C(O)NH(CH₂)₃N(CH₂CH₃)₂, —C(O)(N-pyrrolidinyl), —C(O)NH(CH₂)₂(40CH₃phenyl), or —NHCH₂phenyl; R⁸ is H; or R¹ and R² taken together are methylenedioxy; W is selected from the group consisting of —(CH₂)₁₋₃-phenyl, —CH₂-(2-CN-phenyl), —(CH₂)₁₋₂-cyclopropyl, —CH₂(2-CH₃-cycloprop-1-yl), —(CH₂)-cyclobutyl, —(CH₂)-cyclopentyl, —(CH₂)-cyclohexyl, —CH₂-(2-tetrahydrofuryl), —CH₂-(3-tetrahydrofuryl), —CH₂-(3-pyridyl), —CH₂-(6-NH₂-3-pyridyl), —CH₂-(4pyridyl), —CH₂-(2-NH₂-4-pyridyl), —CH₂-(2-CH₃-4-pyridyl), Hr₂-(4-bromophenyl), —CH₂-(3-bromophenyl), —CH₂-3-NO₂-phenyl), —CH₂-(3-furyl), —(CH₂)₂-(2-thienyl), —(CH₂)₂-(3-thienyl), —(CH₂)₂CH(CH₃)₂, —(CH₂)₂C(CH₃)₃, —(CH₂)₂CH(CH₃)CH₂CH₃, —(CH₂)₂CH(CH₃)(CF₃), —(CH₂)₂CH═CH₂, —CH₂CH═CH₂, —(CH₂)₂CHBr(CH₃), —(CH₂)CH═C(CH₃)₂, —(CH₂)₃CF₃, —(CH₂)₃CN, —(CH₂)₃₋₄OH, —(CH₂)₂CH(CH₃)OCH₃, —(CH₂)₂C≡CH, —(CH₂)₃C—CH, —CO₂CH₂CH₃, —(CH₂)₂CH(CH₃)CH₂CH₃, —(CH₂)₂SCH₃, (CH₂)₃SCH₃, —(CH₂)₂S(O)CH₃, —(CH₂)₂S(O)₂CH₃; X is O; Y is OH; and Z is hydrogen or methyl; provided that when Z, R¹, R², R³, R⁴, R⁵, R⁶, R⁷ and R⁸ are hydrogen: W is not —(CH₂)₂CH(CH₃)₂, —CH₂CH═CH₂, —CH₂CH═CH(CH₃), —(CH₂)₃CN, or —(CH₂)phenyl; or a tautomer thereof, or a pharmaceutically acceptable salt or solvate thereof.

Yet another embodiment of this invention, comprises compounds of Formula I wherein wherein: R¹ and R³ are each independently H or F; R² is hydrogen halogen, —OR^{b′}, —NHR^{b′}, NO₂, where R^{b′} is H or C₁-C₂ alkyl, where the C₁-C₂ alkyl is optionally unsubstituted or substituted by a substituent selected from the group consisting of cyano, —OH, —CO₂H, —CONH₂, —C(O)OC₁-C₂ alkyl, —CONH(C₁-C₂ alkyl), and unsubstituted monocyclic heteroaryl; R⁴, R⁶ and R⁸ are each H; R⁶ is H or —OH; R⁷ is hydrogen, halogen, C₁-C₂ alkyl, C₂ alkenyl, —C(O)OR^{a}, —C(O)R^{a}, —OR^{b}; —NR^{a}R^{d}; C(O)NR^{a}R^{d}; where said alkyl or alkenyl is unsubstituted or substituted with a substituent selected from —NH₂ and —CONH₂, R¹ is H or methyl, R^{b} is H or C₁-C₄ alkyl, where the C₁-C₄ alkyl is optionally unsubstituted or substituted by a substituent selected from the group consisting of cyano, —NH₂, —CO₂H, —CONH₂, —C(O)OC₁-C₂ alkyl, —CON(C₁-C₄ alkyl)(C₁-C₄ alkyl), —CONH(C₁-C₄ alkyl), monocyclic heteroaryl, —C(O)monocyclic heterocycloalkyl, and —C(O)-monocyclic heteroaryl, where said heteroaryl, —C(O)heterocycloalkyl, or —C(O)heteroaryl are unsubstituted or substituted one or more of C₁-C₄ alkyl, halogen, cyano, —OH, —NH₂, and —CONH₂, R^{d′} is H or C₁-C₂ alkyl, where the C₁-C₂ alkyl is unsubstituted or substituted by a substituent selected from the group consisting of cyano and unsubstituted aryl, or R^{a′} and R^{d′} taken together with the nitrogen atom to which they are attached form a 5- or 6-membered heterocycloalkyl ring, which optionally contains an additional nitrogen heteroatom and which is unsubstituted or substituted with —C(O)C₁-C₂ alkyl; or R¹ and R² taken together are alkylenedioxy; W is C₄-C₆ alkyl, C₄ alkenyl, C₄ alkynyl, —(C₁-C₂ alkyl)-(C₃-C₆ cycloalkyl), —(C₁ alkyl)-heterocycloalkyl, —(C₁ alkyl)-aryl, or —(C₁ alkyl)-heteroaryl, where the C₄-C₆ alkyl, C₄ alkenyl or C₄ alkynyl is unsubstituted or substituted with one or more substituents independently selected from halogen, —OH, —OCH₃, —SCH₃, and where the cycloalkyl, heterocycloalkyl, aryl or heteroaryl moiety of the —C₁-C₄ alkyl)-(C₃-C₆ cycloalkyl), —C₁-C₄ alkyl)-heterocycloalkyl, —(C₁-C₄ alkyl)-aryl, or —(C₁-C₄ alkyl)-heteroaryl is unsubstituted or substituted with one or more substituents independently selected from —CH₃, halogen, nitro, cyano, —OR^{a}, —NR^{a}R^{a}; X is O; Y is OH; and Z is hydrogen; provided that when R¹, R², R³, R⁴, R⁵, R⁶, R⁷ and R⁸ are hydrogen: W is not -nC₄H₉, -nC₅H₁₁, -nC₆H₁₃, -nC₇H₁₅, —(CH₂)CH(CH₃)₂, —(CH₂)₂CH(CH₃)₂, —CH₂CH═CH(CH₃), —(CH₂)phenyl, or —(CH₂)pyridin-2-yl; or a tautomer thereof, or a pharmaceutically acceptable salt or solvate thereof.

One embodiment of this invention relates to a compound of Formula II:

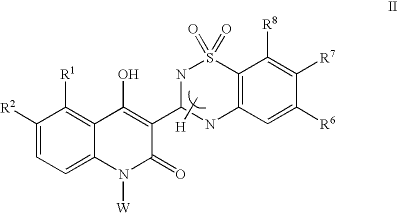

or a tautomer thereof, or a pharmaceutically acceptable salt or solvate thereof,

wherein R¹, R², R⁶, R⁷, R⁸ and W are as defined herein.

Another embodiment of this invention relates to a compound of Formula III:

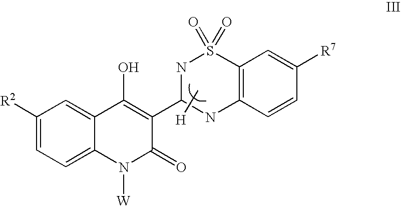

or a tautomer thereof, or a pharmaceutically acceptable salt or solvate thereof, wherein R², R⁷ and W are as defined herein.

It is to be understood that the present invention encompasses all combinations of particular, specific and preferred groups described hereinabove.

If a substituent described herein is not compatible with the synthetic methods of this invention, the substituent may be protected with a suitable protecting group that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999), which is incorporated herein by reference in its entirety. In some instances, a substituent may be specifically selected to be reactive under the reaction conditions used in the methods of this invention. Under these circumstances, the reaction conditions convert the selected substituent into another substituent that is either useful as an intermediate compound in the methods of this invention or is a desired substituent in a target compound.

In the compounds of this invention, various substituents may be a “protected —OH” group. This term refers to a substituent represented as —OR^{P}, where R^{P} refers to a suitable protecting group for an —OH moiety. Hydroxyl protecting groups are well known in the art and any hydroxyl protecting group that is useful in the methods of preparing the compounds of this invention may be used. Exemplary hydroxyl protecting groups include benzyl, tetrahydropyranyl, silyl (trialkyl-silyl, diaryl-alkyl-silyl, etc.) and various carbonyl-containing protecting groups, as disclosed in T. Greene and P. Wuts, supra. For example, in the compounds of this invention, R² may be the protected hydroxyl moiety —OSi(tert-butyl)(CH₃)₂.

The compounds of this invention may contain at least one chiral center and may exist as single stereoisomers (e.g., single enantiomers), mixtures of stereoisomers (e.g. any mixture or enantiomers or diastereomers) or racemic mixtures thereof. All such single stereoisomers, mixtures and racemates are intended to be encompassed within the broad scope of the present invention. Compounds identified herein as single stereoisomers are meant to describe compounds that are present in a form that are at least 90% enantiomerically pure. Where the stereochemistry of the chiral carbons present in the chemical structures illustrated herein is not specified, the chemical structure is intended to encompass compounds containing either stereoisomer of each chiral center present in the compound. Such compounds may be obtained synthetically, according to the procedures described herein using optically pure (enantiomerically pure) or substantially optically pure materials. Alternatively, these compounds may be obtained by resolution/separation of a mixture of stereoisomers, including racemic mixtures, using conventional procedures. Exemplary methods that may be useful for the resolution/separation of mixtures of stereoisomers include chromatography and crystallization/re-crystallization. Other useful methods may be found in “Enantiomers, Racemates, and Resolutions,” J. Jacques et al., 1981, John Wiley and Sons, New York, N.Y., the disclosure of which is incorporated herein by reference.

The compounds of this invention may possess one or more unsaturated carbon-carbon double bonds. All double bond isomers, both the cis (Z) and trans (B) isomers, and mixtures thereof are intended to be encompassed within the scope of the present invention.

The term “pharmaceutically acceptable salt” is intended to describe a salt that retains the biological effectiveness of the free acid or base of a specified compound and is not biologically or otherwise undesirable.

If an inventive compound is a base, a desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycollates, tartrates mandelates, and sulfonates, such as xylenesulfonates, methanesulfonates, propanesulfonates, naphthalene-1-sulfonates and naphthalene-2-sulfonates.

If an inventive compound is an acid, a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium. Particular pharmaceutically acceptable salts of a compound of Formulas I, II or III include the sodium salt and the potassium salt.

Because the compounds of this invention may contain both acid and base moieties, pharmaceutically acceptable salts may be prepared by treating these compounds with an alkaline reagent or an acid reagent, respectively. Accordingly, this invention also provides for the conversion of one pharmaceutically acceptable salt of a compound of this invention, e.g., a hydrochloride salt, into another pharmaceutically acceptable salt of a compound of this invention, e.g., a sodium salt.

The term “solvate” is intended to mean a pharmaceutically acceptable solvate form of a specified compound that retains the biological effectiveness of such compound. Examples of solvates include compounds of the invention in combination with water, isopropanol, ethanol, methanol, DMSO, ethyl acetate, acetic acid, or ethanolamine. In the case of compounds, salts, or solvates that are solids, it is understood by those skilled in the art that the inventive compounds, salts, or solvates may exist in different crystal forms, all of which are intended to be within the scope of the present invention and specified formulas.

Also included within the scope of this invention are prodrugs of the compounds of this invention. The term “prodrug” is intended to mean a compound that is converted under physiological conditions, e.g., by solvolysis or metabolically, to a compound of Formulas I, II or III, or a tautomer thereof, or a pharmaceutically acceptable salt or solvate thereof. A prodrug may be a derivative of one of the compounds of this invention that contains, for example, a carboxylic acid ester or amide moiety or an enol-ester moiety that may be cleaved under physiological conditions. A prodrug containing such a moiety may be prepared according to conventional procedures, for example, by treatment of a compound of Formula I, containing an amino, amido or hydroxyl moiety with a suitable derivatizing agent, for example, a carboxylic acid halide or acid anhydride, or by converting a compound of Formula I, containing a carboxyl moiety to an ester or amide or by converting a compound of Formula I, containing a carboxylic acid ester moiety to an enol-ester. Prodrugs of the compounds of this invention may be determined using techniques known in the art, for example, through metabolic studies. See, e.g., “Design of Prodrugs,” (H. Bundgaard, Ed.) 1985, Elsevier Publishers B. V., Amsterdam, The Netherlands.

The present invention is directed to a method of inhibiting an RNA-containing virus which comprises contacting the virus with an effective amount of a compound of Formulas I, II or III. This invention is also directed to a method of treating infection or disease caused by an RNA-containing virus comprising administering to a subject in need thereof, an effective amount of the compound of Formulas I, II or III. Specifically, this invention is directed to a method of inhibiting HCV activity, comprising contacting the virus with an effective amount of a compound of Formulas I, II or III, or a tautomer thereof, or a pharmaceutically acceptable salt or solvate thereof. For example, HCV activity may be inhibited in mammalian tissue by administering to a subject in need thereof a compound of Formula I or a tautomer thereof, or a pharmaceutically acceptable salt or solvate thereof.

A therapeutically “effective amount” is intended to mean that amount of a compound that, when administered to a mammal in need of such treatment, is sufficient to effect treatment, as defined herein. Thus, e.g., a therapeutically effective amount of a compound of Formula I or a tautomer thereof, or a pharmaceutically acceptable salt or solvate thereof is a quantity of an inventive agent that, when administered to a mammal in need thereof, is sufficient to modulate or inhibit the activity of HCV such that a disease condition which is mediated by that activity is reduced, alleviated or prevented. The amount of a given compound that will correspond to such an amount will vary depending upon factors such as the particular compound (e.g., the potency (IC₅₀), efficacy (EC₅₀), and the biological half-life of the particular compound), disease condition and its severity, the identity (e.g., age, size and weight) of the mammal in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmaceutical characteristics), disease or condition and its severity and the specific composition and method being used, but can nevertheless be determined by one of skill in the art.

In addition, this invention is directed to a method for inhibiting replication of hepatitis C virus comprising inhibiting replication of both positive and negative strand HCV-RNA, which method comprises contacting a cell infected with said virus with an effective amount of a compound of Formulas I, II or III. This invention is also directed to a method of treating infection or disease caused by hepatitis C virus comprising inhibiting replication of both positive and negative strand HCV-RNA, which method comprises administering to a subject in need thereof, an effective amount of a compound of Formulas I, II or III. More specifically, this invention is directed to a method of inhibiting replication of both positive and negative strand HCV-RNA with a compound of Formulas I, II or III, wherein the compounds demonstrate substantially equal inhibition of positive strand HCV-RNA replication and negative strand HCV-RNA replication. That is, for a given compound of this invention, the IC₅₀ for inhibition of positive strand HCV-RNA replication is not statistically different (less than a 2-fold difference) from the IC₅₀ for inhibition of negative strand HCV-RNA replication. Generally, the compounds of this invention demonstrate an IC₅₀ for inhibition of positive strand HCV-RNA replication that is +30% the IC₅₀ for inhibition of negative strand HCV-RNA replication.

“Treating” or “treatment” is intended to mean at least the mitigation of a disease condition (acute, chronic, latent, etc.) in a subject (a mammal, such as a human), where the disease condition is caused by an infectious RNA-containing virus. The methods of treatment for mitigation of a disease condition include the use of the compounds in this invention in any conventionally acceptable manner, for example for prevention, retardation, prophylaxis, therapy or cure of a disease. The compounds of Formula I, Formula II and Formula III of this invention are particularly useful for the treatment of acute, chronic or latent HCV diseases, such as acute and chronic hepatitis infection, hepatocellular carcinoma, liver fibrosis, or other HCV-related diseases. The compounds of Formula I, Formula II and Formula III of this invention may also be useful for treatment of diseases caused by infectious RNA-containing viruses other than HCV, including, but not limited to, Dengue, HIV or picornaviruses. Chronic fatigue syndrome is another disease that may be treatable using the compounds of this invention.

An inventive compound of Formulas I, II or III, or a tautomer thereof, or a pharmaceutically acceptable salt or solvate thereof may be administered to a subject as a pharmaceutical composition in any pharmaceutical form that is recognizable to the skilled artisan as being suitable. Suitable pharmaceutical forms include solid, semisolid, liquid, or lyophilized formulations, such as tablets, powders, capsules, suppositories, suspensions, liposomes, and aerosols. Pharmaceutical compositions of the invention may also include suitable excipients, diluents, vehicles, and carriers, as well as other pharmaceutically active agents, depending upon the intended use or mode of administration. Administration of a compound of the Formulas I, II or III, or a tautomer thereof, or pharmaceutically acceptable salt or solvate thereof, may be performed according to any of the generally accepted modes of administration available to those skilled in the art. The compounds of this invention may be administered by different routes including intravenous, intraperitoneal, subcutaneous, intramuscular, oral, topical, transdermal, or transmucosal administration. For systemic administration, oral administration is preferred. For oral administration, for example, the compounds can be formulated into conventional oral dosage forms such as capsules, tablets and liquid preparations such as syrups, elixirs and concentrated drops. Alternatively, injection (e.g., parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. The compounds of the invention may also be formulated in liposome-containing preparations, particularly liposome-containing preparations useful for delivery of the compounds of this invention to the liver or potentially to nonhepatic reservoirs of infection. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories. For topical administration, the compounds of the invention can be formulated into ointments, salves, gels, or creams, as is generally known in the art.

Compositions containing a compound of Formulas I, If or III, or a tautomer thereof, or pharmaceutically acceptable salt or solvate thereof, which are active when given orally can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include starch, calcium sulfate dihydrate, magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule, any pharmaceutical carrier routinely used for preparing dispersions or suspensions may be considered, for example aqueous gums, celluloses, silicates or oils, and may be incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formulas I, II or III, or a tautomer thereof, or pharmaceutically acceptable salt or solvate thereof, which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is formulated and administered in a unit dosage form. For oral application, for example, one or more tablets or capsules may be administered, for nasal application, a metered aerosol dose may be administered, for transdermal application, a topical formulation or patch may be administered and for transmucosal delivery, a buccal patch may be administered. A dose of the pharmaceutical composition contains at least a therapeutically effective amount of the active compound (i.e., a compound of Formulas I, II or III, or a tautomer thereof, or pharmaceutically acceptable salt or solvate thereof). The selected dose may be administered to a mammal, for example, a human patient, in need of treatment mediated by inhibition of HCV activity by any known or suitable method of administering the dose, including: topically, for example, as an ointment, or cream, orally, rectally, for example, as a suppository, parenterally by injection, or continuously by intravaginal, intranasal, intrabronchial, intraaural, or intraocular infusion.

Treatment of all forms of infection or disease (acute, chronic, latent etc) or as prophylaxis with these compounds (or their salts etc.) may be achieved using the compounds of this invention as a monotherapy, in dual or multiple combination therapy, such as in combination with other antivirals, in combination with an interferon, in combination with an interferon and ribavirin or levovirin, or in combination with one or more agents which include but are not limited to: immunomodulatory agents (such as cytokines, suppressors of cytokines and/or cytokine signalling, or immune modifiers, adjuvants and the like), immunomodulatory agents that enhance the body's immune system (such as vitamins, nutritional supplements, antioxidant compositions, vaccines or immunostimulating complexes, such as vaccines comprising a multimeric presentation of an antigen and adjuvant), other direct antiviral agents, indirect antiviral agents or agents which target viral RNA and impair translation or replication or modulate signalling or cellular host factors, or host-viral interface, immunoglobulins, antisense agents against HCV, peptide-nucleic acid conjugates, oligonucleotides, ribozymes, polynucleotides, anti-inflammatory agents, pro-inflammatory agents, antibiotics, hepatoprotectants, or any anti-infectious agents and the like, or combinations thereof. Moreover, the additional agents may be combined with the compounds of this invention to create a single dosage form. Alternatively, these additional agents may be separately administered as part of a multiple dosage form. As used herein the term “an interferon” is intended to mean any form of interferon, which includes, but is not limited to, natural or recombinant forms of alpha, beta or gamma interferons, albumin-linked interferons, or pegylated interferons.

Representative compounds of this invention include the compounds of Examples 1-227 or a tautomer thereof, or a pharmaceutically acceptable salt or solvate thereof. Representative of preferred compounds of this invention comprise the following: 1-(2-cyclopropylethyl)-6-fluoro-4-hydroxy-3-(7-hydroxy-1,1-dioxo-1,4-dihydro 1-benzo[1,2,4]thiadiazin-3-yl)-1H-quinolin-2-one, (3-[1-(2-cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yloxy}acetonitrile, 3-[4-hydroxy-1-(3-methylbutyl)-2-oxo-1,2-dihydroquinolin-3-yl]-1,1-dioxo-1,4-dihydrobenzo[1,2,4]thiadiazine-7-carboxylic acid amide, 2-{3-[1-(2-cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yloxy}acetamide, 2-[3-(7-carbamoylmethoxy-1,1-dioxo-1,4-dihydro-1-benzo[1,2,4]thiadiazin-3-yl)-4-hydroxy-1-(3-methyl-butyl)-2-oxo-1,2-dihydro-quinolin-6-yloxy]-acetamide, 1-(2-cyclopropylethyl)₃-((1,1-dioxo-1,4-dihydrobenzo[1,2,4]thiadiazin-3-yl)-6-fluoro-4-hydroxy-1H-quinolin-2-one, 2-{3-[4-hydroxy-1-(3-methylbutyl)-2-oxo-1,2-dihydroquinolin-3-yl]-1,1-dioxo-1,4-dihydrobenzo[1,2,4]thiadiazin-7-yloxy}acetamide, 3-{3-[1-(2-cyclopropylethyl)-6-fluoro-4-hydroxy-2-oxo-1,2-dihydroquinolin-3-yl]-1,1-dioxo-1,4-dihydro 1-benzo[1,2,4]thiadiazin-7-yl}propionamide, {3-[6-fluoro-4-hydroxy-1-(3-methylbutyl)-2-oxo-1,2-dihydro-quinolin-3-yl]-1,1-dioxo-1,4-dihydro-1-benzo[1,2,4]thiadiazin-7-yloxy}acetonitrile, 2-[3-(7-carbamoylmethoxy-1,1-dioxo-1,4-dihydro 1-benzo[1,2,4]thiadiazin-3-yl)-1-(2-cyclopropyl-ethyl)-hydroxy-2-oxo-1,2-dihydro-quinolin-6-yloxy]-acetamide, 1-(2-cyclopropyl-ethyl)-3-[1,1-dioxo-7-(tetrazol-5-ylmethoxy)-1,4-dihydro-1-benzo[1,2,4]thiadiazin-3-yl]-6-fluoro-4-hydroxy-1H-quinolin-2-one, 2-{3-[1-(2-cyclopropyl-ethyl)-6-fluoro-4-hydroxy-2-oxo-1,2-dihydro-quinolin-3-yl-3-1,1-dioxo-1,4-dihydro-1-benzo[1,2,4]thiadiazin-7-yloxy)-propionamide, 6-amino-3-(7-amino-1,1-dioxo-1,4-dihydrobenzo[1,2,4]thiadiazin-3-yl)-4-hydroxy-1-(3-methylbutyl)-1H-quinolin-2-one, (3-[4-hydroxy-1-(3-methylbutyl)₂-oxo-1,2-dihydroquinolin-3-yl]-1,1-dioxo-1,4-dihydrobenzo[1,2,4]thiadiazin-7-yloxy 3acetonitrile, (E)₃-{3-[4-hydroxy-1-(3-methylbutyl)₂-oxo-1,2-dihydroquinolin-3-yl]-1,1-dioxo-1,4-dihydrobenzo[1,2,4]thiadiazin-7-yl}acrylamide, 6-fluoro-4-hydroxy-3-(7-hydroxy-1,1-dioxo-1,4-dihydro-1-benzo[1,2,4]thiadiazin-3-yl)-1-(3-methylbutyl)-1H-quinolin-2-one, 2-{3-[1-(2-cyclopropyl-ethyl)-6-fluoro-4-hydroxy-2-oxo-1,2-dihydroquinolin-3-yl]-1,1-dioxo-1,4-dihydro-1-benzo[1,2,4]thiadiazin-7-yloxy}-2-methylpropionamide, [3-(7-cyanomethoxy-1,1-dioxo-1,4-dihydro-1-benzo[1,2,4]thiadiazin-3-yl)-4hydroxy-1-(3-methyl-butyl)-2-oxo-1,2-dihydro-quinolin-6-yloxy]acetonitrile, {3-[1-(2-cyclopropylethyl)-6-fluoro-4-hydroxy-2-oxo-1,2-dihydroquinolin-3-yl]-1,1-dioxo-1,4-dihydrobenzo[1,2,4]thiadiazin-7-yloxy}acetic acid, 2-[3-(7-carbamoylmethoxy-1,1-dioxo-1,4-dihydro-1-benzo[1,2,4]thiadiazin-3-yl)-4-hydroxy-1-(3-methylbutyl)-2-oxo-1,2-dihydroquinolin-6-ylamino]acetamide, 6-fluoro-4-hydroxy-3-(7-hydroxy-1,1-dioxo-1,4-dihydro-1-benzo[1,2,4]thiadiazin-3-yl)-1-pyridin-4-ylmethyl-1H-quinolin-2-one, 1-(2-cyclopropylethyl)-6-fluoro-4-hydroxy-3-[7-(5-methyl-[1,3,4]oxadiazol-2-ylmethoxy)-1,1-dioxo-1,4-dihydro-1-benzo[1,2,4]thiadiazin-3-yl]-1H-quinolin-2-one, 6-chloro-4-hydroxy-3-(7-hydroxy-1,1-dioxo-1,4-dihydro-1-benzo[1,2,4]thiadiazin-3-yl)-1-(3-methylbutyl)-1H-quinolin-2-one, {3-[6-chloro-4-hydroxy-1-(3-methylbutyl)-2-oxo-1,2-dihydroquinolin-3-yl]-1,1-dioxo-1,4-dihydro-1-benzo[1,2,4]thiadiazin-7-yloxy}acetonitrile, 2-(3-[6-chloro-4-hydroxy-1-(3-methyl-butyl)-2-oxo-1,2-dihydro-quinolin-3-yl]-1,1-dioxo-1,4-dihydro-1-benzo[1,2,4]thiadiazin-7-yloxy)acetamide, 3-(7-amino-1,1-dioxo-1,4-dihydro-1-benzo[1,2,4]thiadiazin-3-yl)-6-fluoro-4-hydroxy-113-methylbutyl)-1H-quinolin-2-one, {3-[6-amino-4-hydroxy-1-(3-methylbutyl)-2-oxo-1,2-dihydroquinolin-3-yl]-1,1-dioxo-1,4-dihydro-16-benzo[1,2,4]thiadiazin-7-yloxy)acetonitrile, 3-[1-(2-cyclopropylethyl)-6-fluoro-4-hydroxy-2-oxo-1,2-dihydroquinolin-3-yl]-1,1-dioxo-1,4-dihydrobenzo[1,2,4]thiadiazin-7-yloxy}acetic acid, 2-[3-(7-carbamoylmethoxy-1,1-dioxo-1,4-dihydro-1-benzo[1,2,4]thiadiazin-3-yl)-4-hydroxy-1-(3-methylbutyl)-2-oxo-1,2-dihydroquinolin-6 ylamino]acetamide, 6-fluoro-4-hydroxy-3-(7-hydroxy-1,1-dioxo-1,4-dihydro-1-benzo[1,2,4]thiadiazin-3-yl)-1-pyridin-4-ylmethyl-1H-quinolin-2-one, 1-(2-cyclopropylethyl)-6-fluoro-4-hydroxy-3-[7-(5-methyl-[1,3,4]oxadiazol-2-ylmethoxy)-1,1-dioxo-1,4-dihydro-1-benzo[1,2,4]thiadiazin-3-yl]-1H-quinolin-2-one, 6-chloro-hydroxy-3-(7-hydroxy-1,1-dioxo-1,4-dihydro-1-benzo[1,2,4]thiadiazin-3-yl)-1-(3-methylbutyl)-1H-quinolin-2-one, {3-[6-chloro-4-hydroxy-2-oxo-1-(3-methylbutyl)-2-dihydroquinolin-3-yl]-1,1-dioxo-1,4-dihydrobenzo[1,2,4]thiadiazin-7-yloxy}acetamide, and {3-[6-amino-4-hydroxy-1-(3-methylbutyl)-2-oxo-1,2-dihydroquinolin-3-yl]-1,1-dioxo-1,4-dihydro-16-benzo[1,2,4]thiadiazin-7-yloxy}acetonitrile, or a tautomer thereof, or a pharmaceutically acceptable salt or solvate thereof.

The following compounds possess an IC₅₀ greater than 10 uM and may be useful in the methods described herein: 3-(1,1-dioxo-1,4-dihydrobenzo[1,2,4]thiadiazin-3-yl)₄-hydroxy-1-(2-phenylethyl)-1H-quinolin-2-one, 1-(2-cyanobenzyl)-3-(1,1-dioxo-1,4-dihydrobenzo[1,2,4]thiadiazin-3-yl)-4-hydroxy-1H-quinolin-2-one, 3-(1,1-dioxo-1,4-dihydrobenzo[1,2,4]thiadiazin-3-yl)-4-hydroxy-1-(3-phenylpropyl)-1H-quinolin-2-one, N-[3-(1,1-dioxo-1,4-dihydrobenzo[1,2,4]thiadiazin-3-yl)-4-hydroxy-1-(3-methylbutyl)-2-oxo-1,2-dihydroquinolin-6-yl]-4-methoxybenzamide, 3-(1,1-dioxo-1,4-dihydrobenzo[1,2,4]thiadiazin-3-yl)-4-hydroxy-1-(3-methylbutyl)-2-oxo-1,2-dihydroquinoline-6-carboxylic acid ethyl ester, 3-[1-(2-cyclopropylethyl)-4-hydroxy-2-oxo-1,2-dihydroquinolin-3-yl-]-1,1-dioxo-1,4-dihydrobenzo[1,2,4]thiadiazine-7-carboxylic acid [2-(4-methoxyphenyl)ethyl]amide, 4-[3-(1, 1-dioxo-1,2-dihydrobenzo[1,2,4]thiadiazin-3-yl)-4-hydroxy-2-oxoquinolin-1-yl]butyramide, 3-(1, 1-dioxo-1,2-dihydrobenzo[1,2,4]thiadiazin-3-yl)-4-hydroxy-1-(3-methylbutyl)-2-oxo-1,2-dihydro-quinoline-7-carboxylic acid, 7-bromo-3-(1,1-dioxo-1,2-dihydrobenzo[1,2,4]thiadiazin-3-yl)-4-hydroxy-1-(3-methylbutyl)-1H-quinolin-2-one, 3-(1,1-dioxo-7-phenyl-1,4-dihydro-1-benzo[1,2,4]thiadiazin-3-yl)-4-hydroxy-1-(3-methylbutyl)-1H-quinolin-2-one, thiophene-2-carboxylic acid [3-(1,1-dioxo-1,4-dihydro-1-benzo[1,2,4]thiadiazin-3-yl)-4-hydroxy-1-(3-methylbutyl)-2-oxo-1,2-dihydro-quinolin-6-yl]amide, N-[3-1,1-dioxo-1,4-dihydro-1-benzo[1,2,4]thiadiazin-3-yl)-4-hydroxy-1-(3-methylbutyl)-2-oxo-1,2-dihydro-quinolin-6-yl]-3-trifluorobenzamide, N-[3-(1,1-dioxo-1,4-dihydro-1-benzo[1,2,4]thiadiazin-3-yl)-4-hydroxy-1-(3-methylbutyl)-2-oxo-1,2-dihydro-quinolin-6-yl]-3-fluorobenzamide, N-[3-(1,1-dioxo-1,4-dihydro-1-benzo[1,2,4]thiadiazin-3-yl)-4-hydroxy-1-(3-methylbutyl)-2-oxo-1,2-dihydro-quinolin-6-yl]-nicotinamide, N-[3-(1,1-dioxo-1,4-dihydro-1-benzo[1,2,4]thiadiazin-3-yl)-4-hydroxy-1-(3-methylbutyl)-2-oxo-1,2-dihydro-quinolin-6-yl]acetamide, 4-hydroxy-3-(7-iodo-1,1-dioxo-1,4-dihydro-1-benzo[1,2,4]thiadiazin-3-yl)-1-(3-methylbutyl)-1H-quinolin-2-one, 4-[3-(1,1-dioxo-1,4-dihydro-1-benzo[1,2,4]thiadiazin-3-yl)-4-hydroxy-2-oxo-2H-quinolin-1-yl]-butyric acid ethyl ester, 4-[3-(1,1-dioxo-1,4-dihydro-1-benzo[1,2,4]thiadiazin-3-yl)-4-hydroxy-2-oxo-2H-quinolin-1-yl]-butyric acid, [3-(1,1-dioxo-1,4-dihydro-1-benzo[1,2,4]thiadiazin-3-yl)-4hydroxy-6-(3-methyl-butoxy)-2-oxo-2H-quinolin-1-yl]acetic acid, 3-(1,1-dioxo-1,4-dihydro-1-benzo[1,2,4]thiadiazin-3-yl)-4-hydroxy-6-iodo-1-(3-methylbutyl)-1H-quinolin-2-one, 3-(1, 1-dioxo-1,4-dihydro-1-benzo[1,2,4]thiadiazin-3-yl)-4-hydroxy-8-(3-methylbutyl)-1H-quinolin-2-one, 3-(1,1-dioxo-1,4dihydro-1-benzo(1,2,4]thiadiazin-3-yl)-4-hydroxy-1-(3-methylbutyl)-2-oxo-1,2-dihydro-quinoline-5-carboxylic acid dimethylamide, 3-(1,1-dioxo-1,4-dihydro-1-benzo[1,2,4]thiadiazin-3-yl)-4-hydroxy-1-(3-methylbutyl)-2-oxo-1,2-dihydroquinoline-5-carboxylic acid, 3-(1,1-dioxo-1,4-dihydro-1-benzo[1,2,4]thiadiazin-3-yl)-4-hydroxy-6(4-methoxy-benzyloxy)-1-(3-methylbutyl)-1H-quinolin-2-one, 1-(2-dimethylamino-ethyl)-3-(1,1-dioxo-1,4-dihydro-1-benzo[1,2,4]thiadiazin-3-yl)-4-hydroxy-1H-quinolin-2-one, 4-hydroxy-3-(6-methoxy-1,1-dioxo-1,4-dihydro-1-benzo[1,2,4]thiadiazin-3-yl)-1-(3-methylbutyl)-1H-quinolin-2-one, 6-(6-bromo-pyridin-2-yloxy)-3-(1,1-dioxo-1,4-dihydro-1-benzo[1,2,4]thiadiazin-3-yl)-4-hydroxy-1-(3-methylbutyl)-1H-quinolin-2-one, 3-(1, 1-dioxo-1,4-dihydro-1-benzo[1,2,4]thiadiazin-3-yl)-4-hydroxy-1-(2-hydroxyethyl)-1H-quinolin-2-one, 3-(1,1-dioxo-1,4-dihydro-1-benzo[1,2,4]thiadiazin-3-yl)-4-hydroxy-6(6-methoxy-pyridin-2-yloxy)-1-(3-methylbutyl)-1H-quinolin-2-one, 3-(1,1-dioxo-1,4-dihydro-1-benzo[1,2,4]thiadiazin-3-yl)-1-(5-fluoro-2-methyl-benzyl)-4-hydroxy-1H-quinolin-2-one, and 3-(1,1-dioxo-1,4-dihydro-1-benzo[1,2,4]thiadiazin-3-yl)-4-hydroxy-1-(2-nitro-benzyl)-1H-quinolin-2-one, or a tautomer thereof, or a pharmaceutically acceptable salt or solvate thereof.

The following compounds did not demonstrate biological activity at the screening rate of 10 uM, however, such compounds may demonstrate activity at higher testing rates or when evaluated under different assay conditions: 12-cyano-3,5-dichlorobenzyl)-3-(1,1-dioxo-1,2-dihydrobenzo[1,2,4]thiadiazin-3-yl)-4-hydroxy-1H-quinolin-2-one, 3-(1,1-dioxo-1,2-dihydrobenzo[1,2,4]thiadiazin-3-yl)-4-hydroxy-1-(3-methylbutyl)-7-nitro-1H-quinolin-2-one, -chloro-3-(1,1-dioxo-1,2-dihydrobenzo[1,2,4]thiadiazin-3-yl)-4-hydroxy-1-(3-methylbutyl)-H-quinolin-2-one, 1-(4-aminobutyl)-3-1,1-dioxo-1,2-dihydrobenzo[1,2,4]thiadiazin-3-yl)-4-hydroxy-1-1H-quinolin-2-one, 3-(1,1-dioxo-1,2-dihydrobenzo[1,2,4]thiadiazin-3-yl)-4-hydroxy-1-phenyl-1H-quinolin-2-one, 6-benzyloxy-3-(1,1-dioxo-1,2-dihydrobenzo[1,2,4]thiadiazin-3-yl)-4hydroxy-1-(3-methylbutyl)-1H-quinolin-2-one, 3-(8-bromo-1,1-dioxo-1,4-dihydro-1-benzo[1,2,4]thiadiazin-3-yl)-4-hydroxy-1-(3-methylbutyl)-1H-quinolin-2-one, (E)-3-[3-(1,1-dioxo-1,4-dihydro-1-benzo[1,2,4]thiadiazin-3-yl)-4-hydroxy-1-(3-methylbutyl)-2-oxo-1,2dihydro-quinolin-7-yl]acrylamide, [3-(1,1-dioxo-1,4-dihydro-1-benzo[1,2,4]thiadiazin-3-yl)-4-hydroxy-6-(3-methyl-butoxy)-2-oxo-2H-quinolin-1-yl]acetic acid tert-butyl ester, 3-(1,1-dioxo-1,4-dihydro-1-benzo[1,2,4]thiadiazin-3-yl)-4-hydroxy-1-(3-methylbutyl)-2-oxo-1,2-dihydro-quinoline-6carboxylic acid dimethylamide, 3-(1,1-dioxo-1,4-dihydro-1-benzo[1,2,4]thiadiazin-3-yl)-4-hydroxy-1-(2-pyrrolidin-1-yl-ethyl)-1H-quinolin-2-one, and 1-(4-tert-butylbenzyl)-3-(1,1-dioxo-1,4-dihydro-1-benzo[1,2,4]thiadiazin-3-yl)-4-hydroxy-1H-quinolin-2-one, or a tautomer thereof, or a pharmaceutically acceptable salt or solvate thereof.

General Synthetic Methods

This invention is also directed to methods for the synthesis of the compounds of Formula I and tautomers thereof.

Included in the present invention is a process according to Scheme 1 for the synthesis of the compounds:

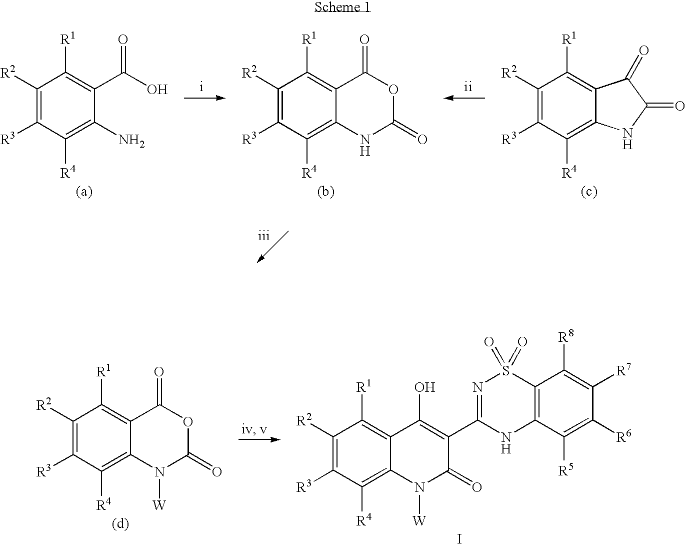

Conditions: i. phosgene, THF; ii. peracetic acid, AcOH; iii. W-X, NaH, DMA or W—OH, PPh₃, DIAD, THF; iv. NaH, THF, reflux or DBU, THF, 40-45° C.; v. AcOH, reflux or acetic acid, then HCl.

An appropriately substituted 2-aminobenzoic acid (a) such as 2-amino-5-fluorobenzoic acid, 2-amino-5-tert-butyldimethylsilyloxybenzoic acid or 2-amino-5-methylbenzoic acid can be treated with phosgene or a phosgene equivalent such as triphosgene or ethyl chloroformate in an appropriate solvent such as tetrahydrofuran to afford the benzo[d][1,3]oxazines (b). Alternatively, an indole-2,3-dione (c) such as 4 bromoindole-2,3-dione may be oxidised with a peracid such as peracetic acid to afford the benzo[d][1,3]oxazines (b). The benzo[d][1,3]oxazines (b) such as 1H-benzo[d][1,3]oxazine-2,4-dione, 6-methyl-1H-benzo[d][1,3]oxazine-2,4-dione or 6-fluoro-1H-benzo[d][1,3]oxazine-2,4-dione may be alkylated with an appropriate alkylating agent such as 3-methyl-1-bromobutane, benzyl bromide or (bromomethyl)cyclopropane in the presence of an appropriate base such as potassium carbonate or sodium hydride in an appropriate solvent such as tetrahydrofuran, dimethylacetamide or dimethylformamide to afford the N-alkylated 1H-benzo[d][1,3]oxazine-2,4-dione (d). Alternatively, compounds (d) can be prepared by the alkylation of benzo[d][1,3]oxazines (b) in a Mitsunobu reaction with an appropriate alcohol such as 2-cyclopropylethanol, 3,3-dimethylbutanol, 2-furancarbinol or 4-pyridinylcarbinol in the presence of a phosphine such as triphenylphosphine and an azodicarboxylate such as diethyl azodicarboxylate or diisopropyl azodicarboxylate in a solvent such as tetrahydrofuran. Compounds of Formula I may be prepared by the coupling of N-alkylated benzo[d][1,3]oxazines (d) with an appropriate thiadiazine such as ethyl 1,1-dioxo-2H-benzo-1,2,4-thiadiazinyl-3-acetate, methyl (7-bromo-1,1-dioxo-1,2-dihydrobenzo[1,2,4]thiadiazin-3-yl)acetate or ethyl (1,1-dioxo-7-hydroxy-1,2-dihydrobenzo[1,2,4]thiadiazin-3-yl)acetate in the presence of a base such as sodium hydride or DBU (1,8-diazabicyclo[5.4.0]undec-7-ene) in an appropriate solvent such as dimethylformamide, dimethylacetamide or tetrahydrofuran followed by acidification with an acid such as acetic acid or acetic acid followed by hydrochloric acid.

Also included in the present invention is a process according to Scheme 2:

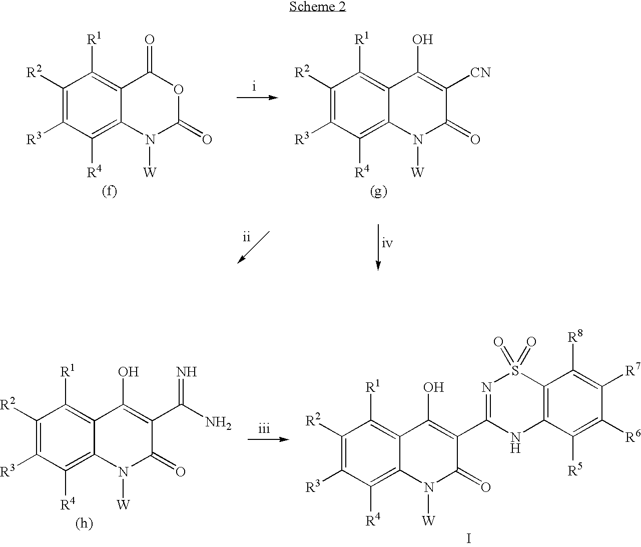

Conditions: i. methyl cyanoacetate, NaH, DMF; ii. NH₄Cl, AlMe₃, THF; iii. 2-chloro-5-nitrobenzenesulfonyl chloride, NaH, THF; iv. 2-aminobenzenesulfonamide, AlMe₃, THF

A benzo[d][1,3]oxazine-2,4-dione (f) such as 1-(3-methylbutyl)-1H-benzo[d][1,3]oxazine-2,4-dione can be treated with a cyanoacetate such as methyl cyanoacetate or ethyl cyanoacetate in the presence of an appropriate base such as sodium hydride in an appropriate solvent such as tetrahydrofuran or dimethylformamide then acidified with an acid such as acetic acid to afford the 3-cyanoquinolines (g). These cyano compounds (g) may be then condensed with an appropriate 2-aminobenzenesulfonamide such as 2-aminobenzenesulfonamide, 2-amino-5-chlorobenzenesulfonamide or 2-amino-4-bromobenzenesulfonamide in the presence of trimethylaluminum in an appropriate solvent such as dioxane, toluene or tetrahydrofuran to afford the compounds of Formula I. Alternatively, compounds (g) may be treated with ammonium chloride and triethylaluminum in an appropriate solvent such as toluene or dioxane to give amidines (h) which may then be coupled with an appropriate 2-chlorobenzenesulfonyl chloride such as 5-nitro-2-chlorobenzenesulfonyl chloride in the presence of a base preferably sodium hydride to give compounds of Formula I.

Also included in the present invention is a process according to Scheme 3:

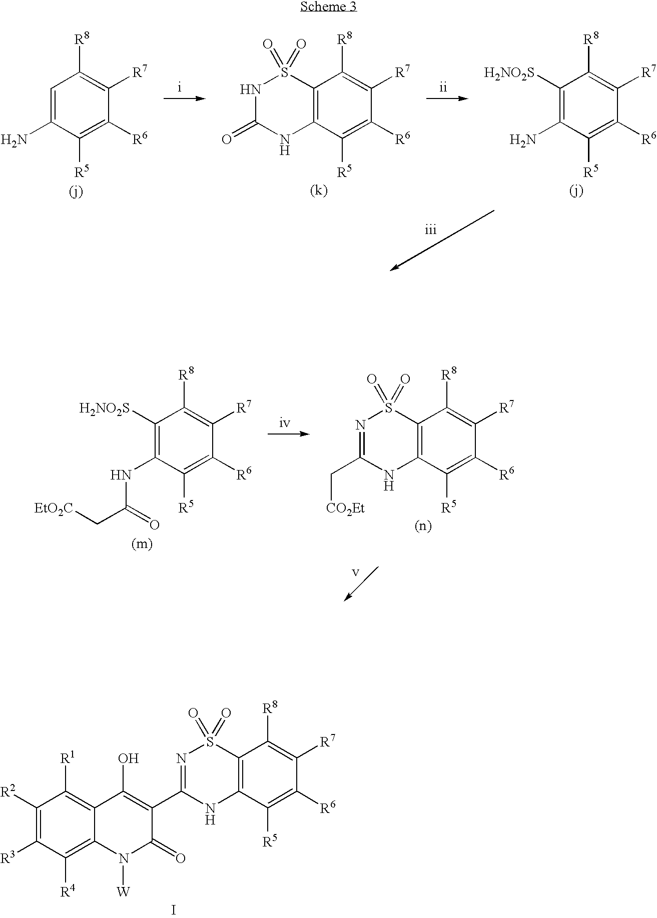

Conditions: i) ClSO₂—N═C═O, nitroethane; then AlCl₃; ii) aqu. H₂SO₄, heat; iii) ethyl chloromalonate, pyridine; or ethyl chloromalonate, triethylamine, THF, 4° C.; or diethyl malonate, heat; iv) POCl₃ (neat), reflux; or Na₂CO₃, water or CsCO₃, ethanol, 77° C.; v) NaH, THF, reflux then AcOH, reflux or DBU, THF, 4045° C., then acetic acid, then HCl.

An aniline (j) such as 4-methoxyaniline or 2-methylaniline can be treated with chlorosulfonylisocyanate in an appropriate solvent such as nitroethane then treated with a acid such as aluminum trichloride to give the cyclized compound (k). Compound (k) can be hydrolysed with an aqueous acid such as aqueous sulfuric acid to afford the 2-aminobenzenesulfonamides (l). Amides (m) can be formed by treating amines (l) with ethyl chloromalonate in the presence of a base such as pyridine, triethylamine or pyridine in a solvent such as tetrahydrofuran or dichloromethane. Cyclisation of amides (m) to afford thiadiazines (n) may occur on treatment with a dehydrating agent, such as phosphorus oxychloride, either neat or in a solvent, such as toluene, or with a base, such as sodium carbonate, cesium carbonate or sodium bicarbonate, in a solvent, such as water or aqueous ethanol. Condensation of the thiadiazines (n) with benzo[d][1,3]oxazines as described in Scheme 1 provides compounds of Formula I.

Also included in the present invention is a process according to Scheme 4:

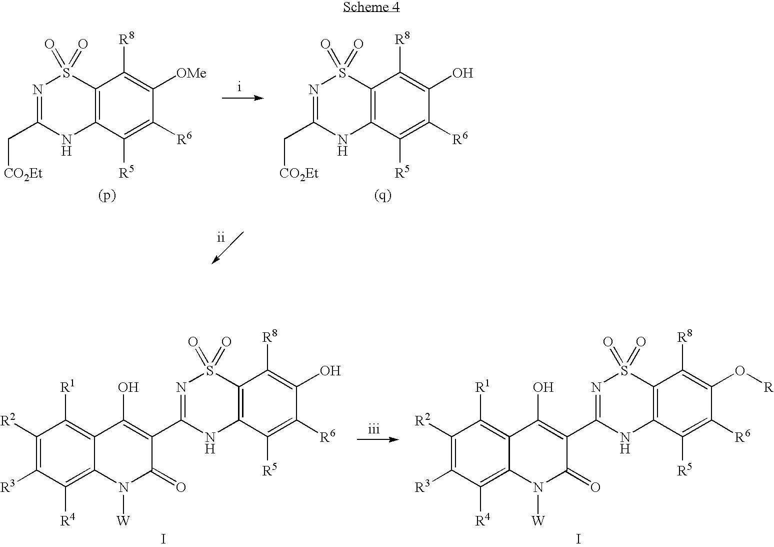

Conditions: i. BBr₃, DCM then EtOH, H₂SO₄; ii. benzo[d][1,3]oxazine, NaH, THF then AcOH; iii. R—Br, NaH.

7-Methoxythiadiazines (p) can be treated with a demethylation reagent, such as boron tribromide or hydrobromic acid, in an appropriate solvent, such as dichloromethane or acetic acid, and any ester hydrolysis product may then be re-esterified by treatment with an alcohol, such as ethanol in the presence of an acid, such as sulfuric acid, to give the 7-hydroxy compounds (q). Compounds (q) may be coupled with benzo[d][1,3]oxazines as described in Scheme 1 to gives hydroxy compounds of Formula I. The free hydroxyl group may then be optionally treated with an alkylating agent, such as bromoacetamide or bromoacetonitrile, in the presence of a base such as sodium hydride or potassium carbonate to give alkylated compounds of Formula I.

Alternate methods for preparing the N-alkylated 1H-benzo[d][1,3]oxazine-2,4-dione (d) are shown in Scheme 5 and Scheme 6.

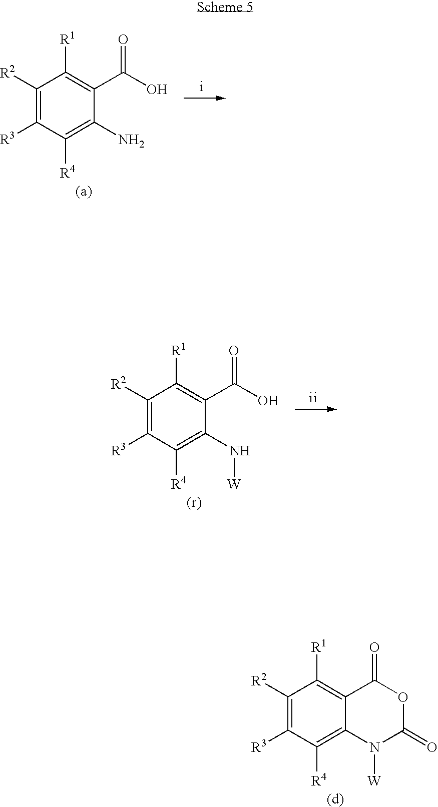

Conditions: i. W—CHO, NaBH₄. THF; ii. triphosgene, THF

One alternate method for the preparation of the N-alkylated 1H-benzo[d][1,3]oxazine-2,4-dione (d), shown in Scheme 5, comprises treating the 2-aminobenzoic acid (a) under reductive amination conditions by treating the 2-aminobenzoic acid with an appropriate aldehyde (W—CHO) in the presence of an appropriate reducing agent, such as sodium borohydride, sodium cyanoborohydride or diborane, in a suitable solvent such as tetrahydrofuran or dichloromethane, to form the N-akylated 2-aminobenzoic acid (r). Treatment of the N-akylated 2-aminobenzoic acid (r) with phosgene or a phosgene equivalent such as triphosgene or ethyl chloroformate in an appropriate solvent such as tetrahydrofuran, as described above, provides the N-alkylated 1H-benzo[d][1,3]oxazine-2,4-dione (d).

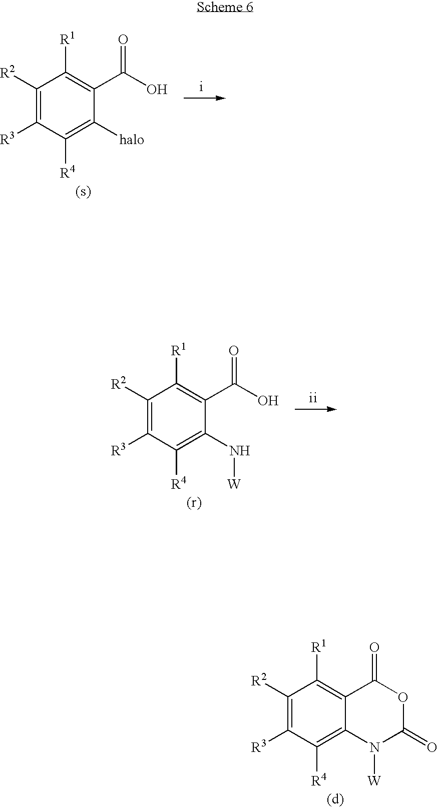

Conditions: i. W—NH₂, 6 mol % CuBr₂, K₂CO₃, THF, 63° C.; ii. triphosgene, THF

Scheme 6 illustrates an alternate method for the preparation of the intermediate N-akylated 2-aminobenzoic acid (r) by coupling of a 2-halobenzoic acid (s), such as a 2-bromobenzoic acid or a 2-chlorobenzoic acid, with an N-substituted amine (W—NH₂) in the presence of an appropriate copper catalyst, for example copper (II) bromide, in the presence of a suitable base, such as potassiuim carbonate or triethylamine, in an appropriate solvent such as tetrahydrofuran or dimethylformamide. Conversion of the N-akylated 2-aminobenzoic acid (r) to the N-alkylated 1H-benzo[d][1,3]oxazine-2,4-dione (d). may be accomplished as described above.

Optionally, a salt of a compound of Formula I, prepared using any of the methods described in Schemes 1-6 above, may be prepared by treating the compound with an appropriate base, such as sodium hydroxide or potassium hydroxide, in an appropriate solvent, such as water or water and methanol.

Also included within the scope of the present invention are intermediate compounds that are useful for the preparation of the compounds of Formulas I, II and/or III. Such useful intermediate compounds include: 1-phenethyl-1H-benzo[d][1,3]oxazine-2,4-dione, 1-(2-cyanobenzyl)-1H-benzo[d][1,3]oxazine-2,4-dione, 1-(3-phenylpropyl)-1H-benzo[d][1,3]oxazine-2,4-dione, 1-cyclopropylmethyl-1H-benzo[d][1,3]oxazine-2,4-dione, 1-(3-methylbutyl)-6-nitrobenzo[d][1,3]oxazine-2,4-dione, 6-chloro-1-(3-methylbutyl)benzo[d][1,3]oxazine-2,4-dione, 6-bromo-1-(3-methylbutyl)-benzo[d][1,3]oxazine-2,4-dione, 6methoxy-(3-methylbutyl)benzo[d][1,3]oxazine-2,4-dione, 1-but-3-enyl-1H-benzo[d][1,3]oxazine-2,4-dione, 1-cyclohexylmethyl-1H-benzo[d][1,3]oxazine-2,4-dione, 1-(3-methylbut-2-enyl)-1H-benzo[d][1,3]oxazine-2,4dione, 1-(3-methylbutyl)-6methylbenzo[d][1,3]oxazine-2,4-dione, 1-(3-methylbutyl)-6-fluorobenzo[d][1,3]oxazine-2,4-dione, 1-(3-methylbutyl)-5-fluorobenzo[d][1,3]oxazine-2,4-dione, 1-(3-methylbutyl)-6,7-difluorobenzo[d][1,3]oxazine-2,4-dione, 1-(3-methylbutyl)-7-fluorobenzo[d][1,3]oxazine-2,4-dione, 1-(4,4,4-trifluorobutyl)-1H-benzo[d ][1,3]oxazine-2,4-dione, 3-cyano-4-hydroxy-1-(3-methylbutyl)-1H-quinolin-2-one, 4-hydroxy-1-(3-methylbutyl)-2-oxo-1,2-dihydroquinoline-3carboxamidine, 1-pent-4-ynyl-1H-benzo[d][1,3]oxazine-2,4-dione, methyl (7-bromo-1,1-dioxo-1,2-dihydrobenzo[1,2,4]thiadiazin-3-yl)acetate, 8-bromo-2-isobutoxybenzo[d][1,3]oxazin-4-one, 1-cyclopropylmethyl-1H-benzo[d][1,3]oxazine-2,4-dione, 1-(3-methylbutyl)-5-methylbenzo[d][1,3]oxazine-2,4-dione, 1-(3-methylbutyl)-5-chlorobenzo[d][1,3]oxazine-2,4-dione, 5-bromo-2H-3,1-benzoxazine-1-(3-methylbutyl)-2,4-dione, 7-bromo-2H-3,1-benzoxazine-1-(3-methylbutyl)-2,4-dione, 4-(6-methoxybenzo[d][1,3]oxazine-2,4-dione-1-yl)butyrontrile, 1-butyn-3-yl-1H-benzo[d][1,3]oxazine-2,4dione, 1-(3,3-dimethylbutyl)-1H-benzo[d][1,3]oxazine-2,4-dione, 1-(2-cyclopropylethyl)-1H-benzo[d ][1,3]oxazine-2,4-dione, 3-cyano-4-hydroxy-1-(3-methylbutyl)-1H-quinolin-2-one, 1-(2-methylthio)ethyl-1H-benzo[d][1,3]oxazine-2,4-dione, 1-(2-methylthio)propyl-1H-benzo[d ][1,3]oxazine-2,4-dione, 1-(3-furanylmethyl)methyl-1H-benzo[d][1,3]oxazine-2,4-dione, 1-[2-(2-thienyl)]ethyl-1H-benzo[d][1,3]oxazine-2,4-dione, 1-[2-(3-thienyl)]ethyl-1H-benzo[d][1,3]oxazine-2,4-dione, 1-(2-cyclopropylethyl)-6-fluorobenzo[d][1,3]oxazine-2,4-dione, 6(tert-butyl-dimethylsilanyloxy)-1-(2-cyclopropylethyl)-1H-benzo[d]oxazine-2,4-dione, 3-cyano-1-(2-cyclopropylethyl)-4-hydroxy-1H-quinolin-2-one, (7-iodo-1,1-dioxo-1,4-dihydro-1-benzo[1,2,4]thiadiazin-3-yl)-acetic acid ethyl ester, 1-(3,3-dimethylbutyl)-6-nitrobenzo[d][1,3]oxazine-2,4dione, 3-(1,1-dioxo-7-iodo-1,4-dihydrobenzo[1,2,4]thiadiazin-3-yl)-4-hydroxy-1-(3-methylbutyl)-6-nitro-1H-quinolin-2-one, 3-[4-hydroxy-1-(3-methylbutyl)-6-nitro-2-oxo-1,2-dihydroquinolin-3-yl]-1,1-dioxo-1,4-dihydrobenzo[1,2,4]thiadiazine-7-carboxylic acid amide, 1-(tetrahydrofuran-3-ylmethyl)-1H-benzo[d][1,3]oxazine-2,4-dione, 6-iodo-1-(3-methylbutyl)-1H-benzo[d][1,3]oxazine-2,4-dione, 1-(tetrahydro-furan-2-ylmethyl)-1H-benzo[d][1,3]oxazine-2,4-dione, 1-(3-methylpentyl)-1H-benzo[d][1,3]oxazine-2,4-dione, 4-hydroxy-3-(7-iodo-1,1-dioxo-1,4-dihydrobenzo[1,2,4]thiadiazin-3-yl)-1-(3-methylbutyl)-1H-quinolin-2-one,

5,6-dimethoxy-1-(3-methyl-butyl)-1H-benzo[d][1,3]oxazine-2,4-dione, 4-methyl-1,1-dioxo-1,4-dihydrobenzo[1,2,4]thiadiazin-3-one, N-methyl-N-(2-sulfamoylphenyl)malonamic acid ethyl ester, (4-methyl-1,1-dioxo-1,4-dihydrobenzo[1,2,4]thiadiazin-3-yl)acetic acid ethyl ester, 3-1,1-dioxo-7-methyl-1,4-dihydrobenzo[1,2,4]thiadiazin-3-yl)-acetic acid ethyl ester, 1-(4-nitrobenzyl)-1H-benzo[d ][1,3]oxazine-2,4-dione, 1-(6-aminopyridin-3-ylmethyl)-1H-benzo[d][1,3]oxazine-2,4-dione, 1-(4-bromobenzyl)-1H-benzo[d][1,3]oxazine-2,4-dione, 1-(3-bromobenzyl)-1H-benzo[d][1,3]oxazine-2,4-dione, 7-methoxy-1,1-dioxo-1,4-dihydrobenzo[1,2,4]thiadiazin-3-one, N-(4-methoxy-2-sulfamoylphenyl)malonic acid ethyl ester, (7-methoxy-1,1-dioxo-1,4-dihydrobenzo[1,2,4]thiadiazin-3-yl)-acetic acid ethyl ester, 1-(2-cyclopropylethyl)-3-(1,1-Dioxo-1,4-dihydrobenzo[1,2,4]thiadiazin-7-iodo-3-yl)-6-fluoro-4-hydroxy-1H-quinolin-2-one, (7-hydroxy-1,1-dioxo-1,4-dihydrobenzo[1,2,4]thiadiazin-3-yl)acetic acid ethyl ether, 1-([(2-methylcyclopropyl)methyl]-1H-benzo[d][1,3]oxazine-2,4-dione, 1-[(2-methylcyclopropyl)methyl]-1H-benzo[d][1,3]oxazine-2,4-dione, 4,6-dihydroxy-3-(7-methoxy-1,1-dioxo-1,4-dihydrobenzo[1,2,4]thiadiazin-3-yl)-1-(3-methyl-butyl)-1H-quinolin-2-one, 1-(2-cyclopropyl-ethyl)-4,6dihydroxy-3-(7-hydroxy-1,1-dioxo-1,4-dihydro-1-benzo[1,2,4]thiadiazin-3-yl)-1H-quinolin-2-one, 6-amino-1-(3-methylbutyl)-1H-benzo[d][1,3]oxazine-2,4-dione, 6-amino-4-hydroxy-3-(7-methoxy-1,1-dioxo-1,2-dihydrobenzo[1,2,4]thiadiazin-3-yl)-1-3-methylbutyl)-1H-quinolin-2-one, 6-amino-4-hydroxy-3-(7-hydroxy-1,1-dioxo-1,2-dihydrobenzo[1,2,4]thiadiazin-3-yl)-1-(3-methylbutyl)-1H-quinolin-2-one, and 1-(3-nitrobenzyl)-1H-benzo[d][1,3]oxazine-2,4-dione, 1-[(2-methylpyridin-4yl)methyl]-1H-benzo[d][1,3]oxazine-2,4-dione, or a tautomer thereof, or a pharmaceutically acceptable salt or solvate thereof.

The activity of the inventive compounds as inhibitors of HCV activity may be measured by any of the suitable methods known to those skilled in the art, including in vivo and in vitro assays. For example, the HCV NS5B inhibitory activity of the compounds of Formulas I, II and III was determined using standard assay procedures described in Behrens et al., EMBO J. 15:12-22 (1996), Lohmann et al., Virology 249:108-118 (1998) and Ranjith-Kumar et al., J. Virology 75:8615-8623 (2001). Unless otherwise noted, the compounds of this invention have demonstrated in vitro HCV NS5B inhibitory activity in such standard assays and have IC₅₀'s in the range of 0.0001 μM to 100 μM. Representative compounds of Formula I, Examples 10-20, 60-75, 110-120, 130-139, and 141-160 have all demonstrated in vitro HCV NS5B inhibitory activity and have IC₅₀'s in the range of 0.0005 μM to 3 μM. Recently, cell-based replicon systems for HCV have been developed, in which the nonstructural proteins stably replicate subgenomic viral RNA in Huh7 cells (Lohmann et al., Science (1999) and Blight et al., Science (2000). In the absence of a purified, functional HCV replicase consisting of viral non-structural and host proteins, our understanding of Flaviviridae RNA synthesis comes from studies using active recombinant RdRps and validation of these studies in the HCV replicon system. Inhibition of recombinant purified HCV polymerase with compounds in in vitro biochemical assays may be validated using the replicon system whereby the polymerase exists within a replicase complex, associated with other viral and cellular polypeptides in appropriate stoichiometry. Demonstration of cell-based inhibition of HCV replication may be more predictive of in vivo function than demonstration of HCV NS5B inhibitory activity in in vitro biochemical assays.

Advantageously, the compounds of this invention inhibit both positive and negative strand HCV-RNA replication. The following methods have been developed and used for determining the positive and negative strand HCV-RNA replication inhibition activity of the compounds of this invention.

### Test Method 1

Method for Positive Strand Replicon HCV-RNA Detection in Replicon Cells

Replicon cells were plated at 3×10³ cells per well in a 96-well plate plates at 37° and 5% CO₂ in DMEM (Dulbecco's Minimal Essential Medium) containing 10% FCS (fetal calf serum), 1% NEAA (nonessential amino acids) and 1 mg/ml Geneticin (G418 neomycin). After allowing 4 h for cell attachment, 1 μl of a solution of candidate antiviral agent was added to the medium (n=8 wells per dilution). Briefly, eleven 2.5-fold dilutions of 1 mM stock test compound in DMSO (dimethylsulfoxide) were prepared with final concentration ranging from 10000 nM to 1.0 nM. Plates were incubated for 40 h, until reaching 80% confluence. After removal of medium, 150 μl Buffer RLT (Qiagen, Valencia, Calif., US) was added to each well and RNA purified according to manufacturer's recommendations (Qiagen RNAeasy) and were eluted twice in 45 μl dH₂O prior to RT-PCR. Approximately 40 μl of TaqMan EZ RT-PCR (Applied Biosystems, Foster City, Calif., US) master mix (1× TaqMan EZ Buffer, 3 mM Mn(OAc)₂, 0.3 mM dATP, 0.3 mM dCTP, 0.3 mM dGTP, 0.6 mM dUIP, 0.2 mM neo-forward, 0.2 mM neo-reverse, 0.1 mM neo-probe, 1× Cyclophilin Mix, 0.1 Unit/μl rTth DNA Polymerase, 0.01 Unit/μl AmpErase UNG, and H₂O to 40 μl) was added to each tube of 96-tube optical plate along with 10 μl of RNA elution. Primers and probes specific for the positive strand RNA detection of neomycin gene were: neo-forward: 5′CCGGCTACCTGCCCATTC3′ (SEQ ID NO 1); neo-reverse: 5′CCAGATCATCCTGATCGACAAG3′ (SEQ ID NO 2); neo-probe: 5′FAMACATCGCATCGAGCGAGCACGTAC-TAMRA3′ (SEQ ID NO 3). For negative strand RNA detection, the cDNA primer used was 5′ACA TGC GCG GCA TCT AGA CCG GCT ACC TGC CCA TTC3′ (SEQ ID NO 4) whereby the first 18 bases represent SEQ ID NO 5 linked to neo sequences; neo-forward tag: 5′ACA TGC GCG GCA TCT AGA3′ (SEQ ID NO 5); neo reverse 5′CCAGATCATCCTGATCGACAAG3′ (SEQ ID NO 6); neo probe: 5′FAM-ACA TCG CAT CGA GCG AGC ACG TAC-TAMRA3′ (SEQ ID NO 3). Additionally, the PDAR control reagent human cyclophilin was used for normalization. Samples were mixed briefly and placed in an ABI7700 (Applied Biosystems) at 50° C., 2 min; 60° C., 30 min; and 95° C., 5 min, with cycling parameters set to 94° C., 20 s; 55° C., 1 min for 40 cycles. The relative cDNA levels for neo and cyclophilin were determined compared to DMSO-only treated controls and the ratio of neo:cyclophilin was used for IC₅₀ calculation (n=8).

### Test Method 2

Method for Negative Strand Replicon HCV-RNA Detection in Replicon Cells

To achieve strand-specific detection, a primer containing HCV RNA (or replicon RNA sequences such as neomycin gene) and an 18 base tag of nonrelated sequence at the 5′ end was for the reverse transcription (RT) reaction, 5′ACATGCGCGGCATCTAGACCGGCTACCTGCCCATTC3′ (SEQ ID NO 4). A Thermoscript-RT-PCR system (Invitrogen) was used for the RT reaction according to the manufacturer's protocol, with approximately 9 μl of the cell-harvested RNA and 1 μl of primer (10 μM incubated with RT at 60° C. for 1 h. Following that incubation, 2 μl of cDNA product containing the 5′ tag was amplified for TaqMan quantification using the 48 μl of TaqMan Universal Master Mix (Applied Biosystems) as well as primers, neo-forward tag: 5′ACA TGC GCG GCA TCT AGA3′ (SEQ ID NO 5); neo reverse: 5′CCAGATCATCCTGATCGACAAG3′ (SEQ ID NO 6); and neo probe: 5′FAM-ACA TCG CAT CGA GCG AGC ACG TiAC-TAMRA3′ (SEQ ID NO 3). Samples were mixed briefly and placed in an ABI7700 (Applied Biosystems) at 50° C., 2 min; 95° C., 10 min, with cycling parameters set to 94° C., 15 s; 55° C., 1 min for 40 cycles. The negative strand copy number in each reaction was determined using linear regression analysis based on the slope and intercept generated with a negative strand copy standard curve. The negative strand copies per cell were determined by dividing the total negative strand copies per reaction by the total cells per reaction.

Through routine experimentation, including appropriate manipulation and protection of any chemical functionality, synthesis of the compounds of Formulas I, II and m is accomplished by methods analogous to those above and to those described in the following Experimental section.

### EXAMPLE 1

3-1,1-Dioxo-1,2-dihydrobenzo[1,2,4]thiadiazin-3-yl)-4-hydroxy-1-2-phenethyl)-1H-quinolin-2-one

a) 1-Phenethyl-1H-benzo[d][1,3]oxazine-2,4-dione

A suspension of 1H-benzo[d][1,3]oxazine-2,4-dione (0.122 g, 0.747 mmol) in anhydrous dimethylformamide (2.0 mL) was treated with sodium hydride (60% dispersion in mineral oil) (0.032 g; 0.8 mmol). After stirring at room temperature for 30 min, a solution of (2-bromoethyl)benzene (0.169 g, 0.911 mmol) in dimethylformamide (1.0 mL) was added and the reaction was heated at 70° C. for 2h. The reaction was cooled to room temperature and poured into 1M HCl. The product was extracted with ethyl acetate, washed with brine, dried (Na₂SO₄) and concentrated on a rotary evaporator to give the title compound (127 mg, 64%) as an off-white solid (127 mg, 64%).

b) 3-(1,1-Dioxo-1,2-dihydrobenzo[1,2,4]thiadiazin-3-yl)-4-hydroxy-1-(2-phenethyl)-1H-quinolin-2-one

A solution of the compound from Example 1a) (0.127 g, 0.46 mmol) and ethyl 1,1-dioxo-2H-benzo-1,2,4-thiadiazinyl-3-acetate (prepared by the method of Kovalenko, S. N.; Chernykh, V. P; Shkarlat, A. E.; Ukrainets, I. V.; Gridasov, V. I.; Rudnev, S. A., Chem. Heterocylc. Compd. (Engl. Transl.), 1998, 34(7), 791) (0.123 g, 0.46 mmol) in tetrahydrofuran (4.0 mL) was treated with sodium hydride (60% dispersion in mineral oil) (0.072 g; 1.84 mmol). The reaction was heated under reflux for 1 h, cooled to room temperature, and acetic acid (1.0 mL) was added. The reaction was again heated under reflux for 1 h then cooled to room temperature and poured into 1M aqueous hydrochloric acid. The resulting precipitate was filtered and crystallized from warm dimethyl sulfoxide to give the title compound as pale-yellow needles (75.3 mg, 35%). ¹HNMR (d₆-DMSO) δ 15.2 (br s, 1H), 14.3 (br s, 1H), 8.2 (d, 1H), 7.95 (m, 1H), 7.85 (m, 1H), 7.8 (m, 2H), 7.7 (m, 1H), 7.55 (t, 1H), 7.45 (m, 1H), 7.4-7.3 (m, 4H), 7.24 (m, 1H), 4.54 (t, 2H), 2.97 (t, 2H). MS(ES+) m/e 446 [M+H]⁺.

### EXAMPLE 2

3-(1,1-Dioxo-1,2-dihydrobenzo[1,2,4]thiadiazin-3-yl)-4-hydroxy-1-(2-cyanobenzyl)-1H-quinolin-2-one

Following the procedures of Examples 1a) and 1b), except substituting 2-(bromomethyl)benzonitrile for (2-bromoethyl)benzene, the title compound was prepared as a pale yellow solid (17.1 mg, 10%). ¹H NMR (d₆-DMSO) δ 15.3 (br s, 1H), 14.1 (br s, 1H), 8.25 (d, 1H), 7.95 (m, 2H), 7.75 (m, 3H), 7.5 (m, 5H), 7.0 (m, 1H), 5.8 (s, 2H). MS(ES+) m/e 457 [M+H]⁺.

### EXAMPLE 3

3-1,1-Dioxo-1,2-dihydrobenzo[1,2,4]thiadiazin-3-yl)-4-hydroxy-1-(3-phenylpropyl)-1H-quinolin-2-one

Following the procedure of Examples 1a) and 1b), except substituting (3-bromopropyl)benzene for (2-bromoethyl)benzene, the title compound was prepared as a pale yellow solid (79.2 mg, 46%). ¹H NMR (dr-DMSO) δ 15.2 (br s, 1H), 14.3 (br s, 1H), 8.2 (m, 1H), 8.0-7.7 (m, 51H), 7.55 (m, 1H), 7.45 (m, 1H), 7.25 (m, 5H), 4.4 (m, 2H), 2.75 (m, 2H), 2.0 (m, 2H). MS(ES+) m/e 460 [M+H].

### EXAMPLE 4

3-(1,1-Dioxo-1,2-dihydrobenzo[1,2,4]thiadiazin-3-yl)-4-hydroxy-1-(1-cyclopropylmethyl)-1H-quinolin-2-one

Following the procedure of Examples 1a) and 1b), except substituting (bromomethyl)cyclopropane for (2-bromoethyl)benzene, the title compound was prepared as pale yellow needle (161.0 mg, 35%). ¹HNMR (d₆-DMSO) δ 15.2 (br s, 1H), 14.4 (br s, 1H), 8.2 (d, 1H), 8.0-7.7 (m, 5H), 7.6 (m, 1H), 7.5 (m, 1H), 4.35 (m, 2H), 1.3 (m, 11, 0.55 (m, 4H). MS(ES+) m/e 396 [M+H]⁺.

### EXAMPLE 5

3-(1,1-Dioxo-1,2-dihydrobenzo[1,2,4]thiadiazin-3-yl)-4-hydroxy-1-(3-methylbutyl)-6-nitro-1H-quinolin-2-one

a) 1-(3-Methylbutyl)-6-nitrobenzo[d][1,3]oxazine-2,4-dione.

6-Nitro-1H-benzo[d][1,3]oxazine-2,4-dione (1.4 g, 6.7 mmol) was added portionwise to a suspension of sodium hydride (60% suspension in mineral oil) (300 mg, 7.5 mmol) in anhydrous dimethylformamide. After 15 min, 1-bromo-3-methylbutane (0.82 ml, 6.7 mmol) was added and the mixture was stirred at 700C for 6 h, then at ambient for 72 h.

The mixture was poured onto ice, acidified with acetic acetate, extracted into ethyl acetate and purified by chromatography (silica gel, 20% ethyl acetate in hexanes) to give the title compound (800 mg, 43%). ¹H NMR (300 MHz, d₆-DMSO) δ 8.62 (m, 1H), 8.58 (dd, 1H), 7.63 (d, 1H), 4.04-4.12 (m, 2H), 1.65-1.81 (m, 1H), 1.50 (m, 2H), 0.95 (d, 6H).

b) 3-(1,1-Dioxo-1,2-dihydrobenzo[1,2,4]thiadiazin-3-yl)-4-hydroxy-1-(3-methylbutyl)-6-nitro-1H-quinolin-2-one

Sodium hydride (165 mg of a 60% suspension in mineral oil, 4.0 mmol) was added to a mixture of the compound from Example 5a) (280 mg, 1.0 mmol) and ethyl 1,1-dioxo-2H-benzo-1,2,4-thiadiazinyl-3-acetate (270 mg, 1.0 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (250 mg, 55%). ¹H NMR (300 MHz, d₆-DMSO) δ 15.60 (s, 1H), 8.91 (d, J=3 Hz, 1H), 8.34 (dd, J=9 and 3 Hz, 1H), 7.69 (m, 1H), 7.58 (m, 1H), 7.45 (d, J=10 Hz, 1H), 4.15 (m, 2H), 1.7-1.95 (m, 3H), 1.0 (d, J=6.5 Hz, 6H). Anal. (C₂₁H₂₀N₄O₆S) calcd. C, 55.26; H, 4.42; N, 12.27; S, 7.02. found: C, 55.41; H, 4.63; N, 11.17; S, 6.99.

### EXAMPLE 6

6-Chloro-3-(1,1-dioxo-1,2-dihydrobenzo[1,2,4]thiadiazin-3-yl)-4-hydroxy-113-methylbutyl)-1H-quinolin-2-one

a) 6-chloro-1-(3-methylbutyl)benzo[d][1,3]oxazine-2,4-dione

6-Chloro-1H-benzo[d][1,3]oxazine-2,4-dione (1.27 g, 6.43 mmol) was added portionwise to a suspension of sodium hydride (60% suspension in mineral oil) (284 mg, 7.1 mmol) in anhydrous dimethylformamide. After 15 min, 1-bromo-3-methylbutane (0.9 ml, 7.1 mmol) was added and the mixture was stirred at 80° C. for 16 h.

The mixture was poured onto ice, acidified with acetic acid, and extracted into ethyl acetate. Purification using flash chromatography (20% ethyl acetate in hexanes) gave the title compound (1.0 g, 58%). ¹H NMR (300 MHz, CDCl₃) δ 8.35 (d, 1H), 7.85 (dd, 1H), 7.04 (d, 1H), 4.04 (m, 2H), 1.72 (m, 1H), 1.60 (m, 2H), 1.02 (d, J=6.5 Hz, 611).

b) 6-Chloro-3-(1,1-dioxo-1,2-dihydrobenzo[1,2,4]thiadiazin-3-yl)-4-hydroxy-1-(3-methylbutyl)-1H-quinolin-2-one

Sodium hydride (165 mg of a 60% suspension in mineral oil, 4.0 mmol) was added to a mixture of the compound from Example 6a) (267 mg, 1.0 mmol) and ethyl 1,1-dioxo-2H-benzo-1,2,4-thiadiazinyl-3-acetate (270 mg, 1.0 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (180 mg, 41%). ¹H NMR (300 MHz, d₆-DMSO) δ 15.94 (s, 1H), 8.06 (d, J=3 Hz, 1H), 7.68 (dd, J=8 and 1 Hz, 1H), 7.54-7.59 (m, 2H), 7.26-7.32 (m, 3H), 4.01 (m, 2H), 1.7 (m, 1H), 1.45 (m, 2H) 0.98 (d, J=6.5 Hz, 6H). Anal. (C₂₁H₂₀ClN₃O₄S) calcd. C, 56.56; H, 4.52; Cl, 7.95 N, 9.42; S, 7.19. found: C, 56.37; H, 4.25; N, 9.30; S, 7.50.

### EXAMPLE 7

6-Bromo-3-(1,1-dioxo-1,2-dihydrobenzo[1,2,4]thiadiazin-3-yl)-4-hydroxy-1-(3-methylbutyl)-1H-quinolin-2-one

a) 6-Bromo-1-(3-methylbutyl)-benzo[d][1,3]oxazine-2,4-dione

6-Bromo-1H-benzo[d][1,3]oxazine-2,4-dione (1.62 g, 6.69 mmol) was added portionwise to a suspension of sodium hydride (60% suspension in mineral oil) (296 mg, 7.4 mmol) in anhydrous dimethylformamide. After 15 min, 1-bromo-3-methylbutane (0.9 ml, 7.1 mmol) was added and the mixture was stirred at 80° C. for 16 h.

The mixture was poured onto ice, acidified with acetic acid, and extracted into ethyl acetate. Purification using flash chromatography (20% ethyl acetate in hexanes) gave the title compound (1.0 g, 48%). ¹H NMR (300 MHz, CDCl₃) δ 8.11 (d, J=3 Hz, 1H), 7.70 (dd, J=9 and 3 Hz, 1H), 7.10 (d, J=9 Hz, 1H), 4.04 (m, 2H), 1.62 (m, 1H) 1.60 (m, 2H), 1.02 (d, J=6.5 Hz, 6H).

b) 6-Bromo-3-(1,1-dioxo-1,2-dihydrobenzo[1,2,4]thiadiazin-3-yl)-4-hydroxy-1-(3-methylbutyl)-1H-quinolin-2-one

Sodium hydride (160 mg of a 60% suspension in mineral oil, 4.0 mmol) was added to a mixture of the compound from Example 7a) (312 mg, 1.0 mmol) and ethyl 1,1-dioxo-2H-benzo-1,2,4-thiadiazinyl-3-acetate (270 mg, 1.0 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound, (400 mg, 81%). ¹H NMR (300 MHz, d₆-DMSO) δ 15.93 (s, 1H), 8.20 (d, J=2.5 Hz, 1H), 7.67 (m, 2H), 7.59 (m, 1H), 7.30 (m, 3H), 4.10 (m, 2H), 1.75 (m, 1H), 1.45 (m, 2H), 1.0 (d, J=6.5 Hz, 6H). Anal. (C₂₁H₂₀BrN₃O₄S) calcd. C, 51.44; H, 4.11; Br, 16.29; N, 8.57; S, 6.54. found: C, 51.51; H, 3.84; Br, 16.35; N, 8.47; S, 6.70.

### EXAMPLE 8

3-(1,1-Dioxo-1,2-dihydrobenzo[1,2,4]thiadiazin-3-yl)-4-hydroxy-6-methoxy-1-(3-methylbutyl)-H-quinolin-2-one

a) 6-Methoxyl-(3-methylbutyl)benzo[d][11,3]oxazine-2,4-dione

6-Methoxy-1H-benzo[d][1,3]oxazine-2,4-dione (1.16 g, 6.0 mmol) was added portionwise to a suspension of sodium hydride (60% suspension in mineral oil) (265 mg, 6.6 mmol) in anhydrous dimethylformamide. After 30 min, 1-bromo-3-methylbutane (0.8 ml, 6.7 mmol) was added and the mixture was stirred at 80° C. for 16 h.

The mixture was poured onto ice, acidified with acetic acid, and extracted into ethyl acetate. Purification using flash chromatography (20% ethyl acetate in hexanes) gave the title compound (850 mg, 54%). ¹H NMR (300 MHz, CDCl₃) δ 7.56 (d, J=3 Hz, 1H), 7.32 (dd, J=9 and 3 Hz, 1H), 7.08 (d, J=9 Hz, 1H) 4.03 (m, 2H), 3.87 (s, 3H) 1.75 (m, 1H), 1.62 (m, 2H) 1.01 (d, J=6.5 Hz, 6H).

b) 3-(1,1-Dioxo-1,2-dihydrobenzo[1,2,4]thiadiazin-3-yl)-4-hydroxy-6-methoxy-1(3-methylbutyl)-H-quinolin-2-one

Sodium hydride (160 mg of a 60% suspension in mineral oil, 4.0 mmol) was added to a mixture of the compound from Example 8a) (267 mg, 1.0 mmol) and ethyl 1,1-dioxo-2H-benzo-1,2,4-thiadiazinyl-3-acetate (270 mg, 1.0 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (330 mg, 75%) mp 273-50C. ¹H NMR (300 MHz, d₆-DMSO) δ 15.20 (s, 1H), 15.07 (s, 1H), 7.4-8.0 (m, 7H), 4.30 (m, 2H), 3.86 (s, 3H), 1.76 (m, 1H), 1.54 (m, 2H), 1.0 (d, J=6.5 Hz, 6H). Anal. (C₂₂H₂₃N₃O₅S) calcd. C, 59.85; H, 5.25; N, 9.52; S, 7.26. found: C, 59.77; H, 5.25; N, 9.38; S, 7.30.

### EXAMPLE 9

1-But-3-enyl-3-1,1-dioxo-1,2-dihydrobenzo[1,2,4]thiadiazin-3-yl)-4-hydroxy-1H-quinolin-2-one

Following the procedures of Examples 1a) and 1b) except substituting 4-bromobut-1-ene for (2-bromoethyl)benzene, the title compound was prepared (550 mg, 60%) as a tan solid. mp 233.9-237.0° C. (dec.). ¹H-NMR (d₆DMSO) δ 15.23 (br s, 1H); 14.35 (s, 1H); 8.22 (dd, 1H, J=8.1, 1.4 Hz); 7.69-7.87 (m, 2H); 7.82-7.71 (m, 3H); 7.57 (td, 1H, J=8.1, 1.2 Hz); 7.47 (t, 1H, J=7.5 Hz); 5.96 (ddt, 1H, J=17.2, 10.1, 6.7 Hz); 5.17-5.06 (m, 2H); 4.43 (t, 2H, J=7.6 Hz); 2.43-2.51 (m, 2H). MS(ES+) m/e 396 [M+H]⁺.

### EXAMPLE 10

1-(3-Bromobutyl)-3-(1,1-dioxo-1,2-dihydrobenzo[1,2,4]thiadiazin-3-yl)-4-hydroxy-1-1H-quinolin-2-one

A stirred suspension of the compound from Example 9 (150 mg, 0.379 mmol) in glacial acetic acid (2.5 mL) was treated dropwise with hydrobromic acid (30% solution in glacial acetic acid) (1.5 mL). The mixture was then heated under reflux for 2.5h, cooled to room temperature and partitioned between chloroform and water. The separated organic layer was washed with saturated sodium hydrogen carbonate solution, dried over sodium sulfate and evaporated to afford the title compound (150 mg; 83%) as a tan solid. mp 190-194.5° C. (dec.). ¹H-NMR (d₆-DMSO) δ 15.22 (br s, 1H); 14.27 (s, 1H); 8.23 (dd, 1H, J=8.1, 1.5 Hz); 7.69-7.89 (m, 2H); 7.82-7.69 (m, 3H); 7.57 (td, 1H, J=8.1, 1.0); 7.48 (t, 1H, J=7.5 Hz); 4.60-4.51 (m, 2H); 4.464.37 (m, 1H); 2.14-2.22 (m, 2H); 1.78 (d, 3H, J=6.6 Hz). MS(ES+) m/e 478 [M+H]⁺.

### EXAMPLE 11

1-Cyclohexylmethyl-3-(1,1-dioxo-1,2-dihydrobenzo[1,2,4]thiadiazin-3-yl)-4-hydroxy-1H-quinolin-2-one

Following the procedure of Example 1a) and 1b) except substituting (bromomethyl)cyclohexane for (2-bromoethyl)benzene, the title compound was prepared as a tan powder. mp 232.0-235.0° C. ¹H-NMR (d₆-DMSO) δ 15.26 (br s, 1H); 14.42 (br s, 1H); 8.21 (dd, 1H, J=8.1, 1.4 Hz); 7.96-7.69 (m, 5H); 7.57 (td, 1H, J=8.0, 1.1 Hz); 7.46 (t, 1H, J=7.4 Hz); 4.354.19 (m, 2H); 1.92-1.81 (m, 1H); 1.69-1.53 (m, 5H); 1.30-1.15 (m, 5H). MS(ES+) m/e 438 [M+H]⁺.

### EXAMPLE 12

3-(1,1-Dioxo-1,2-dihydrobenzo[1,2,4]thiadiazin-3-yl)-4-hydroxy-1-(3-methylbut-2-eneyl)-1H-quinolin-2-one

Following the procedure of Example 1a) and 1b) except substituting 4-bromo-2-methyl-2-butene for (2-bromoethyl)benzene, the title compound was prepared as a tan powder. mp 249.1-251.1° C. ¹H-NMR (d₆-DMSO) δ 15.19 (bs, 1H); 14.31 (bs, 1H); 8.21 (dd, 1H, J=8.1, 1.3 Hz); 7.96-7.54 (m, 6H); 7.46 (t, 1H, J=7.5 Hz); 5.19-5.09 (m, 1H); 5.05-4.93 (m, 2H); 1.89 (s, 3H); 1.71 (m, 3H). MS(ES+) m/e 410 [M+H]⁺.

### EXAMPLE 13

3-(1,1-Dioxo-1,2-dihydrobenzo[1,2,4]thiadiazin-3-yl)+hydroxy-6-methyl-1-(3-methylbutyl)-1H-quinolin-2-one

a) 6-Methylbenzo[d][1,3]oxazine-2,4-dione

A solution of 5-methylanthranilic acid (1.0 g, 6.6 mmol) in tetrahydrofuran (20 mL) was treated with a 20% solution of phosgene in toluene (4.0 mL) and stirred overnight. Saturated sodium hydrogen carbonate solution was added and the mixture extracted with ethyl acetate. Evaporation of the organic layer gave the title compound (1.01 g, 86%). ¹H NMR (300 MHz, d₆-DMSO) δ 11.66 (br s, 1H), 7.72 (d, J=2 Hz, 1H), 7.57 (dd, J=8 and 2 Hz 1H) 7.06 (d, J=8 Hz, 1H), 2.33 (s, 3H).

b) 1-(3-Methylbutyl)-6-methylbenzo[d][1,3]oxazine-2,4dione

The compound from Example 13a) (1.0 g, 5.6 mmol) was added portionwise to a suspension of sodium hydride (60% suspension in mineral oil) (250 mg, 6.25 mmol) in anhydrous dimethylformamide. After 30 min, 1-bromo-3-methylbutane (0.9 ml, 7.1 mmol) was added and the mixture was stirred at 80° C. for 16 h.

The mixture was poured onto ice, acidified with acetic acid, and extracted into ethyl acetate. Purification using flash chromatography (20% ethyl acetate in hexanes) gave the title compound, (592 mg, 43%). ¹H NMR (300 MHz, d₆-DMSO) δ 7.82 (d, J=1 Hz, 1H), 7.68 (dd, J=8 and 2 Hz, 1H) 7.33 (d, J=8 Hz, 1H), 4.00 (m, 2H), 2.37 (s, 1H), 1.70 (m, 1H), 1.52 (m, 2H) 0.96 (d, J=6.5 Hz, 6H).

c) 3-(1,1-Dioxo-1,2-dihydrobenzo[1,2,4]thiadiazin-3-yl)-4-hydroxy-6-methyl-1-(3-methylbutyl)-1H-quinolin-2-one

Sodium hydride (130 mg of a 60% suspension in mineral oil, 3.25 mmol) was added to a mixture of the compound from Example 13b) (200 mg, 0.8 mmol) and ethyl 1,1-dioxo-2H-benzo-1,2,4-thiadiazinyl-3-acetate (215 mg, 0.8 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (152 mg, 45%). ¹H NMR (300 MHz, d₆-DMSO) δ 15.12 (s, 1H), 14.39 (s, 1H), 7.94 (d, J=8 Hz, 1N), 7.50-7.78 (m, 6H), 4.29 (m, 2H), 2.38 (s, 1H), 1.81 (m, 1H), 1.53 (m, 2H) 0.99 (d, J=6.5 Hz, 6H). Anal. (C₂₂H₂₃N₃O₄S) calcd: C, 62.10; H, 5.45; N, 9.88; S, 7.54. Found: C, 62.11; H, 5.36; N, 9.68; S, 7.63.

### EXAMPLE 14

3-(1,1-Dioxo-1,2-dihydrobenzo[1,2,4]thiadiazin-3-yl)-6-fluoro-4-hydroxy-1-(3-methylbutyl)-1H-quinolin-2-one

a) 6-Fluorobenzo[d][1,3]oxazine-2,4-dione

A solution of 5-fluoroanthranilic acid (1.0 g, 6.44 mmol) in tetrahydrofuran (20 mL) was treated with a 20% solution of phosgene in toluene (4.0 mL) and stirred overnight. Saturated sodium hydrogen carbonate solution was added and the mixture extracted with ethyl acetate. Evaporation of the organic layer gave the title compound that crystallized from ether. Filtered, washed with diethyl ether and hexanes, (960 mg, 82%). ¹H NMR (300 MHz, CDCl₃) δ 11.81 (s, NH), 7.70 (m, 2H), 7.67 (m, 1H).

b) 1-(3-Methylbutyl)-6-fluorobenzo[d][1,3]oxazine-2,4-dione

The compound from Example 13a) (940 mg, 5.2 mmol) was added portionwise to a suspension of sodium hydride (60% suspension in mineral oil) (240 mg, 6.0 mmol) in anhydrous dimethylformamide. After 30 min, 1-bromo-3-methylbutane (0.9 ml, 7.1 mmol) was added and the mixture was stirred at 80° C. for 16h.

The mixture was poured onto ice, acidified with acetic acid, and extracted into ethyl acetate. The title compound crystallized from hexanes (380 mg, 23%). ¹H NMR (300 MHz, d₆-DMSO) δ 7.72-7.81 (m, 2H), 7.47-7.51 (m, 1H), 4.02 (m, 2H) 1.73 (m, 1H), 1.54 (m, 2H) 0.96 (d, J=6.5 Hz, 6H).

c) 3-(1,1-Dioxo-1,2-dihydrobenzo[1,2,4]thiadiazin-3-yl)-6-fluoro-4-hydroxy-1-(3-methylbutyl)-1H-quinolin-2-one

Sodium hydride (130 mg of a 60% suspension in mineral oil, 3.25 mmol) was added to a mixture of 1-(3-methylbutyl)-6-fluorobenzo[d][1,3]oxazine-2,4-dione (250 mg, 0.8 mmol) and ethyl 1,1dioxo-2H-benzo-1,2,4-thiadiazinyl-3-acetate (215 mg, 0.8 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the tide compound (220 mg, 64%). ¹H NMR (300 MHz, d₆-DMSO) δ 15.10 (br s, 1H), 14.21 (s, 1H), 7.93 (d, J=7 Hz, 1H), 7.59-7.54 (m, 5H) 7.55 (dd, 1H), 4.29 (m, 2H), 1.78 (m, 1H), 1.52 (m, 2H) 0.99 (d, J=6.5 Hz, 6H). Anal. (C₂₁H₂₀FN₃O₄S) calcd: C, 58.73; H, 4.69; F, 4.42; N, 9.78; S, 7.47. Found: C, 58.54; H, 4.54; F, 4.83; N, 9.59; S, 7.41.

### EXAMPLE 15

3-(1,1-Dioxo-1,2-dihydrobenzo[1,2,4]thiadiazin-3-yl)-5-fluoro-4-hydroxy-1-(3-methylbutyl)-1H-quinolin-2-one

a) 5-Fluorobenzo[d][1,3]oxazine-2,4-dione

A solution of 6-fluoroanthranilic acid (1.4 g, 9.0 mmol) in tetrahydrofuran (20.0 mL) was treated with triphosgene (2.7 g, 9.0 mmol) and stirred at 50° C. overnight. Saturated sodium hydrogen carbonate solution was added and the mixture extracted with ethyl acetate. Evaporation of the organic layer gave the title compound (1.58 g, 97%). ¹H NMR (300 MHz, d₆-DMSO) δ 11.89 (s, 1H), 7.74 (m, 1H) 7.0 (m, 2H).

b) 1-(3-Methylbutyl)-5-fluorobenzo[d][1,3]oxazine-2,4-dione

The compound from Example 15a) (500 mg, 2.75 mmol), triphenyphosphine (721 mg, 2.75 mmol) and 3-methylbutanol (0.3 ml, 2.75 mmol) were stirred together in dichloromethane and treated with diisopropyl azodicarboxylate (0.54 ml, 2.75 mmol). The reaction was stirred under nitrogen overnight, evaporated onto silica and purified by chromatography (silica gel, ethyl acetate-hexanes) to give the title compound (520 mg, 75%). ¹H NMR (300 MHz, d₆-DMSO) 87.85 (m, 1H), 7.24 (d, J=9 Hz, 1H) 7.16 (dd, J=10 and 9 Hz, 1H), 4.00 (m, 2H), 1.73 (m, 1H), 1.52 (m, 2H) 0.96 (d, J=6.5 Hz, 6H).

c) 3-(1,1-Dioxo-1,2-dihydrobenzo[1,2,4]thiadiazin-3-yl)-5-fluoro-4-hydroxy-1-(3-methylbutyl)-1H-quinolin-2-one

Sodium hydride (130 mg of a 60% suspension in mineral oil, 3.25 mmol) was added to a mixture of 1-(3-methylbutyl)-5-fluorobenzo[d][1,3]oxazine-2,4-dione (200 mg, 0.8 mmol) and ethyl 1,1-dioxo-2H-benzo-1,2,4-thiadiazinyl-3-acetate (215 mg, 0.8 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (186 mg, 55%). ¹H NMR (300 MHz, d₆-DMSO) δ 15.60 (br s, 1H), 14.29-(s, 1H), 7.45-7.95 (m, 6H), 7.20 (m, 1H), 4.30 (m, 2H), 1.81 (m, 1H), 1.52 (m, 2H) 1.00 (d, J=6.5 Hz, 6H). Anal. (C₂₁H₂₀FN₃O₄S) calcd: C, 58.73; H, 4.69; F, 4.42; N, 9.78; S, 7.47. Found: C, 59.00; H, 4.85; F, 4.49; N, 9.28; S, 7.40.

### EXAMPLE 16

6,7-Difluoro-3-(1,1-dioxo-1,2-dihydrobenzo[1,2,4]thiadiazin-3-yl)-4-hydroxy-1-(3-methylbutyl)-1H-quinolin-2-one

a) 6,7-Difluorobenzo[d][1,3]oxazine-2,4-dione

A solution of 5,6-difluoroanthranilic acid (1.0 g, 5.8 mmol) in tetrahydrofuran (20 mL) was treated with triphosgene (1.73 g, 5.8 mmol) and stirred at 50° C. overnight. Saturated sodium hydrogen carbonate solution was added and the mixture extracted with ethyl acetate. Evaporation of the organic solution gave the title compound (1.03 g, 88%). ¹H NMR (300 MHz, d₆-DMSO) δ 11.91 (s, 1H), 7.97 (m, 1H) 7.10 (m, 1H).

b) 1-(3-Methylbutyl)-6,7-difluorobenzo[d][1,3]oxazine-2,4-dione

The compound from Example 16a) (547 mg, 2.75 mmol), triphenylphosphine (721 mg, 2.75 mmol) and 3-methylbutanol (0.3 ml, 2.75 mmol) were stirred together in dichloromethane and treated with diisopropyl azodicarboxylate (0.54 ml, 2.75 mmol). The reaction was stirred under nitrogen overnight, evaporated onto silica and purified by chromatography (silica gel, ethyl acetate-hexanes) to give the title compound (300 mg, 40%). ¹H NMR (300 MHz, d₆-DMSO) δ 7.90 (m, 1H), 6.95 (m, 1H), 3.98 (m, 2H), 1.73 (m, 1H), 1.62 (m, 2H) 0.99 (d, J=6.5 Hz, 6H).

c) 6,7-Difluoro-3-1,1-dioxo-1,2-dihydrobenzo[1,2,4]thiadiazin-3-yl)-4-hydroxy-1-(3-methylbutyl)-1H-quinolin-2-one

Sodium hydride (130 mg of a 60% suspension in mineral oil, 3.25 mmol) was added to a mixture of the compound from Example 16b) (215 mg, 0.8 mmol) and ethyl 1,1-dioxo-2H-benzo-1,2,4-thiadiazinyl-3-acetate (215 mg, 0.8 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (122 mg, 35%). ¹H NMR (300 MHz, d₆-DMSO) δ 15.20 (br s, 1H), 14.06 (s, 1H), 8.13 (m, 1H), 7.93 (d, J=8 Hz, 1H), 7.75-7.86 (m, 2H), 7.68 (d, J=8 Hz, 1H), 7.59 (m, 1H), 4.29 (m, 2H), 1.80 (m, 1H), 1.53 (m, 2H) 1.00 (d, J=6.5 Hz, 6H). Anal. (C₂₁H₁₉F₂N₃O₄S) calcd: C, 56.37; H, 4.28; F, 8.46; N, 9.39; S, 7.17. Found: C, 56.28; H, 4.12; F, 8.55; N, 9.37; S, 7.07.

### EXAMPLE 17

3-(1,1-Dioxo-1,2-dihydrobenzo[1,2,4]thiadiazin-3-yl)-7-fluoro-4-hydroxy-1-(3-methylbutyl)-1H-quinolin-2-one

a) 7-Fluorobenzo[d][1,3]oxazine-2,4-dione

A solution of 4-fluoroanthranilic acid (1.2 g, 7.7 mmol) in tetrahydrofuran (20 mL) was treated with triphosgene (2.3 g, 7.7 mmol) and stirred at 50° C. overnight. Saturated sodium hydrogen carbonate solution was added and the mixture extracted with ethyl acetate. Evaporation of the organic solution gave the title compound (1.09 g, 78%). ¹H NMR (300 MHz, d₆-DMSO) δ 11.88 (s, 1H), 8.00 (m, 1H) 7.11 (m, 1H), 6.88 (m, 1H).

b) 1-(3-Methylbutyl)-7-fluorobenzo[d][1,3]oxazine-2,4-dione

The compound from Example 17a) (500 mg, 2.75 mmol), triphenylphosphine (721 mg, 2.75 mmol) and 3-methylbutanol (0.3 ml, 2.75 mmol) were stirred together in dichloromethane and treated with diisopropyl azodicarboxylate (0.54 ml, 2.75 mmol). The reaction was stirred under nitrogen overnight, evaporated onto silica and purified by chromatography (silica gel, ethyl acetate-hexanes) to give the title compound (448 mg, 65%). ¹H NMR (300 MHz, d₆-DMSO) δ 8.09 (m, 1H), 7.37 (m, 1H), 7.20 (m, 1H), 4.00 (m, 2H), 1.71 (m, 1H), 1.52 (m, 2H) 0.96 (d, J=6.5 Hz, 6H).

c) 3-(1,1-Dioxo-1,2-dihydrobenzo[1,2,4]thiadiazin-3-yl)-7-fluoro-4-hydroxy-1-(3-methylbutyl)-1H-quinolin-2-one

Sodium hydride (130 mg of a 60% suspension in mineral oil, 3.25 mmol) was added to a mixture of the compound from Example 17b) (200 mg, 0.8 mmol) and ethyl 1,1-dioxo-2H-benzo-1,2,4-thiadiazinyl-3-acetate (215 mg, 0.8 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (46 mg, 15%). ¹H NMR (300 MHz, d₆-DMSO) δ 15.22 (br s, 1H), 14.11 (s, 1H), 8.13 (dd, J=6.5 and 9 Hz, 1H), 7.78 (m, 1H), 7.69 (d, J=8 Hz, 1H), 7.56 (m, 2H), 7.30 (m, 1H), 4.29 (m, 2H), 1.80 (m, 1H), 1.54 (m, 2H) 1.00 (d, I=6.5 Hz, 6H). Anal. (C₂₁H₂₀FN₃O₄S) calcd. C, 58.73; H, 4.69; F, 4.42; N, 9.78; S, 7.47. found: C, 58.59; H, 4.57; F, 4.99; N, 9.63; S, 7.35.

### EXAMPLE 18

3-(1, l-Dioxo-1,2-dihydrobenzo[1,2,4]thiadiazin-3-yl)-4-hydroxy-1-(4,4,4-trifluorobutyl)-1H-quinolin-2-one

Following the procedure of Example 1a) and 1b) except substituting 1-bromo-4,4,4-trifluorobutane for (2-bromoethyl)benzene, the title compound was prepared as white crystals. mp 248.9-250.0° C. ¹H-NMR (CDCl₃) δ 15.35 (s, 1H); 14.40 (br s, 1H); 8.34 (dd, 1H, J=8.1, 1.5 Hz); 8.00 (ddd, 1H, J=7.9, 1.4, 0.7); 7.79 (td, 1H, J=7.3, 1.5 Hz); 7.65 (td, 1H, J=7.4, 1.5 Hz); 7.50-7.29 (m, 4H); 4.42 (t, 2H, J=7.6 Hz); 2.39-2.27 (m, 2H); 2.12-2.00 (m, 2H). MS(ES+) m/e 452 [M+H]⁺.

### EXAMPLE 19

6-Amino-3-(1,1-dioxo-1,2-dihydrobenzo[1,2,4]thiadiazin-3-yl)-4-hydroxy-1-(3-methylbutyl)-1H-quinolin-2-one

A solution of the compound from Example 5b) (78 mg, 0.17 mmol) in dimethylformamide (20.0 mL) and acetic acid (2.0 mL) with 10% palladium on charcoal was shaken under an atmosphere of hydrogen at 50 psi for 2h. The mixture was filtered through Celite®, washed through with methanol and evaporated to a solid. The title compound was recrystallized from ethanol-4M HCl in dioxane as the hydrochloride salt. Yield (35 mg, 48% o). ¹H NMR (300 MHz, d₆-DMSO) δ 14.49 (s, 1H), 7.94 (d, J=8 Hz, 1H), 7.54-7.80 (m, 6H), 4.29 (m, 2H), 1.77 (m, 1H), 1.53 (m, 2H) 1.01 (d, J=6.5 Hz, 6H). Anal. (C21H22N4O4S, 1.4 HCl) calcd: C, 52.82; H, 4.94; N, 11.73; S, 6.71. found: C, 53.04; H, 4.89; N, 11.45; S. 7.11.

### EXAMPLE 20

3-(1,1-Dioxo-1,2-dihydrobenzo[1,2,4]thiadiazin-3-yl)-4-hydroxy-1-(4-hydroxybutyl)-1H-quinolin-2-one

A solution of the compound from Example 9 (150 mg, 0.38 mmol) in tetrahydrofuran (3.0 mL) was treated dropwise with a solution of 9-borabicyclo[3.3.1]nonane (0.5 M in tetrahydrofuran) (1.52 mL; 0.76 mmol) and the resulting solution was stirred at room temperature for 18 h. The reaction mixture was then quenched with a mixture of ethanol 10% aqu. sodium hydroxide/30% aqueous hydrogen peroxide solution and stirring was continued for additional 4h. The mixture was poured onto a mixture of ice and 1M aqu. hydrochloric acid and filtered and the residue purified by chromatography [ODS silica, gradient elution, 10-90% acetonitrile/water (0.1% TFA)]. to afford the title compound as a white powder. mp 238.4-239.6° C. ¹H-NMR (d₆-DMSO) δ 15.22 (bs, 1H); 14.42 (bs, 1H); 8.23 (dd, 1H, J=8.1, 1.3 Hz); 7.96-7.87 (m, 2H); 7.83-7.71 (m, 3H); 7.57 (td, 1H, 7.9, 1.1 Hz); 7.48 (t, 1H, J=7.7 Hz); 4.55 (bs, 1H); 4.37 (t, 2H, J=7.4 Hz); 3.48 (t, 1H, J=6.2 Hz); 1.80-1.70 (m, 2H); 1.69-1.51 (m, 2H). MS (ES+) m/e 414 [M+H]⁺.

### EXAMPLE 21

3-(1,1-Dioxo-7-nitro-1,2-dihydrobenzo[1,2,4]thiadiazin-3-yl)-4-hydroxy-1-(3-methylbutyl)-1H-quinolin-2-one

a) 1-(3-Methylbutyl)-1H-benzo[d][1,3]oxazine-2,4-dione

Di-isopropyl azodicarboxylate (0.602 mL, 3.06 mmol) was added dropwise to a stirred mixture of 1H-benzo[d][1,3]oxazine-2,4-dione (500 mg, 3.06 mmol), 3-methyl-1-butanol (0.33 mL, 3.06 mmol), triphenylphosphine (803 mg, 3.06 mmol) and dichloromethane (20.0 mL) at room temperature under argon. The mixture was stirred at room temperature for 18h, then the mixture was evaporated and the residue was purified by chromatography (silica gel, 25% ethyl acetate/hexanes) to give the title compound (490 mg, 69%) as an amorphous solid. ¹H NMR (CDCl₃) δ 8.17 (1H, m), 7.77 (1H, m), 7.30 (1H, t, J=8.0 Hz), 7.16 (1H, d, J=8.5 Hz), 4.07 (2H, m), 1.76 (1H, m), 1.64 (2H, m), 1.03 (6H, d, J=6.5 Hz).

b) 3-Cyano-4-hydroxy-1-(3-methylbutyl)-1H-quinolin-2-one

Sodium hydride (60% dispersion in mineral oil) (2.0 g; 50.0 mmol) was added to a stirred solution of the compound from Example 21a) (5.27 g, 22.6 mmol) and methyl cyanoacetate (4.0 mL, 45.3 mmol) in dimethylformamide (50.0 mL) under argon. When the gas evolution had stopped, the mixture was stirred at 100° C. for 3 h, then cooled. Acetic acid (10.0 mL, 175 mmol) was added carefully, and the mixture heated once more at 100° C. for 1 h, then cooled and poured into water (500 mL). The pH was adjusted to 1 with dilute aqueous hydrochloric acid. Ether (50.0 mL) was added and the mixture shaken gently while cooling in ice for 1 h. The solid was filtered, washed (water, then diethyl ether) and dried to give the title compound (3.68 g, 64% o) as a cream solid. ¹H NMR (d₆-DMSO) δ 8.12 (1H, m), 7.78 (1H, m), 7.53 (1H, d, J=8.6 Hz), 7.33 (1H, t, J=7.4 Hz), 4.18 (2H, m), 1.69 (1H, m), 1.46 (2H, m), 0.97 (6H, d, J=6.6 Hz).

c) 4-Hydroxy-1-(3-methylbutyl)-2-oxo-1,2-dihydroquinoline-3-carboxamidine

A solution of trimethylaluminum (2M in hexanes) (0.585 mL, 1.17 mmol) was added dropwise to a stirred mixture of ammonium chloride (63 mg, 1.17 mmol) and toluene (2.0 mL) under argon and the mixture stirred at room temperature for 1 h. The compound from Example 21b) (100 mg, 0.390 mmol) was added and the mixture heated at 50° C. for 18h, then cooled. Water (0.40 mL, 22.2 mmol) was added dropwise, followed by ethyl acetate (10.0 mL). The mixture was stirred for 10 min., then excess aqueous sodium hydrogen carbonate was added and stirring continued for 1 h. The mixture was filtered and the filtrate evaporated under reduced pressure. The residue was purified by chromatography (silica gel, ethyl acetate) to give the title compound (46 mg, 43%) as a white amorphous solid. ¹H NMR (d₆-DMSO) δ 10.72 (2H, br s), 8.11 (1H, m), 7.61 (1H, m), 7.45 (2H, br d), 7.34 (1H, d, J=8.3 Hz), 7.16 (1H, t, J=7.3 Hz), 4.14 (2H, m), 1.71 (1H, m), 1.46 (2H, m), 0.98 (6H, d, J=6.6 Hz).

d) 2-Chloro-5-nitrobenzenesulfonyl Chloride

A stirred mixture of 2-chloro-5-nitrobenzenesulfonic acid (1.0 g, 4.21 mmol), thionyl chloride (10.0 mL, 137 mmol) and dimethylformamide (0.05 mL, 0.65 mmol) was heated under reflux for 4h, cooled and poured on to ice. The mixture was extracted with ether, and the extracts washed (water, saturated aqueous sodium chloride), dried and evaporated under reduced pressure to give the title compound (840 mg, 78%) as an oil. ¹H NMR (CDCl₃) δ 9.01 (1H, d, J=2.6 Hz), 8.52 (1H, dd, J=8.7 Hz), 7.88 (1H, d, J=8.7 Hz).

e) 3-(1,1-Dioxo-7-nitro-1,2-dihydrobenzo[1,2,4]thiadiazin-3-yl)-4-hydroxy-1-(3-methylbutyl)-1H-quinolin-2-one

Sodium hydride (60% dispersion in mineral oil) (44 mg; 1.10 mmol) was added to a stirred solution of 4-hydroxy-1-(3-methylbutyl)-2-oxo-1,2-dihydroquinoline-3-carboxamidine (100 mg, 0.366 mmol) in tetrahydrofuran (3.0 mL) under argon. After 5 min., 2-chloro-5-nitrobenzenesulfonyl chloride (98 mg, 0.384 mmol) was added and the mixture stirred at room temperature for 18h, then partitioned between aqueous hydrochloric acid and ethyl acetate. The extracts were washed (water, saturated aqueous sodium chloride), dried and evaporated under reduced pressure. The residue was purified by chromatography (silica gel, 30-50% ethyl acetate/hexane) to give the sulfonylated amidine intermediate. Sodium hydride (60% dispersion in mineral oil) (36 mg; 0.90 mmol) was added to a stirred solution of this intermediate in tetrahydrofuran (5.0 mL) under argon and the mixture heated under reflux for 2h, then cooled. Dilute aqueous hydrochloric acid was added and the solid filtered, washed with water and dried. The residue was purified by chromatography (silica gel, 30-50% ethyl acetate/hexane, then 5% methanol/dichloromethane) to give the title compound (16 mg, 10%) as a white amorphous solid. ¹H NMR (dr-DMSO) δ 8.51 (1H, d, J=2.1 Hz), 8.44 (1H, m), 8.19 (1H, m), 7.73 (2H, m), 7.48 (1H, m), 7.29 (1H, m), 4.23 (2H, m), 1.77 (1H, m), 1.51 (2H, m), 1.01 (6H, d, J=6.6 Hz).

### EXAMPLE 22

3-(1,1-Dioxo-1,2-dihydrobenzo[1,2,4]thiadiazin-3-yl)-4-hydroxy-1-pent-4-ynyl-1H-quinolin-2-one

a) 1-Pent-4-ynyl-1H-benzo[d][1,3]oxazine-2,4-dione

Diisopropyl azodicarboxylate (0.663 mL, 3.37 mmol) was added dropwise to a stirred suspension of 1H-benzo[d][1,3]oxazine-2,4dione (500 mg, 3.06 mmol), triphenylphosphine (883 mg, 3.37 mmol) and 4-pentyn-1-ol (0.313 mL, 3.37 mmol). The resulting solution was stirred at room temperature for 18 h, reduced in volume and purified by flash chromatography (hexanes/ethyl acetate 7:3) to afford the product (17%).

b) 3-(1,1-Dioxo-1,2-dihydrobenzo[1,2,4]thiadiazin-3-yl)-4-hydroxy-1-pent-4-ynyl-1H-quinolin-2-one

Following the procedure of Example 9, except substituting the compound from Example 22a) for 1-but-3-enyl-1H-benzo[d][1,3]oxazine-2,4-dione, the title compound was prepared as a white crystalline solid. MS (ES+) m/e 408 [M+H⁺. mp 221.8 (dec.); 254.1-255.6° C. ¹H-NMR (CDCl₃) δ 15.29 (s, 1H); 14.51 (bs, 1H); 8.32 (dd, 1H, J=8.1, 1.5 Hz); 8.01 (dd, 1H, J=7.7, 1.3 Hz); 7.78 (td, 1H, J=8.6, 1.6 Hz); 7.66-7.29 (m, 5H); 4.46 (t, 2H, J=7.8 Hz); 2.43 (td, 2H, J=6.7, 2.5 Hz); 2.13 (t, 1H, J=2.5 Hz); 2.06-1.98 (m, 2H).

### EXAMPLE 23

3-(7-Bromo-1,1-dioxo-1,2-dihydrobenzo[1,2,4]thiadiazin-3-yl)-4-hydroxy-1-(3-methylbutyl)-1H-quinolin-2-one

a) Methyl (7-bromo-1,1-dioxo-1,2-dihydrobenzo[1,2,4]thiadiazin-3-yl)acetate

Ethyl 3-chloro-3-oxopropionate (1.31 mL, 10.2 mmol) was added over 5 min into an ice-cooled, stirred solution of 2-amino-5-bromobenzenesulfonamide (2.57 g, 10.2 mmol) (A. H. Gouliaev et. al., PCT Application WO9942456, 1999), triethylamine (4.20 mL, 30.1 mmol) and dimethylaminopyridine (61 mg, 0.5 mmol) in tetrahydrofuran (40.0 mL) under argon. After stirring at room temperature for 3h, volatiles were removed under reduced pressure and the residue was partitioned between 1M aqueous hydrochloric acid and ethyl acetate. The extracts were washed (water, saturated aqueous sodium chloride), dried and evaporated under reduced pressure. The residue was stirred in 10% aqueous sodium carbonate (200 mL) for 2h, then the mixture acidified to pH 1 with concentrated aqueous hydrochloric acid. The mixture was filtered and the filtrate extracted with ethyl acetate. The extracts were combined with the filtration residue and the solvent removed under reduced pressure. The residue was heated under reflux in methanol (50.0 mL) and concentrated sulfuric acid (3.0 mL) for 18h, then cooled. Most of the solvent was removed under reduced pressure, then saturated aqueous sodium chloride (200 mL) was added and the mixture extracted with ethyl acetate. After washing (saturated aqueous sodium chloride) and drying the extracts were evaporated under reduced pressure and the residue was purified by chromatography (silica gel, 50-100% ethyl acetate/hexane) to give the tide compound (0.51 g, 14%) as an amorphous solid. ¹H NMR (d₆-DMSO) δ 12.47 (1H, br s), 8.00 (1H, d, J=2.2 Hz), 7.89 (1H, dd, J=8.8, 2.3 Hz), 7.31 (1H, d, J=8.8 Hz), 3.75 (2H, s), 3.71 (3H, s).

b) 3-(7-Bromo-1,1-dioxo-1,2-dihydrobenzo[1,2,4]thiadiazin-3-yl)-4-hydroxy-1-(3-methylbutyl)-1H-quinolin-2-one

Sodium hydride (52 mg of a 60% oil suspension, 1.29 mmol) was added to a stirred solution of 1-(3-methylbutyl)-1H-benzo[d][1,3]oxazine-2,4-dione (100 mg, 0.429 mmol) and methyl (7-bromo-1,1-dioxo-1,2-dihydrobenzo[1,2,4]thiadiazin-3-yl)acetate (143 mg, 0.429 mmol) in tetrahydrofuran (3.0 mL) under argon. After 5 min., the mixture was heated under reflux for 2h, then cooled. Acetic acid (2.0 mL, 35 mmol) was added carefully and the mixture heated under reflux again for 1 h and cooled. Water (50.0 mL) was added, the mixture stirred for 0.5h and filtered. The solid was washed with water and ether and dried to give the title compound (120 mg, 57%) as a cream powder. ¹H NMR (d₆-DMSO) δ 14.92 (1H, br s), 14.45 (1H, br s), 8.22 (1H, m), 8.12 (1H, d, J=2.1 Hz), 7.98-7.89 (2H, m), 7.73-7.67 (2H, m), 7.47 (1H, m), 4.33 (2H, m), 1.78 (1H, m), 1.56 (2H, m), 1.02 (6H, d, J=6.6 Hz).

### EXAMPLE 24

3-(7-Amino-1,1-dioxo-1,2,4-dihydrobenzo[1,2,4]thiadiazin-3-yl)-4-hydroxy-1-(3-methylbutyl)-1H-quinolin-2-one

a) 1-[(3-Methyl)butyl)-3-(1,1-dioxo-2H-benzo-1,2,4-thiadiazin-3-yl)-4-hydroxy-2-quinolone

Following the procedure of Example 1a) and 1b), except substituting 3-methyl-1-bromobutane for (2-bromoethyl)benzene, the title compound was prepared as a pale yellow solid after recrystallization. MS (ES+) m/e 412 [M+H]⁺.

b) 3-(7-Amino-1,1-dioxo-1,2-dihydrobenzo[1,2,4]thiadiazin-3-yl)-4-hydroxy-1-(3-methylbutyl)-1H-quinolin-2-one;

Sodium nitrate (23.0 mg, 0.271 mmol) was added to an ice-cooled, stirred solution of the compound from Example 24a) (100 mg, 0.243 mmol) in concentrated sulfuric acid (1.5 mL) followed by sodium nitrite (0.5 mg, 0.007 mmol). The mixture was stirred at room temperature for 15h, then iced water (20.0 mL) added and the solid filtered, washed with water and dried. A solution of this solid in 20% ethyl acetate/methanol was stirred with 5% palladium-on-charcoal (14 mg) under hydrogen at room temperature and atmospheric pressure for 18 h, then hydrogen removed and the mixture filtered through Celite®. Solvent was removed under reduced pressure and the residue was purified by chromatography (silica gel, gradient elution 2-5% methanol/dichloromethane) to give the title compound (16 mg, 15%) as a yellow powder. ¹H NMR (d₆-DMSO) δ 15.53 (1H, br s), 14.11 (1H, br s), 8.21 (1H, dd, J=8.1, 1.4 Hz), 7.91 (1H, m), 7.69 (d, J=8.6 Hz), 7.49-7.41 (2H, m), 6.996.94 (2H, m), 5.88 (2H, br s), 4.35 (2H, m), 1.80 (1H, m), 1.56 (2H, m), 1.02 (6H, d, J=6.6 Hz).

### EXAMPLE 25

3-(7-Cyano-1,1-dioxo-1,2-dihydrobenzo[1,2,4]thiadiazin-3-yl)-4-hydroxy-1-(3-methylbutyl)-1H-quinolin-2-one

A mixture of the compound from Example 23b) (93 mg, 0.19 mmol), zinc cyanide (45 mg, 0.38 mmol), tetrakis(triphenylphosphine)palladium (0) (25 mg, 0.022 mmol) and dimethylformamide (1.0 mL) was stirred at 100° C. under argon for 18 h, then cooled and diluted with water (50.0 mL). The solid was filtered, washed with water and dried, then was purified by chromatography (silica gel, 0-2% methanol/dichloromethane). The product isolated was triturated with diethyl ether, filtered and dried to give the title compound (26 mg, 31%) as an off-white powder. ¹H NMR (d₆-DMSO) δ 14.61 (1H, br s), 8.58 (1H, d, J=1.7 Hz), 8.24-8.16 (2H, m), 7.94-7.88 (2H, m), 7.69 (1H, d, J=8.6 Hz), 7.47 (1H, t, J=7.6 Hz), 4.35 (2H, m), 1.81 (1H, m), 1.56 (2H, m), 1.02 (6H, d, J=6.6 Hz).

### EXAMPLE 26

8-Bromo-3-(1,1-dioxo-1,2-dihydrobenzo[1,2,4]thiadiazin-3-yl)-4-hydroxy-1H-quinolin-2-one

a) 8-Bromo-2H-3,1-benzoxazine-2,4(1H)-dione

A solution of 7-bromoindole-2,3-dione (prepared by the method of Katsifis and McPhee; Aust. J. Chem. 1999, 52, 1061-1069) (2.0 g) in glacial acetic acid (15.0 mL) was treated with 30% aqueous hydrogen peroxide solution (0.80 mL) The suspension was stirred at 50° C. for 5h and then concentrated. The residue was washed with diethyl ether to give the title compound as a yellow powder (1.8 g, 85%). MS(ES) m/e 244,242 [M+H]⁺.

b) 8-bromo-2-isobutoxybenzo[d][1,3]oxazin-4-one.

A solution of triphenylphosphine (1.26 g, 4.8 mmol) and 3-methylbutanol (0.42g, 4.8 mmol) was treated with the compound from Example 26a) (1.16g, 4.8 mmol). Diisopropyl azodicarboxylate (0.97g, 4.8 mmol) was then added dropwise to the resulting suspension and the solution was stirred at room temperature overnight then concentrated to give a gum. The gum was purified by chromatography (silica gel, 10% ethyl acetate/hexanes) to give the title compound as colorless gum (0.55g, 37%). ¹H NMR (300 MHz, CDCl₃) δ 8.1 (d, 1H), 8.0 (d, 1H), 7.2 (t, 1H), 4.6 (t, 2H), 1.8 (m, 3H), 1.0 (d, 6H).

c) 3-(1,1-Dioxo-1,2-dihydrobenzo[1,2,4]thiadiazin-3-yl)-8-bromo-4-hydroxy-1H-quinolin-2-one.

Sodium hydride (60% dispersion in mineral oil) (100 mg; 2.6 mmol) was added to a mixture of 8-bromo-2-isobutoxy-benzo[d][1,3]oxazin-4-one (200 mg, 0.6 mmol) and ethyl (1,1-dioxo-1,2-dihydrobenzo[1,2,4]thiadiazin-3-yl)acetate (172 mg, 0.6 mmol) in tetrahydrofuran (15.0 mL). The mixture was heated under reflux for 1.5h, cooled and acidified with glacial acetic acid. The mixture was then heated under reflux for an additional 1.5 h, cooled then quenched with water. The product was collected, washed with water, diethyl ether then hexanes to give the title compound (180 mg, 60%) as white powder. ¹H NMR (300 MHz, dr-DMSO) δ 14.3 (br, OH), 11.2 (br, NH), 8.2 (dd, J=8.1 Hz, J=1, 3 Hz, 1H) 8.0 (dd, J=7.8 Hz, J=1, 3 Hz, 1H), 7.9 (d, J=7.4 Hz, 1H), 7.8 (td, J=7.4 Hz, J=1.3 Hz, 1H), 7.7 (d, J=8.2 Hz, 1H), 7.6 (t, J=8.0 Hz, 1H), 7.4 (t, J=8.0 Hz, 1H). MS(ES+) m/de 422, 420 [M+H]⁺.

### EXAMPLE 27

6-Amino-3-(7-amino-1,1-dioxo-1,2-dihydrobenzo[1,2,4]thiadiazin-3-yl)-4-hydroxy-1-3-methylbutyl)-1H-quinolin-2-one

The procedure as described in Example 24b) also provided the title compound (9 mg, 8%). ¹H NMR (d₆-DMSO) δ 15.30 (1H, br s), 14.56 (1H, br s), 7.45-7.36 (2H, m), 7.29 (1H, d, J=2.6 Hz), 7.21 (1H, dd, J=9.0, 2.7 Hz), 6.99-6.93 (2H, m), 5.85 (2H, br s), 5.52 (2H, br s), 4.28 (2H, m), 1.77 (1H, m), 1.54 (2H, m), 1.00 (6H, d, J=6.6 Hz).

### EXAMPLE 28

1-Cyclopentylmethyl-3-(1,1-dioxo-1,2-dihydrobenzo[1,2,4]thiadiazin-3-yl)-4-hydroxy-1-1H-quinolin-2-one

a) 1-Cyclopropylmethyl-1H-benzo[d][1,3]oxazine-2,4-dione

Diisopropyl azodicarboxylate (0.663 mL, 3.37 mmol) was added dropwise to a stirred suspension of 1H-benzo[d][1,3]oxazine-2,4-dione (500 mg, 3.06 mmol), triphenylphosphine (883 mg, 3.37 mmol) and cyclopentanemethanol (0.365 mL, 3.37 mmol). The resulting solution was stirred at room temperature for 18h, evaporated and the residue purified by chromatography [silica, hexanes/ethyl acetate (3:1)] to afford the title compound (356 mg; 47%).

b) 1-Cyclopentylmethyl-3-(1,1-dioxo-1,2-dihydrobenzo[1,2,4]thiadiazin-3-yl)-4-hydroxy-1H-quinolin-2-one

Following the procedure of Example 9b), the title compound was prepared as a white crystalline solid after trituration with ethyl acetate. MS (ES+) m/e 424 [M+H]⁺. mp 254.6-255.8° C. ¹H-NMR (CDCl₃) δ 15.27 (s, 1H); 14.64 (bs, 1H); 8.30 (dd, 1H, J=8.0, 1.5 Hz); 7.98 (dd, 1H, J=7.9, 1.4 Hz); 7.74 (td, 1H, J=8.2, 1.6 Hz); 7.63 (td, 1H, J=8.0, 1.3 Hz); 7.48-7.31 (m, 4H); 4.35 (d, 2H, J=7.4 Hz); 2.55-2.31 (m, 1H); 1.79-1.39 (m, 8H).

### EXAMPLE 29

1-Cyclobutylmethyl-3-(1,1-dioxo-1,2-dihydrobenzo[1,2,4]thiadiazin-3-yl)-4-hydroxy-1-1H-quinolin-2-one

Following the procedure of Example 1a) and 1b) except substituting (bromomethyl)cyclobutane for (2-bromoethyl)benzene, the title compound was prepared as white crystals after trituration with ethyl acetate. MS (ES+) m/e 410 [M+H]⁺. mp 235.8-237.5° C. ¹H-NMR (CDCl₃) δ 15.26 (s, 1H); 14.63 (bs, 1H); 8.30 (dd, 1H, J=8.1, 1.5 Hz); 7.99 (d, 1H, J=7.8 Hz); 7.73 (td, 1H, J=8.0, 1.4 Hz); 7.62 (td, 1H, J=8.4, 1.4 Hz); 7.48-7.31 (m, 4H); 4.43 (d, 2H, J=7.1 Hz); 2.88-2.75 (m, 1H); 2.15-1.90 (m, 6H).

### EXAMPLE 30

3-1,1-Dioxo-1,2-dihydrobenzo[1,2,4]thiadiazin-3-yl)-4-hydroxy-5-methyl-1-(3-methylbutyl)-1H-quinolin-2-one

a) 5-Methylbenzo[d][1,3]oxazine-2,4-dione

A solution of 6-methylanthranilic acid (978 mg, 6.46 mmol) in tetrahydrofuran (20 mL) was treated with triphosgene (960 mg, 3.2 mmol) and stirred at 50° C. overnight. Saturated sodium hydrogen carbonate solution was added and the mixture extracted with ethyl acetate. Evaporation of the organic solution gave the title compound (810 mg, 70%). ¹H NMR (300 MHz, d₆-DMSO) δ 11.60 (s, 1H), 7.55 (t, J=8 Hz, 1H), 7.04 (d, J=7 Hz, 1H) 6.98 (d, J=8 Hz, 1H), 2.58 (s, 3H).

b) 1-(3-Methylbutyl)-5-methylbenzo[d][1,3]oxazine-2,4-dione

The compound from Example 30a) (530 mg, 3.0 mmol), triphenylphosphine (787 mg, 3.0 mmol) and 3-methylbutanol(0.32 ml, 3.0 mmol) were stirred together in dichloromethane and treated with diethyl azodicarboxylate (0.47 ml, 3.0 mmol). The reaction was stirred under nitrogen overnight, evaporated onto silica and purified by chromatography (silica gel, ethyl acetate-hexanes) to give the title compound (360 mg, 48%). ¹H NMR (300 MHz, CDCl₃) δ 7.58 (t, J=8 Hz, 1H), 7.10 (d, J=7 Hz, 1H), 7.00 (d, J=9 Hz, 1H), 4.06 (m, 2H), 2.75 (s, 3H), 1.80 (m, 1H), 1.61 (m, 2H) 1.02 (d, J=6.5 Hz, 6H).

c) 3-(1,1-Dioxo-1,2-dihydrobenzo[1,2,4]thiadiazin-3-yl)-4-hydroxy-5-methyl-1-(3-methylbutyl)-1H-quinolin-2-one

Sodium hydride (226 mg of a 60% suspension in mineral oil, 3.25 mmol) was added to a mixture of the compound from Example 30b) (350 mg, 1.4 mmol) and ethyl 1,1-dioxo-2H-benzo-1,2,4-thiadiazinyl-3-acetate (380 mg, 1.4 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (420 mg, 70%). ¹H NMR (300 MHz, d₆-DMSO) δ 15.92 (s, 1H), 14.57 (s, 1H), 7.78 (t, J=7 Hz, 1H), 7.53-7.68 (m, 3H), 7.43 (d, J=8 Hz, 1H), 7.12 (d, J=8 Hz, 1H), 4.27 (m, 2H), 2.77 (s, 3H), 1.77 (m, 1H), 1.52 (m, 2H) 0.99 (d, J=6.5 Hz, 6H). Anal. (C₂₂H₂₃N₃O₄S) calcd. C, 62.10; H, 5.42; N, 9.88; S, 7.53. found: C, 61.77; H, 5.70; N, 9.27; S, 7.40.

### EXAMPLE 31

5-Chloro-3-1,1-dioxo-1,2-dihydrobenzo[1,2,4]thiadiazin-3-yl)-4-hydroxy-1-(3-methylbutyl)-1H-quinolin-2-one

a) 5-Chlorobenzo[d][1,3]oxazine-2,4-dione

A solution of 6-chloroanthranilic acid (1.15 g, 6.73 mmol) in tetrahydrofuran (20 mL) was treated with triphosgene (1.0 g, 3.3 mmol) and stirred at 50° C. overnight. Saturated sodium hydrogen carbonate solution was added and the mixture extracted with ethyl acetate. Evaporation of the organic solution gave the title compound (1.25 g, 94%). ¹H NMR (300 MHz, d₆-DMSO) δ 11.85 (s, 1H), 7.65 (t, J=8 Hz, 1H), 7.30 (d, J=8 Hz, 1H) 7.10(d, J=9 Hz, 1H).

b) 1-(3-Methylbutyl)-5-chlorobenzo[d][1,3]oxazine-2,4-dione

The compound from Example 31a) (517 mg, 2.62 mmol), triphenylphosphine (688 mg, 2.62 mmol) and 3-methylbutanol(0.3 ml, 2.75 mmol) were stirred together in dichloromethane and treated with diethyl azodicarboxylate (0.42 ml, 2.62 mmol). The reaction was stirred under nitrogen overnight, evaporated onto silica and purified by chromatography (silica gel, ethyl acetate-hexanes) to give the title compound (440 mg, 63%). ¹H NMR (300 MHz, CDCl₃) δ 7.62 (t, J=8 Hz, 1H), 7.33 (d, J=8 Hz, 1H), 7.08 (d, J=9 Hz, 1H), 4.06 (m, 2H), 1.78 (m, 1H), 1.61 (m, 2H) 1.02 (d, J=6.5 Hz, 6H).

c) 5-Chloro-3-(1,1-dioxo-1,2-dihydrobenzo[1,2,4]thiadiazin-3-yl)-4-hydroxy-1-(3-methylbutyl)-1H-quinolin-2-one

Sodium hydride (263 mg of a 60% suspension in mineral oil, 6.56 mmol) was added to a mixture of the compound from Example 31b) (440 mg, 1.64 mmol) and ethyl 1,1-dioxo-2H-benzo-1,2,4-thiadiazinyl-3-acetate (440 mg, 1.64 mmol) in tetrahydrofuran (20 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (520 mg, 71%). ¹H NMR (300 MHz, d₆-DMSO) δ 16.10 (br. s, 1H), 14.43 (s, 1H), 7.94 (d, J=8 Hz, 1H), 7.57-7.82 (m, 5H), 7.48 (d, J=8 Hz, 1H), 4.33 (m, 2H), 1.80 (m, 1H), 1.53 (m, 2H) 1.00 (d, J=7 Hz, 611). Anal. (C₂₁H₂₀ClN₃O₄S) calcd. C, 56.56; H. 4.52; Cl, 7.95; N, 9.42; S, 7.19. found: C, 56.58; H, 4.60; Cl, 7.98; N, 9.22; S, 7.19.

### EXAMPLE 32

5-Bromo-3-(1,1-dioxo-1,2-dihydrobenzo[1,2,4]thiadiazin-3-yl)-4-hydroxy-1-(3-methylbutyl)-1H-quinolin-2-one

a) 5-Bromo-2H-3,1-benzoxazine-2,4(1H)-dione and 7-bromo-2H-3,1-benzoxazine-2,4(1H)-dione.

A solution of a 1:1 mixture of 4-bromoindole-2,3-dione and 6-bromoindole-2,3-dione (prepared by the method of Katsifis and McPhee; Aust. J. Chem. 1999, 52, 1061-1069) (3.7 g; 0.016 mol.) in glacial acetic acid (25.0 mL) was treated with 30% aqueous hydrogen peroxide solution (5.0 mL). The suspension was then stirred at 50° C. for 5h. The mixture was concentrated and the residue was washed with diethyl ether to give a mixture of 7-bromo-2H-3,1-benzoxazine-2,4(1H)-dione (˜17% measured by ¹H NMR) and 5-bromo-2H-3,1-benzoxazine-2,4(1H)-dione (˜83% measured by ¹H NMR) (4.0 g, yield 100%) as a yellow solid. MS(ES+) m/e 244,242 [M+H]⁺.

b) 5-Bromo-2H-3,1-benzoxazine-1-(3-methylbutyl)-2,4-dione and 7-bromo-2H-3,1-benzoxazine-1-(3-methylbutyl)-2,4-dione.

A solution of triphenylphosphine (1.24 g, 4.7 mmol) and 3-methylbutanol (0.47 mL, 4.3 mmol) was treated with the mixture of the compound from Example 32a) (1.1 g; 4.5 mmol) then treated dropwise with diisopropyl azodicarboxylate (1.0 mL, 4.8 mmol). The solution was stirred at room temperature and then concentrated to give a gum. The gum was purified by chromatography (silica gel, 10% ethyl acetate/hexanes) to give 5-bromo-2H-3,1-benzoxazine-1-(3-methylbutyl)-2,4-dione (0.57 g; 41%), ¹H NMR (300 MHz, CDCl₃) δ 7.6 (dd, 1H), 7.5 (t, 1H), 7.1 (dd, 1H), 4.1 (m, 2H), 1.8 (m, 1H), 1.7 (m, 2H), 1.0 (d, 6H) MS(ES+) m/e 314, 312 (M+H); followed by 7-bromo-2H-3,1-benzoxazine-1-(3-methylbutyl)-2,4-dione (0.10 g; 7%), ¹H NMR (300 MHz, CDCl₃) δ 8.0 (d, 1H), 7.4 (dd, 1H), 7.3 (d, 1H), 4.6 (m, 2H), 1.8 (m, 3H), 1.0 (d, 6H). MS(ES+) m/e 314, 312 [M+H]⁺.

c) 3-(1,1-Dioxo-1,2-dihydrobenzo[1,2,4]thiadiazin-3-yl)-5-bromo-4-hydroxy-1-(3-methylbutyl)-1H-quinolin-2-one

A solution of 1-(3-methylbutyl)-5-bromobenzo[d][1,3]oxazine-2,4-dione (200 mg, 0.6 mmol) and ethyl (1,1-dioxo-1,2-dihydrobenzo[1,2,4]thiadiazin-3-yl)acetate (172 mg, 0.6 mmol) in tetrahydrofuran (15.0 nL) was treated with sodium hydride (60% dispersion in mineral oil) (100 mg; 2.6 mmol) and heated under reflux for 1.5h. The mixture was then cooled to room temperature and acidified with excess glacial acetic acid. The mixture was then heated under reflux for an additional 1.5 h, cooled and quenched with water. The product was collected by filtration, washed with water, ether then hexanes to give the title compound (180 mg; 60%) as white powder. ¹H NMR (300 MHz, d₆-DMSO) δ 8.0 (d, J=7.5 Hz, 1H), 7.8 (t, J=8.0 Hz, 1H), 7.7 (m, 4H), 7.6 (t, J=8.0 Hz, 1H), 4.4 (m, 2H), 1.8 (m, 1H), 1.5 (m, 2H), 1.0 (d, J=7.0 Hz, 6H).

### EXAMPLE 33

4-[3-(1,1-Dioxo-1,4-dihydrobenzo[1,2,4]thiadiazin-3-yl)-4-hydroxy-6-methoxy-2-oxo-2 H-quinolin-1-yl]butyronitrile

a) 4-(6-Methoxy-benzo[d][1,3]oxazine-2,4-dione-1-yl)-butyronitrile

6-Methoxy-1H-benzo[d][1,3]oxazine-2,4-dione (500 mg, 2.6 mmol) was added portionwise to a suspension of sodium hydride (60% suspension in mineral oil) (124.3 mg, 3.1 mmol) in anhydrous N,N-dimethylacetamide. After addition of 4-bromobutyronitrile (0.309 ml, 3.1 mmol) the mixture was stirred at room temperature for 48 h and then poured onto ice/H₂O. The solid formed was collected by filtration, washed with water, dried and triturated in CHCl₃ to afford 362 mg (54%) of product. ¹H NMR (300 MHz, d₆-DMSO) δ 7.48-7.45 (m, 3H); 4.10 (t, J=6.9 Hz, 2H); 3.85 (s, 3H); 2.66 (t, J=7.2 Hz, 2H); 1.95 (quin, J=7.2 Hz, 2H). MS(ES+) m/e 261 [M+H]⁺.

b) 4-[3-(1,1-Dioxo-1,4-dihydrobenzo[1,2,4]thiadiazin-3-yl)-4-hydroxy-6-methoxy-2-oxo-2H-quinolin-1-yl]butyronitrile

Sodium hydride (123 mg of a 60% suspension in mineral oil, 3.07 mmol) was added to a mixture of the compound from Example 33a) (200 mg, 0.77 mmol) and ethyl 1,1-dioxo-2H-benzo-1,2,4-thiadiazinyl-3-acetate (206 mg, 0.77 mmol) in tetrahydrofuran (8.0 mL). The mixture was heated under reflux for 2.5 h, cooled, acidified with acetic acid and heated under reflux for 1.0 h, cooled to room temperature and poured into 1 N HCl. The product was collected, washed with water, diethyl ether and hexanes and recrystallized from DMSO to give the title compound (195 mg, 56%).

¹H NMR (300 MHz, d₆-DMSO) δ 15.26 (s, 1H); 14.47 (s, 1H); 7.95 (d, J=7.9 Hz, 1H); 7.81-7.50 (m, 6H); 4.43 (t, J=7.1 Hz, 2H); 3.90 (s, 3H); 2.71 (t, J=7.1 Hz, 2H), 1.98 quin, J=7.1 Hz, 2H). MS(ES+) m/e 439 [M+H]⁺.

### EXAMPLE 34

1-But-3-ynyl-3-(1,1-dioxo-1,4-dihydrobenzo[1,2,4]thiadiazin-3-yl)-4-hydroxy-1H-quinolin-2-one

Following the procedures of Examples 28a) and 28b) except substituting 3-butyn-1-ol for cyclopentanemethanol, the title compound was prepared (205 mg, 62%) as a white solid after trituration in Et₂O. ¹H-NMR (d₆-DMSO) δ 15.23 (br s, 1H); 14.27 (s, 1H); 8.22 (dd, J=8.0, 1.2 Hz, 1H); 7.95 (d, J=7.1 Hz, 1H); 793-7.71 (m, 4H); 7.57 (td, J=8.1, 1.2 Hz, 1H); 7.47 (td, J=7.9, 1.2 Hz, 1H); 4.53 (t, J=7.6 Hz, 2H); 2.95 (t, J=2.5 Hz, 1H); 2.68-2.63 (m, 2H). MS(ES+) m/e 394 [M+H]⁺.

### EXAMPLE 35

1-(3,3-Dimethylbutyl)-3-(1,1-dioxo-1,4-dihydrobenzo[1,2,4]thiadiazin-3-yl)-4-hydroxy-1H-quinolin-2-one

Following the procedures of Examples 28a) and 28b) except substituting 3,3-dimethyl-1-butanol for cyclopentanemethanol, the title compound was prepared (211 mg, 61%) as a white solid after trituration in Et₂O. ¹H-NMR (d₆-DMSO) δ 15.18 (br s, 1H); 14.29 (s, 1H); 8.19 (dd, J=8.1, 1.3 Hz, 1H); 7.94 (d, J=7.8 Hz, 1H); 792-7.68 (m, 4H); 7.59-7.54 (m, 1H); 7.44 (t, J=7.6 Hz, 1H); 4.364.31 (m, 2H); 1.59-1.53 (m, 2H); 1.07 (s, 9H). MS(ES+) m/e 426 [M+H]⁺.

### EXAMPLE 36

1-(2-Cyclopropylethyl)-3-(1,1-dioxo-1,4-dihydrobenzo[1,2,4]thiadiazin-3-yl)-4-hydroxy-1H-quinolin-2-one

Following the procedures of Examples 28a) and 28b) except substituting 2-cyclopropylethanol for cyclopentanemethanol, the title compound was prepared (206 mg, 58%) as a white solid. ¹H-NMR (CDCl₃) δ 15.24 (s, 1H); 14.60 (s, 1H); 8.31 (dd, J=8.1, 1.4 Hz, 1H); 8.01 (dd, J=8.0, 0.6 Hz, 1H); 7.75 (ddd, J=8.6, 7.2, 1.6 Hz, 1H); 766-7.61 (m, 1H); 7.48-7.31 (m, 4H); 4.43 (t, J=7.7 Hz, 2H); 1.68 (q, J=7.6 Hz, 2H); 0.84-0.79 (m, 1H); 0.55-0.49 (m, 2H); 0.17-0.12 (m, 2H). MS(ES+) m/e 410 [M+H]⁺.

### EXAMPLE 37

N-[3-(1,1-Dioxo-1,4-dihydrobenzo[1,2,4]thiadiazin-3-yl)-4-hydroxy-1-(3-methylbutyl)-2-oxo-1,2dihydroquinolin-6-yl]4-methoxybenzamide

4-Methoxybenzoyl chloride (38 mg, 0.22 mmol) was added to a stirred suspension of the compound from Example 19 (85 mg, 0.2 mmol) and pyridine (18 ul, 0.22 mmol) in dichloromethane (10 ml). After 30 minutes, 1.0M hydrochloric acid was added, the solid collected, washed with 11.0M hydrochloric acid, water, ether and hexane to give the title compound (85 mg, 76%). ¹H NMR (300 MHz, d₆-DMSO) δ 15.20 (br s, 1H), 14.51 (s, 1H), 10.46 (s, NH), 8.65 (s, 1H), 8.32 (d, J=9 Hz, 1H), 8.02 (d, J=9 Hz, 2H), 7.95 (d, J=8 Hz, 1H), 7.70-7.80 (m, 2H), 7.57 (m, 1H), 7.10 (d, J=9 Hz, 2H), 4.36 (m, 2H), 3.86 (s, 3H), 1.81 (m, 1H), 1.60 (m, 2H), 1.02 (d, 6H).

### EXAMPLE 38

Furan-2-carboxylic acid [3-(1,1dioxo-1,4-dihydrobenzo[1,2,4]thiadiazin-3-yl)-4-hydroxy-1-3-methylbutyl)-2-oxo-1,2-dihydroquinolin-6-yl]amide

Following the procedure of Example 37, except substituting 2-furoyl chloride for 4-methoxybenzoyl chloride, the title compound was prepared as a pale yellow solid (40 mg, 30%). ¹H NMR (300 MHz, d₆-DMSO) δ 15.21 (br.s, 1H), 14.48 (s, 1H), 10.60 (s, NH), 8.64 (d, J=3 Hz, 1H), 8.32 (dd, J=3 and 9 Hz, 1H), 7.96 (m, 2H), 7.75 (m, 2H), 7.57 (m, 1H), 7.40 (d, J=3 Hz, 1H), 6.75 (m, 1H), 4.35 (m, 2H), 1.83 (m, 1H), 1.57 (m, 2H), 1.02 (d, 6H). Anal. (C₂₆H₂₄N₄O₆S 0.2H₂O) calcd. C, 59.58; H, 4.69; N, 10.69; S, 6.12. Found: C, 59.25; H, 4.47; N, 11.01; S, 6.12.

### EXAMPLE 39

3-Cyano-N-[3(1,1-dioxo-1,4-dihydrobenzo[1,2,4]thiadiazin-3-yl)-4-hydroxy-1-(3-methylbutyl)-2-oxo-1,2dihydroquinolin-6-yl]benzamide

Following the procedure of Example 37, except substituting 3-cyanobenzoyl chloride for 4-methoxybenzoyl chloride, the title compound was prepared as a pale yellow solid (35 mg, 25%). ¹H NMR (300 MHz, d₆-DMSO) δ 15.22 (br.s, 1H), 14.45 (s, 1H), 10.77 (s, NH), 8.64 (s, 1H), 8.47 (s, 1H), 8.31 (d, J=9 Hz, 2H), 8.10 (d, J=7 Hz, 1H), 7.94 (d, J=8 Hz, 1H), 7.74 (m, 4H), 7.59 (m, 1H), 4.35 (m, 2H), 1.83 (m, 1H), 1.60 (m, 2H), 1.03 (d, 6H).

### EXAMPLE 40

Cyclopropane carboxylic acid [3-(1,1-dioxo-1,4-dihydrobenzo[1,2,4]thiadiazin-3-yl)-4-hydroxy-1-(3-methylbutyl)-2-oxo-1,2dihydroquinolin-6-yl]amide

Following the procedure of Example 37, except substituting cyclopentanecarbonyl chloride for 4-methoxybenzoyl chloride, the title compound was prepared as a pale yellow solid (20 mg, 15%). ¹H NMR (300 MHz, d₆-DMSO) δ 15.19 (br.s, 1H), 14.51 (s, 1H), 10.26 (s, NH), 8.51 (s, 1H), 8.11 (d, J=3 Hz, 1H), 7.94 (d, J=8 Hz, 1H), 7.54-7.81 (m, 4H), 4.33 (m, 2H), 2.75 (m, 1H), 1.83 (m, 1H), 1.57-1.91 (m, 10H), 1.01 (d, 6H).

### EXAMPLE 41

N-[3-(1,1-Dioxo-1,4-dihydrobenzo[1,2,4]thiadiazin-3-yl)-4-hydroxy-1-(3-methylbutyl)-2-oxo-1,2-dihydroquinolin-6-yl]-3-methoxybenzamide

Following the procedure of Example 37, except substituting 3-methoxybenzoyl chloride for 4methoxybenzoyl chloride, the title compound was prepared as a pale yellow solid (35 mg, 25%). ¹H NMR (300 MHz, d₆-DMSO) δ 15.19 (s, 1H), 14.46 (s, 1H), 10.55 (s, NH), 8.64 (s, 1H), 8.29 (d, J=2 Hz, 1H), 7.94 (d, J=8 Hz, 2H), 7.44-7.76 (m, 7H), 7.19 (m, 1H), 4.33 (m, 2H), 3.86 (s, 3H), 1.81 (m, 1H), 1.57 (m, 2H), 1.02 (d, 6H).

### EXAMPLE 42

N-[3-(1,1-Dioxo-1,4-dihydrobenzo[1,2,4]thiadiazin-3-yl)-4-hydroxy-1-(3-methylbutyl)-2-oxo-1,2-dihydroquinolin-6-yl]benzamide

Following the procedure of Example 37, except substituting benzoyl chloride for 4-methoxybenzoyl chloride, the title compound was prepared as a pale yellow solid (20 mg, 15%). ¹H NMR (300 MHz, d₆-DMSO) δ 15.15 (s, 1H), 14.51 (s, 1H), 10.62 (s, NH), 8.70 (s, 1H), 8.29 (d, J=3 Hz, 1H), 7.94 (d, J=8 Hz, 2H), 7.906 (d, 1H), 7.52-7.80 (m, 7H), 4.32 (m, 2H), 1.81 (m, 1H), 1.61 (m, 2H), 1.02 (d, 6H).

### EXAMPLE 43

N-[3-(1,1-Dioxo-1,4-dihydrobenzo[1,2,4]thiadiazin-3-yl)-4-hydroxy-1-(3-methylbutyl)-2-oxo-1,2-dihydroquinolin-6-yl]-4-nitrobenzamide

Following the procedure of Example 37, except substituting 4-nitrobenzoyl chloride for 4-methoxybenzoyl chloride, the title compound was prepared as a pale yellow solid (70 mg, 49%). ¹H NMR (300 MHz, d₆-DMSO) δ 15.19 (s, 1H), 14.41 (s, 1H), 10.85 (s, NH), 8.60 (s, 1H), 8.36 (d, J=9 Hz, 2H), 8.22 (m, 2H), 7.94 (m, 2H), 7.51-7.77 (m, 4H), 4.32 (m, 2H), 1.83 (m, 1H), 1.56 (m, 2H), 1.02 (d, 6H).

### EXAMPLE 44

3-[4-Hydroxy-1-(3-methylbutyl)-2-oxo-1,2-dihydroquinolin-3-yl]-1,1-dioxo-1,4-dihydrobenzo[1,2,4]thiadiazine-7-carboxylic Acid Amide

Water (0.75 mL) was added over 10 min to a solution of 3-(7-cyano-1,1-dioxo-1,2-dihydrobenzo[1,2,4]thiadiazin-3-yl)-4-hydroxy-1-(3-methylbutyl)-1H-quinolin-2-one (Example 25) (83 mg, 0.19 mmol) in sulfuric acid (5 mL) stirred at room temperature. After stirring for 18h, iced water (50 mL) was added and the precipitate filtered, washed with water and air-dried. Half of the material was set aside for use in a later experiment and the remainder was purified by chromatography on silica gel (5-10% methanol/dichloromethane) to give the title compound (35 mg; 81%) as a white powder. ¹H NMR (300 MHz, DMSO-d₆) δ 15.02 (1H, br s), 14.55 (1H, br s), 8.47 (1H, d, J=1.7 Hz), 8.32 (1H, br s), 8.2&8.22 (2H, m), 7.91 (1H, m), 7.79 (1H, d, J=8.7 Hz), 7.70-7.65 (2H, m), 7.46 (1H, t, J=7.6 Hz), 4.35 (2H, m), 1.81 (1H, m), 1.56 (2H, m), 1.02 (6H, d, J=6.6 Hz).

### EXAMPLE 45

3-(6-Bromo-1,1-dioxo-1,4-dihydrobenzo[1,2,4]thiadiazin-3-yl)-4-hydroxy-1-(3-methylbutyl)-1H-quinolin-2-one

a) 2-Amino-4-bromobenzenesulfonamide

Chlorosulfonylisocyanate (0.957 mL, 11.0 mmol) was injected dropwise into a stirred solution of 3-bromoaniline (1.72 g, 10.0 mmol) in nitroethane (10 mL) at −50° C. under argon. The mixture was stirred at 0° C. for 10 min, then aluminum chloride (1.60 g, 12.0 mmol) added and the mixture heated under reflux for 20 min. After cooling to room temperature, it was poured onto ice and the precipitate filtered off and air dried. The solid was heated under reflux in concentrated aqueous hydrochloric acid (35 mL) for 5 h. After cooling and filtering, the filtrate was neutralised with aqueous NaOH, and extracted with ethyl acetate. The extracts were washed with water, brine, dried over MgSO₄, and evaporated under reduced pressure. The residue was purified by chromatography on silica gel twice (30-50% ethyl acetate/hexane, then 1-10% methanol/dichloromethane) to give the title compound (104 mg, 4%) as an amorphous solid. ¹H NMR (300 MHz, DMSO-d₆) δ 7.45 (1H, d, J=8.5 Hz), 7.35 (2H, br s), 7.02(1H, d, J=1.9 Hz), 6.77 (1H, dd, J=8.5, 1.9 Hz), 6.07 (2H, br s).

b) 3Cyano-4-hydroxy-1-(3-methylbutyl)-1H-quinolin-2-one

Sodium hydride (2.00 g of a 60% oil suspension, 50.0 mmol) was added to a stirred solution of 1-(3-methylbutyl)-1H-benzo[d][1,3]oxazine-2,4dione (Example 21a, 5.27 g, 22.6 mmol) and methyl cyanoacetate (4.00 mL, 45.3 mmol) in dimethylformamide (50 mL) under argon, and, after the hydrogen had all been released, the mixture was heated at 100° C. for 3 h, then cooled. Acetic acid (10 mL) was added slowly, and the mixture reheated at 100° C. for 1 h, then cooled again. Water (500 mL) was added and the pH adjusted to 1 (dilute aqueous hydrochloric acid). Ether (50 mL) was added and the mixture cooled in ice, then filtered. The solid was washed with water, ether and dried to leave the title compound (3.68 g, 64%) as a white solid. ¹H NMR (300 MHz, DMSO-d₆) δ 8.12 (1H, dd, J=8.1, 1.5 Hz), 7.78 (1H, m), 7.53 (1H, d, J=8.6 Hz), 7.33 (1H, m), 4.20 (2H, m), 1.70 (1H, m), 1.46 (2H, m), 0.97 (6H, d, J=6.6 Hz).

c) 3-6-Bromo-1,1-dioxo-1,4-dihydrobenzo[1,2,4]thiadiazin-3-yl)-4-hydroxy-1-(3-methylbutyl)-1H-quinolin-2-one

Trimethylaluminum (0.406 mL of a 2M hexane solution, 0.812 mmol) was injected into a mixture of 2-amino-4bromobenzenesulfonamide (102 mg, 0.406 mmol), 3-cyano-4-hydroxy-1(3-methylbutyl)-1H-quinolin-2-one (104 mg, 0.406 mmol) and dioxane (6 nL) stirred under argon at room temperature. After 10 min, the mixture was heated under reflux for 3 h, then at 70-C for 18 h. After cooling to room temperature, water (0.27 mL, 15 mmol) was added, followed by ethyl acetate (10 mL), and the mixture stirred 15 min. Excess sodium hydrogencarbonate was added and stirring continued 1 h, then the mixture filtered through magnesium sulfate and the filtrate evaporated under reduced pressure. The residue was purified by chromatography on silica gel (dichloromethane) to give an intermediate, which was heated under reflux in 1M aqueous NaOH for 90 min. After cooling and adjusting to pH 1 with dilute aqueous HCl, the solid was filtered, washed with water, dried and purified by chromatography on silica gel (dichloromethane) to give the title compound (4 mg, 2%) as an amorphous solid. ¹H NMR (300 MHz, DMSO-d₆) δ 15.00 (1H, br s), 14.41 (1H, br s), 8.22 (1H, m), 8.10 (1H, s), 7.94-7.87 (2H, m), 7.75-7.67 (2H, m), 7.46 (1H, m), 4.34 (2H, m), 1.81 (1H, m), 1.56 (2H, m), 1.02 (6H, d, J=6.6 Hz).

### EXAMPLE 46

N-[3-(1,1-Dioxo-1,4-dihydrobenzo[1,2,4]thiadiazin-3-yl)-4-hydroxy-1-(3-methylbutyl)-2-oxo-1,2-dihydroquinolin-6-yl]-3-methylbutyramide

Following the procedure of Example 37, except substituting isovaleryl chloride for 4-methoxybenzoyl chloride, the title compound was prepared as a pale yellow solid (15 mg, 12%). ¹H NMR (300 MHz, CDCl₃) δ 15.03 (s, 1H), 14.67 (s, 1H), 8.40 (m, 1H), 7.98 (m, 2H), 7.85 (s, NH), 7.66 (m, 1H), 7.48 (m, 1H), 7.33 (m, 2H), 4.26 (m, 2H), 2.23 (m, 2H), 1.79 (m, 1H), 1.59 (m, 3H), 1.04 (m, 12H).

### EXAMPLE 47

N-[3-(1,1-Dioxo-1,4-dihydrobenzo[1,2,4]thiadiazin-3-yl)-4-hydroxy-1-(3-methylbutyl)-2-oxo-1,2-dihydroquinolin-6-yl]acetamide

A mixture of the compound from Example 19 (85 mg, 0.2 mmol), acetic anhydride (0.2 ml, 2.1 mmol) and pyridine (0.2 ml, 2.5 mmol) was stirred in chloroform for several days then concentrated. Purification by chromatography (silica gel, gradient, hexanes/ethyl acetate 20%-100%) gave the title compound (15 mg, 16%). ¹H NMR (300 MHz, CDCl₃-d₄-methanol) δ 8.20 (dd, J=2 and 9 Hz, 1H), 8.07 (d, J=2 Hz, 1H), 7.85 (d, J=8 Hz, 1H), 7.56 (m, 1H), 7.37 (m, 1H), 7.29 (m, 2H), 4.21 (m, 2H), 2.07 (s, 3H), 1.70 (m, 1H), 1.52 (m, 3H), 0.95 (d, 6H).

### EXAMPLE 48

4-Hydroxy-1-(3-methylbutyl)-3-(5-methyl-1,1-dioxo-1,4-dihydrobenzo[1,2,4]thiadiazin-3-yl)-1H-quinolin-2-one

a) 2-Amino-3-methylbenzenesulfonamide

A solution of 2-methylaniline (1.07 g, 10.0 mmol) in nitroethane (3 mL) was injected over 5 min into a stirred solution of chlorosulfonylisocyanate (0.957 mL, 11.0 mmol) in nitroethane (7 mL) at −78° C. under argon. The mixture was warmed to 0° C., then aluminum chloride (1.60 g, 12.0 mmol) added and the mixture heated under reflux for 10 min. After cooling, the mixture was poured on to ice and the precipitate filtered and washed with water and ether and dried. The solid was heated under reflux in 9M aqueous sulfuric acid/dioxane (3:1, 26 mL) for 18 h, cooled and the mixture filtered. Dioxane was removed under reduced pressure and the solution neutralised with 4M aqueous NaOH, filtered and extracted with ethyl acetate. The extracts were washed with water, brine, dried over MgSO₄ and evaporated under reduced pressure. The residue was purified by chromatography on silica gel (50% ethyl acetate/hexane) to give the title compound (352 mg, 19%) as a tan solid. ¹H NMR (300 MHz, DMSO-d₆) δ 7.46 (1H, dd, J=8.0, 1.3 Hz), 7.26 (2H, br s), 7.18 (1H, d, J=7.0 Hz), 6.58 (1H, m), 5.58 (1H, br s), 2.14 (3H, s).

b) 4Hydroxy-1-(3-methylbutyl)-3-(5-methyl-1,1-dioxo-1,4-dihydrobenzo[1,2,4]thiadiazin-3-yl)-1H-quinolin-2-one

The procedure of Example 45c) was followed, using 2-amino-3-methylbenzenesulfonamide in place of 2-amino-4-bromobenzenesulfonamide, to give the title compound as a pale yellow solid. ¹H NMR (300 MHz, DMSO-d₆) δ 15.35 (1H, br s), 14.64 (1H, br s), 8.23 (1H, dd, J=8.1, 1.3 Hz), 7.91 (1H, m), 7.80 (1H, d, J=7.9 Hz), 7.72-7.67 (2H, m), 7.51-7.45 (2H, m), 4.38 (2H, m), 2.54 (3H, s), 1.79 (1H, m), 1.57 (2H, m), 1.02 (6H, d, J=6.6 Hz).

### EXAMPLE 49

C-Dimethylamino-N-[3-(1,1-dioxo-1,4-dihydrobenzo[1,2,4]thiadiazin-3-yl)-4-hydroxy-1-(3-methylbutyl)-2-oxo-1,2-dihydroquinolin-6-yl]acetamide

a) 2-Bromo-N-[3-(1,1-dioxo-1,4-dihydrobenzo[1,2,4]thiadiazin-3-yl)-4-hydroxy-1-(3-methylbutyl)-2-oxo-1,2-dihydroquinolin-6-yl]acetamide

Bromoacetyl bromide (0.44 ml, 0.5 mmol) was added to a mixture of the compound from Example 19 (200 mg, 0.47 mmol) and pyridine (0.4 ml, 5.0 mmol) in chloroform, stirred for 2 hours, washed with water and evaporated. Trituration with ether gave the title compound (160 mg, 62%). ¹H NMR (300 MHz, d₆-DMSO) δ 15.10 (br. s, 1H), 14.42 (br.s, 1H), 9.10 (d, 1H), 8.45 (m, 2H), 8.23 (m, 2H), 7.74 (m, 1H), 7.69 (m, 1H), 7.53 (m, 1H), 5.70 (s, 2H), 4.35 (m, 2H), 1.55 (m, 1H), 1.28 (m, 2H), 1.00 (d, 6H).

b) C-Dimethylamino-N-[3-(1,1 dioxo-1,4-dihydrobenzo[1,2,4]thiadiazin-3-yl)-4-hydroxy-1-(3-methylbutyl)-2-oxo-1,2-dihydroquinolin-6-yl]acetamide

Dimethylamine (2.0 ml of a 2.0 molar solution in tetrahydrofuran) was added to a solution of the compound from Example 49a (80 mg, 0.18 mmol) in chloroform and stirred for 2 hours. Purification using flash chromatography (2% methanol in dichloromethane) gave the title compound (5 mg, 6%). ¹H NMR (300 MHz, CDCl₃-d₄-methanol) δ 8.20 (dd, J=2 and 9 Hz, 1H), 8.07 (d, J=2 Hz, 1H), 7.85 (d, J=8 Hz, 1H), 7.56 (m, 1H), 7.37 (m, 1H), 7.29 (m, 2H), 4.21 (m, 2H), 2.07 (s, 3H), 1.70 (m, 1H), 1.52 (m, 3H), 0.95 (d, 6H).

### EXAMPLE 50

3-(1,1-Dioxo-1,4-dihydrobenzo[1,2,4]thiadiazin-3-yl)-4-hydroxy-1-(2-methylsulfanylethyl)-1H-quinolin-2-one

Following the procedures of Examples 28a) and 28b) except substituting 2-(methylthio)ethanol for cyclopentanemethanol, the title compound was obtained (130 mg, 39%) as a white solid after washing the precipitate with H₂O, hexanes, Et₂O and 0.5 mL of CH₃CN (to clean yellow impurity). ¹H-NMR (CDCl₃) δ 15.32 (s, 1H); 14.43 (s, 1H); 8.32 (dd, J=8.1, 1.6 Hz, 1H); 8.00 (d, J=8.1 Hz, 1H); 7.78 (ddd, J=8.6, 7.1, 1.6 Hz, 1H); 764 (ddd, J=8.1,7.5, 1.3 Hz, 1H); 7.49-7.33 (m, 3H); 7.32 (d, J=8.5 Hz, 1H); 4.57-4.51 (m, 2H); 2.88-2.82 (m, 2H); 2.30 (s, 3H). MS(ES+) m/e 416 [M+H]⁺.

### EXAMPLE 51

3-(1,1-Dioxo-1,4-dihydrobenzo[1,2,4]thiadiazin-3-yl)-4-hydroxy-1-(3-methylsulfanylpropyl)-1H-quinolin-2-one

Following the procedures of Examples 28a) and 28b) except substituting 3-(methylthio)propanol for cyclopentanemethanol, the title compound was obtained (198 mg, 39%) as pale yellow solid after washing the precipitate with H₂O, hexanes, Et₂O and 0.5 mL of CH₃CN. ¹H-NMR (CDCl₃) δ 15.28 (s, 1H); 14.52 (s, 1H); 8.33 (dd, J=8.1, 1.5 Hz, 1H); 7.99 (dd, J=8.0, 1.4 Hz, 1H); 7.78 (ddd, J=8.6, 7.2, 1.6 Hz, 1H); 7.64 (td, J=8.1, 1.4 Hz, 1H); 7.55 (d, J=8.6 Hz, 1H); 7.45 (td, J=7.6, 1.1 Hz, 1H); 7.39 (td, J=7.6, 0.6 Hz, 1H); 7.31 (d, J=8.0 Hz, 1H); 4.48-4.43 (m, 2H); 2.69 (t, J=6.7 Hz, 2H); 2.19 (s, 3H); 2.07 (quin, J=7.3 Hz, 2H). MS(ES+) m/e 430 [M+H]⁺.

### EXAMPLE 52

3-(1,1-Dioxo-1,4-dihydrobenzo[1,2,4]thiadiazin-3-yl)-1-furan-3-ylmethyl-4-hydroxy-1H-quinolin-2-one

Following the procedures of Examples 28a) and 28b) except substituting 3-furanmethanol for cyclopentanemethanol, the title compound was obtained (126 mg, 36%) as white crystals after washing the precipitate with H₂O, hexanes, Et₂O and 0.5 mL of CH₃CN. ¹H-NMR (CDCl₃) δ 15.36 (s, 1H); 14.44 (s, 1H); 8.32 (dd, J=8.1, 1.5 Hz, 1H); 7.99 (dd, J=8.0, 0.7 Hz, 1H); 7.73 (ddd, J=8.6, 7.1, 1.5 Hz, 1H); 7.64 (ddd, J=8.1, 7.5, 1.3 Hz, 1H); 7.51-7.35 (m, 5H); 7.31 (dd, J=8.4, 0.7 Hz, 1H); 6.41-6.40 (m, 1H); 5.41 (s, 2H). MS(ES+) m/e 422 [M+H]⁺.

### EXAMPLE 53

3-(1,1-Dioxo-1,4dihydrobenzo[1,2,4]thiadiazin-3-yl)-4-hydroxy-1-(2-thiophen-2-yl-ethyl)-1-quinolin-2-one

Following the procedures of Examples 28a) and 28b) except substituting 2-(2-thienyl)ethanol for cyclopentanemethanol, the title compound was obtained (140 mg, 42%) as pale yellow crystals after washing the precipitate with H₂O, 0.5 mL MeOH, 0.5 mL of CH₃CN and hexanes. ¹H-NMR (CDCl₃) δ 15.32 (s, 1H); 14.48 (s, 1H); 8.33 (dd, J=8.3, 1.5 Hz, 1H); 8.01 (d, J=7.9 Hz, 1H); 7.76 (td, J=8.5, 1.5 Hz, 1H); 7.65 (td, J=8.5, 1.4 Hz, 1H); 7.49-7.32 (m, 4H); 7.21 (dd, J=5.0, 1.3 Hz, 1H); 7.00-6.94 (m, 2H); 4.60-4.55 (m, 2H); 3.32-3.26 (m, 2H). MS(ES+) m/e 452 [M+H]+

### EXAMPLE 54

3-(1,1-Dioxo-1,4-dihydrobenzo[1,2,4]thiadiazin-3-yl)-4-hydroxy-1-(2-thiophen-3-yl-ethyl)-1H-quinolin-2-one

Following the procedures of Examples 28a) and 28b) except substituting 2-(3-thienyl)ethanol for cyclopentanemethanol, the title compound was obtained (84 mg, 25%) as pale yellow crystals after washing the precipitate with H₂O, Et₂O, 0.5 mL of CH₃CN and hexanes. ¹H-NMR (CDCl₃) δ 15.29 (s, 1H); 14.51 (s, 1H); 8.32 (dd, J=8.1, 1.4 Hz, 1H); 8.00 (d, J=8.0 Hz, 1H); 7.74 (ddd, J=8.4, 7.1, 1.5 Hz, 1H); 7.65 (td, J=8.5, 1.5 Hz, 1H); 7.49-7.31 (m, 5H); 7.13-7.12 (m, 1H); 7.07 (dd, J=5.8, 1.3 Hz, 1H); 4.58-4.52 (m, 2H); 3.13-3.07 (m, 2H). MS(ES+) m/e 452 [M+H]⁺

### EXAMPLE 55

3-(7-Chloro-1,1-dioxo-1,4-dihydrobenzo[1,2,4]thiadiazin-3-yl)-4-hydroxy-1-(3-methylbutyl)-1H-quinolin-2-one

a) 2-Amino-5-chlorobenzenesulfonamide

A solution of 2-aminobenzenesulfonamide (861 mg, 5.00 mmol) and N-chlorosuccinimide (668 mg, 5.00 mmol) in chloroform (15 mL) was heated under reflux for 24 h, then cooled and evaporated under reduced pressure. Ether was added to the residue and the mixture filtered. The filtrate was evaporated under reduced pressure and the residue purified by chromatography on silica gel (50% ethyl acetate/hexane) to give the title compound (574 mg, 56%) as a solid. ¹H NMR (300 MHz, DMSO-d₆) δ 7.51 (1H, d, J=2.6 Hz), 7.41 (2H, br s), 7.28 (1H, dd, J=8.8, 2.5 Hz), 6.83 (1H, d, J=8.8 Hz), 6.01 (2H, br s).

b) 3-(7-Chloro-1,1-dioxo-1,4-dihydrobenzo[1,2,4]thiadiazin-3-yl)-4-hydroxy-1-(3-methylbutyl)-1H-quinolin-2-one

Trimethylaluminum (0.257 mL of a 2M hexane solution, 0.513 mmol) was injected into a stirred mixture of 2-amino-5-chlorobenzenesulfonamide (106 mg, 0.513 mmol), 3-cyano-4-hydroxy-1-(3-methylbutyl)-1H-quinolin-2-one (131 mg, 0.513 mmol) and dioxane (6 mL) under argon. The resulting solution was stirred 1 h at room temperature and 24 h under reflux, then cooled. 1M aqueous NaOH (10 mL) was added and the mixture heated under reflux for 1 h, then carefully acidified to pH 1 with 1M aqueous hydrochloric acid and cooled. The solid was filtered, washed with water and dried, then purified by chromatography on silica gel (dichloromethane) to give the title compound (74 mg, 32%) as a white solid. ¹H NMR (300 MHz, DMSO-d₆) δ 14.91 (1H, br s), 14.46 (1H, br s), 8.22 (1H, dd, J=8.1, 1.4 Hz), 8.03 (1H, d, J=2.2 Hz), 7.92 (1H, m), 7.85 (1H, dd, J=8.8, 2.2 Hz), 7.79 (1H, d, J=8.9 Hz), 7.69 (1H, d, J=8.7 Hz), 7.47 (1H, t, J=7.6 Hz), 4.35 (2H, m), 1.81 (1H, m), 1.56 (2H, m), 1.02 (6H, d, J=6.6 Hz).

### EXAMPLE 56

3-[4-Hydroxy-1-(3-methylbutyl)-2-oxo-1,2-dihydroquinolin-3-yl]-1,1-dioxo-1,4-dihydrobenzo[1,2,4]thiadiazine-7-carboxylic Acid, Methyl Ester

A solution of 3-(7-cyano-1,1-dioxo-1,2-dihydrobenzo[1,2,4]thiadiazin-3-yl)-4-hydroxy-1(3-methylbutyl)-1H-quinolin-2-one (Example 25, 94 mg, 0.216 mmol) in methanol (10 mL) and sulfuric acid (9 mL) was heated under reflux for 16 h, then cooled and diluted with water (200 mL). The precipitate was filtered and dried, then purified by chromatography on silica gel (1% methanol/dichloromethane). The product was warmed in ether, filtered and dried to give the title compound (69 mg, 68%) as a white solid. ¹H NMR (300 MHz, 3:2 CDCl₃/DMSO-d₆) δ 15.05 (1H, br s), 14.85 (1H, br s), 8.40 (1H, d, J=1.8 Hz), 8.27-8.23 (2H, m), 7.86 (1H, t, J=7.6 Hz), 7.72 (1H, d, J=8.6 Hz), 7.58 (1H, d, J=8.5 Hz), 7.42 (1H, t, J=7.6 Hz), 4.34 (2H, m), 3.92 (3H, s), 1.80 (1H, m), 1.59 (2H, m), 1.03 (6H, d, J=6.6 Hz).

### EXAMPLE 57

3-[4-Hydroxy-1-(3-methylbutyl)-2-oxo-1,2-dihydroquinolin-3-yl]-1,1-dioxo-1,4-dihydrobenzo[1,2,4]thiadiazine-7-carboxylic Acid

A stirred solution of 3-[4-hydroxy-1-(3-methylbutyl)-2-oxo-1,2-dihydroquinolin-3-yl]-1,1-dioxo-1,4-dihydrobenzo[1,2,4]thiadiazine-7-carboxylic acid, methyl ester (Example 56,42 mg, 0.090 mmol) in methanol (5 mL) and 1M aqueous sodium hydroxide (9 mL) was heated under reflux for 1 h, then carefully acidified to pH 1 with 1M aqueous hydrochloric acid, and allowed to cool. The solid was filtered, washed with water and ether and dried to give the title compound (38 mg, 93%) as a cream solid. ¹H NMR (300 MHz, DMSO-d₆) δ 14.88 (1H, br s), 14.61 (1H, br s), 13.59 (1H, br s), 8.34 (1H, d, J=1.8 Hz), 8.26-8.22 (2H, m), 7.92 (1H, m), 7.81 (1H, d, J=8.6 Hz), 7.70 (1H, d, J=8.7 Hz), 7.48 (1H, m), 4.36 (2H, m), 1.81 (1H, m), 1.57 (2H, m), 1.02 (6H, d, J=6.6 Hz).

### EXAMPLE 58

3-(1,1-Dioxo-1,4-dihydrobenzo[1,2,4]thiadiazin-3-yl)4,6-dihydroxy-1-(3-methylbutyl)-1H-quinolin-2-one

a) 6-(tert-Butyl-dimethyl-silanyloxy)-1H-benzo[d]oxazine-2,4dione

tert-Butyldimethylsilyl chloride (5.05 g, 33.5 mmol) was added to 6-hydroxy-1H-benzo[d]oxazine-2,4-dione (6.0 g, 33.5 mmol) and imidazole (2.28 g, 33.5 mmol) in chloroform. The mixture was stirred overnight and evaporated onto silica gel. Purification using flash chromatography (silica gel, 0-40% ethyl acetate/hexanes) gave the title compound (5.5 g, 56%) as a white solid. ¹H NMR (300 MHz, CDCl₃) δ 9.29 (s, NH), 7.27 (d, J=2 Hz, 1H), 6.99 (dd, J=2 and 8 Hz, 1H), 6.80 (d, J=8 Hz, 1H), 0.77 (s, 9H), 0.00 (s, 6H).

b) 6-(tert-Butyl-dimethylsilanyloxy)-1-(3-methylbutyl)-1H-benzo[d]oxazine-2,4-dione

The compound from Example 58a (324 mg, 1.1 mmol), triphenylphosphine (289 mg, 1.1 mmol) and isoamyl alcohol (0.18 ml, 1.1 mmol) were stirred together in chloroform and treated with diethyl azodicarboxylate (0.12 ml, 1.1 mmol). The reaction was stirred under nitrogen overnight, evaporated onto silica and purified by flash chromatography (silica gel, ethyl acetate-hexanes) to give the title compound (175 mg, 43.7%). ¹H NMR (300 MHz, CDCl₃) δ 7.53 (d, J=3 Hz, 1H), 7.23 (dd, J=3 and 8 Hz, 1H), 7.03 (d, J=8 Hz, 1H), 4.01 (m, 2H), 1.63 (m, 1H), 1.60 (m, 2H), 1.00 (d, J=6 Hz, 6H), 0.97 (s, 9H), 0.20 (s, 6H), 0.50 (m, 2H), 0.22 (s, 6H).

c) 6-(tert-Butyl-dimethylsilanyloxy)-1-(3-methylbutyl)-3-(1,1-dioxo-1,4-dihydrobenzo[1,2,4]thiadiazin-3-yl)-4-hydroxy-1H-benzo[d]oxazine-2,4-dione

Sodium hydride (75 mg of a 60% suspension in mineral oil, 1.88 mmol) was added to a mixture of the compound from Example 58b (170 mg, 0.46 mmol) and ethyl 1,1-dioxo-2H-benzo-1,2,4-thiadiazinyl-3-acetate (125 mg, 0.46 mmol) in tetrahydrofuran (20 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (108 mg, 43%). ¹H NMR (300 MHz, d₆-DMSO) δ 15.17 (br. s, 1H), 14.48 (br.s, 1H), 7.94 (d, J=7 Hz, 1H), 7.43-7.78 (m, 6H), 4.30 (m, 2H), 1.80 (m, 1H), 1.53 (m, 2H), 1.00 (m, 15H), 0.25 (s, 6H). Anal. (C₂₇H₃₅N₃O₅SSi) calcd. C, 59.86; H, 6.55; N, 7.76; S, 5.92. Found: C, 59.79; H, 6.55; N, 7.43; S, 6.09.

### EXAMPLE 59

1-(3-Methylbutyl)-3-(1,1-dioxo-1,4-dihydrobenzo[1,2,4]thiadiazin-3-yl)4,6-dihydroxy-1H-benzo[d]oxazine-2,4dione

0.5 ml of a 1.0 Molar solution of tetrabutylammonium fluoride in tetrahydrofuran was added to a suspension of the compound from Example 58c (70 mg, 0.13 mmol) in tetrahydrofuran (5 ml) and the mixture was stirred until a clear yellow solution was obtained. After 10 minutes, 3N hydrochloric acid (10 ml) was added, followed by water until a precipitate was obtained. The solid was collected, washed with water, ether and hexane to give the title compound as a yellow solid (53 mg, 96%). ¹H NMR (300 MHz, d₆-DMSO) δ 15.12 (br.s, 1H), 14.63 (br.s, 1H), 10.00 (br.s, OH), 7.93 (d, J=8 Hz, 1H), 7.77 (dd, J=8 Hz, 1H), 7.66 (d, J=8 Hz, 1H), 7.54 (m, 3H), 7.36 (dd, J=3 and 8 Hz, 1H), 4.29 (m, 2H), 1.80 (m, 1M), 1.54 (m, 2H), 1.00 (d, J=3 Hz, 121). Anal. (C₂₁H₂₁N₃O₅S 0.2H₂O) calcd. C, 58.51; H, 5.30; N, 9.75; S, 7.44. Found: C, 58.55; H, 5.30; N, 9.41; S, 7.45.

### EXAMPLE 60

3-(1,1-Dioxo-1,4-dihydrobenzo[1,2,4]thiadiazin-3-yl)-4-hydroxy-1-(4,4,4-trifluoro-3-methylbutyl)-1H-quinolin-2-one

a) 3-(Trifluoromethyl)-1-butanol

Ethyl 3-(trifluoromethyl)butyrate (2.0 g, 1.74 nL, 10.86 mmol) was added dropwise to a cooled solution (0° C.) of LiAlH₄ (1 M solution in THF, 8.14 mL, 8.14 mmol) in THF (10 mL). The reaction mixture was the stirred at room temperature for 20 h, quenched with water and extracted with Et₂O. The organic extracts were washed with brine, dried over MgSO₄ and evaporated at reduced pressure to afford the desired product as a yellow oil (1.3 g, 84%). ¹H-NMR (CDCl₃) δ 3.81-3.66 (m, 2H); 2.48-2.39. (m, 1H); 2.00-1.88 (m, 2H); 1.57-1.49 (m, 1H); 1.12 (d, J=6.9 Hz, 3H).

b) 3-(1,1-Dioxo-1,4dihydrobenzo[1,2,4]thiadiazin-3-yl)-4-hydroxy-1-(4,4,4-trifluoro-3-methylbutyl)-1H-quinolin-2-one

Following the procedures of Examples 28a) and 28b) except substituting 3-(trifluoromethyl)-1-butanol for cyclopentanemethanol, the title compound was obtained (205 mg, 63%) as pale yellow powder after washing the precipitate with H₂O, Et₂O, and hexanes. A portion of the material was further purified by flash chromatography (0.25% MeOH in CHCl₃) followed by recrystallization from AcOH. ¹H-NMR (CDCl₃) S, 15.31 (s, 1H); 14.42 (s, 1H); 8.32 (dd, J=8.0, 1.4 Hz, 1H); 8.00 (d, J=7.9 Hz, 1H); 7.78 (ddd, J=8.4, 7.3, 1.4 Hz, 1H); 7.64 (td, J=8.4, 1.4 Hz, 1H); 7.49-7.30 (m, 4H); 4.43-4.38 (m, 2H); 2.50-2.41 (m, 1H); 2.15-2.04 (m, 1H); 1.87-1.76 (m, 1H); 1.33 (d, J=6.9 Hz, 3H). MS(ES+) m/e 466 [M+H]⁺.

### EXAMPLE 61

4-Hydroxy-3-(7-methoxy-1,1-dioxo-1,4-dihydrobenzo[1,2,4]thiadiazin-3-yl)-1-(3-methylbutyl)-1H-quinolin-2-one

a) 2-Amino-5-methoxybenzenesulfonamide

The procedure of Example 48a) was followed, using 4-methoxyaniline in place of 2-methylaniline, to give the title compound as a solid. ¹H NMR (300 MHz, DMSO-d₆) δ 7.25 (2H, br s), 7.11 (1H, d, J=3.0 Hz), 6.94 (1H, dd, J=8.9, 3.0 Hz), 6.77 (1H, d, J=8.9 Hz), 5.45 (2H, m), 3.68 (3H, s).

b) 4-Hydroxy-3-(7-methoxy-1,1-dioxo-1,4-dihydrobenzo[1,2,4]thiadiazin-3-yl)-1-(3-methylbutyl)-1H-quinolin-2-one

The procedure of Example 55b) was followed, using 2-amino-5-methoxybenzenesulfonamide in place of 2-amino-5-chlorobenzenesulfonamide, to give the title compound as a solid. ¹H NMR (300 MHz, DMSO-d₆) δ 15.25 (1H, br s), 14.28 (1H, br s), 8.21 (1H, dd, J=8.1, 1.4 Hz), 7.91 (1H, m), 7.74-7.67 (2H, m), 7.46 (1H, t, J=7.6 Hz), 7.40-7.36 (2H, m), 4.35 (2H, m), 3.90 (3H, s), 1.81 (1H, m), 1.56 (2H, m), 1.02 (6H, d, J=6.6 Hz).

### EXAMPLE 62

[3-(1,1-Dioxo-1,4-dihydrobenzo[1,2,4]thiadiazin-3-yl)-4-hydroxy-1-(3-methylbutyl)-1H-dihydroquinolin-6-ylamino]acetic acid

a) (3-(1,1-Dioxo-1,4-dihydrobenzo[1,2,4]thiadiazin-3-yl)-4-hydroxy-1-(3-methylbutyl)-1H-dihydroquinolin-6-ylamino]acetic acid tert-butyl ester

A mixture of the compound from Example 19 (300 mg, 0.7 mmol), pyridine (0.6 ml, 74 mmol) and tert-butyl bromoacetate (0.35 ml, 2.37 mmol) were heated together in DMPU (10 ml) at 90° C. for 2.5 hours. Purification using flash chromatography (silica gel, hexanes/ethyl acetate), then trituration with ether gave the title compound as a yellow solid (40 mg, 10.5%). m.p. 218-220° C. ¹H NMR (300 MHz, CDCl₃) δ 14.85 (s, 1H), 14.77 (s, 1H), 9.94 (br t, NH), 7.78 (d, J=7 Hz, 1H), 7.47 (m, 1H), 7.26 (m, 1H), 6.95-7.18 (m, 4H), 4.10 (m, 2H), 3.70 (d, J=5 Hz, 2H), 1.61 (m, 1H), 1.42 (m, 21), 1.34 (s, 9H), 0.88 (d, 6H). Anal. (C₂₃H₃₂N₄O₆S) calcd. C, 59.98; H, 5.97; N, 10.36; S, 5.93. Found: C, 59.92; H, 5.89; N, 10.25; S, 5.64.

b) 3-(1,1-Dioxo-1,4-dihydrobenzo[1,2,4]thiadiazin-3-yl)-4-hydroxy-1-3-methylbutyl)-1H-dihydroquinolin-6-ylamino]acetic Acid

The compound from Example 62a (27 mg, 0.05 mmol) was stirred in 50% trifluoroacetic acid in dichloromethane (3 ml) for 3 hours. The mixture was diluted with water until a solid formed. The solid was collected and washed successively with water, ether and hexane to give the title compound (12 mg, 50%). m.p. 180-184° C. (deg.). ¹H NMR (300 MHz, d₆-DMSO) δ 15.11 (s, 1H), 14.82 (s, 1H), 7.93 (d, J=7 Hz, 1H), 7.75 (m, 1H), 7.70 (m, 1H), 7.53 (m, 2H), 7.33 (m, 1H), 7.13 (d, 1H), 4.31 (m, 2H), 3.92 (s, 2H), 1.78 (m, 1H), 1.55 (m, 2H), 1.00 (d, 6H).

### EXAMPLE 63

3-(1, l-Dioxo-1,4-dihydrobenzo[1,2,4]thiadiazin-3-yl)-4-hydroxy-1-(3-methylbutyl)-5-phenyl-1H-quinolin-2-one

A mixture of the compound from Example 32c) (112 mg, 0.23 mmol), phenylboronic acid (35 mg, 0.29 mmol), 2M aqueous sodium carbonate (0.30 mL) and a catalytic amount of tetrakis(triphenylphosphine) palladium(0) in dioxane (10 mL) was stirred at 100° C. for 18 h, then cooled and quenched with water (10 mL) and 3N HCl solution (10 mL). The resulting reaction mixture was extracted with chloroform three times. The chloroform solution was concentrated to give a residue which was purified by chromatography (silica gel, 12% ethyl acetate/hexanes) to give the title compound 85% pure. Crystallization from ethanol and CHCl₃ (2:1) gave the title compound (10 mg, 10%). MS(ES+) m/e 488 (M+H)⁺.

### EXAMPLE 64

4-Hydroxy-3-(7-hydroxy-1,1-dioxo-1-dihydrobenzo[1,2,4]thiadiazin-3-yl)-1-(3-methylbutyl)-1H-quinolin-2-one

Boron tribromide (0.353 mL, 3.73 mmol) was added dropwise to an ice-chilled stirred suspension of 4-hydroxy-3-(7-methoxy-1,1-dioxo-1,4-dihydrobenzo[1,2,4]thiadiazin-3-yl)-1-(3-methylbutyl)-1H-quinolin-2-one (Example 61, 550 mg, 1.25 mmol) in dichloromethane (10 mL) under argon. The mixture was stirred for 10 min at 0° C. and for 16 h at room temperature. 1M aqueous hydrochloric acid (50 mL) was added and the organic solvent removed under reduced pressure. The solid was filtered, washed with water and dried, then heated 5 min in methanol/ethyl acetate (1:1, 20 mL). After cooling, the solid was filtered, washed with ethyl acetate and dried to give the title compound (255 mg, 48%) as a white solid. ¹H NMR (300 MHz, DMSO-d₆) δ 15.32 (1H, br s), 14.22 (1H, br s), 10.47 (1H, s), 8.21 (1H, dd, J=8.1, 1.5 Hz), 7.90 (1H, m), 7.68 (1H, d, J=8.7 Hz), 7.61 (1H, m), 7.46 (1H, t, J=8.6 Hz), 7.21-7.17 (2H, m), 4.34 (2H, m), 1.80 (1H, m), 1.55 (2H, m), 1.01 (6H, d, J=6.6 Hz).

### EXAMPLE 65

{3-[4-Hydroxy-1-(3-methylbutyl)-2-oxo-1,2-dihydroquinolin-3-yl]-1,1-dioxo-1,4-dihydrobenzo[1,2,4]thiadiazin-7-yloxy)-acetic Acid Methyl Ester

Methyl bromoacetate (0.021 mL, 0.222 mmol) was added to a mixture of 4-hydroxy-3-7-hydroxy-1,1-dioxo-1,4-dihydrobenzo[1,2,4]thiadiazin-3-yl)-1-(3-methylbutyl)-1H-quinolin-2-one (Example 64, 80 mg, 0.187 mmol), potassium carbonate (84 mg, 0.608 mmol), and DMF (2 mL) and the mixture stirred under argon at 50° C. for 45 min, then cooled and diluted with water (20 mL). The solid was filtered, washed with water and ether and dried to give the title compound (80 mg, 86%) as a solid. ¹H NMR (300 MHz, DMSO-d₆) δ 15.22 (1H, br s), 14.29 (1H, br s), 8.22 (1H, dd, J=8.1, 1.5 Hz), 7.91 (1H, m), 7.74-7.67 (2H, m), 7.49-7.40 (3H, m), 5.02 (2H, s), 4.35 (2H, m), 3.73 (3H, s), 1.81 (1H, m), 1.56 (2H, m), 1.02 (6H, d, J=6.6 Hz).

### EXAMPLE 66

{3-[4-Hydroxy-1-(3-methylbutyl)-2-oxo-1,2-dihydroquinolin-3-yl]-1,1-dioxo-1,4-dihydrobenzo[1,2,4]thiadiazin-7-yloxy}-acetic Acid

1M Aqueous sodium hydroxide (2 mL, 2.00 mmol) was added dropwise to a stirred suspension of {3-[4-hydroxy-1-(3-methylbutyl)-2-oxo-1,2-dihydroquinolin-3-yl]-1,1-dioxo-1,4-dihydrobenzo[1,2,4]thiadiazin-7-yloxy}-acetic acid methyl ester (Example 65, 56 mg, 0.112 mmol) in methanol at room temperature. The mixture was stirred 5 min, then heated under reflux for 30 min, and cooled again. The pH was adjusted to 1 with 1M aqueous hydrochloric acid, water (30 mL) was added and the solid filtered, washed with water and ether, then dried to give the title compound (50 mg, 92%) as a white solid. ¹H NMR (300 MHz, DMSO-d₆) δ 15.23 (1H, br s), 14.29 (1H, br s), 13.23 (1H, br s), 8.22 (1H, dd, J=8.1, 1.4 Hz), 7.91 (1H, m), 7.74-7.67 (2H, m), 7.47 (1H, t, J=7.6 Hz), 7.41-7.35 (2H, m), 4.90 (2H, s), 4.36 (2H, m), 1.81 (1H, m), 1.56 (2H, m), 1.02 (6H, d, J=6.6 Hz).

### EXAMPLE 67

1-(2-Cyclopropylethyl)-3-(1,1-dioxo-1,4-dihydrobenzo[1,2,4]thiadiazin-3-yl)-hydroxy-6-nitro-1H-quinolin-2-one

a) 1-(2-Cyclopropyl-ethyl)-6-nitro-1H-benzo[d][1,3]oxazine-2,4-dione

Following the procedure in Example 28a, except substituting 6-nitro-1H-benzo[d][1,3]oxazine-2,4-dione for 1H-benzo[d][1,3]oxazine-2,4-dione and cyclopropyl ethanol for cyclopentanemethanol provided the title compound as a pale yellow crystalline solid. ¹H NMR (d-CDCl₃) δ 8.9 (d, J=2.66 Hz, 1H), 8.45 (dd, J=9.2 and 2.66 Hz, 1H), 7.2 (d, J=9.2 Hz, 1H), 4.1 (t, J=7.4 Hz, 2H), 1.58 (q, J=0.02, 2H), 0.6 (m, 1H), 0.4 (m, 4H). MS(ES+) m/e 277 [M+H]⁺.

b) 1-(2-Cyclopropylethyl)-3-(1,1-dioxo-1,4-dihydrobenzo[1,2,4]thiadiazin-3-yl)-4-hydroxy-6-nitro-1H-quinolin-2-one

Following the procedure in Example 1b, except substituting the compound obtained in Example 67a for I-Phenethyl-1H-benzo[d][1,3]oxazine-2,4-dione, the title compound was obtained as an orange crystaline powder after recrystallization from dimethylsulfoxide (1.04 g, 63%). ¹H NMR (d-CDCl₃) δ 15.6 (s, 1H), 14.1 (s, 1H), 9.1 (d, J=2.64 Hz, 1H), 8.5 (dd, J=9.4 and 2.67 Hz, 1H) 8.0 (d, J=7.52 Hz, 1H), 7.6 (t, J=8.36 Hz, 1H), 7.57 (d, J=9.45 Hz, 1H), 7.52 (t, J=8.0 Hz, 1H) 7.3 (d, J=8.7 Hz, 1H), 4.47 (t, J=7.7 Hz, 2H), 1.7 (q, J=7.0 Hz, 2H), 0.7-0.9 (m, 1H), 0.51-0.58 (m, 2H) 0.14 (q, J=4.9 Hz, 2H). MS(ES+) m/e 455 [M+H]+.

### EXAMPLE 68

3-[4-Hydroxy-1-(3-methylbutyl)-2-oxo-1,2-dihydroquinolin-3-yl]-1,1-dioxo-1,4-dihydrobenzo[1,2,4]thiadiazine-7-carboxylic acid dimethylamide

a) 1-(3-Methylbutyl)-4-hydroxy-3-(7-iodo-1,1-dioxo-1,4-dihydrobenzo[1,2,4]thiadiazin-3-yl)-1H-quinolin-2-one

Trimethylaluminum chloride (2 M solution in toluene, 8.30 mL, 16.6 mmol) was added dropwise to a stirred suspension of 3-cyanohydroxy-1-(3-methylbutyl)-1H-quinolin-2-one (Example 21b) (3.87 g, 15.09 mmol) and 2-iodo-5-amino-benzensulfonamide (4.50 g, 15.09 mmol) in dioxane (120 mL). The mixture was stirred at room temperature for 1 h and then heated under reflux for 8 h, treated with additional 5 mL of trimethylaluminum chloride solution and heated under reflux for an additional 18 h. The reaction mixture was then cooled, treated with 10 equivalents of sodium hydroxide (dissolved in minimum water) and heated under reflux for 1.5 h. After cooling, 3M aqu. hydrochloric acid was added to pH=1. The mixture was warmed slightly and diluted with water. The solid formed was collected by filtration, washed with water and air dried. The solid was suspended in dichloromethane, and, after filtration of the insoluble material, the solution was concentrated under reduced pressure. The residue was then taken up in EtOAc, boiled for 15 min, cooled and the solid collected by filtration to afford 1.48 g (18%) of the desired product. ¹H-NMR (CDCl₃) δ 15.03 (s, 1H); 14.76 (s, 1H); 8.30 (dd, J=8.1, 1.5 Hz, 1H); 8.27 (d, J=1.8 Hz, 1H); 7.90 (dd, J=8.6, 1.9 Hz, 1H); 7.77 (ddd, J=8.6, 7.1, 1.6 Hz, M1); 7.42-7.35 (m, 2H); 7.08 (d, J=8.6 Hz, 1); 4.34-4.29 (m, 2H); 1.87-1.73 (m, 1H); 1.68-1.61 (m, 2H); 1.07 (d, J=6.5 Hz, 6H). MS(ES+) m/e 438 [M+H]⁺.

b) 3-[4-Hydroxy-1-(3-methylbutyl)-2-oxo-1,2dihydroquinolin-3-yl]-1,1-dioxo-1,4 dihydrobenzo[1,2,4]thiadiazine-7-carboxylic Acid Dimethylamide

The iodide obtained in Example 68a) (150 mg, 0.28 mmol), PdCl₂(Ph₃P)₂ (52.3 mg, 0.07 mmol) and N,N-diisopropylethylamine (0.156 mL, 0.896 mmol) were suspended in 1-methyl-2-pyrrolidinone (3.5 mL) and the mixture was degassed, then purged with carbon monoxide gas (2×) and left under a carbon monoxide atmosphere. Dimethylamine (0.100 mL of a 40% w/w in water) was then added and the reaction mixture was stirred at 90° C. for 18 h, cooled to room temperature, poured into sat. sodium hydrogen carbonate solution and extracted with chloroform. The collected organic washes were washed with brine, dried over Na₂SO₄ and evaporated at reduced pressure. The residue was triturated in ethyl acetate to give 70.5 mg (52%) of product as a white powder. A portion of the material was recrystallized from AcOH. ¹H-NMR (CDCl₃) δ 15.09 (s, 1H); 14.82 (s, 1H); 8.33 (dd, J=8.3, 1.5 Hz, 1H); 8.04 (d, J=1.7 Hz, 1H); 7.82-7.74 (m, 2H); 7.43-7.36 (m, 3H); 4.384.30 (m, 2H); 3.13-3.08 (2 s, 6H); 1.84 (sept, J=6.7 Hz, 1H); 1.69-1.60 (m, 2H); 1.08 (d, J=6.7 Hz, 6H). MS(ES+) m/e 483 [M+H]⁺.

### EXAMPLE 69

6-Amino-1-(2-cyclopropylethyl)-3-(1,1-dioxo-1,4-dihydrobenzo[1,2,4]thiadiazin-3-yl)-4-hydroxy-1H-quinolin-2-one

A solution of the compound from Example 67b (1.04 g, 2.29 mMol) in ethanol (100 ml) and 2M aqu. sodium hydroxide (10.0 nl) with 10% palladium on charcoal was shaken under an atmosphere of hydrogen at 50 psi for 30 min. The mixture was filtered through Celite®, which was washed through with ethanol. The resulting filtrate was poured into cold 3N HCl and a yellow precipitate formed immediately. Filtration gave the title compound (688 mg, 71%, 95% pure). ¹H NMR (300 MHz, CDCl₃) δ 15.1 (s, 1H), 14.88 (s, 1H), 8.0 (d, J=7.46 Hz, 1H), 7.62 (t, J=8.37 Hz, 1H), 7.51 (d, J=2.73 Hz, 1H) 7.44 (t, J=7.73 Hz, 1H) 7.33 (s, 2H) 7.30 (d, J=2.84, 1H) 7.15 (d, J=2.75 Hz, 1H), 7.13 (d, J=2.82 Hz, 1H) 4.37 (t, J=7.63 Hz, 2H), 1.65 (q, J=7.43 Hz, 2H), 0.75-0.95 (m, 1H) 0.45-0.55 (m, 2H), 0.1-0.15 (m, 2H). MS(ES+) m/e 425 [M+H]⁺.

### EXAMPLE 70

3-(1,1-Dioxo-1,4-dihydrobenzo[1,2,4]thiadiazin-3-yl)-4-hydroxy-1-(2-methylsulfinyl-ethyl)-1H-quinolin-2-one

a) 1-(2-Methylsulfanyl-ethyl)-1H-benzo[d][1,3]oxazine-2,4-dione

To a solution of isatoic anhydride (1.0 g, 6.13 mmol) in methylene chloride(20 mL) was added sequentially triphenyl phosphine (1.77 g, 6.74 mmol) and 2-methylsulfinyl-ethanol (0.62 g, 6.74 mmol). Diisopropyl azodicarboxylate (1.33 mL, 6.74 mmol) was added dropwise to the reaction mixture. The reaction was stirred under nitrogen overnight, evaporated onto silica and purified by chromatography (silica gel, ethyl acetate-hexanes) to give the title compound (193 mg, 13%). MS(ES+) m/e 238 [M+H]+

b) 3-1,1-Dioxo-1,4-dihydrobenzo[1,2,4]thiadiazin-3-yl)-4-hydroxy-1-(2-methylsulfanylethyl)-1 H-quinolin-2-one

Sodium hydride (128 mg of a 60% suspension in mineral oil, 3.2 mmol) was added to a mixture of the compound from Example 70a) (190 mg, 0.8 mmol) and ethyl 1,1-dioxo-2H-benzo-1,2,4-thiadiazinyl-3-acetate (215 mg, 0.8 mmol) in tetrahydrofuran (15.0 mL). The mixture was heated under reflux for 1.5 h, cooled and acidified with acetic acid. The mixture was then heated under reflux for an additional 1.5 h, cooled and water added. The resulting precipitate was collected by filtration, washed with water, diethyl ether and hexanes to give the title compound (43 mg, 12.9%). MS(ES+) m/e 416 [M+H]⁺

c) 3-1,1-Dioxo-1,4-dihydrobenzo[1,2,4]thiadiazin-3-yl)-4-hydroxy-1-(2-methylsulfinyl-ethyl)-1H-quinolin-2-one

To a solution of Example 70b) (43 mg, 0.1 mmol) in chloroform (10 mL), cooled to −78° C., was added 3-chloroperoxybenzoic acid (23.5 mg, 76.2% purity, 0.10 mmol). After stirring at −78° C. for 2 hours, the temperature of the reaction mixture was slowly raised to room temperature. The precipitate was filtered and the filtrate was concentrated and purified by chromatography (silica gel, ethyl acetate-hexanes) to give the title compound (5.1 mg, 12%). ¹H NMR (400 MHz, d₆-DMSO) δ 14.8 (s, 1H), 14.3 (s, 1H), 8.2 (dd, J=1.5, 8 Hz, 1H), 7.86-7.95 (m, 2H), 7.7-7.8 (m, 1), 7.69-7.67 (m, 2H), 7.44-7.59 (m, 2H), 4.73 (t, J=7.1 Hz, 2H), 3.10 (t, J=7.1 Hz, 2H1), 2.66 (s, 3H). MS(ES+) m/e 432 [M+H]⁺.

### EXAMPLE 71

3-1,1-Dioxo-1,4dihydrobenzo[1,2,4]thiadiazin-3-yl)-4-hydroxy-1-(2-methylsulfonyl-ethyl)-1H-quinolin-2-one

To a solution of Example 70b) (43 mg, 0.1 mmol) in chloroform(10 mL), cooled to −78° C., was added 3-chloroperoxybenzoic acid (47.7 mg, 76.2% purity, 0.21 mmol). After stirring at −78° C. for 2 hours, the temperature was slowly raised to room temperature. The precipitate was filtered and the filtrate was concentrated and purified by chromatography (silica gel, methanol-chloroform) to give the title compound (20 mg, 44%). ¹H NMR (400 MHz, d₆-DMSO) δ 14.8 (s, 1H), 14.3 (s, 1H), 8.3 (dd, J=1.5, 8 Hz, 1H), 7.94-8.0 (m, 2H), 7.80-7.88 (m, 2H), 7.70-7.80 (m, 1H), 7.54-7.65 (m, 2H), 4.73 (t, J=7.1 Hz, 2H), 3.20 (t, J=7.1 Hz, 2H), 2.71 (s, 3H). MS(ES+) m/e 448 [M+H]⁺.

### EXAMPLE 72

(2-Cyclopropylethyl)-6-dimethylamino-(1,1-dioxo-1,4-dihydrobenzo[1,2,4]thiadiazin-3-yl)-4-hydroxy-1H-quinolin-2-one

To a suspension of the compound obtained in Example 69 (140 mg, 0.33 mmol), sodium acetate (27 mg, 0.33 mMol), and glacial acetic acid (38 μd, 0.66 mMol) in methanol (4 ml) was added formaldehyde (32 μl, 0.396 mmol) and sodium cyanoborohydride (20.7 mg, 0.33 mmol). The reaction mixture was stirred at room temperature for 1 hour. During the hour, the mixture changed in color form pale yellow to orange. The solvent was removed by rotary evaporation and the resulting residue was redissolved in ethyl acetate, washed with water and brine, dried over MgSO₄, filtered and concentrated. The crude material was recrystallized from DMSO. The first batch of crystals precipitated as orange needles, which were isolated and identified as the pure title compound. (95 mg, 64% o). ¹H NMR (300 MHz, CDCl₃) δ 15.1 (s, 1H), 14.9 (s, 1H), 8.0 (d, J=8.63 Hz, 1H), 7.62 (t, J=8.35 Hz, 1H), 7.43 (t, J=8.39 Hz, 1H) 7.37 (d, J=2.96 Hz, 1H) 7.32 (t, J=8.25 Hz, 1H) 7.25 (d, J=2.99 Hz, 1H) 7.20 (d, 1H), 4.38 (t, J=7.65 Hz, 2H), 3.0 (s, 6H) 1.65 (q, J=7.16 Hz, 2H), 0.7-0.9 (m, 1H) 0.47-0.54 (m, 2H), 0.13 (m, 2H). MS(ES+) m/e 453 [M+H]⁺.

### EXAMPLE 73

1-(2-Cyclopropylethyl)-3-(1,1-dioxo-1,4-dihydrobenzo[1,2,4]thiadiazin-3-yl)-4-hydroxy-6-methylamino-1H-quinolin-2-one

In the same procedure described in Example 72, the second batches of crystals precipitated out after an hour from the first batch (pale yellow) and were characterized as the title compound. ¹H NMR (300 MHz, CDCl₃) δ 15.16 (s, 1H), 14.86 (s, 1H), 8.0 (d, J=8.23 Hz, M1), 7.60 (m, 1H), 7.44 (m, 2H) 7.32 (m, 2H) 7.20 (d, J=2.61 Hz, 1H) 7.16 (d, J=2.76 Hz, 1H), 4.38 (t, J=7.60 Hz, 2H), 2.96 (s, 3H) 1.64 (q, J=7.41 Hz, 211), 0.7-0.85 (m, 1H) 0.45-0.52 (m, 2H), 0.09-0.12 (m, 21i). MS(ES+) m/e 439 [M+H]⁺.

### EXAMPLE 74

1-(2-Cyclopropylethyl)-3-(1,1-dioxo-1,4-dihydrobenzo[1,2,4]thiadiazin-3-yl)-6-fluoro-4-hydroxy-1-quinolin-2-one

a) 6-Fluorobenzo[d][1,3]oxazine-2,4-dione—(See Example 14a)

A solution of 5-fluoroanthranilic acid (1.26 g, 8.12 mmol) in tetrahydrofuran (20 mL) was treated with triphosgene (1.2 g, 4.04 mmol) and stirred at 50° C. overnight. Ice-cold dilute sodium hydrogen carbonate solution was added and the solid was collected, washed with water, then ether and dried to give the title compound (1.31 g, 89%). ¹H NMR (300 MHz, d₆-DMSO) δ 11.80 (s, NH), 7.67 (m, 2H), 7.18 (m, 1H).

b) 1-(2-Cyclopropylethyl)-6-fluorobenzo[d][1,3]oxazine-2,4-dione

The compound from Example 14a (1.0 g, 5.58 mmol), triphenylphosphine (1.44 g, 5.58 mmol) and 2-cyclopropylethanol (1.0 g, 11.6 mmol) were stirred together in chloroform and treated with diethyl azodicarboxylate (0.875 ml, 5.58 mmol). The reaction was stirred under a nitrogen atmosphere overnight, evaporated onto silica and purified by chromatography (silica gel, ethyl acetate-hexanes) to give the title compound (722 mg, 51%). ¹H NMR (300 MHz, CDCl₃) δ 7.80 (dd, J=3 Hz and 7 Hz, 1H), 7.48 (m, 1H), 7.08 (dd, J=4 Hz and 9 Hz, 1H), 4.15 (t, 2H), 1.65 (m, 2H) 0.74 (m, 1H), 0.51 (m, 2H), 0.09 (m, 2H).

c) 1-(2Cyclopropylethyl)-3-(1,1-dioxo-1,4-dihydrobenzo[1,2,4]thiadiazin-3-yl)-6-fluoro-4 hydroxy-1-quinolin-2-one

Sodium hydride (463 mg of a 60% suspension in mineral oil, 6.56 mmol) was added to a mixture of the compound from Example 74b (722 mg, 2.90 mmol) and ethyl 1,1-dioxo-2H-benzo-1,2,4-thiadiazinyl-3-acetate (777 mg, 2.90 mmol) in tetrahydrofuran (30 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (800 mg, 65%). ¹H NMR (300 MHz, d₆-DMSO) δ 15.19 (br. s, 1H), 14.31 (s, 1H), 7.57-7.96 (m, 7H), 4.43 (m, 2H), 1.60 (m, 2H), 0.86 (m, 1H), 0.43 (m, 2H), 0.08 (m, 2H). A portion converted to the sodium salt gave: Anal. (C₂₁H₁₇FNaN₃O₄S.0.5H₂O) calcd. C, 55.02; H, 3.96; N, 9.17; Na, 5.01. found: C, 55.00; H, 4.03; N, 9.12; Na, 5.03.

Alternatively, 1-(2-cyclopropylethyl)-3-(1,1-dioxo-1,4-dihydrobenzo[1,2,4]thiadiazin-3-yl)-6-fluoro-4-hydroxy-1-quinolin-2-one may be prepared using the following method:

a′) Ethyl 3-[2-(aminosulfonyl)anilino]-3-oxopropanoate

Ethylmalonylchloride (2.3 kg) was added to a solution of 2-aminobenzenesulfonamide (2 kg) and triethylamine (1.8 L) in THF (17.4 L) at such a rate that the temperature was maintained below 4° C. The mixture was stirred at <4° C. for 15 minutes. The reaction was quenched by addition of water (2.9 L) over approximately 5 min. The layers were separated and the organic solution was distilled under reduced pressure (80 Torr) to remove nine liters of solvent. Toluene (17.4 L) was added and another 7-8 L of solvent were removed by distillation. The suspension was cooled at 2-3° C. for 1b. The resulting solid was filtered, washed with toluene (2 L) and dried overnight under reduced pressure at 50-60° C. Yield: 2.86 kg (86%). ¹H NMR (300 MHz, d₆-DMSO) δ 9.6 (br s, 1H), 8.0 (m, 1H), 7.9 (m, 1H), 7.6 (m, 1H), 7.3 (m, 2H), 7.4 (m, 1H), 4.18 (q, 2H), 3.6 (s, 1H), 1.22 (t, 3H).

b′) Ethyl (1,1-dioxido-4H-1,2,4-benzothiadiazin-3-yl)acetate

A suspension of ethyl 3-[2-(aminosulfonyl)anilino]-3-oxopropanoate (1.44 kg) and cesium carbonate (0.33 kg) in absolute ethanol (7.2 L) was heated at 72±2° C. for 3h. The reaction mixture was cooled to 20° C. The solution was treated with acetic acid (144 mL) followed by addition of water (9.4 L) over 30 minutes. The suspension was cooled at 2° C. for 45 minutes and filtered. The cake was washed with cold water (1.5 L) and dried under reduced pressure at 60° C. for 48 h. Yield: 1.19 kg (88%). ¹H NMR (300 MHz, d₆-DMSO) δ 12.3 (brs, 1H), 7.8 (m, 1H), 7.7 (m, 1H), 7.5 (m, 1H), 7.3 (m, 1H), 4.16 (q, 2H), 3.71 (s, 2H), 3.33 (s, 1H), 1.22 (t, 3H).

c′) 2-Cyclopropylethylamine Hydrochloride

Borane (1M in tetrahydrofuran) (25.4 L) was charged under nitrogen and heated to 50° C. Cyclopropylacetonitrile (1.8 kg) was added at a rate to keep the reaction temperature below 55° C. Upon completion of the addition, the reaction was held at 50° C. for an additional 4 h. The intermediate borazine was decomposed at 50° C. by addition to a solution of methanol (6.6 L) and 4 M HCl in dioxane (6.8 L) at a rate to control hydrogen evolution. The solution was held at 50° C. for one hour, chilled to 20° C. and placed under −250 mbar vacuum. The solution was heated to 50° C. and concentrated to 9-12 L via vacuum distillation. The resulting suspension was cooled to 20° C., held at 20° C. for 30 min and ethyl acetate (12 L) was added. The reaction mixture was cooled to 5° C., held for 1.5 h, and the title product collected by vacuum filtration. The product was washed with cold ethyl acetate (6 L) and dried at 50° C. under vacuum. Yield: 2.0 kg (74%). ¹H NMR (300 MHz, CDCl₃) δ 8.34 (br s, 3H), 3.11 (q, 2H), 1.70 (q, 2H), 0.79 (m, 1H), 0.55 (m, 2H), 0.17 (m, 2H).

d′) 2-[(2-Cyclopropylethyl)amino]-5-fluorobenzoic Acid

A mixture of 2-bromo-5-fluorobenzoic acid (2.0 kg), 2-cyclopropylethylamine hydrochloride (1.17 kg), potassium carbonate (3.79 kg), and copper(II) bromide (0.1 kg) in tetrahydrofuran (20 L) was heated to a temperature to 63° C. at a rate of 1° C. min⁻¹. The reaction was rapidly stirred at 63° C. until all 2-bromo-5-fluorobenzoic acid had been consumed (4-5 h), was cooled to 30° C. and treated with water (12 L) and ethylenediaminetetraacetic acid (0.19 kg). The tetrahydrofuran was removed by vacuum distillation and the mixture was cooled to 20° C. Hydrochloric acid (6N, 8 L) was added to the solution over 30 min. The mixture was rapidly stirred for 15 min and the precipitated solids were collected by filtration. The solids were washed with water (8 L), with isooctane (4 L) and dried under reduced pressure at 50° C. Yield: 1.76 kg (86%). ¹H NMR (300 MHz, CD₃OD) δ 7.53 (m, 1H), 7.13 (m, 1H), 6.72 (m, 1H), 3.28 (t, 2H), 1.55 (t, 2H), 0.80 (m, 1H), 0.48 (m, 2H), 0.13 (m,2H).

e′) 1-(2-Cyclopropylethyl)-6-fluoro-2H-3,1-benzoxazine-2,4(1H)dione

A mixture of 2-[(2-cyclopropylethyl)amino]-5-fluorobenzoic acid (1.75 kg) and potassium carbonate (1.09 kg) in ethyl acetate (14.3 L) was warmed to 30° C. A solution of triphosgene (1.05 kg) in ethyl acetate (5.2 L) was added over 20 minutes while maintaining the temperature between 30-35° C. The reaction mixture was stirred for 45 min at 30-35° C., chilled to 5° C., and water (6.1 L) was added. The layers were separated and the organic layer concentrated at atmospheric pressure to 5 to 6 L. Isooctane (15.7 L) was slowly added to the warm ethyl acetate solution while keeping the temperature at 45-50° C. The solution was cooled to 20° C. over approximately 45 min. Once crystallization had occurred, the suspension was further distilled under vacuum (200 mbar, 36-37° C. distillate, jacket 40-50° C.) removing 5 L of solvent. The resulting suspension was cooled to 5° C. and held overnight. The product was filtered, washed with isooctane(2×1.8 L) and dried in vacuo at 50° C. Yield: 1.95 kg (85.9%). ¹H NMR (300 MHz, CD₃OD) δ 7.83 (m, 1H), 7.47 (m, 1H), 7.18 (m, 1H), 4.15 (t, 2H), 1.66 (t, 2H), 0.74 (m, 1H), 0.50 (m, 2H), 0.09 (m, 2H).

f′) 1-(2-Cyclopropylethyl)₃-(1,1-dioxido-4H-1,2,4-benzothiadiazin-3-yl)-6-fluoro-4-hydroxy-2(1H)-quinolinone

A suspension of 1-(2-cyclopropylethyl)-6-fluoro-2H-3,1-benzoxazine-2,4(1H)-dione (1.61 kg) and ethyl (1,1-dioxido-4H-1,2,4-benzothiadiazin-3-yl)acetate (1.85 kg) in ethyl acetate (10 L) was cooled to 15° C. and 1,8-diazabicyclo[5.4.0]undec-7-ene(DBU; 1.97 kg) was added over 15 min. The resulting solution was stirred for 3.5 hours at 15° C. and glacial acetic acid (0.97 kg) was added over 15 min. The solution was stired for 15 min and filtered. To the mixture was added dilute hydrochloric acid (3N, 6 L) over 20 min at 20° C. The resulting suspension was stirred for 1 h. The product was collected by vacuum filtration, and washed successively with water (3 L), ethanol (3 L), and methyl-tert-butyl ether (3 L). The collected solid was dried under vacuum at 50° C. Yield: 2.25 kg (81%). See step c), above, for spectral data.

g′) 1-(2-Cyclopropylethyl)-3-(1,1-dioxido-4H-1,2,4-benzothiadiazin-3-yl)-6-fluoro-4-hydroxy-2(1H)-quinolinone, Sodium

A stirred suspension of 1-(2-cyclopropylethyl)-3-(1,1-dioxido-4H-1,2,4-benzothiadiazin-3-yl)-6-fluoro-4-hydroxy-2(1H)-quinolinone (2.25 kg) in methanol (33.8 L) and water (11.3 L) was heated to 62° C. over 45 min. To the hot mixture was added a solution of 4N NaOH (2.22 kg) over 15 min. The temperature was maintained at 62° C. for 15 min and cooled to 5° C. over 2 hr. The slurry was stirred at 5° C. for 30 min and the solids were isolated by vacuum filtration. The filter cake was washed with water (2×6.6 L) and with methanol(2×6.6 L). The solids were dried under vacuum at 30-35° C. Yield: 2.16 kg (93%). See step c), above, for spectral data.

### EXAMPLE 75

1-(2-Cyclopropylethyl)-6(2-dimethylaminoethoxy)-3-(1,1-dioxo-1,4-dihydrobenzo[1;2,4]thiadiazin-3-yl)-4-hydroxy-1H-quinolin-2-one

a) 6-(tert-Butyl-dimethylsilanyloxy)-1-(2-cyclopropylethyl)-1H-benzo[d]oxazine-2,4-dione

The compound from Example 58a (2.01 g, 6.85 mmol), triphenylphosphine (1.80 g, 6.85 mmol) and 2-cyclopropylethanol (1.0 g, 11.6 mmol) were stirred together in chloroform and treated with diethyl azodicarboxylate (1.08 ml, 6.85 mmol). The reaction was stirred under nitrogen overnight, evaporated onto silica and purified by flash chromatography (silica gel, ethyl acetate-hexanes) to give the title compound (1.5 g, 60.5%). ¹H NMR (300 MHz, CDCl₃) δ 7.50 (d, J=3 Hz, 1H), 7.31 (d, J=8 Hz, 1H), 7.23 (dd, J=3 and 8 Hz, 1H), 4.48 (t, J=7 Hz, 2H), 1.70 (dt, J=7 Hz, 2H), 0.99 (s, 9H), 0.85 (m, 1H), 0.50 (m, 2H), 0.22 (s, 6H), 0.14 (m, 2H).

b) 6-(tert-Butyl-dimethylsilanyloxy)-1-(2-cyclopropylethyl)-3-(1,1-dioxo-1,4-dihydrobenzo[1,2,4]thiadiazin-3-yl)-4-hydroxy-1H-benzo[d]oxazine-2,4-dione

Sodium hydride (664 mg of a 60% suspension in mineral oil, 16.6 mmol) was added to a mixture of the compound from Example 75a (1.50 g, 4.15 mmol) and ethyl 1,1dioxo-2H-benzo-1,2,4thiadiazinyl-3-acetate (1.11 mg, 4.15 mmol) in tetrahydrofuran (30 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (1.45 g, 65%). ¹H NMR (400 MHz, d₆-DMSO) δ 15.20 (br. s, 1H), 14.52 (br.s, 1H), 7.92 (m, 1H), 7.69 (m, 2H), 7.53 (m, 2H), 7.42 (d, J=9 Hz, 1H), 4.39 (t J=7 Hz, 2H), 1.60 (m, 1H), 0.99 (s, 9H), 0.85 (m, 1H), 0.41 (m, 2H), 0.25 (s, 6H), 0.09 (m, 2H).

c) 1-(2-Cyclopropylethyl)-3-(1,1-dioxo-1,4-dihydrobenzo[1,2,4]thiadiazin-3-yl)4,6-dihydroxy-1H-benzo[d]oxazine-2,4-dione

Tetrabutylammonium fluoride in tetrahydrofuran (1.5 ml of a 1.0 M solution) was added to a suspension of the compound from Example 75b (1.21 g, 2.24 mmol) in tetrahydrofuran (30 ml) and the mixture was stirred until a clear yellow solution was obtained. After 10 minutes, 3N hydrochloric acid (100 ml) was added, followed by water until a precipitate was obtained. The solid was collected, washed with water, ether and hexane to give the title compound as a yellow solid (850 mg, 89%). ¹H NMR (300 MHz, d₆-DMSO) δ 15.13 (s, 1H), 14.67 (s, 1H), 10.02 (s, OH), 7.93 (d, J=8 Hz, 1H), 7.79 (dd, J=2 and 8 Hz, 1H), 7.69-7.49 (m, 4H), 7.36 (dd, J=2 and 9 Hz, 1H), 4.39 (t J=7 Hz, 2H), 1.58 (m, 2H), 0.82 (m, 1H), 0.43 (m, 2H), 0.10 (m, 2H).

d) 1-(2-Cyclopropylethyl)-6-(2-dimethylaminoethoxy)-3-(1,1-dioxo-1,4-dihydrobenzo[1,2,4]thiadiazin-3-yl)+hydroxy-1H-quinolin-2-one

A mixture of the compound from Example 75c (429 mg, 1.01 mmol), 2-(dimethylamino)ethyl chloride hydrochloride (225 mg, 1.56 mmol), potassium iodide (167 mg, 1.01 mmol) and excess anhydrous potassium carbonate were vigorously stirred in dimethylformamide (55 ml) at 75° C. overnight. The mixture was evaporated onto silica gel and purified by flash chromatography (silica gel, ethyl acetate/hexanes) to give the title compound as a tan colored solid (120 mg, 24%). ¹H NMR (300 MHz, d₆-DMSO) δ 16.10 (s, 1H), 7.56 (m, 2H), 7.46 (m, 1H), 7.22-7.07 (m, 4H), 4.17 (t J=5 Hz, 2H), 4.05 (m, 2H), 3.15 (m, 2H), 2.40 (s, 6H), 1.36 (m, 2H), 0.67 (m, 1H), 0.31 (m, 2H), 0.00 (m, 2H).

### EXAMPLE 76

3-[7-(2-Dimethylaminoethoxy)-1,1-dioxo-1,4-dihydrobenzo[1,2,4]thiadiazin-3-yl]4 hydroxy-1-(3-methylbutyl)-1H-quinolin-2-one

A mixture of of 4-hydroxy-3-(7-hydroxy-1,1-dioxo-1,4-dihydrobenzo[1,2,4]thiadiazin-3-yl)-1-(3-methylbutyl)-1H-quinolin-2-one (Example 64, 68 mg, 0.159 mmol), 2-(dimethylamino)ethyl chloride hydrochloride (81 mg, 0.562 mmol), potassium iodide (52 mg, 0.319 mmol), potassium carbonate (259 mg, 1.87 mmol) and dimethoxyethane/water (4:1, 2.5 nL) was heated under reflux with stirring for 18 h. After cooling, water (20 nL) was added and the mixture neutralized with 1M aqueous hydrochloric acid, then extracted with ethyl acetate. The extracts were washed with brine, dried over MgSO₄, and evaporated under reduced pressure. The residue was purified by chromatography on silica gel (10% methanol/dichloromethane) to give the title compound (14 mg, 18%) as a solid. ¹H NMR (300 MHz, DMSO-d₆) δ 16.12 (1H, br s), 8.14 (1H, dd, J=7.9, 1.6 Hz), 7.59 (1H, m), 7.36-7.22 (4H, m), 7.14 (1H, t, J=7.5 Hz), 4.38 (2H, m), 4.14 (2H, m), 3.44 (2H, m), 2.82 (6H, s), 1.74 (1H, m), 1.47 (2H, m), 1.00 (6H, d, J=6.6 Hz).

### EXAMPLE 77

3-(1,1-Dioxo-1,4-dihydrobenzo[1,2,4]thiadiazin-3-yl)-4-hydroxy-1-(3-methylbutyl)-2-oxo-1,2-dihydroquinoline-6-carboxylic Acid Methylamide

a) 6-Iodo-1H-benzo[d][1,3]oxazine-2,4-dione

Triphosgene (5.64g, 19 mmol) was added portionwise to a stirred solution of 2-amino-5-iodobenzoic acid (10 g, 38 mmol) in tetrahydrofuran (60 ml). The mixture was stirred for 1 hour then a mixture of water/ice/sodium hydrogen carbonate solution was added in portions to the mixture. The solid was collected, washed with water, ether and dried to give the title compound (9.68 g, 88%). ¹H NMR (300 MHz, d₆-DMSO) δ 11.83 (s, 1H), 8.12 (d, 1H), 8.00 (dd, 1H), 6.95 (d, 1H).

b) 6-Iodo-1-(3-methylbutyl)-1H-benzo[d][1,3]oxazine-2,4-dione

Diethyl azodicarboxylate (3.76 ml, 23.88 mmol) in chloroform (50 ml) was added dropwise to a stirred suspension of 6-Iodo-1H-benzo[d][1,3]oxazine-2,4-dione (6.90 g, 23.88 mmol), triphenylphosphine (6.26 g, 23.88 mmol) and isoamyl alcohol (2.5 ml, 23.88 mmol) in chloroform (150 ml). The mixture was stirred overnight and evaporated onto silica gel. Purification using flash chromatography (silica gel, gradient, hexanes/ethyl acetate 0-15%) gave the title compound as a white solid (5.45 g, 53%). ¹H NMR (300 MHz, CDCl₃) δ 8.42 (d, 1H), 8.00 (dd, 1H), 6.92 (d, 1H), 4.02 (m, 2H), 1.60 (m, 1H), 1.57 (m, 2H), 1.01 (d, 6H).

c) 3-1,1-Dioxo-1,4-dihydrobenzo[1,2,4]thiadiazin-3-yl)-4-hydroxy-6-iodo-1-(3-methylbutyl)-1H-quinolin-2-one

Sodium hydride (2.4 g of a 60% suspension in mineral oil, 60 mmol) was added to a mixture of the compound from Example 77b) (5.39 g, 15 mmol) and ethyl 1,1-dioxo-2H-benzo-1,2,4-thiadiazinyl-3-acetate (4.02 g, 15 mmol) in tetrahydrofuran (100 mL). The mixture was heated under reflux for 1.5 h, cooled and acidified with acetic acid. The mixture was then heated under reflux for an additional 1.5 h, cooled and water added. The precipitate was collected, washed with water, diethyl ether and hexanes to give the title compound (5.5 g, 68%). ¹H NMR (300 MHz, d₅-pyridine) a 15.24 (br.s, 1H), 14.50 (br.s, 1H), 8.59 (d, 1H), 8.25 (m, 1H), 8.05 (m, 1H), 7.89 (m, 1H), 7.79 (m, 1H), 7.69 (m, 1H), 7.59 (m, 1H), 4.44 (m, 2H), 1.94 (m, 1H), 1.70 (m, 2H), 1.16 (d, 6H).

d) 3-(1,1-dioxo-1,4-dihydrobenzo[1,2,4]thiadiazin-3-yl)-4-hydroxy-1-(3-methylbutyl)-2-oxo-1,2-dihydroquinoline-6-carboxylic Acid Methylamide

A solution of compound from Example 77c) (450 mg, 0.84 mmol), Hunig's base (0.45 mL, 2.69 mmol), palladium chloride triphenylphosphine (140 mg, 0.20 mmol) in N-methylpyrrolidine (5.0 mL) was purged with nitrogen and then carbon monoxide. The solution was treated with methylformamide (300 uL of 40% aqueous solution, 2.58 mmol) and then sealed with a carbon monoxide balloon. The resulting reaction mixture was stirred at 95° C. overnight before cooled down and poured into saturated aqueous solution of sodium bicarbonate. The solution was extracted with chloroform (20 mL). A yellow powder precipitated, which was filtered and slurried with a solution of 1:1 acetonenitrile and methanol and filtered. The acetonitrile/methanol solution was evaporated to give a residue which was then triturated with ethyl acetate to give the product as light yellow powder (200 mg, 51%). ¹H NMR (300 MHz, DMSO-d₆) δ 8.7 (d, J=2.2 Hz, 1H), 8.5 (m, 1H), 8.0 (dd, J=7.8 Hz, 1H), 7.7 (dd, J=1.0 Hz, 2H), 7.6 (m, 1H), 7.3 (m, 3H), 4.2 (m, 2H), 2.9 (d, J=3.0 Hz, 3H), 1.8 (n, 1H), 1.5 (m, 2H), 1.0 (d, J=7.0 Hz, 6H). MS(ES+) m/e 469 (M+H).

### EXAMPLE 78

1-(2-Cyclopropylethyl)-4-hydroxy-3-(7-methoxy-1,1-dioxo-1,4-dihydrobenzo[1,2,4]thiadiazin-3-yl)-1H-quinolin-2-one

a) 3-Cyano-1-(2-cyclopropylethyl)-4-hydroxy-1H-quinolin-2-one

The procedures of Examples 28(a), 45(b) were followed, using 2-cyclopropylethanol in place of cyclopentanemethanol, to give the title compound as a white solid. ¹H NMR (300 MHz, DMSO-d₆) δ 8.11 (1H, dd, J=8.1, 1.5 Hz), 7.75 (1H, m), 7.60 (1H, d, J=8.5 Hz), 7.31 (1H, t, J=7.3 Hz), 4.28 (2H, m), 1.50 (2H, m), 0.78 (1H, m), 0.39 (2H, m), 0.04 (2H, m).

b) 1-(2-Cyclopropylethyl)-4-hydroxy-3-(7-methoxy-1,1-dioxo-1,4-dihydrobenzo[1,2,4]thiadiazin-3-yl)-1H-quinolin-2-one

The procedure of Example 55(b) was followed, using 2-amino-5-methoxybenzenesulfonamide in place of 2-amino-5-chlorobenzenesulfonamide, and 3-cyano-1-(2-cyclopropylethyl)-4-hydroxy-1H-quinolin-2-one in place of 3-cyanohydroxy-1-(3-methylbutyl)-1H-quinolin-2-one to give the title compound as a solid. ¹H NMR (300 MHz, DMSO-d₆) δ 15.26 (1H, br s), 14.32 (1H, br s), 8.21 (1H, dd, J=8.2, 1.4 Hz), 7.89 (1H, m), 7.80-7.70 (2H, m), 7.46 (1H, t, J=7.6 Hz), 7.40-7.36 (2H, m), 4.44 (2H, m), 3.90 (3H, s), 1.61 (2H, m), 0.85 (1H, m), 0.43 (1H, m), 0.10 (1H, m).

### EXAMPLE 79

1-(2-Cyclopropylethyl)-3-(1,1-dioxo-1,4-dihydrobenzo[1,2,4]thiadiazin-3-yl)-6-[(furan-2-ylmethyl)amino]4-hydroxy-1H-quinolin-2-one

A mixture of the compound from Example 69 (140 mg, 0.33 mmol), sodium acetate (29.0 mg, 0.33 mmol), acetic acid (38 ul, 0.66 mmol) and 2-furaldehyde (27.3 ul, 0.33 mmol) was stirred in methanol (4.0 ml) and treated with sodium cyanoborohydride (20.7 mg, 0.33 mmol). Stirring was continued for 72 hours and the mixture then purified by prep. HPLC to give the title compound as an off white solid (15 mg, 9.0%). ¹H NMR (300 MHz, d₆-DMSO) δ 15.14 (s, 1H), 14.85 (s, 1H), 7.93 (d, J=8 Hz, 1H), 7.80-7.53 (m, 4H), 7.34 (m, 3H), 6.63 (m, NH), 6.40 (d, 2H), 4.36 (m, 4H), 1.60 (m, 2H), 0.83 (m, 1H), 0.41 (m, 2H), 0.09 (m, 2H).

### EXAMPLE 80

3-[1-(2-Cyclopropylethyl)-4-hydroxy-2-oxo-1,2-dihydroquinolin-3-yl]-1,1-dioxo-1,4-dihydro-1-benzo[1,2,4]thiadiazine-7-carboxylic Acid Dimethylamide

The iodide prepared as in Example 81b (228 mg, 0.426 mmol), PdCl₂(Ph₃)₂ (60 mg, 0.085 mmol), and N,N-diisopropylethylamine (176 mg, 1.36 mmol) were suspended in 1-methyl-2-pyrrolidinone (5 mL) and the mixture was degassed, purged with CO, and left under a CO balloon. Dimethylamine (0.155 mL of a 40% w/w solution in water) was then added and the reaction mixture was stirred at 95° C. for 2 h, cooled to room temperature, poured into saturated sodium bicarbonate solution, and extracted into chloroform. The collected organic extracts were washed with brine, dried (Na₂SO₄), and concentrated in vacuo. The residue was triturated with ethyl acetate and crystallized from hot DMSO (2 mL) to yield the title compound as a pale yellow crystalline solid (78.5 mg, 39%). ¹H NMR (d₆-DMSO) δ 15.0 (br s, 1H); 14.4 (br s, 1H); 8.21 (m, 1H); 7.92 (s, 1H); 7.85 (m,1H); 7.75 (m,3H); 7.45 (m, 1H); 4.43 (m,2H); 3.00 (br m, 6H); 1.6 (m,2H); 0.84 (m,1H); 0.41 (m, 2H); 0.09 (m, 2H). MS(ES+) m/e 481 [M+H]⁺. Mp: 214-215° C.

### EXAMPLE 81

1-(2-Cyclopropylethyl)-4-hydroxy-3-(7-iodo-1,1-dioxo-1,4-dihydrobenzo[1,2,4]thiadiazin-3-yl)-1H-quinolin-2-one

a) (7-Iodo-1,1-dioxo-1,4-dihydro-1-benzo[1,2,4]thiadiazin-3-yl)-acetic Acid Ethyl Ester

2-Amino-5-iodo-benzenesulfonamide (5.0 g, 16.77 mmol), pyridine (1.38 g, 16.77 mmol), and ethylchloro malonate (2.53 g, 16.77 mmol) were dissolved in methylene chloride (60 mL) and stirred for 18 h at room temperature. The reaction was concentrated in vacuo and the residue was triturated with ethyl acetate and filtered. The filtered solid was washed with 5% HCl and water and dried. The resulting white solid was dissolved in a mixture of 10% sodium carbonate (200 mL) and THF (100 mL) and stirred overnight at room temperature. The reaction volume was reduced to 200 mL in vacuo, acidified with 6N HCl, and the resulting precipitate was filtered and recrystallized from ethanol to give the title compound as an off-white solid (1.49 g, 15%). ¹H NMR (d₆-DMSO) δ 12.4 (br s, 1H); 8.0 (m, 2H), 7.1 (m, 1H); 4.2 (q, 2H); 3.7 (s, 2H); 1.15 (t, 3H). MS(ES+) m/e 395 [M+H]⁺.

b) 1-(2-Cyclopropylethyl)-4hydroxy-3-(7-iodo-1,1-dioxo-1,4 dihydrobenzo[1,2,4]thiadiazin-3-yl)-1H-quinolin-2-one

Sodium hydride (292 mg. of a 60% oil dispersion, 7.3 mmol) was added to a stirred suspension of 1-(2-Cyclopropyl-ethyl)-1H-benzo[d][1,3]oxazine-2,4-dione (422.0 mg, 1.82 mmol) and (7-Iodo-1,1-dioxo-1,4-dihydro-1-benzo[1,2,4]thiadiazin-3-yl)-acetic acid ethyl ester (719 mg, 1.82 mmol) in freshly distilled THF (10 mL). The mixture was stirred under reflux for 1.0 h, cooled, and acetic acid (2 mL) added. The mixture was heated under reflux for 1.0 h, cooled, and poured into 1.0 N HCl. The precipitate was filtered, washed with hexanes, and crystallized from hot DMSO (6.0 mL) to give the title compound as a white, crystalline solid (410 mg, 42%). ¹H NMR (d₆-DMSO) δ 15.0 (br s, 1H); 14.4 (br s, 1H); 8.15 (m, 2H); 8.05 (m, 1H); 7.85 (m, 1H); 7.75 (m, 1H); 7.7 (m, 1H); 7.4 (m, 1H); 4.4 (t, 2H); 1.6 (m,2H); 0.83 (m, 1H); 0.42 (m, 2H); 0.07 (m, 2H). MS(ES+) m/e 536 [M+H]⁺. mp 234-236° C.

### EXAMPLE 82

1-(2-Cyclopropylethyl)-3-(1,1-dioxo-1,4-dihydrobenzo[1,2,4]thiadiazin-3-yl)-4-hydroxy-6-(2-hydroxyethoxy)-1H-quinolin-2-one

A mixture of the compound from Example 75d (150 mg, 0.353 mmol), 2-bromoethanol (57 ul, 0.46 mmol) and potassium carbonate (138 mg, 1.0 mmol) in dimethylformamide (2.0 mL) was vigorously stirred at 80° C. in a nitrogen atmosphere for 72 hours. The mixture was cooled and poured into water and the precipitate was purified by chromatography (silica gel, hexanes/ethyl acetate) and washed with ethanol to give the title compound as a pale yellow solid (20 mg, 12%). ¹H NMR (300 MHz, d₆-DMSO) δ 15.11 (s, 1H), 14.48 (s, 1H), 7.42-7.71 (m, 6H), 7.27 (m, 1H), 4.32 (m, 2H), 4.03 (m, 2H), 3.68 (m, 2H), 1.50 (m, 1H), 0.73 (m, 2H), 0.33 (m, 2H), 0.00 (m, 2H).

### EXAMPLE 83

3-[1-(2-Cyclopropylethyl)-4-hydroxy-2-oxo-1,2-dihydroquinolin-3-yl]-1,1-dioxo-1,4-dihydro-1-benzo[1,2,4]thiadiazine-7-carboxylic Acid Methylamide

Following the procedure of Example 80, except substituting 40% methylamine for 40% dimethylamine, gave the title compound as pale yellow crystals (31.2 mg, 24%). ¹H NMR (d₆-DMSO) δ 15.0 (br s, 1H); 14.5 (br s, 1H); 8.75 (m, 1H); 8.35 (m, 1H); 8.2 (m, 2H); 7.85 (m,1H); 7.80 (m, 2H); 7.45(m, 1H); 4.40 (m, 2H); 2.80 (d, 3H); 1.7 (m, 2H); 0.8 (m, 1H); 0.4 (m, 2H); 0.09 (m, 2H). MS (ES+) m/e 467 [M+H]⁺.

### EXAMPLE 84

3-(1,1-Dioxo-1,4-dihydrobenzo[1,2,4]thiadiazin-3-yl)-4-hydroxy-1-(3-methylbutyl)-2-oxo-1,2-dihydroquinoline-5-carboxylic Acid Methylamide

A solution of the compound from Example 32c) (210 mg, 0.43 mmol), Hunig's base (0.3 mL, 3.2 mmol) and palladium chloride triphenylphosphine (50 mg, 0.07 mmol) in N-methylpyrrolidine (5.0 mL) was purged with nitrogen and then carbon monoxide. The solution was treated with methylformamide (100 uL of 40% aqueous solution, 0.86 mmol), then sealed with a carbon monoxide balloon, and stirred at 95° C. overnight. The resulting reaction mixture was cooled and poured into a saturated aqueous solution of sodium bicarbonate. The solution was extracted with chloroform (3×20 mL). The combined chloroform washings were washed with water and brine, dried over magnesium sulfate and evaporated. The residue was placed under high vacuum for 3 h and then triturated with ethyl acetate. The colorless powder was filtered and washed with ethyl acetate and diethyl ether to give the title product (50 mg, 25%). ¹H NMR (300 MHz, DMSO-d₆) δ 8.0 (d, 1H), 7.7 (m, 2H), 7.5 (m, 2H), 7.3 (m, 2H), 5.8 (m, 1H), 4.4 (m, 2H), 3.1 (d, J=4.0 Hz, 3H), 1.8 (m, 1H), 1.5 (m, 2H), 1.0 (d, 6H). MS(ES+) m/e 469 (M+H).

### EXAMPLE 85

1-(3,3-Dimethylbutyl)-3-(1,1-dioxo-1,4-dihydrobenzo[1,2,4]thiadiazin-3-yl)-4-hydroxy-6-nitro-1H-quinolin-2-one

a) 1-(3,3-Dimethylbutyl)-6-nitrobenzo[d][1,3]oxazine-2,4-dione

A stirred mixture of 6-nitrobenzo[d][1,3]oxazine-2,4-dione (3.96 g, 19.04 mmol), triphenylphosphine (5.0 g, 19.04 mmol) and 3,3dimethyl-1-butanol (2.0 ml, 19.04 mmol) in chloroform was cooled over an ice-bath and diethyl azodicarboxylate (3.0 ml, 19.04 mmol) added dropwise. The mixture was stirred at room temperature overnight and evaporated onto silica gel. Purification using flash chromatography (silica gel, ethyl acetate/hexanes) gave the title compound as a yellow solid (483 mg, 17.4%). ¹H NMR (300 MHz, CDCl₃) δ 9.01 (d, J=3 Hz, 1H), 8.6 (dd, J=3 and 9 Hz, 1H), 7.27 (d, J=9 Hz, 1H), 4.14 (m, 2H), 1.66 (m, 2H), 1.08 (s, 9H).

b) 1-(3,3-Dimethylbutyl)-3-(1,1-dioxo-1,4-dihydrobenzo[1,2,4]thiadiazin-3-yl)-4-hydroxy-6-nitro-1H-quinolin-2-one

Sodium hydride (250 mg of a 60% suspension in mineral oil, 6.28 mmol) was added to a mixture of the compound from Example 85a (483 mg, 1.57 mmol) and ethyl 1,1-dioxo-2H-benzo-1,2,4-thiadiazinyl-3-acetate (421 mg 1.57 mmol) in tetrahydrofuran (25 mL). The mixture was heated under reflux for 1.5 h, cooled and acidified with acetic acid. The mixture was then heated under reflux for an additional 1.5 h, cooled and water added. The product was collected, washed with water, diethyl ether and hexanes. Recrystallization from dimethylsulfoxide gave the title compound (219 mg, 30.0%). ¹H NMR (300 MHz, CDCl₃) δ 15.56 (s, 1H), 14.07 (s, 1H), 9.13 (d, J=3 Hz, 1H), 8.53 (dd, J=3 and 9 Hz, 1H), 7.98 (d, J=8 Hz, 1H), 7.65 (m, 1H), 7.49 (m, 2H), 7.33 (d, J=8 Hz, 1H), 4.39 (m, 2H), 1.63 (m, 2H), 1.13 (s, 9H).

### EXAMPLE 86

3-[6-Amino-4-hydroxy-1-(3-methylbutyl)-2-oxo-1,2-dihydroquinolin-3-yl]-1,1-dioxo-1,4-dihydrobenzo[1,2,4]thiadiazine-7-carboxylic acid amide

a) 3-(1,1-Dioxo-7-iodo-1,4-dihydrobenzo[1,2,4]thiadiazin-3-yl)-4-hydroxy-1-(3-methylbutyl)-6-nitro-1H-quinolin-2-one

The procedure of Example 45(b) was followed using 1-(3-methylbutyl)₆-nitrobenzo[d][1,3]oxazine-2,4-dione (Example 5a) in place of 1-(3-methylbutyl)-1H-benzo[d][1,3]oxazine-2,4-dione to give the 3-cyanoquinoline intermediate which was coupled with 2-amino-5-iodobenzenesulfonamide (A. Gouliaev et. al., WO 99/42456, 1999) using the method of Example 55(b) to give the title compound as a solid. ¹H NMR (300 MHz, DMSO-d₆) δ 13.89 (1H, br s), 8.90 (1H, d, J=2.7 Hz), 8.55 (1H, dd, J=9.4, 2.7 Hz), 8.15 (1H, d, J=1.8 Hz), 8.05 (1H, dd, J=8.6, 1.9 Hz), 7.80 (1H, d, J=9.4 Hz), 7.43 (1H, d, J=8.7 Hz), 4.33 (2H, m), 1.79 (1H, m), 1.54 (2H, m), 1.01 (6H, d, J=6.6 Hz).

b) 3-(7-Cyano-1,1-dioxo-1,2dihydrobenzo[1,2,4]thiadiazin-3-yl)-4-hydroxy-1-(3-methylbutyl)-6-nitro-1H-quinolin-2-one

The procedure of Example 25 was followed using 3-(1,1-dioxo-7-iodo-1,4-dihydrobenzo[1,2,4]thiadiazin-3-yl)-4-hydroxy-1-(3-methylbutyl)-6-nitro-1H-quinolin-2-one in place of 3-(7-bromo-1,1-dioxo-1,2-dihydrobenzo[1,2,4]thiadiazin-3-yl)-4-hydroxy-1-(3-methylbutyl)-1H-quinolin-2-one to give the title compound as a solid. LCMS (ES+) m/e 482 [M+H]⁺.

c) 3-[4-Hydroxy-1-(3-methylbutyl)-6-nitro-2-oxo-1,2-dihydroquinolin-3-yl]-1,1-dioxo-1,4-dihydrobenzo[1,2,4]thiadiazine-7-carboxylic Acid Amide

The procedure of Example 44 was followed using 3-(7-cyano-1,1-dioxo-1,2-dihydrobenzo[1,2,4]thiadiazin-3-yl)+hydroxy-1-(3-methylbutyl)$nitro-1H-quinolin-2-one in place of 3-(7-cyano-1,1-dioxo-1,2-dihydrobenzo[1,2,4]thiadiazin-3-yl)-4-hydroxy-1-(3-methylbutyl)-1H-quinolin-2-one to give the title compound as a solid, contaminated with 6% of the starting material. LCMS (ES+) m/e 500 [M+H]⁺.

d) 3-[6-Amino-4-hydroxy-1-(3-methylbutyl)-2-oxo-1,2-dihydroquinolin-3-yl]-11,1-dioxo-1,4-dihydrobenzo[1,2,4]thiadiazine-7-carboxylic Acid Amide

A suspension of 3-[4-hydroxy-1-(3-methylbutyl)-6-nitro-2-oxo-1,2-dihydroquinolin-3-yl]-1,1α-1,4-dihydrobenzo[1,2,4]thiadiazine-7-carboxylic acid amide (189 mg, 0.378 mmol) and palladium-on-charcoal (170 mg of 5%, 0.080 mmol) in methanol (20 mL) containing 1M aqueous hydrochloric acid (2 mL) was stirred under hydrogen (1 atm.) for 18 h, then the hydrogen removed. The mixture was neutralized with aqueous sodium bicarbonate and extracted with ethyl acetate. The extracts were washed with brine, dried (MgSO₄) and evaporated under reduced pressure. The residue was slurried in ether, then filtered and dried to give the title compound (130 mg, 75%) as a solid. ¹H NMR (300 MHz, DMSO-d₆) δ 15.07 (1H, br s), 8.47 (1H, d, 3=1.8 Hz), 8.32 (1H, br s), 8.23 (1H, dd, 3=8.6, 1.9 Hz), 7.78 (1H, d, J=8.6 Hz), 7.65 (1H, br s), 7A45 (1H, d, 3=9.2 Hz), 7.32 (1H, d, J=2.3 Hz), 7.24 (1H, dd, 3=9.0, 2.6 Hz), 5.73 (2H, br s), 4.29 (2H, m), 1.78 (1H, m), 1.54 (2H, m), 1.01 (6H, d, J=6.6 Hz).

### EXAMPLE 87

6-Amino-1-(3,3-dimethylbutyl)-3-(1,1-dioxo-1,4-dihydrobenzo[1,2,4]thiadiazin-3-yl)-4-hydroxy-1H-quinolin-2-one

A solution of the compound from Example 85b (190 mg, 0.4 mmol) and 10% palladium on charcoal (catalytic amount) in dimethylformamide (30 ml) was shaken under a hydrogen atmosphere at 50 psi for 4 hours. The mixture was filtered through Celite®D and evaporated to a yellow solid (1110 mg, 63%). ¹H NMR (300 MHz, CDCl₃) δ 7.90 (d, 3=8 Hz, 1H), 7.58 (dd, 1H), 7.44 (d, =3 Hz, 1H), 7.40 (m, 1H), 7.30 (d, 38 Hz, 1H), 7.14 (m, 2H), 4.24 (m, 2H), 3.01 (s, NH, 2H), 1.54 (m, 2H), 1.03 (s, 9H). Anal. (C₂₂H₂₄N₄O₄S) calcd. C, 59.98; H, 5A49; N, 12.72; S, 7.28. Found: C, 59.67; H, 5.46; N, 12.32; S, 7.24.

### EXAMPLE 88

3-(1,1-Dioxo-1,4-dihydrobenzo[1,2,4]thiadiazin-3-yl)-4-hydroxy-2-oxo-2H-quinoline-1-carboxylic Acid Ethyl Ester

a) 2-Ethoxybenzo[d][1,3]oxazin-4-one

Ethyl chloroformate (4.30 mL, 45.0 mmol) was injected over 10 min into an ice-cooled, stirred solution of 2-aminobenzoic acid (1.37 g, 10.0 mmol) in pyridine (10 mL) under argon. The mixture was stirred at 0° C. for 15 min, at room temperature for 3 h, then poured into ice-cold water (200 mL). After stirring 30 min, the solid was filtered, washed with water and dried to give the title compound (1.12 g, 59%) as a solid. ¹H NMR (300 MHz, CDCl₃) δ 8.12 (1H, m), 7.72 (1H, m), 7.42 (1H, m), 7.35 (1H, m), 4.53 (2H, q, J=7.1 Hz), 1.46 (3H, t, J=7.1 Hz).

b) 3-(1,1-Dioxo-1,4-dihydrobenzo[1,2,4]thiadiazin-3-yl)-4-hydroxy-2-oxo-2H-quinoline-1-carboxylic acid ethyl ester

Sodium hydride (125 mg of a 60% suspension in oil, 3.13 mmol) was added to a stirred suspension of 2-ethoxybenzo[d][1,3]oxazin-4-one (191 mg, 1.00 mmol) and ethyl 1,1-dioxo-2H-benzo[1,2,4]thiadiazinyl-3-acetate (268 mg, 1.00 mmol) in tetrahydrofuran (5 mL) under argon. The mixture was heated under reflux for 2 h, then cooled. Acetic acid (1 mL) was added, and the mixture heated under reflux again for 1 h. After cooling, water (50 mL) was added and the solid filtered, washed with water and ether and dried. The impure solid was partitioned between 1M aqueous hydrochloric acid and ethyl acetate. The mixture was filtered, and the organic extract was washed with water, dried (MgSO₄) and evaporated under reduced pressure. The residue was slurried with ether, filtered and dried to give the title compound (20 mg, 5%) as a white solid. ¹H NMR (300 MHz, DMSO-d₆) δ 13.46 (1H, br s), 8.22 (1H, dd, J=8.1, 1.2 Hz), 7.93-7.74 (3H, m), 7.64 (1H, d, J=7.8 Hz), 7.57-7.48 (2H, m), 7.34 (1H, d, J=8.3 Hz), 4.62 (2H, q, J=7.1 Hz), 1.42 (3H, t, J=7.1 Hz).

### EXAMPLE 89

1-(3,3-Dimethylbutyl)-3-(1,1-dioxo-1,4-dihydrobenzo[1,2,4]thiadiazin-3-yl)-4-hydroxy-6-(2-hydroxyethylamino)-1H-quinolin-2-one

A mixture of the compound from Example 87 (80 mg, 0.182 mmol), bromoethanol (33 ul, 0.455 ul) and pyridine (20 ul, 0.25 mmol) in N,N′-dimethylpropyleneurea (DMPU) (5 ml) was heated together at 92° C. under nitrogen for 2.5 hours. The mixture was cooled, diluted with water and the solid collected. Recrystallization from ethanol gave the title compound (26 mg, 29.5%). ¹H NMR (400 MHz, d₆-DMSO) δ 7.01 (d, J=8 Hz, 1H), 6.82 (dd, 1H), 6.62 (dd, 1H), 6.55 (d, J=8 Hz, 1H), 6.37 (d, 1H), 6.34 (s, 1H), 6.23 (d, 1H), 3.40 (m, 2H), 2.87 (m, 2H), 0.80 (m, 2H), 0.19 (s, 9H).

### EXAMPLE 90

6-Benzyloxy-3-(1,1-dioxo-1,4-dihydrobenzo[1,2,4]thiadiazin-3-yl)-4-hydroxy-1-(3-methylbutyl)-2-oxo-1,2-dihydroquinolin-2-one

A mixture of the compound from Example 59 (154 mg, 0.36 mmol), potassium iodide (72 mg, 0.43 mmol), excess potassium carbonate and benzyl chloride (0.5 ml, 0.43 mmol) was stirred vigorously in dimethylformamide (5 ml) at 85° C. overnight. The mixture was filtered through Celite® and evaporated. Trituration with ethanol gave a solid that was collected, washed successively with ethanol, water, ethanol, ether and hexane to give the title compound as an off white solid (25 mg, 13%). ¹H NMR (300 MHz, d₆-DMSO) δ 7.71 (m, 2H), 7.59 (m, 1H), 7.50 (m, 2H), 7.40 (m, 2H), 7.32 (m, 5H), 5.15 (s, 2H), 4.10 (m, 2H), 1.70 (m, 1H), 1.45 (m, 2H), 0.98 (d, 6H).

### EXAMPLE 91

{3-[4-Hydroxy-1-(3-methylbutyl)-2-oxo-1,2-dihydroquinolin-3-yl]-1,1-dioxo-1,4-dihydrobenzo[1,2,4]thiadiazin-7-yloxy}acetonitrile

A mixture of 4-hydroxy-3-(7-hydroxy-1,1-dioxo-1,4-dihydrobenzo[1,2,4]thiadiazin-3-yl)-1-(3-methylbutyl)-1H-quinolin-2-one (Example 64, 202 mg, 0.473 mmol), potassium carbonate (196 mg, 1.42 mmol), bromoacetonitrile (0.050 mL, 0.709 mmol) and DMF (4 mL) was stirred under argon at 60° C. for 3 b, then cooled, diluted with water (50 mL) and acidified to pH 2 with 1M aqueous hydrochloric acid. The solid was filtered, washed with water and dried. The material was slurried with ether, filtered and dried then purified by chromatography on silica gel (1-1.5% methanol/dichloromethane) to give the title compound (106 mg, 48%) as a solid. ¹H NMR (300 MHz, DMSO-d₆) δ 15.14 (br s), 14.36 (1H, br s), 8.23 (1H, d, J=8.0 Hz), 7.92 (1H, m), 7.81 (1H, d, J=9.1 Hz), 7.69 (1H, d, J=8.7 Hz), 7.63 (1H, d, J=2.7 Hz), 7.53-7.45 (2H, m), 5.39 (2H, s), 4.36 (2H, m), 1.81 (1H, m), 1.57 (2H, m), 1.03 (6H, d, J=6.5 Hz).

### EXAMPLE 92

3-[7-(2-Aminoethoxy)-1,1-dioxo-1,4-dihydrobenzo[1,2,4]thiadiazin-3-yl]4-hydroxy-1-(3-methylbutyl)-1H-quinolin-2-one

A mixture of {3-[4-hydroxy-1-(3-methylbutyl)-2-oxo-1,2-dihydroquinolin-3-yl]-1,1-dioxo-1,4-dihydrobenzo[1,2,4]thiadiazin-7-yloxy}acetonitrile (Example 91, 37 mg, 0.079 mmol), acetic acid (10 mL), 1M aqueous hydrochloric acid (1 mL) and palladium-on-charcoal (50 mg of 5%, 0.023 mmol) was shaken under hydrogen (50 psi) for 18 h, then the hydrogen removed. The mixture was diluted with methanol/dichloromethane (1:1, 20 mL) and filtered through Celite®. The filtrate was evaporated under reduced pressure and the residue redissolved in methanol/dichloromethane (1:1, 10 mL) and filtered again. The filtrate was evaporated to dryness once more, and the residue slurried with aqueous hydrochloric acid. The solid was filtered off, washed with water and dried, then warmed in methanol (1 mL). After adding ethyl acetate/ether (1:1, 5 mL), the mixture was cooled and the solid filtered, washed with ether and dried to give the title compound (22 mg, 55%) as a solid. ¹H NMR (300 MHz, DMSO-d₆) δ 15.20 (1H, br s), 14.34 (1H, br s), 8.23 (1H, dd, J=8.1, 1.4 Hz), 8.09 (3H, br s), 7.92 (1H, t, J=8.0 Hz), 7.76 (1H, d, J=9.0 Hz), 7.69 (1H, 8.7 Hz), 7.50-7.41 (3H, m), 4.34-4.31 (4H, m), 3.27 (2H, m), 1.81 (1H, m), 1.56 (2H, m), 1.02 (6H, d, J=6.6 Hz).

### EXAMPLE 93

2-{3-[4-Hydroxy-1-(3-methylbutyl)-2-oxo-1,2-dihydroquinolin-3-yl]-1,1-dioxo-1,4-dihydrobenzo[1,2,4]thiadiazin-7-yloxy}acetamide

The procedure of Example 44 was followed, using 3-[4-hydroxy-1-(3-methylbutyl)-2-oxo-1,2-dihydroquinolin-3-yl]-1,1-dioxo-1,4-dihydrobenzo[1,2,4]thiadiazin-7-yloxy}acetonitrile (Example 91) in place of 3-(7-cyano-1,1-dioxo-1,2-dihydrobenzo[1,2,4]thiadiazin-3-yl)-4-hydroxy-1-(3-methylbutyl)-1H-quinolin-2-one to give the title compound as a solid. ¹H NMR (300 MHz, DMSO-d₆) δ 15.24 (1H, br s), 14.31 (1H, br s), 8.22 (1H, dd, J=8.1, 1.4 Hz), 7.91 (1H, t, J=7.2 Hz), 7.75-7.67 (3H, m), 7.49-7.39 (4H, m), 4.61 (2H, s), 4.36 (2H, m), 1.81 (1H, m), 1.56 (2H, m), 1.02 (6H, d, J=6.6 Hz).

### EXAMPLE 94

3-(1, l-Dioxo-1,4-dihydrobenzo[1,2,4]thiadiazin-3-yl)-4-hydroxy-1-(tetrahydrofuran-3-ylmethyl)-1H-quinolin-2-one

a) 1-(Tetrahydrofuran-3-ylmethyl)-1H-benzo[d][1,3]oxazine-2,4-dione

To a solution of isatoic anhydride (1.0 g, 6.13 mmol) in methylene chloride (20 mL) was added sequentially triphenyl phosphine (1.77 g, 6.74 mmol) and (tetrahydrofuran-3-yl)-methanol (0.65 mL, 6.74 mmol). Diisopropyl azodicarboxylate (1.33 mL, 6.74 mmol) was added dropwise to the reaction mixture. The reaction was stirred under nitrogen overnight, evaporated onto silica and purified by flash column chromatography (1050% ethyl acetate in hexanes) to give the title compound (220 mg, 15%). MS(ES+) m/e 248 [M+H]⁺

b) 3-(1,1-Dioxo-1,4-dihydrobenzo[1,2,4]thiadiazin-3-yl)-4-hydroxy-1-(tetrahydro-furan-3-ylmethyl)-1H-quinolin-2-one

Sodium hydride (65 mg of a 60% suspension in mineral oil, 1.6 mmol) was added to a mixture of the compound from Example 94a) (100 mg, 0.40 mmol) and ethyl 1,1-dioxo-2H-benzo-1,2,4-thiadiazinyl-3-acetate (107 mg, 0.40 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (43 mg, 25%). ¹H NMR (400 MHz, d₆-DMSO) δ 15.3 (s, 1H), 14.4 (s, 1H), 8.30(dd, J=1.5, 8 Hz, 1H), 7.90-8.0 (m, 2H), 7.80-7.90 (m, 2H), 7.74 (d, J=8.2 Hz, 1H), 7.51 (t, J=7.5 Hz, 1H), 7.60 (t, J=7.5 Hz, 1H), 4.50 (m, 2H), 3.95 (m, 1H), 3.70 (m, 3H), 2.80 (m, 1H), 2.05 (m, 1H), 1.85 (m, 1H), 1.29 (m, 2H). MS(ES+)m/e 426 [M+H]⁺.

### EXAMPLE 95

3-(1, i-Dioxo-1,4-dihydrobenzo[1,2,4]thiadiazin-3-yl)-4-hydroxy-1-(3-methylbutyl)-2-oxo-1,2-dihydroquinoline-6-carboxylic acid ethyl ester

a) 6-Iodo-1H-benzo[d][1,3]oxazine-2,4-dione

Triphosgene (5.64 g, 19 mmol) was added portionwise to a solution of 2-amino-5-iodobenzoic acid in tetrahydrofuran (60 ml). The mixture was stirred for 1 hour then a mixture of ice/water/sodium hydrogen carbonate solution was added in portions. The solid was collected, washed with water then ether to give the title compound (9.68 g, 88%). ¹H NMR (300 MHz, d₆-DMSO) δ 11.83 (s, NH), 8.12 (d, J=2 Hz, 1H), 8.02 (dd, J=2 and 9 Hz, 1H), 6.95 (d, J=9 Hz, 1H).

b) 6-Iodo-1-(3-methylbutyl)-1H-benzo[d][1,3]oxazine-2,4-dione

A solution of diethyl azodicarboxylate (3.76 ml, 23.9 mmol) in chloroform was added dropwise to a suspension of the compound from Example 95a (6.90 g, 23.9 mmol), triphenylphosphine (6.26 g, 23.9 mmol) and isoamyl alcohol (2.5 ml, 23.9 mmol) stirred in chloroform. The mixture was stirred overnight and evaporated onto silica gel. Purification using flash chromatography (silica gel, hexanes/ethyl acetate) gave the title compound as a white solid (5.45 g, 63%). ¹H NMR (300 MHz, d₆-DMSO) δ 8.42 (m, 1H), 8.00 (m, 1H), 6.94 (m, 1H), 4.02 (m, 2H), 1.61 (m, 1H), 1.58 (m, 2H), 1.01 (d, 6H).

c) 3-(1,1-Dioxo-1,4-dihydrobenzo[1,2,4]thiadiazin-3-yl)-4-hydroxy-6-iodo-1-(3-methylbutyl)-1H-quinolin-2-one

Following the procedure of Example 94b, except substituting the compound from Example 95b for the compound from Example 94a, gave the title compound as a pale yellow solid (5.5 g, 68%). ¹H NMR (400 MHz, d₆-DMSO) δ 15.25 (br.s, 1H), 14.54 (br.s, 1H), 8.59 (d,1H), 8.25 (m, 1H), 8.06 (m, 1H), 7.90 (m, 1H), 7.49-7.79 (m, 3m), 4.44 (m, 2H), 1.9 (m, 1H), 1.67 (m, 2H), 1.16 (d, 6H).

d) 3-(1,1-Dioxo-1,4dihydrobenzo[1,2,4]thiadiazin-3-yl)-4-hydroxy-1-(3-methylbutyl)-2-oxo-1,2-dihydroquinoline-6-carboxylic Acid Ethyl Ester

A solution of the compound from Example 95c) (537 mg, 1.0 mmol), formamide (90 mg, 2.0 mmol), DMAP (129 mg, 1.1 mmol), palladium chloride triphenylphosphine (140 mg, 0.2 mmol) and dioxane (15 mL) was purged with nitrogen and then carbon monoxide and sealed with a carbon monoxide balloon. The resulting reaction mixture was stirred at 92° C. for two days before it was cooled and filtered. The residue was heated under reflux with ethanol (5 mL) and 4 drops of concentrated sulfuric acid overnight. The colorless precipitate from the reaction was filtered to give the desired product (30 mg) ¹H NMR (300 MHz, DMSO-d₆) δ 8.7 (d, 1H), 8.3 (dd, 1H), 8.0 (d, 1H), 7.8 (m, 2H), 7.7(d, 1H), 7.6 (m, 1H), 4.4 (m, 4H), 1.8 (m, 1H), 1.5 (m, 2H), 1.4 (t, 3H), 1.0 (d, 6H). MS(ES+) m/e 484 (M+H).

### EXAMPLE 96

3-(1,1-Dioxo-1,4-dihydrobenzo[1,2,4]thiadiazin-2-yl)-4-hydroxy-1-(tetrahydrofuran-2-ylmethyl)-1H-quinolin-2-one

a) 1-(Tetrahydro-furan-2-ylmethyl)-1H-benzo[d][1,3]oxazine-2,4-dione

To a solution of isatoic anhydride (1.0 g, 6.13 mmol) in methylene chloride (20 mL) was added sequentially triphenyl phosphine (1.77g, 6.74 mmol) and (tetrahydro-furan-3-yl)-methanol (0.65 mL, 6.74 mmol). Diisopropyl azodicarboxylate (1.33 ml, 6.74 mmol) was added dropwise to the reaction mixture. The reaction was stirred under nitrogen overnight, evaporated onto silica and purified by flash column chromatography (10-50% ethyl acetate in hexanes) to give the title compound (220 mg, 15%). MS(ES+) m/e 248 [M+H]⁺

b) 3-(1,1-Dioxo-1,4-dihydrobenzo[1,2,4]thiadiazin-2-yl)-4-hydroxy-1-(tetrahydro-furan-2-ylmethyl)-1H-quinolin-2-one

Sodium hydride (65 mg of a 60% suspension in mineral oil, 1.6 mmol) was added to a mixture of the compound from Example 96a) (100 mg, 0.40 mmol) and ethyl 1,1-dioxo-2H-benzo-1,2,4-thiadiazinyl-3-acetate (107 mg, 0.40 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (51 mg, 29%). ¹H NMR (400 MHz, d₆-DMSO) δ 15.2 (s, 1H), 14.3 (s, 1H), 8.13 (d, J=7.8 Hz, 1H), 7.85-7.88 (m, 1H), 7.78-7.79 (m, 2H), 7.68-7.71 (m, 1H), 7.61(d, J=8.1 Hz, 1H), 7.50 (t, J=7.8 Hz, 1H), 7.37(m, 1H), 4.39 (d, J=5.7 Hz, 2H), 4.18 (m, 1H), 3.75 (m, 1H), 3.55 (m, 1H), 1.6-2.0 (m, 4H), 1.85 (m, 1H), 1.29 (m, 2H). MS(ES+) m/e 426 [M+H]⁺.

### EXAMPLE 97

3-(7-Fluoro-1,1-dioxo-1,4-dihydrobenzo[1,2,4]thiadiazin-3-yl)-4-hydroxy-1-(3-methylbutyl)-1H-quinolin-2-one

a) 2-Amino-5-fluorobenzenesulfonamide

The procedure of Example 48(a) was followed, using 4-fluoroaniline in place of 2-methylaniline, to give the title compound as a solid. ¹H NMR (300 MHz, DMSO-d₆) δ 7.39 (2H, br s), 7.29 (1H, dd, J=9.1, 3.1 Hz), 7.17 (1H, m), 6.82 (1H, dd, J=9.0, 4.7 Hz), 5.75 (2H, br s).

b) 3-(7-Fluoro-1,1-dioxo-1,4-dihydrobenzo[1,2,4]thiadiazin-3-yl)-4-hydroxy-1-3-methylbutyl)-1H-quinolin-2-one

The procedure of Example 55(b) was followed, using 2-amino-5-fluorobenzenesulfonamide in place of 2-amino-5-chlorobenzenesulfonamide, to give the title compound as a white solid. ¹H NMR (300 MHz, DMSO-d₆) δ 15.01 (1H, br s), 14.43 (1H, br s), 8.22 (1H, dd, J=8.1, 1.5 Hz), 7.94-7.83 (3H, m), 7.73-7.67 (2H, m), 7.47 (1H, t, J=7.6 Hz), 4.35 (2H, m), 1.81 (1H, m), 1.56 (2H, m), 1.02 (6H, d, J=6.6 Hz).

### EXAMPLE 98

4-[3-(1,1-dioxo-1,4-dihydrobenzo[1,2,4]thiadiazin-3-yl)-4-hydroxy-1-(3-methylbutyl)-2-oxo-1,2-dihydroquinolin-6-yloxy]butyronitrile

A suspension of the compound from Example 87 (150 mg, 0.383 mmol) in dimethylformamide (10 ml) was treated with sodium hydride (60% suspension in oil) (40 mg, 1 mmol) and stirred for 10 mins under nitrogen. 4-Bromobutyronitrile (0.1 ml, 1.0 mmol) was added and the mixture was stirred at 85° C. for 16 hours then allowed to cool to room temperature. The mixture was diluted with water, filtered and washed successively with water, ethanol, ether and hexane to give the title compound as a pale yellow solid (81.5 mg, 43%). ¹H NMR (400 MHz, d₆-DMSO) δ 15.20 (br.s, 1H), 14.51 (br.s, 1H), 7.91 (d, J=8 Hz, 1H), 7.42-7.85 (m, 7H), 4.35 (m, 2H), 4.16 (m, 2H), 2.74 (m, 2H), 2.11 (m, 2H), 1.80 (m, 1H), 1.54 (m, 2H), 1.00 (d, J=3 Hz, 12H).

### EXAMPLE 99

3-(1,1-Dioxo-1,4-dihydrobenzo[1,2,4]thiadiazin-3-yl)-4-hydroxy-6(2-methoxyethoxy)-1-(3-methylbutyl)-1H-quinolin-2-one

Following the procedure of Example 98 except substituting 2-bromoethylmethyl ether for 4-bromobutyronitrile, the title compound was prepared and recrystallised from dimethylsulfoxide (30 mg, 16%). ¹H NMR (300 MHz, d₆-DMSO) δ 15.21 (br.s, 1H), 14.54 (br.s, 1H), 7.98 (d, J=8 Hz, 1H), 7.51-7.89 (m, 7H), 4.34 (m, 2H), 4.23 (m, 2H), 3.72 (m, 2H), 3.34 (s, 3H), 1.78 (m, 1H), 1.53 (m, 2H), 1.00 (d, J=7 Hz, 12H). Anal. (C₂₅H₃₁N₃O₆S) calcd. C, 59.37; H, 5.60; N, 8.65. Found: C, 59.07; H, 5.46; N, 8.57.

### EXAMPLE 100

3-(1,1-Dioxo-1,4dihydrobenzo[1,2,4]thiadiazin-3-yl)-4-hydroxy-1-(3-methylbutyl)-2-oxo-1,2-dihydroquinoline-6-carboxylic Acid

A solution of the compound from Example 95d) in methanol (2.0 mL) and LiOH solution (2M, 2 mL) was stirred at room temperature for 18 h. The solution was neutralized with 3N HCl until pH was 4. The resulting precipitate was filtered and washed with water to give the product as colorless powder (10 mg, 71%). ¹H NMR (300 MHz, DMSO-d₆) δ 8.7 (d, 1H), 8.3 (m, 1H), 8.0 (m, 1H), 7.8-7.5 (m, 4H), 4.4 (m, 2H), 1.8 (m, 1H), 1.5 (m, 2H), 1.0 (d, 6H). MS(ES+) m/e 456 (M+H).

### EXAMPLE 101

3-[112-Cyclopropylethyl)-4-hydroxy-2-oxo-1,2-dihydroquinolin-3-yl]-1,1-dioxo-1,4-dihydroenzo[1,2,4]thiadiazine-7-carboxylic acid (3-diethylaminopropyl)amide

The iodide obtained as in Example 81 (100 mg, 0.19 mmol), PdCl₂(Ph₃P)₂ (26.2 mg, 0.02 mmol), N,N-diisopropyletbylamine (0.104 mL, 0.60 mmol), few drops of water and 3-(diethylamine)propylamine (0.06 mL, 0.38 mmol) were suspended in 1-methyl-2-pyrrolidinone (2.5 mL) and the mixture was purged with N₂ (2×), then with CO (2×) and left under a CO atmosphere. The reaction mixture was stirred at 92° C. for 18 h. After removal of the solvent at reduced pressure, the residue was suspended in DMSO and the solid formed collected by filtration and triturated in EtOAC to give 20 mg (19%) of product as a yellow powder. ¹H-NMR (d₆-DMSO) δ 14.85 (br s, 1H); 14.61 (br s, 1H); 8.97 (br s, 1H); 8.78-8.90 (m, 1H); 8.32 (s, 1H); 8.11 (d, J=7.8 Hz, 2H); 7.78-7.56 (m, 3H); 7.34 (t, J=7.3 Hz, 1H); 4.39-4.25 (m, 2H); 3.33-3.21 (m, 2H); 3.13-3.05 (m, 6H); 1.88-1.74 (m, 2H); 1.59-1.42 (m, 2H); 1.09 (t, J=7.0 Hz, 6H); 0.78-0.69 (m, 1H); 0.38-0.27 (m, 2H); 0.02-(−)0.05 (m, 2H). MS(ES+) m/e 566 [M+H]⁺.

### EXAMPLE 102

3-[1-(2-Cyclopropylethyl)-4-hydroxy-2-oxo-1,2-dihydroquinolin-3-yl]-1,1-dioxo-1,4 dihydroenzo[1,2,4]thiadiazine-7-carboxylic acid [2-(4-methoxyphenyl)ethyl]amide

Following the procedures of Example 101 except substituting 4-methoxyphenethylamine for 3-(diethylamine)propylamine, the title compound was obtained (18 mg, 16%) as grey powder. ¹H-NMR (d₆-DMSO) δ 15.04 (br s, 1H); 14.45 (br s, 1H); 8.77 (t, J=5.4 Hz, 1H); 8.31 (d, J=1.7 Hz, 1H); 8.13-8.09 (m, 2H); 7.79 (t, J=8.5 Hz, 1H); 7.68 (d, J=8.5 Hz, 2H); 7.36 (t, J=8.6 Hz, 1H); 7.06 (d, J=8.5 Hz, 2H); 6.76 (d, J=8.6 Hz, 2H); 4.34 (t, J=7.6 Hz, 2H); 3.62 (s, 3H); 3.38 (q, J=7.0 Hz, 2H); 2.71 (t, J=7.4 Hz, 2H); 1.51 (q, J=7.3 Hz, 2H); 0.79-0.69 (m, 1H); 0.34-0.28 (m, 2H); 0.01-(−)0.02 (m, 2H). MS(ES+) m/e 587 [M+H]⁺.

### EXAMPLE 103

3-[112-Cyclopropylethyl)-4-hydroxy-2-oxo-1,2-dihydroquinolin-3-yl]-1,1-dioxo-1,4-dibydrobenzo[1,2,4]thiadiazine-7-carboxylic acid benzylamide

Following the procedures of Example 101 except substituting benzylamine for 3-(diethylamine)propylamine, the title compound was obtained (21 mg, 21%) as a white powder. ¹H-NMR (d₆-DMSO) δ 14.93 (br s, 1H); 14.47 (br s, 1H); 9.27 (t, J=5.8 Hz, 1H); 8.39 (d, J=1.8 Hz, 1H); 8.16 (dd, J=8.7, 1.9 Hz, 1H); 8.11 (dd, J=8.1, 1.4 Hz, 1H); 7.78 (td, J=8.5, 1.4 Hz, 1H); 7.69 (t, J=8.6 Hz, 2H); 7.35 (t, J=7.6 Hz, 1H); 7.30-7.21 (m, 4H); 7.19-7.13 (m, 1H); 4.41 (d, J=5.7 Hz, 2H); 4.34 (t, J=7.5 Hz, 2H); 1.51 (q, J=7.3 Hz, 2H); 0.77-0.69 (m, 1H); 0.34-0.30 (m, 2H); 0.01-(−)0.02 (m, 2H). MS(ES+) m/e 543 [M+H]⁺.

### EXAMPLE 104

1-(2-Cyclopropylethyl)-3-[1,1-dioxo-7-(1-pyrrolidin-1-yl-methanoyl)-1,4-dihydrobenzo[1,2,4]thiadiazin-3-yl]4hydroxy-1H-quinolin-2-one

The iodide obtained as in Example 81 (100 mg, 0.19 mmol), PdCl₂(Ph₃P)₂ (26.2 mg, 0.02 mmol), N,N-diisopropylethylamine (0.104 mL, 0.60 mmol), few drops of water and pyrrolidine (0.03 mL, 0.38 mmol) were suspended in 1-methyl-2-pyrrolidinone (2.5 mL) and the mixture was purged with N₂ (2×), then with CO (2×) and left under a CO atmosphere. The reaction mixture was stirred at 92° C. for 18 h. After removal of the solvent at reduced pressure, the residue was suspended in CHCl₃ and filtered to remove the residual palladium. The solution was then washed with water, brine, dried over Na₂SO₄ and the solvent evaporated at reduced pressure. The residue was triturated in EtOAc to afford the final product (48 mg, 63%) as a tan powder. ¹H-NMR (d₆-DMSO) δ 14.95 (br s, 1H); 14.39 (br s, 1H); 8.10 (d, J=8.0 Hz, 2H); 7.92 (s, 1H); 7.82-7.76 (m, 2H); 7.68-7.63 (m, 2H); 7.35 (t, J=7.5 Hz, 1H); 4.354.32 (m, 2H); 3.41-3.35 (m, 4H); 1.85-1.72 (m, 4H); 1.51 (q, J=6.9 Hz, 2H); 0.78-0.70 (m, 1H); 0.34-0.29 (m, 2H); 0.02-(−)0.03 (m, 2H). MS(ES+) m/e 507 [M+H]⁺.

### EXAMPLE 105

3-{7-[1-(4-Acetylpiperazin-1-yl)-methanoyl]-1,1-dioxo-1,4-dihydrobenzo[1,2,4]thiadiazin-3-yl}-1-(2-cyclopropylethyl)-4-hydroxy-1H-quinolin-2-one

Following the procedures of Example 101 except substituting 1-acetylpiperazine for 3-(diethylamine)propylamine, the title compound was obtained (30 mg, 28%) as a white powder. ¹H-NMR (d₆-DMSO) δ 14.95 (br s, 1H); 14.43 (br s, 1H); 8.10 (dd, J=8.1, 1.1 Hz, 1H); 7.87 (s, 1H); 7.78 (t, J=7.4 Hz, 1H); 7.72-7.65 (m, 3H); 7.35 (t, J=7.5 Hz, 1H); 4.33 (t, J=7.4 Hz, 2H); 3.50-3.35 (m, 8H); 1.93 (s, 3H); 1.50 (q, J=7.3 Hz, 2H); 0.79-0.66 (m, 1H); 0.34-0.30 (m, 2H); 0.01-(−)0.02 (m, 2H). MS(ES+) m/e 564 [M+H]+

### EXAMPLE 106

3-[1-(2-Cyclopropylethyl)-4-hydroxy-2-oxo-1,2-dihydroquinolin-3-yl]-1,1-dioxo-1,4-dihydrobenzo[1,2,4]thiadiazine-7-carboxylic acid (tetrahydrofuran-2-ylmethyl)-amide

Following the procedures of Example 104 except substituting tetrahydrofurfurylamine for pyrrolidine, the title compound was obtained (45 mg, 45%) as a yellow powder. ¹H-NMR (d₆-DMSO) δ 14.97 (br s, 1H); 14.46 (br s, 1H); 8.81 (t, J=5.7 Hz, 1H); 8.36 (d, J=1.6 Hz, 1H); 8.14-8.11 (m, 2H); 7.87 (s, 1H); 7.81-7.77 (m, 1H); 7.68 (d, J=8.6 Hz, 2H); 7.36 (t, J=7.5 Hz, 1H); 4.34 (t, J=7.4 Hz, 2H); 3.90 (quin, J=6.3 Hz, 1H); 3.70 (q, J=7.4 Hz, 1H); 3.67 (q, J=6.7 Hz, 1H); 3.49-3.15 (m, 2H); 1.88-1.63 (m, 3H); 1.57-1.48 (m, 3H); 0.82-0.70 (m, 1H); 0.34-0.30 (m, 2H); 0.01-(−)0.02 (m, 2H). MS(ES+) m/e 537 [M+H]⁺

### EXAMPLE 107

3-[1-(2-Cyclopropylethyl)-4-hydroxy-2-oxo-1,2-dihydroquinolin-3-yl]-1,1-dioxo-1,4-dihydrobenzo[1,2,4]thiadiazine-7-carboxylic acid (2,2-dimethoxyethyl)amide

Following the procedures of Example 101 except substituting aminoacetaldehyde for 3-(diethylamine)propylamine, the title compound was obtained (24 mg, 24%) as a white powder. ¹H-NMR (d₆-DMSO) δ 14.91 (br s, 1H); 14.47 (br s, 1H); 8.82 (t, J=5.8 Hz, 1H); 8.36 (d, J=1.8 Hz, 1H); 8.13-8.10 (m, 2H); 7.81 (td, J=8.3, 1.4 Hz, 1H); 7.70-7.66 (m, 2H); 7.35 (t, J=7.5 Hz, 1H); 4.45 (t, J=5.5 Hz, 1H); 4.34 (t, J=7.5 Hz, 2H); 3.31-3.24 (m, 2H); 3.21 (s, 6H); 1.51 (q, J=7.2 Hz, 2H); 0.78-0.67 (m, 1H); 0.35-0.30 (m, 2H); 0.01-(−)0.02 (m, 2H). MS(ES+) m/e 541 [M+H]⁺.

### EXAMPLE 108

3-[1-(2-Cyclopropylethyl)-4-hydroxy-2-oxo-1,2-dihydroquinolin-3-yl]-1,1-dioxo-1,4-dihydrobenzo[1,2,4]thiadiazine-7-carboxylic Acid (3-imidazol-1-ylpropyl)amide

Following the procedures of Example 101 except substituting 1-(3-aminopropyl)-imidazole for 3-(diethylamine)propylamine, the title compound was obtained (11 mg, 10%) as a yellow powder. ¹H-NMR (d₆-DMSO) δ 14.91 (br s, 1H); 14.45 (br s, 1H); 8.83 (t, J=5.7 Hz, 1H); 8.69 (s, 1H); 8.32 (d, J=1.8 Hz, 1H); 8.18 (dd, J=8.6, 1.8 Hz, 1H); 8.11 (dd, J=8.0, 1.3 Hz, 1H); 7.75-7.60 (m, 2H); 7.59-7.40 (m, 3H); 7.21 (t, J=7.6 Hz, 1H); 4.35-4.15 (m, 4H); 3.42-3.19 (m, 2H); 2.15-2.04 (m, 2H); 1.60-1.49 (m, 2H); 0.89-0.78 (m, 1H); 0.50-0.42 (m, 2H); 0.15-0.09 (m, 2H). MS(ES+) m/e 561 [M+H]⁺.

### EXAMPLE 109

4-Hydroxy-3-(5-methoxy-1,1-dioxo-1,4-dihydrobenzo[1,2,4]thiadiazin-3-yl)-1-(3-methylbutyl)-1H-quinolin-2-one

a) 2-Amino-3-methoxybenzenesulfonamide

The procedure of Example 48(a) was followed, using 2-methoxyaniline in place of 2-methylaniline, to give the title compound as a solid. ¹H NMR (300 MHz, DMSO-d₆) δ 7.29 (2H, br s), 7.19 (1H, dd, J=8.2, 1.3 Hz), 6.99 (1H, dd, J=7.9, 1.2 Hz), 6.62 (1H, t, J=8.0 Hz), 5.48 (2H, br s), 3.83 (3H, s).

b) 4-Hydroxy-3-(5-methoxy-1,1-dioxo-1,4-dihydrobenzo[1,2,4]thiadiazin-3-yl)-1-(3-methylbutyl)-1H-quinolin-2-one

The procedure of Example 55(b) was followed, using 2-amino-3-methoxybenzenesulfonamide in place of 2-amino-5-chlorobenzenesulfonamide, to give the title compound as a white solid. ¹H NMR (300 MHz, DMSO-d₆) δ 15.32 (1H, br s), 14.56 (1H, br s), 8.23 (1H, dd, J=8.1, 1.4 Hz), 7.91 (1H, m), 7.69 (1H, d, J=8.7 Hz), 7.56-7.44 (4H, m), 4.37 (2H, m), 4.07 (3H, s), 1.80 (1H, m), 1.56 (2H, m), 1.03 (6H, d, J=6.6 Hz).

### EXAMPLE 110

4-Hydroxy-3-(5-hydroxy-1,1-dioxo-1,4-dihydrobenzo[1,2,4]thiadiazin-3-yl)-1-(3-methylbutyl)-1H-quinolin-2-one

A solution of 4-hydroxy-3-(5-methoxy-1,1-dioxo-1,4-dihydrobenzo[1,2,4]thiadiazin-3-yl)-1-(3-methylbutyl)-1H-quinolin-2-one (Example 109, 396 mg, 0.897 mmol) in acetic acid (40 mL) and 48% aqueous hydrobromic acid (10 mL) was stirred while heating under reflux for 64 h. After cooling, the mixture was diluted with water (200 mL) and filtered. The solid was washed with water and dried, then heated in dichloromethane (20 mL). The solid in the warm mixture was filtered off, washed with dichloromethane and dried to give the title compound (38 mg, 10%) as a solid. ¹H NMR (300 MHz, DMSO-d₆) δ 15.42 (1H, br s), 14.42 (1H, br s), 11.48 (1H, s), 8.23 (1H, dd, J=8.1, 1.5 Hz), 7.91 (1H, m), 7.68 (1H, d, J=8.7 Hz), 7.49-7.35 (3H, m), 7.26 (1H, dd, J=7.5, 1.7 Hz), 4.36 (2H, m), 1.81 (1H, m), 1,56 (2H, m), 1.02 (6H, d, J=6.6 Hz).

### EXAMPLE 111

3-(1,1-Dioxo-1,4-dihydrobenzo[1,2,4]thiadiazin-3-yl)-4-hydroxy-1-(3-methylpentyl)-1H-quinolin-2-one

a) 1-(3-Methylpentyl)-1H-benzo[d][1,3]oxazine-2,4-dione

A solution of isatoic anhydride (1.0 g, 6.13 mmol) in methylene chloride (20 mL) was added sequentially triphenyl phosphine (1.77 g, 6.74 mmol) and 3-methyl pentan-1-ol(0.84 mL, 6.74 mmol). Diisopropyl azodicarboxylate (1.33 mL, 6.74 mmol) was added dropwise to the reaction mixture. The reaction was stirred under nitrogen overnight, evaporated onto silica and purified by flash column chromatography (10-50% ethyl acetate in hexanes) to give the title compound (220 mg, 15%). MS(ES+) m/e 248 [M+H]⁺.

b) 3-(1,1-Dioxo-1,4-dihydrobenzo[1,2,4]thiadiazin-3-yl)-4-hydroxy-1-(3-methylpentyl)-1H-quinolin-2-one

Sodium hydride (24 mg of a 60% suspension in mineral oil, 0.60 mmol) was added to a mixture of the compound from Example 11a) (36.8 mg, 0.15 mmol) and ethyl 1, 1-dioxo-2H-benzo-1,2,4-thiadiazinyl-3-acetate (40 mg, 0.15 mmol) in tetrahydrofuran (15.0 mL). The mixture was heated under reflux for 1.5 h, cooled and acidified with acetic acid. The mixture was then heated under reflux for an additional 1.5 h, cooled and water added. The product was collected, washed with water, diethyl ether and hexanes to give the title compound (25 mg, 39%). ¹H NMR (400 MHz, d₆-DMSO) δ 15.3 (s, 1H), 14.4 (s, 1H), 8.30(dd, J=1.5, 8 Hz, 1H), 7.95-8.0 (m, 2H), 7.80-7.85 (m, 1H), 7.75 (t, J=8.3 Hz, 2H), 7.60 (t, J=7.5 Hz, 1H), 7.52 (t, J=7.6 Hz, 1H), 4.44 (m, 2H), 1.47-1.75 (m, 3H), 1.27-1.35 (m, 2H), 1.07 (d, J=6.4 Hz, 3H), 0.97 (t, J=7.4 Hz, 3H). MS(ES+) m/e 426 [M+H]⁺.

### EXAMPLE 112

3-[4-Hydroxy-1-(3-methylbutyl)-2-oxo-1,2-dihydroquinolin-3-yl]-1,1-dioxo-1,4-dihydrobenzo[1,2,4]thiadiazine-7-carbaldehyde

The iodide obtained in Example 68a) (350 mg, 0.65 mmol), Pd(OAc)₂ (7.3 mg, 0.03 mmol) and 1,3-bis(diphenylphosphino)propane (13.4 mg, 0.03 mmol) were suspended in DMF (2.5 mL). The mixture was degassed, purged with CO (2×) and then left under a CO atmosphere. Triethylamine (0.227 mL, 1.63 mmol) was then added. After 15 min, Et₃SiH (0.208 mL, 1.303 mmol) was added dropwise over 15 min. The reaction mixture was warmed to 70° C. and stirred for 18 h. After cooling to room temperature, the mixture was poured into NaHCO₃ sat. solution and extracted with CHCl₃. The collected organic layers were washed with water, dried over Na₂SO₄ and evaporated at reduced pressure. The residue was triturated with EtOAc to afford 229 mg (80%) of the desired product as a white powder. ¹H-NMR (d₆-DMSO) c 9.91 (s, 1H); 8.27 (d, J=1.6 Hz, 1H); 8.15 (dd, J=7.8, 1.6 Hz, 1H); 8.02 (dd, J=8.5, 1.6 Hz, 1H); 7.57 (t, J=7.0 Hz, 1H); 7.45 (d, J=8.5 Hz, 1H); 7.26 (d, J=8.4 Hz, 1H); 7.12 (t, J=7.5 Hz, 1H); 4.154.10 (m, 2H); 1.82-1.70 (m, 1H); 1.54-1.44 (m, 2H); 1.00 (d, J=6.6 Hz, 6H). MS(ES+) m/e 440 [M+H]⁺.

### EXAMPLE 113

3-(7-Aminomethyl-1,1-dioxo-1,4-dihydrobenzo[1,2,4]thiadiazin-3-yl)-1-(2-cyclopropylethyl)-4-hydroxy-1H-quinolin-2-one

a) 3-7-Cyano-1,1-dioxo-1,2-dihydrobenzo[1,2,4]thiadiazin-3-yl)-1-(2-cyclopropylethyl)-4-hydroxy-1H-quinolin-2-one

A mixture of the compound from Example 81 (450 mg, 0.84 mmol), zinc cyanide (295 mg, 2.52 mmol), tekakis(triphenylphosphine)palladium (0) (97 mg, 0.084 mmol) and dimethylformamide (9.0 mL) was stirred at 120° C. under nitrogen for 20 h, then cooled and diluted with water (50.0 mL). The solid was filtered, washed with water and dried. The product isolated was triturated with EtOAc, filtered and dried to give the title compound (345 mg, 77%) as a white powder. ¹H NMR (d₆-DMSO) δ 8.14-8.11 (m, 2H); 7.97 (d, J=7.1 Hz, 1H); 7.62 (t, J=7.8 Hz, 1H); 7.49 (d, J=8.6 Hz, 1H); 7.42-7.35 (m, 1H); 7.18 (t, J=7.4 Hz, 1H); 4.294.11 (m, 2H); 1.59-1.42 (m, 2H); 0.85-0.73 (m, 1H); 0.49-0.40 (m, 2H); 0.19-0.08 (m, 2H).

b) 3-(7-Aminomethyl-1,1-dioxo-1,4-dihydrobenzo[1,2,4]thiadiazin-3-yl)-1-(2-cyclopropylethyl)-4-hydroxy-1H-quinolin-2-one

Palladium (10% wt. on activated carbon, ca. 20 mg) was added to a suspension of the compound from Example 113a) in 6 mL of AcOH and 0.5 mL of 1 N HCl and the mixture was shaken under H₂ (45 psi) for 20 h. The reaction mixture was diluted with 10% MeOH in CHCl₃ (20 mL), made basic (pH=14) by the addition of sodium hydroxyde (2.5 N solution in water), and filtered through Celite®. The pH of the filtrate was adjusted to 7.0 by the addition of 3 N HCl and the precipitate was collected by filtration and dried. The solid was recrystallized form DMSO and triturated with diethyl ether/EtOAc to give the title compound as a tan powder (25 mg, 31%). ¹H-NMR (d₆-DMSO) δ 16.47 (br s, 1H); 8.20 (br s, 2H); 8.13 (dd, J=8.0, 1.6 Hz, 1H); 7.86 (d, J=1.7 Hz, 1H); 7.60-7.50 (m, 2H); 7.33 (d, J=8.4 Hz, 1H); 7.11 (d, J=7.6 Hz, 1H); 4.17-4.15 (m, 2H); 4.11 (s, 2H); 1.55-1.44 (m, 2H); 0.90-0.78 (m, 1H); 0.47-0.39 (m, 2H); 0.10-0.19 (m,2H). MS(ES+) m/e 439 [M+H]⁺.

### EXAMPLE 114

4-Hydroxy-3-(6-hydroxy-1,1-dioxo-1,4-dihydrobenzo[1,2,4]thiadiazin-3-yl)-1-(3-methylbutyl)-1H-quinolin-2-one

a) 2-Amino-4-methoxybenzenesulfonamide A 2M solution of ammonia in methanol (10 mL, 20.0 mmol) was added dropwise to a stirred ice-cooled solution of 4-methoxy-2-nitrobenzensulfonyl chloride (2.52 g, 10.0 mmol) in tetrahydrofuran (30 mL) under nitrogen. After stirring 1 h, the solvent was removed under reduced pressure. The residue was stirred in methanol (30 mL) with palladium-on-charcoal (200 mg of 5%, 0.094 mmol) under hydrogen (1 atm) for 18 h. The hydrogen was removed and the mixture filtered through Celite®. The solvent was removed from the filtrate under reduced pressure to give the title compound (867 mg, 43%) as a white solid. ¹H NMR (300 MHz, DMSO-d₆) δ 7.45 (1H, d, J=8.8 Hz), 7.08 (2H, br s), 6.31 (1H, d, 2.5 Hz), 6.21 (1H, dd, J=8.8, 2.5 Hz), 5.86 (2H, br s), 3.72 (3H, s).

b) 4-Hydroxy-3-6-methoxy-1,1-dioxo-1,4-dihydrobenzo[1,2,4]thiadiazin-3-yl)-1-(3-methylbutyl)-1H-quinolin-2-one

The procedure of Example 55(b) was followed, using 2-amino-4-methoxybenzenesulfonamide in place of 2-amino-5-chlorobenzenesulfonamide, to give the title compound as a solid. ¹H NMR (300 MHz, DMSO-d₆) δ 15.39 (1H, br s), 14.27 (1H, br s), 8.23 (1H, d, J=8.1 Hz), 7.92 (1H, m), 7.84 (H, d, J=8.9 Hz), 7.68 (1H, d, J=8.6 Hz), 7.47 (1H, t, J=7.6 Hz), 7.30 (1H, s), 7.11 (1H, dd, J=8.9, 2.4 Hz), 4.35 (2H, m), 3.93 (3H, s), 1.81 (1H, m), 1.57 (2H, m), 1.02 (6H, d, J=6.6 Hz).

c) 4-Hydroxy-3-(6-hydroxy-1,1 dioxo-1,4-dihydrobenzo[1,2,4]thiadiazin-3-yl)-1-(3-methylbutyl)-1H-quinolin-2-one

The procedure of Example 110 was followed, using 4-hydroxy-3-(6-methoxy-1,1-dioxo-1,4-dihydrobenzo[1,2,4]thiadiazin-3-yl)-1-(3-methylbutyl)-1H-quinolin-2-one in place of 4-hydroxy-3-5-methoxy-1,1-dioxo-1,4-dihydrobenzo[1,2,4]thiadiazin-3-yl)-1-(3-methylbutyl)-1H-quinolin-2-one to give the title compound as a solid. ¹H NMR (300 MHz, DMSO-d₆) δ 15.35 (1H, br s), 14.12 (1H, br s), 10.85 (1H, s), 8.22 (1H, dd, J=8.1, 1.5 Hz), 7.91 (1H, m), 7.76 (1H, d, J=8.7 Hz), 7.68 (1H, d, J=8.7 Hz), 7.47 (1H, t, J=7.6 Hz), 6.96 (1H, dd, J=8.7, 2.2 Hz), 6.87 (1H, d, J=2.2 Hz), 4.35 (2H, m), 1.80 (1H, m), 1.56 (2H, m), 1.02 (6H, d, J=6.6 Hz).

### EXAMPLE 115

(E)-3-{3-[4-Hydroxy-1-(3-methylbutyl)-2-oxo-1,2-dihydroquinolin-3-yl]-1,1-dioxo-1,4-dihydrobenzo[1,2,4]thiadiazin-7-yl}acrylamide

a) 4-Hydroxy-3-(7-iodo-1,1-dioxo-1,4-dihydrobenzo[1,2,4]thiadiazin-3-yl)-1-(3-methylbutyl)-1H-quinolin-2-one

A 1M solution of dimethylaluminum chloride in hexane (1 mL, 1.00 mmol) was injected into a stirred solution of 2-amino-5-iodobenzenesulfonamide (298 mg, 1.00 mmol, A. Gouliaev et. al., WO 99/42456, 1999) in dioxane (8 mL) under argon. The mixture was stirred at 100° C. for 2 min, cooled and 3-cyano-hydroxy-1-(3-methylbutyl)-1H-quinolin-2-one (Example 45b, 256 mg, 1.00 mmol) added. The mixture was stirred while heating under reflux for 6 h, then cooled. 1M aqueous NaOH (10 mL) was added and the mixture heated under reflux again for 1 h, then cooled slightly and 1M aqueous hydrochloric acid (20 mL) added. After allowing to cool to room temperature, the mixture was diluted with water (20 mL) and the solid filtered, washed with water, dried and purified by chromatography on silica gel (dichloromethane) to give the title compound (121 mg, 23%) as a white solid. ¹H NMR (300 MHz, DMSO-d₆) δ 14.99 (1H, br s), 14.42 (1H, br s), 8.23-8.19 (2H, m), 8.09 (1H, dd, J=8.7, 1.9 Hz), 7.92 (1H, m), 7.69 (1H, d, J=8.7 Hz), 7.54 (1H, d, J=8.7 Hz), 7.47 (1H, t, J=7.6 Hz), 4.35 (2H, m), 1.80 (1H, m), 1.56 (2H, m), 1.02 (1H, d, J=6.6 Hz).

b) (E)-3-{3-[4-Hydroxy-1-(3-methylbutyl)-2-oxo-1,2-dihydroquinolin-3-yl]-1,1-dioxo-1,4-dihydrobenzo[1,2,4]thiadiazin-7-yl}acrylamide

A mixture of 4-hydroxy-3-(7-iodo-1,1-dioxo-1,4-dihydrobenzo[1,2,4]thiadiazin-3-yl)-1-(3-methylbutyl)-1H-quinolin-2-one (100 mg, 0.186 mmol), acrylamide (26 mg, 0.372 mmol), palladium acetate (8 mg, 0.036 mmol), sodium acetate (46 mg, 0.558 mmol), dimethylformamide (2 mL) and water (0.5 mL) was stirred at 90° C. under argon for 18 h, then cooled and diluted with 1M aqueous hydrochloric acid (20 mL). The solid was filtered and dried, then dissolved in 1M aqueous potassium carbonate (1 mL) and dimethylformamide (5 mL) and filtered. The filtrate was acidified (1M aqueous hydrochloric acid) and the solid filtered, washed with water and dried. The product was further purified by heating in 1:1 dimethylsulfoxide/dimethylformamide (2 mL), then precipitating with water. After filtering from the cooled mixture, the solid was washed with water and ether, then dried to give the title compound (39 mg, 44%) as a solid. ¹H NMR (300 MHz, DMSO-d₆) δ 15.09 (1H, br s), 14.48 (1H, br s), 8.23 (1H, dd, J=8.1, 1.4 Hz), 8.11 (1H, s), 7.98-7.89 (2H, m), 7.77 (1H, d, J=8.5 Hz), 7.69 (1H, d, J=8.5 Hz), 7.5&7.44 (3H, m), 7.25 (1H, br s), 6.79 (1H, d, J=15.9 Hz), 4.35 (2H, m), 1.81 (1H, m), 1.57 (2H, m), 1.02 (6H, d, J=6.6 Hz).

### EXAMPLE 116

3-(1,1-Dioxo-1,4-dihydrobenzo[1,2,4]thiadiazin-3-yl)4,5,6-trihydroxy-1-(3-methylbutyl)-1H-quinolin-2-one

a) 5,6-Dimethoxy-1H-benzo[d][1,3]oxazine-2,4-dione

A solution of 6-tert-Butoxycarbonylamino-2,3-dimethoxy-benzoic acid (prepared by the method of Bengtsson, S.; Högberg, T. J. Org. Chem. 1989, 54, 4549) (3.89 g, 13.1 mmol) in anhydrous benzene (30.0 mL) was treated with oxalyl chloride (1.26 mL; 14.4 mmol) and the reaction was heated to reflux for 30 min prior to concentration in vacuo. ¹H NMR (300 MHz, CDCl₃) δ 8.17 (br s, 1H), 7.01 (d,J=9 Hz, 1H), 6.72 (d, J=9 Hz, 1H), 4.00 (s, 3H), 3.90 (s, 3H). MS(ES+) m/e 224 [M+H]⁺.

b) 5,6-Dimethoxy-1-(3-methyl-butyl)-1H-benzo[d][1,3]oxazine-2,4-dione

A solution of the compound from Example 116a) (3 g, 13.1 mmol) in anhydrous dimethylformamide (20.0 mL) was treated with sodium hydride (60% dispersion in mineral oil) (0.681 g; 17.0 mmol) followed by 1-bromo-3-methylbutane (2.35 mL, 19.7 mmol). The reaction was stirred at room temperature for 18h and then poured into 1M aqueous hydrochloric acid. The resulting precipitate was filtered, washed with H₂O, and dried in vacuo to give the title compound as a pale-yellow solid (1.3 g, 33%, 2 steps). ¹H NMR (400 MHz, CDCl₃) δ 7.31 (d, J=9 Hz, 1H), 6.82 (d, J=9 Hz, 1H), 4.04-3.98 (m, 2H), 3.98 (s, 3H), 3.90 (s, 3H), 1.74 (dq, J=7, 13 Hz, 1H), 1.65-1.58 (m, 2H), 1.01 (d, J=7 Hz, 6H). MS(S+) m/e 294 [M+H]⁺.

c) 3-(1,1-Dioxo-1,4-dihydrobenzo[1,2,4]thiadiazin-3-yl)-4-hydroxy-5,6-dimethoxy-1-(3-methylbutyl)-1H-quinolin-2-one

A solution of the compound from Example 116b) (0.328 g, 1.12 mmol) and ethyl 1,1-dioxo-2H-benzo-1,2,4-thiadiazinyl-3-acetate (0.300 g, 1.12 mmol) in anhydrous tetrahydrofuran (10.0 mL) was treated with sodium hydride (60% dispersion in mineral oil) (0.179 g; 4.47 mmol). The reaction was heated under reflux for 2.5h, cooled to room temperature, and acetic acid (2.0 mL) was added. The reaction was again heated under reflux for 1 h then cooled to room temperature and poured into 1M aqueous hydrochloric acid. The resulting precipitate was filtered and washed with warm methanol to give the title compound as a pale-yellow solid (0.267 g, 51%). ¹H NMR (300 MHz, CDCl₃) δ 14.8 (s, 1H), 14.3 (s, 1H), 7.93 (d, J=8 Hz, 1H), 7.78 (t, J=8 Hz, 1H), 7.70 (d, J=9 Hz, 1H), 7.63 (d, J=8 Hz, 1H), 7.56 (t, J=8 Hz, 1H), 7.40 (d, J=9 Hz, 1H), 4.31 (m, 2H), 3.91 (s, 6H), 1.79 (m, 1H), 1.54 (m, 2H), 1.01 (d, J=6 Hz, 6H). MS(ES+) m/e 472 [M+H]⁺.

d) 3-(1,1-Dioxo-1,4-dihydrobenzo[1,2,4]thiadiazin-3-yl)4,5,6-trihydroxy-1-(3 methylbutyl)-1H-quinolin-2-one

A solution of the compound from Example 116c) (0.200 g, 0.424 mmol) in glacial acetic acid (20.0 mL) was treated with aqueous hydrogen bromide (48%, 4 mL) and heated to reflux for 18h. The reaction was cooled to room temperature and treated with H₂O (25 mL). The resulting precipitate was filtered and dried in vacuo to give the title compound as a yellow solid (0.175 g, 93%). ¹H NMR (300 MHz, CDCl₃) δ 14.1 (s, 1H), 7.89 (d, J=8 Hz, 1H), 7.74 (t, J=7 Hz, 1H), 7.65 (d, J=8 Hz, 1H), 7.51 (t, J=7 Hz, 1H), 7.25 (d, J=8 Hz, 1H), 6.93 (d, J=8 Hz, 1H), 4.22 (m, 2H), 1.76-1.71 (m, 1H), 1.57 (m, 2H), 0.98 (d, J=6 Hz, 6H). MS(ES+) m/e 444 [M+H]⁺.

### EXAMPLE 117

3-(1,1-Dioxo-1,4-dihydrobenzo[1,2,4]thiadiazin-3-yl)-4-hydroxy-1-3-methoxybutyl)-1H-quinolin-2-one

Following the procedures of Examples 28a) and 28b) except substituting 3-methoxy-1-butanol for cyclopentanemethanol, the title compound was obtained (388 mg, 56%) as pale yellow crystals after washing the precipitate with H₂O, hexanes and Et₂O. ¹H-NMR (CDCl₃) δ 15.23 (s, 1H); 14.59 (s, 1H); 8.30 (dd, J=8.0, 1.5 Hz, 1H); 8.01 (d, J=7.9 Hz, 1H); 7.77 (ddd, J=8.6, 7.2, 1.5 Hz, 1H); 7.66-7.60 (m, 1H); 7.57 (d, J=8.6 Hz, 1H); 7.48-7.29 (m, 3H); 4.524.35 (m, 2H); 3.54-3.47 (m, 1H); 3.41 (s, 3H); 1.94-1.83 (m, 2M); 1.24 (d, J=6.1 Hz, 3H). MS(ES+) m/e 428 [M+H]⁺.

### EXAMPLE 118

3-(1,1-Dioxo-1,4-dihydrobenzo[1,2,4]thiadiazin-3-yl)-4-hydroxy-1-(pyridin-3-ylmethyl)-1H-quinolin-2-one

Following the procedures of Examples 28a) and 28b) except substituting 3-pyridylcarbinol for cyclopentanemethanol, the title compound was obtained (185 mg, 50%) as the hydrochloride salt as a white powder after washing the precipitate with H₂O, Et₂O and hexane. ¹H-NMR (d₆-DMSO) δ 14.12 (s, 1H); 8.83 (br s, 1H); 8.69 (br s, 1H); 8.27 (dd, J=8.1, 1.5 Hz, 1H); 8.10 (d, J=7.7 Hz, 1H); 7.97 (d, J=7.9 Hz, 1H); 7.85-7.76 (m, 2H); 7.70-7.54 (m, 4H); 7.49 (t, J=7.5 Hz, 1H); 5.77 (s, 2H). MS(ES+) m/e 433 [M+H]⁺. Anal. (C₂₂H₁₆N₄O₄S.HCl) calcd. C, 56.35; H, 3.65; N, 11.95. Found: C, 56.15; H, 3.73; N, 11.91.

### EXAMPLE 119

3-(1,1-Dioxo-1,4-dihydrobenzo[1,2,4]thiadiazin-3-yl)-4-hydroxy-1-(pyridin-4-ylmethyl)-1H-quinolin-2-one

Following the procedures of Examples 28a) and 28b) except substituting 4-pyridylcarbinol for cyclopentanemethanol, the title compound was obtained (151 mg, 41%) as the hydrochloride salt as a white powder after washing the precipitate with H₂O, Et₂O, 2 mL EtOAc and hexane. ¹H-NMR (d₆-DMSO) δ 14.04 (s, 1H); 8.76 (d, J=6.5 Hz, 2H); 8.29 (dd, J=8.0, 1.3 Hz, 1H); 7.97 (d, J=7.8 Hz, 1H); 7.84-7.76 (m, 4H); 7.67 (d, J=8.0 Hz, 1H); 7.60-7.47 (m, 3H); 5.86 (s, 2H). MS(ES+) m/e 433 [M+H]⁺. Anal. (C₂₂H₁₆N₄O₄S.HCl) calcd. C, 56.35; H, 3.65; N, 11.95. Found: C, 56.30; H, 3.63; N, 11.89.

### EXAMPLE 120

3-(1,1-Dioxo-1,4-dihydrobenzo[1,2,4]thiadiazin-3-yl)+hydroxy-1-(3-methylbutyl)-(5,6-methylenedioxy)-1H-quinolin-2-one

A solution of the compound from Example 116d) (0.037 g, 0.083 mmol) in anhydrous dimethylformamide (3.0 mL) was treated with potassium carbonate (0.115 g, 0.834 mmol) and methylene chloride (0.027 mL, 0.417 mmol) and heated to 105° C. for 18h. Additional methylene chloride (0.020 mL, 0.312 mmol) was added and the reaction was heated at 105° C. for an additional 6h. The reaction was cooled to room temperature and quenched with 1M aqueous hydrochloric acid and diluted with brine. The product was extracted with ethyl acetate, dried over magnesium sulfate, filtered, concentrated in vacuo, and purified via flash column chromatography (50-100% ethyl acetate in hexanes) to give the title compound as a light yellow solid (0.020 g, 53%). ¹H NMR (300 MHz, CDCl₃) δ 15.4 (s, 1H), 14.4 (s, 1H), 7.80 (d, J=8 Hz, 1H), 7.72 (t, J=8 Hz, 1H), 7.65 (d, J=9 Hz, 1H), 7.51 (d, J=8 Hz, 1H), 7.27 (t, J=8 Hz, 1H), 7.22 (d, J=9 Hz, 1H), 6.17 (s, 2H), 4.14 (m, 2H), 1.74-1.67 (m, 1H), 1.48 (m, 2H), 0.98 (d, J=7 Hz, 6H). MS(ES+) m/e 456 [M+H]⁺.

### EXAMPLE 121

3-(1,1-Dioxo-7-(2-oxo-2-pyridin-3-ylethoxy)-1,4-dihydrobenzo[1,2,4]thiadiazin-3-yl)-4-hydroxy-1-(3-methylbutyl)-1H-quinolin-2-one

Sodium hydride (48 mg of a 60% oil suspension, 1.20 mmol) was added to a stirred solution of 4-hydroxy-3-(7-hydroxy-1,1-dioxo-1,4-dihydrobenzo[1,2,4]thiadiazin-3-yl)-1-(3-methylbutyl)-1H-quinolin-2-one (Example 64, 100 mg, 0.234 mmol) in dimethylformamide (1 mL) at 70° C. under argon. After 5 min, 3-(bromoacetyl)pyridine hydrobromide (105 mg, 0.374 mmol) was added in one portion and the mixture stirred at 70° C. for 2 h, then cooled, diluted with aqueous hydrochloric acid and extracted with ethyl acetate. The extracts were washed with 0.1M aqueous potassium carbonate, water, 1M aqueous hydrochloric acid, and brine, then dried (magnesium sulfate) and evaporated under reduced pressure. The residue was slurried with hot ether, cooled and the solid filtered, washed with ether and dried to give the title compound (42 mg, 33%) as a solid. ¹H NMR (300 MHz, DMSO-d₆) δ 15.25 (1H, br s), 14.29 (1H, br s), 9.24 (1H, d, J=1.7 Hz), 8,88 (1H, dd, J=4.8, 1.6 Hz), 8.39 (1H, m), 8.22 (1H, dd, J=8.1, 1.5 Hz), 7.91 (1H, m), 7.75-7.59 (4H, m), 7.50-7.44 (2H, m), 5.86 (2H, s), 4.36 (2H, m), 1.81 (1H, m), 1.57 (2H, m), 1.02 (6H, d, J=6.6 Hz).

### EXAMPLE 122

4-Hydroxy-1-(3-methylbutyl)-3-(4-methyl-1,1-dioxo-1,4-dihydrobenzo[1,2,4]thiadiazin-3-yl)-1-quinolin-2-one

a) 4-methyl-1,1-dioxo-1,4-dihydrobenzo[1,2,4]thiadiazin-3-one

N-Methylaniline (4.21 g, 39.3 mmol) in nitroethane (10 ml) was added dropwise to a solution of chlorosulfonyl isocyanate (4.2 ml, 48.45 mmol) in nitroethane (50 ml) stirred at −40° C. After 5 minutes, aluminum chloride (6.53 g, 49 mmol) was added and the mixture was immediately transferred to an oil bath, previously heated to 10° C. The mixture was stirred at this temperature for 20 minutes then poured onto ice to give the title compound as a solid (5.3 g, 63%). ¹H NMR (300 MHz, d₆-DMSO) δ 7.86 (dd, 1H), 7.79 (m,1H), 7.53 (d, 1H), 7.39 (m, 1H), 3.43 (s, 3H).

b) 2-Methylaminobenzene Sulfonamide

The compound from Example 122a (5.25 g, 24 mmol) was heated in 50% sulfuric acid (25 ml) at 130° C. until dissolved. The mixture was poured onto ice and taken to pH 8 with sodium hydroxide solution. The title compound slowly crystallized (2.25 g, 50%). ¹H NMR (300 MHz, d₆-DMSO) δ 7.79 (dd, J=1 and 8 Hz, 1H), 7.45 (m, 1H), 6.79 (m, 1H), 5.70 (br.s, NH), 4.85 (br. s, NH_{2, 2}H), 2.90 (s, 3H).

c) N-Methyl-N-(2-sulfamoylphenyl)malonamic acid ethyl ester

A mixture of the compound from Example 122b (1.0 g, 5.36 mmol) and pyridine (0.5 ml, 38 mmol) was stirred in chloroform (10 ml) and treated with ethyl 3-chloro-3-oxopropionate (0.75 ml, 5.85 mmol). The mixture was stirred overnight, poured into 1 molar hydrochloric acid and extracted into dichloromethane and concentrated. Purification using flash chromatography (silica gel, 2% methanol in dichloromethane) gave the title compound (1.3 g, 81%). ¹H NMR (300 MHz, CDCl₃) δ 8.10 (m, 1H), 7.63 (m, 1H), 7.53 (m, 1H), 7.33 (m, 1H), 5.72 (s, NH), 5.61 (s, NH), 4.28 (dq, 1H), 4.10 (q, 1H), 3.56-3.77 (4 lines, 1H), 3.31 (d, 3H), 3.0-3.23 (4 lines, 1H), 1.32 (t, 1.5H), 1.20 (t, 1.5H).

d) (4-Methyl-1,1-dioxo-1,4-dihydrobenzo[1,2,4]thiadiazin-3-yl)acetic Acid Ethyl Ester

The compound from Example 122c (1.1 g, 7.6 mmol) was heated under reflux in phosphorus oxychloride (5 ml) for 2 hours. The mixture was poured onto ice, neutralized with sodium hydrogen carbonate solution and extracted with dichloromethane. Evaporation gave an oil that crystallized to give the title compound (650 mg, 63%). ¹H NMR (300 MHz, d₆-DMSO) δ 8.12 (m, 1H), 8.05 (m, 1H), 7.91 (d, 1H), 7.80 (m, 1H), 4.41 (q, 2H), 4.31 (s, 2H), 3.86 (s, 3H), 3.26 (s, 2H), 1.45 (t, 3H).

e) 4-Hydroxy-1-(3-methylbutyl)-3-(4-methyl-1,1-dioxo-1,4-dihydrobenzo[1,2,4]thiadiazin-3-yl)-1-quinolin-2-one

Sodium hydride (160 mg of a 60% suspension in oil, 4.0 mmol) was added to a suspension of the compound from Example 122d (295 mg, 1.0 mmol) in anhydrous dioxane (10 ml). After 10 minutes, 1-3-methylbutyl)-1H-benzo[d][1,3]oxazine-2,4-dione (233 mg, 1.0 mmol) was added and the mixture was heated under reflux for 2 hours. The mixture was cooled, acidified with acetic acid and heated under reflux for an additional 3 hours. Dilution with water gave the title compound as a solid (150 mg, 35%). ¹H NMR (300 MHz, d₆-DMSO) δ 11.94 (br. s, 1H), 8.12 (dd, 1H), 7.95 (m, 1H), 7.50-7.86 (m, 5H), 7.35 (m, 1H), 4.20 (m, 2H), 3.54 (d, 3H), 1.72 (m, 1H), 1.51 (m, 2H), 0.98 (m, 6H).

### EXAMPLE 123

3-(1,1-Dioxo-7-methyl-1,4-dihydrobenzo[1,2,4]thiadiazin-3-yl)-4-hydroxy-1-(3-methylbutyl)-1H-quinolin-2-one

a) 3-(1,1-Dioxo-7-methyl-1,4-dihydrobenzo[1,2,4]thiadiazin-3-yl)-acetic Acid Ethyl Ester

A suspension of 2-amino-5-methyl-benzensulfonamide (Girard, Y.; Atkinson, J. G.; Rokach, J. J. Chem. Soc., Perkin 1 1979, 1043-1047) (500 mg, 2.69 mmol) and pyridine (0.218 mL, 2.69 mmol) in CH₂Cl₂ (10 mL) cooled at 0° C. was treated with ClCOCH₂COOEt, warmed to room temperature and stirred for 18 h. The solvent was then evaporated at reduced pressure and the residue was partitioned between 1 N HCl and EtOAc. The organic layer was separated, washed with water, brine, dried over MgSO₄ and evaporate at reduced pressure. The residue was triturated with hexane/EtOAc 1:1 to afford 560 mg of N-(4methyl-2-sulfamoyl-phenyl)-malonamic acid ethyl ester. MS(ES+) m/e 301 [M+H]⁺.

A suspension of N-(4-Methyl-2-sulfamoyl-phenyl)-malonamic acid ethyl ester (560 mg, 1.86 mmol) in 10% aq. NaHCO₃ (10 mL) was warmed to 45° C. until all the solid went into solution. The reaction mixture was then stirred at room temperature for 20 min, treated with 1 N HCl to pH=7 and the solid formed was collected by filtration to afford 70 mg (13%) of the title compound as a white powder.

b) 3-(1,1-Dioxo-7-methyl-1,4-dihydrobenzo[1,2,4]thiadiazin-3-yl)-4-hydroxy-113-methylbutyl)-1H-quinolin-2-one

A suspension of the compound obtained in Example 123a) (66 mg, 0.234 mmol), 1-(3-methyl-butyl)-1H-benzo[d][1,3]oxazine-2,4-dione (54.5 mg, 0.234 mmol) and NaH (37.4 mg of a 60% suspension in mineral oil, 0.936 mmol) in TBF (5 mL) was heated under reflux for 2.5 h, cooled to room temperature, treated with AcOH (1 mL) and heated under reflux for 1.5 h. After cooling, the reaction mixture was poured into 1 N HCl and diluted with water. The solid formed was collected by filtration, washed with water, Et₂O and triturated with EtOAc (2×) to afford 15 mg (15%) of the title compound as a tan powder. ¹H-NMR (d₆-DMSO) δ 15.28 (s, 1H); 14.51 (s, 1H); 8.21 (d, J=8.0 Hz, 1H); 7.90 (t, J=7.9 Hz, 1H); 7.76 (s, 1H); 7.68 (d, J=8.6 Hz, 1H); 7.63-7.58 (m, 2H); 7.46 (t, J=7.5 Hz, 1H); 4.36-4.32 (m, 2H); 2.44 (s, 3H); 1.80 (sept, J=6.7 Hz, 1H); 1.58-1.52 (m, 2H); 1.01 (d, J=6.6 Hz, 6H). MS(ES+) mr/e 426 [M+H]⁺.

### EXAMPLE 124

3-(1,1-Dioxo-1,4-dihydrobenzo[1,2,4]thiadiazin-3-yl)-4-hydroxy-1-(3-hydroxypropyl)-1H-quinolin-2-one

The procedure of Examples 9a) and 9b) (except substituting 3-(bromopropoxy)-tert-butyldimethylsilane for 4-bromo-1-butene) was followed for the preparation of the title compound with the following modification: after addition of AcOH and heating under reflux for 1.5 h, the reaction mixture was cooled, treated with 1 mL of water and 2.5 mL of AcOH and heated under reflux for 18 h. The suspension was then cooled, poured into 1 N HCl and the solid collected by filtration and purified by flash chromatography on silica gel (3% to 10% gradient 10% MeOH in CHCl₃/CHCl₃) to give the title compound (51 mg, 14%) as white crystals. ¹H-NMR (d₆-DMSO) δ 15.20 (br s, 1H); 14.41 (br s, 1H) 8.22 (dd, J=8.1, 1.4 Hz, 1H); 7.98-7.69 (m, 5H); 7.57 (t, J=7.5 Hz, 1H); 7.47 (t, J=7.5 Hz, 1H); 4.75 (br s, 1H); 4.41 (t, J=7.4 Hz, 2H); 3.59 (t, J=6.0 Hz, 1H); 1.89-1.85 (m, 2H). MS(ES+) m/e 400 [M+H]⁺.

### EXAMPLE 125

3-(1,1-Dioxo-1,4-dihydrobenzo[1,2,4]thiadiazin-3-yl)-4-hydroxy-6-(2-hydroxybutoxy)-1-(3-methylbutyl)-1H-quinolin-2-one

2-Ethyloxirane (0.02 mL, 0.24 mmol) was added to a mixture of the compound from Example 59c) (50 mg, 0.12 mmol), potassium carbonate(138 mg, 1.0 mmol) and potassium iodide(64.5 mg, 0.4 mmol) in dimethylformamide (5.0 mL). The mixture was heated at 85° C. under nitrogen for overnight. The mixture was cooled, water added and extracted by ethyl acetate. The organic extracts was washed with water, dried over magnesium sulfate, filtered and evaporated to give a brown oil. Purification by flash chromatography (0-10% methanol in chloroform) gave the title compound. (25 mg, 39%).

¹H NMR (400 MHz, d₆-DMSO) δ 8.44 (s, 1H), 7.50-7.60 (m, 2H), 7.35-7.45 (m, 2H), 7.15 (q, 2H), 3.95 (m, 2H), 3.72 (d, 2H), 3.60 (m, 114), 1.0-1.60 (m, 5H), 0.85 (m, 9H). MS(ES+) m/e 500 [M+H]⁺.

### EXAMPLE 126

3-(7-Dimethylarino-1,1-dioxo-1,4-dihydrobenzo[1,2,4]thiadiazin-3-yl)-4-hydroxy-1-(3-methylbutyl)-1H-quinolin-2-one

a) 3-(7-Amino-1,1-dioxo-1,4-dihydrobenzo[1,2,4]thiadiazin-3-yl)-4-hydroxy-1-(3-methylbutyl)-1H-quinolin-2-one

Following the procedure in Example 69, except substituting the product of Example 21e (3-(1,1-dioxo-7-nitro-1,2-dihydrobenzo[1,2,4]thiadiazin-3-yl)-4-hydroxy-1-(3-methylbutyl)-1H-quinolin-2-one) for that of 67b (1-(2-cyclopropylethyl)-3-(1,1-dioxo-1,4-dihydrobenzo[1,2,4]thiadiazin-3-yl)-4-hydroxy-6-nitro-1H-quinolin-2-one), the title compound was obtained in 69%. ¹H NMR (d₆-DMSO) δ 15.5 (s, 1H), 14.1 (s, 1H), 8.2 (dd, 1H), 7.89 (m, 1H), 7.68 (d, 1H), 7.4-7.5 (2H, m), 6.91-6.97 (m, 2H), 5.85 (s, 2H), 4.38 (m, 2H), 1.8 (m, 1H), 1.5 (m, 2H), 1.02 (d, 6H). MS(ES+) m/e 427 [M+H]⁺.

b) 3-(7-Dimethylamino-1,1-dioxo-1,4-dihydrobenzo[1,2,4]thiadiazin-3-yl)-4-hydroxy-1-(3-methylbutyl)-1H-quinolin-2-one

Following the procedure reported for Example 72, except using the product of Example 126a in place of the product of Example 69, the title compound was isolated and purified by chromatography (ODS silica, gradient 10-90% acetonitrile/water, 0.01% TFA) (15%). ¹HNMR (d₆-DMSO) δ 15.5 (s, 1H), 14.1 (s, 1H), 8.2 (1H), 7.89 (m, 1H), 7.68 (d, 1H), 7.4-7.5 (2H, m), 6.91-6.97 (m, 2H), 4.38 (m, 2H), 3.6 (s, 6H), 1.8 (m, 1H), 1.5 (m, 2H), 1.02 (d, 6H). MS(ES+) m/e 455 [M+H]⁺.

### EXAMPLE 127

3-(7-[(2R)-Aminobutoxy]-1,1-dioxo-1,4-dihydrobenzo[1,2,4]thiadiazin-3-yl}4-hydroxy-1-(3-methylbutyl)-1H-quinolin-2-one

a) (R)—N-tert-Butyl-2-amino-1-butanol

A solution of (R)-2-amino-1-butanol (4.95 g, 55.5 mmol) and di-tert-butyl-dicarbonate (12.73g, 58.3 mmol) in dichloromethane (50 mL) was stirred at room temperature for 18 h. The resulting reaction mixture was then washed with water and the organic layer was concentrated to give the title compound as a clear gum (10.50g, 100%). ¹H NMR (300 MHz, DMSO-d₆) δ 4.5 (t, 1H), 3.4-3.2 (m, 3H), 1.4 (s, 9H), 0.8 (t, 3H).

b) Methanesulfonic Acid (R)-2-tert-butoxycarbonylamino-butyl Ester

A solution of the compound from Example 127a) (0.946 g, 5.0 mmol), triethyl amine (0.51 g, 5.0 mmol) and methanesulfonyl chloride (0.573 g, 5.00 mmol) was stirred at room temperature for 18 h. The solution was washed with water and the organic layer was dried and concentrated to give the title compound as white crystals (100 mg, 75%).

c) 3-{7-[(2R)-Aminobutoxy]-1,1-dioxo-1,4-dihydrobenzo[1,2,4]thiadiazin-3-yl}-4-hydroxy-1-(3-methylbutyl)-1H-quinolin-2-one

A solution of the compound from Example 64 (86 mg, 0.2 mmol) and the compound from Example 127b) (52 mg, 0.2 mmol) in anhydrous DMF (1.5 mL) was treated with sodium hydride (24 mg of a 60% suspension in mineral oil, 0.6 mmol) and potassium iodide (12 mg, 0.07 mmol). The resulting reaction mixture was stirred at 100° C. for 18 h and concentrated to give a gum. The gum was purified by HPLC to give the title compound as white powder (20 mg, 20%). ¹H NMR (300 MHz, DMSO-d₆) δ 8.2 (d, 1H), 7.9 (t, 1H), 7.7 (m, 2H), 7.5 (t, 1H), 7.4(m, 1H), 6.8 (d, 1H), 4.4 (m, 2H), 4.0(d, 2HI), 1.8-1.5 (m, 4H), 1.0 (m, 6H), 0.9 (t, 3H), MS(ES+) m/e 499 (M+H).

### EXAMPLE 128

3-(1,1-Dioxo-1,4-dihydrobenzo[1,2,4]thiadiazin-3-yl)-4-hydroxy-14-nitrobenzyl)-1H-quinolin-2-one

Following the procedure of Examples 9a) and 9b) except substituting 4-nitrobenzyl bromide for 4-bromo-1-butene, the title compound (109 mg, 34%) was obtained as a yellow powder after trituration in EtOAc. ¹H-NMR (CDCl₃) δ 15.31 (br s, 1H); 14.17 (bs, 1H); 8.27 (dd, J=8.0, 1.4 Hz, 1H); 8.19 (d, J=8.8 Hz, 2H); 7.96 (d, J=7.5 Hz, 1H); 7.83-7.68 (m, 3H); 7.60-7.44 (m, 5H); 5.50 (s, 2H). MS (ES+) m/e 477 [M+H]⁺.

### EXAMPLE 129

1-(6-Aminopyridin-3-ylmethyl)-3-(1,1-dioxo-1,4-dihydrobenzo[1,2,4]thiadiazin-3-yl)-4-hydroxy-1H-quinolin-2-one

Following the procedure of Examples 9a) and 9b) except substituting (5-bromomethyl-pyridin-2-yl)-carbamic acid tert-butyl ester (Linschoten, M.; Polla, M.; Svensson, P. PCT Int. Application WO 0066557, 2000) for 4-bromo-1-butene, the title compound was obtained after removal of the protecting group. The solid was suspended in EtOAc (3 mL) and 3 M HCl (2 mL) for 72 h, the solvent was removed under reduced pressure and the residue was taken up in 4 M HCl in dioxane (8 mL) and stirring was continued for 48 h. After pouring the mixture in water, collecting the solid by filtration and triturating the solid in EtOAc, the title compound was obtained as a tan powder (164 mg, 31%) as the hydrochloride salt. ¹H-NMR (d₆-DMSO) δ 14.63 (br s, 1H); 14.17 (s, 1H); 8.25 (dd, J=8.1, 1.5 Hz, 1H); 8.04 (br s, 2H); 8.01 (d, J=1.5 Hz, 1H); 7.95 (dd, J=8.0, 1.2 Hz, 1H); 7.91 (dd, J=9.2, 2.2 Hz, 1H); 7.84 (ddd, J=8.6, 7.1, 1.5 Hz, 1H); 7.79 (ddd, J=8.3, 7.4, 1.3 Hz, 1H); 7.68 (dd, J=7.4, 2.0 Hz, 1H); 7.58 (ddd, J=7.9, 7.2, 0.9 Hz, 1H); 7.49-7.45 (m, 1H); 6.97 (d, J=9.2 Hz, 1H); 5.52 (s, 2H). MS (ES+) m/e 448 [M+H]⁺.

### EXAMPLE 130

1-(4-Bromobenzyl)-3-(1,1-dioxo-1,4-dihydro-1-benzo[1,2,4]thiadiazin-3-yl)-4-hydroxy-1H-quinolin-2-one

Following the procedure of Examples 9a) and 9b) except substituting 4-bromobenzyl bromide for 4-bromo-1-butene, the title compound (274 mg, 51%) was obtained as white crystals after recrystallization from DMSO. ¹H-NMR (d₆-DMSO) δ 15.28 (br s, 1H); 14.10 (s, 1H); 8.13 (dd, J=8.1, 1.1 Hz, 1H); 7.85 (d, J=7.8 Hz, 1H); 7.70-7.65 (m, 2H); 7.57 (d, J=8.1 Hz, 1H); 7.47 (t, J=7.8 Hz, 1H); 7.44-7.40 (m, 3H); 7.34 (t, J=7.6 Hz, 1H); 7.18 (d, J=8.4 Hz, 2H); 5.51 (s, 2H). MS (ES+) m/e 510 [M+H]⁺.

### EXAMPLE 131

1-(3-Bromobenzyl)-3-(1,1-dioxo-1,4-dihydro-1-benzo[1,2,4]thiadiazin-3-yl)-4-hydroxy-1H-quinolin-2-one

Following the procedure of Examples 9a) and 9b) except substituting 3-bromobenzyl bromide for 4-bromo-1-butene, the title compound (232 mg, 43%) was obtained as white crystals after trituration in EtOAc. ¹H-NMR (d₆-DMSO) δ 15.25 (br s, 1H); 14.12 (s, 1H); 8.17 (dd, J=8.0, 1.4 Hz, 1H); 7.89 (d, J=7.5 Hz, 1H); 7.75-7.69 (m, 2H); 7.61 (d, J=7.8 Hz, 1H—); 7.52-7.46 (m, 3H); 7.42-7.35 (m, 2H); 7.24-7.18 (m, 2H); 5.58 (s, 2H). MS (ES+) m/e 510 [M+H]⁺.

### EXAMPLE 132

1-(2-Cyclopropylethyl) 6-fluoro-4-hydroxy-3-(7-methoxy-1,1-dioxo-1,4-dihydrobenzo[1,2,4]thiadiazin-3-yl)-1-quinolin-2-one

a) 7-methoxy-1,1-dioxo-1,4-dihydrobenzo[1,2,4]thiadiazin-3-one

4-Anisidine (20 g, 162 mmol) in nitroethane (100 ml) was added dropwise to a solution of chlorosulfonyl isocyanate (17 ml, 195 mmol) in nitroethane (150 ml) striired at −40° C. After 5 minutes, aluminum chloride (28 g, 210 mmol) was added and the mixture was immediately transferred to an oil bath, previously heated to 1 100C. The mixture was stirred at this temperature for 20 minutes then poured onto ice to give the title compound as a purple solid (24 g, 65% o). ¹H NMR (300 MHz, d₆-DMSO) δ 11.14 (s, NH), 7.20 (m, 3H), 3.81 (s, 3H).

b) 2-Amino-5-methoxybenzene Sulfonamide

The compound from Example 132a (3.2 g, 14 mmol) was heated in 50% sulfuric acid (25 ml) at 130° C. until dissolved. The mixture was poured onto ice, neutralized and extracted into ethyl acetate. Evaporation of the solvent gave the title compound (2.0 g, 70%). ¹H NMR (300 MHz, d₆-DMSO) δ 7.25 (s, NH 21H), 7.11 (d, J=3 Hz, 1H), 6.94 (dd, J=3 and 9 Hz, 1H), 6.77 (d, J=9 Hz, 1H), 5.44 (s, NH_{2, 2}H), 3.67 (s, 3H).

c) N-(4-Methoxy-2-sulfamoylphenyl)malonamic Acid Ethyl Ester

A mixture of the compound from Example 134b (9.1 g, 45 mmol) and diethyl malonate (14 ml, 92 mmol) were heated together at 160° C. for 1 hour. The mixture was cooled and diluted with ether to give the title compound as a solid (8.5 g, 60%). ¹H NMR (300 MHz, d₆-DMSO) δ 9.44 (s, NH), 7.75 (d, J=9 Hz, 1H) 7.51 (s, NH_{2, 2}H), 7.37 (d, J=3 Hz, 1H), 7.20 (dd, J=3 and 9 Hz, 1H), 4.14 (q, 2H), 3.81 (s, 3H), 3.57 (s, 2H), 1.23 (t, 3H).

d) (7-Methoxy-1,1-dioxo-1,4-dihydrobenzo[1,2,4]thiadiazin-3-yl)-acetic Acid Ethyl Ester

The compound from Example 132c (2.4 g, 7.6 mmol) was heated under reflux in phosphorus oxychloride (50 ml) for 2.5 hours. The solvent was evaporated and the residue dissolved in ethyl acetate. The solution was neutralized, washed with 2M hydrochloric acid, dried and evaporated to a solid. Trituration with ether gave the title compound (1.8 g, 79%). ¹H NMR (300 MHz, d₆-acetone) δ 11.10 (s, NH), 7.26-7.80 (m, 3H), 4.32 (q, 2H), 3.79 (s, 3H), 3.26 (s, 2H), 1.49 (t, 3H).

e) 1-(2-Cyclopropylethyl) 6-fluoro-4-hydroxy-3-(7-methoxy-1,1-dioxo-1,4-dihydrobenzo[1,2,4]thiadiazin-3-yl)-1-quinolin-2-one

A mixture of the compound from Example 132d (130 mg, 0.44 mmol) and the compound from Example 74b (109 mg, 0.44 mmol) in anhydrous tetrahydrofuran (10 ml) under argon was treated with sodium hydride (60% suspension in mineral oil) (80 mg, 2.0 mmol) and heated under reflux for 1 hour. The mixture was acidified with acetic acid and heated under reflux for an additional 1 hour. The solvent was partially removed and the mixture diluted with water. The solid was collected, washed with water, ether and hexane to give the title compound (110 mg, 55%). ¹H NMR (300 MHz, d₆-DMSO) δ 15.21 (br.s, 1H), 14.54 (br.s, 1H), 7.71-7.82 (m, 2H), 7.58-7.69 (m, 2H), 7.27 (m, 2H), 4.32 (m, 2H), 3.78 (s, 3H), 3.34 (s, 3H), 1.49 (m, 2H), 0.72 (m, 1H), 0.30 (m, 2H), 0.00 (m, 2H).

### EXAMPLE 133

3-(7-Benzylamino-1,1-dioxo-1,4-dihydro-1-benzo[1,2,4]thiadiazin-3-yl)-4-hydroxy-1-(3-methylbutyl)-1H-quinolin-2-one

In a dried sealed tube under argon was added copper iodide (10 mg, 0.05 mMol), K₃PO₄ (425 mg, 2.0 mMol), isopropanol (1 ml), ethylene glycol (111 μl, 2.0 mMol), benzyl amine (131 μl, 1.2 mMol) and 4-hydroxy-3-(7-iodo-1,1-dioxo-1,4-dihydro-1-benzo[1,2,4]thiadiazin-3-yl)-1-(3-methylbutyl)-1H-quinolin-2-one. The tube was sealed and heated at 80° C. for 14h. Water was added and the resulting mixture extracted 3 times with diethyl ether. The organic phase was washed with brine, dried over MgSO₄, filtered and evaporated to residue. The crude material was purified by chromatography (silica, gradient 0-10% methanol/chloroform) to give the product as a white solid (25.9 mg, 25%). MS(ES+) m/e 517 [M+H]⁺.

### EXAMPLE 134

(1)-3-{3-[1-(2-Cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yl}acrylamide

a) 1-(2-Cyclopropylethyl)-3-(1,1-Dioxo-1,4-dihydrobenzo[1,2,4]thiadiazin-7-iodo-3-yl)-6-fluoro-4-hydroxy-1H-quinolin-2-one

Sodium hydride (133 mg of 60% suspension in mineral oil, 2.0 mmol) was added to a mixture of 3-(2-cyclopropylethyl)-6-fluorobenzo[d][1,3]oxazin-4-one (125 mg, 0.5 mmol) and ethyl (7-iodo-1,1-dioxo-1,2dihydrobenzo[1,2,4]thiadiazin-3-yl)acetate (197 mg, 0.5 mmol) in tetrahydrofuran (15.0 mL). The mixture was heated under reflux for 1.5h, cooled and acidified with acetic acid. The mixture was then heated under reflux for an additional 1.5h, cooled then quenched with water. The product was collected, washed with water, diethyl ether then hexanes to give the title compound (150 mg, 54%) as yellow powder. MS(ES+) m/e 554 [M+H]⁺.

b) (E)-3-{3-[1-(2-Cyclopropylethyl)-6-fluoro-4-hydroxy-2-oxo-1,2-dihydro-quinolin-3-yl]-1,1-dioxo-1,4-dihydro-1-benzo[1,2,4]thiadiazin-7-yl}acrylamide

The mixture of the compound from Example 134a) (103 mg, 0.18 mmol), acrylamide (26 mg, 0.36 mmol), sodium acetate trihydrate (46 mg, 0.36 mmol), palladium acetate (8 mg), water (0.5 mL) and DMF (2 mL) was stirred at 80° C. for 18 h before cooled and diluted with water and filtered. The grey precipitate was washed with water and diethyl ether to give the desired product (87 mg, 94%). ¹H NMR (300 MHz, DMSO-d₆) δ 14.5 (broad, 1H), 14.0 (s, 1H), 8.0 (s, 1H), 7.9-7.4 (m, 5H), 7.1 (s, 1H), 6.7 (d, 1H), 4.4 (m, 2H), 1.5 (m, 2H), 0.8 (m, 2H), 0.4 (d, 2H), 0.0 (d, 2H). MS(ES+) m/e 497 (M+H).

### EXAMPLE 135

3-{3-[1-(2-Cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yl}propionamide

The mixture of the compound from Example 134b) (25 mg, 0.05 mmol) in DMF (5 mL) with 10% palladium on charcoal (100 mg) was shaken under an atmosphere of hydrogen at 50 psi for 4 days. The mixture was filtered through Celite®, washed through DMF and evaporated to a solid. The residue was purified by chromatography (ODS silica, gradient, 0-90% acetonitrile/water (0.01% TFA)) to give the title compound as a white powder (5 mg, 20%). MS(ES+) m/e 499 (M+H).

### EXAMPLE 136

1-(2-Cyclopropylethyl)-6-fluoro-4-hydroxy-3-(7-hydroxy-1,1-dioxo-1,4-dihydrobenzo[1,2,4]thiadiazin-3-yl)-1H-quinolin-2-one

a) (7-Hydroxy-1,1-dioxo-1,4-dihydrobenzo[1,2,4]thiadiazin-3-yl)acetic Acid Ethyl Ester

A mechanically stirred suspension of the compound from Example 132d (17.0 g, 57 mmol) in 1,2-dichloroethane (800 ml) was cooled in an ice bath (5-10° C.) under nitrogen. A solution of boron tribromide (200 ml of a 1 molar solution in dichloromethane) was added dropwise over 20 minutes, the ice bath removed and the mixture stirred at ambient temperature for 3 hours. The mixture was poured onto ice, the organic layer separated and the aqueous layer extracted thrice with ethyl acetate. The combined organic solutions were washed with brine, dried and evaporated to a foam. The product was dissolved in ethanol (100 ml) and sulfuric acid (1 ml) was added. The mixture was heated under reflux for 45 minutes, partially evaporated and diluted with ethyl acetate. The solution was washed with water and the aqueous layer extracted with ethyl acetate. The combined organic solutions were dried and evaporated to an oil. Crystallisation from dichloromethane gave the title compound as a grey solid (6.3 g, 39%). ¹H NMR (400 MHz, d₆-DMSO) δ 12.07 (s, 1H), 10.17 (s, 1H), 7.02-7.29 (m, 3H), 4.15 (q, 2H), 1.21 (t, 3H).

b) 1-(2Cyclopropylethyl)-6-fluoro-4-hydroxy-3-(7-hydroxy-1,1-dioxo-1,4-dihydrobenzo[1,2,4]thiadiazin-3-yl)-1H-quinolin-2-one

Following the procedure for Example 132, except substituting the compound from Example 135a for the compound from Example 136a, gave the title compound as a yellow solid (105 mg, 28%). ¹H NMR (400 MHz, d₆-DMSO) δ 15.25 (s, 1H), 14.14 (s, 1H), 10.31 (s, 1H), 7.62-7.81 (m, 3H), 7.50 (d, 1H), 7.08 (m, 2H), 4.33 (m, 2H), 1.50 (m, 2H), 0.73 (m, 1H), 0.33 (m, 2H), 0.00 (m, 2H).

### EXAMPLE 137

{3-[1-(2-Cyclopropylethyl)-6-fluoro-4-hydroxy-2-oxo-1,2-dihydroquinolin-3-yl]-1,1-dioxo-1,4-dihydrobenzo[1,2,4]thiadiazin-7-yloxy}acetonitrile

A suspension of the compound from Example 136b (95 mg, 0.21 mmol) in dimethylformamide (5 ml) was treated with sodium hydride (24 mg of a 60% suspension in oil, 0.6 mmol) and stirred (with gentle warming) until the anion formed. Bromoacetonitrile (30 ul, 0.43 mmol) was slowly added and the mixture was stirred at 600C for 2 hours. The mixture was acidified with acetic acid, diluted with water and the title compound collected as a tan solid (90 mg, 87%). ¹H NMR (400 MHz, d₆-DMSO) δ 15.25 (s, 1H), 14.16 (s, 1H), 7.60-7.82 (m, 4H), 7.46 (d, 1H), 7.40 (m, 1H), 5.26 (s, 2H), 4.33 (m, 2H), 1.50 (m, 2H), 0.73 (m, 1H), 0.33 (m, 2H), 0.00 (m, 2H).

### EXAMPLE 138

{3-[1-(2-Cyclopropylethyl)-6-fluoro-4-hydroxy-2-oxo-1,2-dihydroquinolin-3-yl)]1,1-dioxo-1,4-dihydrobenzo[1,2,4]thiadiazin-7-yloxy}acetamide

Following the procedure of Example 137, except substituting 2-bromoacetamide for bromoacetonitrile, gave the title compound as a yellow solid (45 mg, 39%). ¹H NMR (400 MHz, d₆-DMSO) δ 15.30 (s, 1H), 14.15 (s, 1H), 7.50-7.85 (m, 5H), 7.29 (m, 3H), 4.50 (s, 2H), 4.33 (m, 2H), 1.51 (m, 2H), 0.73 (m, 1H), 0.31 (m, 2H), 0.00 (m, 2H).

### EXAMPLE 139

3-(1,1-Dioxo-1,4-dihydro-1-benzo[1,2,4]thiadiazin-3-yl)-4-hydroxy-12-methylcyclopropylmethyl)-1H-quinolin-2-one

Following the procedures of Examples 28a) and 28b) except substituting 2-methylcyclopropanemethanol for cyclopentanemethanol, the title compound was obtained (385 mg, 54%) as a white powder, after washing the precipitate with H₂O, hexanes and Et₂O and trituration from EtOAc (2×). NMR analysis showed a 94:6 ratio of methyl stereoisomers with the data for the major stereoisomer (relative stereochemistry unknown) given below. ¹H-NMR (CDCl₃) δ 15.26 (s, 1H); 14.59 (s, 1H); 8.31 (dd, J=8.1, 1.4 Hz, 1H); 7.98 (dd, J=7.9, 0.5 Hz, 1H); 7.75 (ddd, J=8.5, 7.1, 1.5 Hz, 1H); 7.62 (ddd, J=8.2, 7.4, 1.4 Hz, 1H); 7.53 (d, J=8.6 Hz, 1H); 7.46-7.42 (m, 1H); 7.38-7.34 (m, 1H); 7.30 (d, J=8.1 Hz, 1H); 4.28 (d, J=6.5 Hz, 2H); 1.01 (s, 3H); 0.99-0.95 (m, 2H); 0.73-0.69 (m, 1H); 0.34-0.30 (m, 1H). MS(ES+) m/e 410 [M+H]⁺.

### EXAMPLE 140

1-(2-Aminopyridin-4-ylmethyl)-3-(1,1-dioxo-1,4-dihydro-1-benzo[1,2,4]thiadiazin-3-yl)-4-hydroxy-1H-quinolin-2-one

Following the procedures of Examples 28a) and 28b) except substituting (4-hydroxymethyl-pyridin-2-yl)-carbamic acid tert-butyl ester (Linschoten, M.; Polla, M.; Svensson, P. PCT Int. Application WO 0066557, 2000) for cyclopentanemethanol, the title compound was obtained (33 mg, 11%) as the hydrochloride salt as a white crystals after removal of the protecting group (10 mL of 4M HCl in dioxane, 5 days, room temperature) and recrystallization from AcOH. ¹H-NMR (d₆-DMSO) δ 13.96 (br s, 1H); 8.09 (dd, J=8.1, 1.4 Hz, 1H); 7.74 (d, J=6.9 Hz, 2H); 7.64-7.55 (m, 4H); 7.45 (d, J=8.2 Hz, 1H); 7.36 (t, J=7.6 Hz, 1H); 7.31-7.27 (m, 2H); 6.69 (dd, J=6.7, 1.5 Hz, 1H); 6.39 (bs, 1H); 5.45 (s, 2H). MS(ES+) m/e 448 [M+H]⁺.

### EXAMPLE 141

6-Fluoro-4-hydroxy-3-(7-hydroxy-1,1-dioxo-1,4-dihydro-1-benzo[1,2,4]thiadiazin-3-yl)-1-(3-methylbutyl)-1H-quinolin-2-one

Following the procedure of Example 14c, except substituting the compound from Example 136a for ethyl 1,1-dioxo-2H-benzo-1,2,4-thiadiazinyl-3-acetate, the title compound was prepared as a pale yellow solid (650 mg, 37%). ¹H NMR (400 MHz, d₆-DMSO) δ 15.20 (br.s, 1H), 14.05 (br.s, 1H), 10.29 (s, 1H), 7.77 (dd, 1H), 7.62 (m, 2H), 7.44 (d, 1H), 7.04 (m, 2H), 4.21 (m, 2H), 1.66 (m, 1H), 1.40 (m, 2H), 0.88 (d, 6H).

### EXAMPLE 142

{3-[6-Fluoro hydroxy-2-oxo-1-(3-methylbutyl)-2-dihydroquinolin-3-yl]-1,1-dioxo-1,4-dihydrobenzo[1,2,4]thiadiazin-7-yloxy}acetonitrile

Sodium hydride (100 mg of a 60% suspension in mineral oil, 2.55 mmol) was added to a suspension of the compound from Example 141 (377 mg, 0.85 mmol) in anhydrous dimethylformamide at 200C. The mixture was warmed to 50° C. over 15 minutes then bromoacetonitrile (0.1 ml, 4.0 mmol) was added dropwise. The mixture was stirred at 50° C. overnight, cooled, acetic acid (0.5 ml) added, followed by water. The solid was collected, washed with water, then ether and dried to give the title compound as a pale yellow solid (370 mg, 90%). ¹H NMR (400 MHz, d₆-DMSO) δ 14.90 (br.s, 1H), 14.14 (s, 1H), 7.77 (dd, 1H), 7.65 (m, 3H), 7.48 (d, 1H), 7.37 (dd, 1H), 5.24 (s, 2H), 4.22 (m, 2H), 1.68 (m, 1H), 1.42 (m, 2H), 0.88 (d, 6H).

### EXAMPLE 143

4,6-Dihydroxy-3-(7-hydroxy-1,1-dioxo-1,4-dihydro-1-benzo[1,2,4]thiadiazin-3-yl)-1-(3-methyl-butyl)-1H-quinolin-2-one

a) 4,6-Dihydroxy-3-(7-methoxy-1,1-dioxo-1,4-dihydrobenzo[1,2,4]thiadiazin-3-yl)-1-(3-methyl-butyl)-1H-quinolin-2-one

Following the procedures of Examples 58c) except substituting the compound obtained in Example 132d) for ethyl 1,1-dioxo-2H-benzo-1,2,4-thiadiazinyl-3-acetate, the title compound was obtained (210 mg, 69%) as a tan powder after washing the precipitate with H₂O and Et₂O. MS(ES+) m/e 458 [M+H]⁺.

b) 4,&Dihydroxy-3-(7-hydroxy-1,1-dioxo-1,4-dihydro-1-benzo[1,2,4]thiadiazin-3-yl)-1-(3-methyl-butyl)-1H-quinolin-2-one

The compound from Example 143 a) (210 mg, 0.459 mmol) was suspended in 12 nL of AcOH, heated to reflux until a solution was obtained and treated with 48% HBr aq. (3 mL). The reaction mixture was stirred with heating under reflux overnight, followed by addition of 1 mL of 48% HBr. After heating under reflux for 3 h, addition of 0.5 mL 48% HBr and heating under reflux for an additional 2 h, the mixture was cooled slightly and poured in H₂O. After cooling, the solid was collected and washed with H₂O, then Et₂O and dried to give 135 mg (66%) of the title compound as a yellow-green powder.

¹H-NMR (d₆-DMSO) δ 15.27 (s, 1H); 14.54 (s, 1H); 10.43 (s, 1H); 10.01 (s, 1H); 7.62-7.58 (m, 2H); 7.55 (d, J=2.9 Hz, 1H); 7.42 (dd, J=9.1, 2.8 Hz, 1H); 7.23-7.19 (m, 2H); 4.37-4.33 (m, 2H); 1.81 (sept, J=6.7 Hz, 1H); 1.58 (q, J=6.7 Hz, 2H); 1.04 (d, J=6.6 Hz, 6H). MS(ES+) m/e 444 [M+H]⁺.

### EXAMPLE 144

[3-(7-Cyanomethoxy-1,1-dioxo-1,4-dihydro-1-benzo[1,2,4]thiadiazin-3-yl)-4-hydroxy-1-(3-methyl-butyl)-2-oxo-1,2-dihydro-quinolin-6-yloxy]-acetonitrile

Sodium hydride (99.2 mg of a 60% dispersion in mineral oil, 2.48 mmol) was added to a solution of the compound from Example 143b) (110 mg, 0.248 mmol) in DMF (5 mL). After 10 min, bromoacetonitrile (0.086 mL, 1.24 mmol) was added and the reaction mixture was stirred at 39° C. for 16 h, cooled and poured in 1 N HCl. The solid was collected, washed with H₂O, Et₂O, and hexane and dried to give 115 mg (89%) of the title compound as a tan powder. ¹H-NMR (d₁-DMSO) δ 15.05 (br s, 1H); 14.26 (br s, 1H); 7.64-7.60 (m, 2H); 7.56 (d, J=9.3 Hz, 1H); 7.49 (dd, J=9.3, 2.8 Hz, 1H); 7.44 (d, J=2.7 Hz, 1H); 7.33 (dd, J=9.1, 2.8 Hz, 1H); 5.20 (s, 2H); 5.18 (s, 2H); 4.214.16 (m, 2H); 1.63 (sept, J=6.6 Hz, 1H); 1.39 (q, J=7.1 Hz, 2H); 0.84 (d, J=6.6 Hz, 6H). MS(ES+) m/e 522 [M+H]⁺.

### EXAMPLE 145

2-{3-[1-(2-Cyclopropyl-ethyl)-6-fluoro-4-hydroxy-2-oxo-1,2-dihydro-quinolin-3-yl]-1,1-dioxo-1,4-dihydro-1-benzo[1,2,4]thiadiazin-7-yloxy}-propionamide

To a solution of the compound from Example 136b (50 mg, 0.11 mmol) in DMF (3 mL) was added sodium hydride (18 mg of a 60% suspension in mineral oil, 0.45 mmol). The mixture was stirred under N₂ for fifteen minutes, followed by the addition of 2-bromo-propionamide (25.7 mg, 0.17 mmol). The mixture was heated at 60° C. for two hours. After cooled to the ambient temperature, the reaction was quenched by the addition of ice water and extracted with ethyl acetate (3×20 mL). The organic layer was dried over MgSO₄, filtered, concentrated and purified by flash column chromatography (0-10% methanol in chloroform) to give the title compound as a yellow solid (20 mg, 36%). MS(ES+) m/e 515 [M+H]⁺. ¹H NMR (400 MHz, d₆-DMSO) δ 15.1 (s, 1H), 14.3 (s, 1H), 7.8 (dd, J=3, 9 Hz, 1H), 7.70-7.56 (m, 2H), 7.57 (s, 1H), 7.25 (dd, J=3, 9 Hz, 1H), 7.22 (s, 1H), 4.69 (q, J=7 Hz, 1H), 4.32 (t, J=7 Hz, 2H), 1.50 (m, 2H), 1.39 (d, J=7 Hz, 3H), 0.69 (m, 1H), 0.33 (m, 2H), 0.0 (m, 2H).

### EXAMPLE 146

1-(2-Cyclopropyl-ethyl)-3-[1,1-dioxo-7-(tetrazol-5-ylmethoxy)-1,4-dihydro-1-benzo[1,2,4]thiadiazin-3-yl]-6-fluoro-4-hydroxy-1H-quinolin-2-one

To a solution of the compound from Example 137 (21 mg, 0.044 mmol) in toluene (2 mL) was added azidotrimethylsilane (20 mg, 0.17 mmol) and catalytic amount of dibutyltin oxide. The mixture was exposed to microwave irradiation at 1600C for 40 minutes. After the mixture was allowed to cool to ambient temperature, the organic solvent was removed under vacuum. The residue was washed with hexanes and diethyl ether to afford the title compound as a brown solid (13 mg, 56.8%). MS(ES+) m/e 526 [M+H]⁺. ¹H NMR (400 MHz, dr-DMSO) δ 15.1 (s, 1H), 14.3 (s, 1H), 7.8 (dd, J=3, 9 Hz, 1H), 7.70-7.60 (m, 3H), 7.48 (d, J=3 Hz, 1H), 7.38 (dd, J=3, 9 Hz, 1H), 7.29 (s, 1H), 5.57 (s, 2H), 4.33 (t, J=7 Hz, 2H), 1.50 (m, 2H), 1.39 (d, J=7 Hz, 3H), 0.69 (m, 1H), 0.33 (m, 2H), 0.0 (m, 2H).

### EXAMPLE 147

{3-[6-Fluoro-4-hydroxy-2-oxo-1-(3-methylbutyl)-2-dihydroquinolin-3-yl]-1,1-dioxo-1,4-dihydrobenzo[1,2,4]thiadiazin-7-yloxy}acetamide

The compound from Example 142 (358 mg, 0.74 mmol) was dissolved in sulfuric acid (10 ml) and water (1.0 ml) was added, ensuring the compound remained dissolved. The mixture was stirred for 3 days and poured onto ice. The solid was collected, washed with water, then ether and dried to give the title compound as a tan solid (320 mg, 86%). ¹H NMR (400 MHz, d₆-DMSO) δ 15.32 (Br. s, 1H), 14.33 (s, 1H), 8.01 (dd, 1H), 7.80-7.93 (m, 4H), 7.75 (s, 1H, NH), 7.51 (m, 2H), 4.71 (s, 2H), 4.46 (m, 2H), 1.90 (m, 1H), 1.65 (m, 2H), 1.12 (d, 6H).

### EXAMPLE 148

2-[3-(7-Carbamoylmethoxy-1,1-dioxo-1,4dihydro-1-benzo[1,2,4]thiadiazin-3-yl)-4-hydroxy-1-(3-methyl-butyl)-2-oxo-1,2-dihydro-quinolin-6-yloxy]-acetamide

A solution of the compound from Example 144 (70 mg, 0.134 mmol) in H₂SO₄ (5 mL) and H₂O (0.5 mL) was stirred at room temperature for 4 h. The solution was then poured on ice and the precipitate was collected by filtration, air dried, dissolved in hot DMSO and filtered through a 0.2 μm Acrodisc®. After evaporation of the solvent at reduced pressure, the residue was triturated in CHCl₃ to give the title compound (18 mg, 24%) as a tan powder. ¹H-NMR (d₆-DMSO) δ 15.29 (br s, 1H); 14.51 (br s, 1H); 7.78-7.61 (m, 6H); 7.48-7.45 (m, 4H); 4.67 (s, 2H); 4.64 (s, 2H); 4.41-4.37 (m, 2H); 1.85 (sept, J=6.6 Hz, 1H); 1.60 (q, J=6.9 Hz, 2H); 1.07 (d, J=6.6 Hz, 6H). MS(ES+) m/e 558 [M+H]⁺.

### EXAMPLE 149

2-[3-(7-Carbamoylmethoxy-1,1-dioxo-1,4-dihydro-1-benzo[1,2,4]thiadiazin-3-yl)-1-(2-cyclopropyl-ethyl)-4-hydroxy-2-oxo-1,2-dihydro-quinolin-6-yloxy]-acetamide

a) 1-(2-Cyclopropyl-ethyl)4,6-dihydroxy-3-(7-hydroxy-1,1-dioxo-1,4-dihydro-1-benzo[1,2,4]thiadiazin-3-yl)-1H-quinolin-2-one

A suspension of the compound from Example 75a) (500 mg, 1.38 mmol), the compound from Example 136a) (393.2 mg, 1.38 mmol) and NaH (220.8 mg of a 60% dispersion in mineral oil, 5.52 mmol) in THF (10 mL) was heated under reflux for 2.5 h. The reaction mixture was then cooled, treated with AcOH (1.5 mL), heated under reflux for 1.5 h, cooled and poured in 1 N HCl. The precipitate formed was collected, washed with H₂O and Et₂O, dried and then suspended in 1.5 mL of THF. A 1 M solution of TBAF in THF (1.8 mL) was then added and the solution obtained was stirred at room temperature for 3 h, poured in 1 N HCl and stirred for 10 min. The precipitate was collected, washed with H₂O and dried to give the title compound as a tan powder (145 mg, 24%). ¹H-NMR (d₆-DMSO) δ 15.14 (s, 1H); 14.44 (s, 1H); 10.32 (s, 1H); 9.94 (s, 1H); 7.56 (d, J=9.3 Hz, 1H); 7.48 (d, J=9.6 Hz, 1H); 7.42 (d, J=2.7 Hz, 1H); 7.28 (m, 1H); 7.11-7.08 (m, 2H); 4.33-4.29 (m, 2H); 1.28-1.20 (m, 2H); 0.78-0.71 (m, 1H); 0.34-0.31 (m, 2H); 0.03-(−)0.01 (m, 2H).

b) 2-[3-(7-Carbamoylmethoxy-1,1-dioxo-1,4-dihydro-1-benzo[1,2,4]thiadiazin-3-yl)-1-(2-cyclopropyl-ethyl)-4-hydroxy-2-oxo-1,2-dihydro-quinolin-6-yloxy]-acetamide

A suspension of the compound from Example 149a) (103 mg, 0.233 mmol), bromoacetamide (128.7 mg, 0.933 mmol) and K₂CO₃ (322 mg, 2.33 mmol) in CH₃CN (8 mL) was heated under reflux for 4.5 h. After removal of the solvent at reduced pressure, the residue was suspended in 1 N HCl (30 mL) and the mixture was stirred for 40 min. The solid was collected by filtration, washed with H₂O, EtOAc and Et₂O, air dried and recrystallized from AcOH to give the title compound (21 mg, 16%) as yellow crystals. ¹H-NMR (d₆-DMSO) δ 15.25 (s, 1H); 14.50 (s, 1H); 8.02-7.50 (m, 6H); 7.42-7.36 (m, 4H); 4.60 (s, 2H); 4.58 (s, 2H); 4.45-4.41 (m, 2H); 1.63-1.58 (m, 2H); 0.85-0.81 (m, 1H); 0.44-0.39 (m, 2H); 0.03-(−)0.01 (m, 2H). MS(ES+) m/e 556 [M+H]⁺.

### EXAMPLE 150

[3-(7-Cyanomethoxy-1,1-dioxo-1,4dihydro-1-benzo[1,2,4]thiadiazin-3-yl)-4-hydroxy-1-(3-methyl-butyl)-2-oxo-1,2-dihydroquinolin-6-ylamino]acetonitrile

a) 6-Amino-1-(3-methylbutyl)-1H-benzo[d][1,3]oxazine-2,4dione.

Sodium hydride (800 mg of a 60% oil suspension, 20.0 mmol) was added to a stirred, ice-cooled solution of 6-amino-1H-benzo[a][1,3]oxazine-2,4-dione (3.56 g, 20.0 mmol) and 1-iodo-3-methylbutane (3.93 g, 20.0 mmol) in dimethylformamide (20 mL) under argon. After gas evolution had finished, the mixture was stirred 3 days at room temperature, then iced water (250 mL) added. The precipitate was filtered, washed with water and dried, then slurried in dichloromethane (300 mL) and filtered through Celite®. The solvent was removed from the filtrate under reduced pressure and the residue purified by chromatography on silica gel (0-20% ethyl acetate/dichloromethane) to give the title compound (0.45 g, 9%) as a yellow solid. ¹H NMR (400 MHz, d₆-DMSO) δ 7.19-7.10 (3H, m), 5.44 (2H, s), 3.95 (2H, m), 1.70 (1H, m), 1.52 (2H, m), 0.97 (6H, d, J=6.6 Hz).

b) 6-Amino-4-hydroxy-3-(7-methoxy-1,1-dioxo-1,2-dihydrobenzo[1,2,4]thiadiazin-3-yl)-1-(3-methylbutyl)-1H-quinolin-2-one

The procedure of Example 23b was followed using 6-amino-1-(3-methylbutyl)-1H-benzo[d][1,3]oxazine-2,4-dione in place of 1-(3-methylbutyl)-1H-benzo[d][1,3]oxazine-2,4-dione, and (7-methoxy-1,1-dioxo-1,4-dihydrobenzo[1,2,4]thiadiazin-3-yl)-acetic acid ethyl ester (Example 132d) in place of methyl (7-bromo-1,1-dioxo-1,2-dihydrobenzo[1,2,4]thiadiazin-3-yl)acetate, to give the title compound as a solid. ¹H NMR (400 MHz, d₆-DMSO) δ 14.80 (1H, br s), 7.66 (1H, m), 7.42 (1H, m), 7.37-7.31 (3H, m), 7.23 (1H, m), 5.53 (2H, br s), 4.28 (2H, m), 3.89 (3H, s), 1.77 (1H, m), 1.54 (2H, m), 1.00 (6H, d, J=6.6 Hz).

c) 6-Amino-4-hydroxy-3-(7-hydroxy-,1-dioxo-1,2-dihydrobenzo[1,2,4]thiadiazin-3-yl)-1-(3-methylbutyl)-1H-quinolin-2-one

The procedure of Example 110 was followed using 6-amino-4-hydroxy-3-(7-methoxy-1,1-dioxo-1,2-dihydrobenzo[1,2,4]thiadiazin-3-yl)-1-(3-methylbutyl)-1H-quinolin-2-one in place of 4-hydroxy-3-(5-methoxy-1,1-dioxo-1,4-dihydrobenzo[1,2,4]thiadiazin-3-yl)-1-(3-methylbutyl)-1H-quinolin-2-one to give the title compound as a slightly impure solid which was used without further purification in the next step. MS (ES+) m/e 443 [M+H]⁺.

d) [3-(7-Cyanomethoxy-1,1 dioxo-1,4-dihydro-1-benzo[1,2,4]thiadiazin-3-yl)-4-hydroxy-1-(3-methyl-butyl)-2-oxo-1,2-dihydroquinolin-6-ylamino]acetonitrile

A mixture of 6-amino-4-hydroxy-3-(7-hydroxy-1,1-dioxo-1,2-dihydrobenzo[1,2,4]thiadiazin-3-yl)-1-(3-methylbutyl)-1H-quinolin-2-one (0.128 g, 0.289 mmol), bromoacetonitrile (0.060 mL, 0.867 mmol), potassium carbonate (0.120 g, 0.867 mmol) and dimethylformamide (2 mL) was stirred while heating at 1000C in a microwave synthesizer for 1.5 h. After cooling, acetic acid (0.3 mL) was added dropwise followed by water (50 mL) with stirring. The mixture was extracted with ethyl acetate, and the extracts washed with water and evaporated to dryness under reduced pressure. The residue was purified by chromatography on silica gel (2-6% methanolydichloromethane) to give the title compound (0.042 g, 28%) as a yellow solid. ¹H NMR (400 MHz, d₆-DMSO) δ 15.16 (1H, br s), 14.72 (1H, br s), 7.78 (1H, m), 7.61-7.59 (2H, m), 7.51 (1H, dd, J=9.0, 2.7 Hz), 7.41 (2H, m), 6.69 (1H, br s), 5.38 (2H, s), 4.42 (2H, d, J=6.5 Hz), 4.34 (2H, m), 1.80 (1H, m), 1.57 (2H, m), 1.02 (6H, d, J=6.6 Hz).

### EXAMPLE 151

2-{3-[1-(2-Cyclopropyl-ethyl)-6-fluoro-4-hydroxy-2-oxo-1,2-dihydro-quinolin-3-yl]1,1-dioxo-1,4-dihydro-1-benzo[1,2,4]thiadiazin-7-yloxy}-2-methyl-propionamide

To a solution of the compound from Example 136b (50 mg, 0.11 mmol) in DMF (3 mL) was added sodium hydride (18 mg of a 60% suspension in mineral oil, 0.45 mmol). The mixture was stirred under N₂ for fifteen minutes, followed by the addition of 2-bromo-2-methylpropionamide (prepared by the method of Weidner, J. J.; Weintraub, M. P.; Schnettler, A. R.; Peet, P. N. Tetrahedron, 1997, 53(18), 6303) (210 mg, 1.17 mmol). The mixture was exposed to microwave irradiation at 100° C. for 30 minutes. After cooled to ambient temperature, the reaction was quenched by addition of ice water, extracted with ethyl acetate (3×20 mL). The organic layer was dried over MgSO₄, filtered, concentrated and purified by flash column chromatography (0-10% methanol in chloroform) to give the title compound as a yellow solid (38 mg, 64%). MS(ES+) m/e 529 [4+H]⁺. ¹H NMR (400 MHz, d₆-DMSO) δ 15.1 (s, 1H), 14.2 (s, 1H), 7.8 (dd, J=3, 9 Hz, 1H), 7.70-7.56 (m, 3H), 7.30 (s, 1H), 7.24(s, 1H), 4.32 (t, J=7 Hz, 2H), 1.51 (m, 2H), 1.48 (s, 6H), 0.69 (m, 1H), 0.33 (m, 2H), 0.0 (m, 2H).

### EXAMPLE 152

3-(1,1-Dioxo-1,4-dihydro-1-benzo [1,2,4]thiadiazin-3-yl)-4-hydroxy-1-(3-nitro-benzyl)-1H-quinolin-2-one

Following the procedure of Examples 9a) and 9b) except substituting 3-nitrobenzyl bromide for 4-bromo-1-butene, the title compound (465 mg, 58%) was obtained as a light tan powder after trituration in EtOAc (2×). ¹H-NMR (d₆-DMSO) δ 15.41 (br s, 1H); 14.24 (br s, 1H); 8.34 (s, 1H); 8.32 (dd, J=8.2, 1.4 Hz, 1H); 8.20 (dd, J=8.1, 1.3 Hz, 1H); 8.01 (d, J=7.9 Hz, 1H); 7.86-7.61 (m, 7H); 7.52 (t, J=7.5 Hz, 1H); 5.85 (s, 2H). MS (ES+) m/e 477 [M+H]⁺.

### EXAMPLE 153

3-(1,1-Dioxo-1,4-dihydro-1-benzo[1,2,4]thiadiazin-3-yl)-4-hydroxy-1-(2-methyl-pyridin-4-ylmethyl)-1H-quinolin-2-one

Following the procedures of Examples 28a) and 28b) except substituting (2-methyl-pyridin-4-yl)-methanol for cyclopentanemethanol, the title compound was obtained (145 mg, 44%) as white crystals as the hydrochloride salt after washing the precipitate with H₂O, Et₂O and hexane. ¹H-NMR (d₆-DMSO) δ 14.11 (s, 1H); 8.73 (d, J=6.0 Hz, 1H); 8.34 (dd, J=8.0, 1.4 Hz, 1H); 8.01 (d, J=8.0 Hz, 1H); 7.88-7.81 (m, 2H); 7.77 (d, J=5.9 Hz, 1H); 7.72-7.69 (m, 2H); 7.56-7.61 (m, 1H); 7.56-7.52 (m, 2H); 5.88 (s, 2H); 2.67 (s, 3H). MS(ES+) m/e 447 [M+H]⁺.

### EXAMPLE 154

[3-[7-(Cyanomethyl-amino)-1,1-dioxo-1,4-dihydro-1-benzo[1,2,4]thiadiazin-3-yl]-4-hydroxy-1-(3-methyl-butyl)-2-oxo-1,2-dihydroquinolin-6-ylamino]-acetonitrile

The procedure described in Example 150d was followed using 6-amino-3-(7-amino-1,1-dioxo-1,2-dihydrobenzo[1,2,4]thiadiazin-3-yl)-4-hydroxy-1-(3-methylbutyl)-1H-quinolin-2-one (Example 27) in place of 6-amino-4-hydroxy-3-(7-hydroxy-1,1-dioxo-1,2-dihydrobenzo[1,2,4]thiadiazin-3-yl)-1-(3-methylbutyl)-1H-quinolin-2-one to give the title compound as a brown powder. ¹H NMR (400 MHz, d₆-DMSO) δ 15.37 (1H, br s), 14.53 (1H, br s), 7.60-7.58 (2H, m), 7.41-7.39 (2H, m), 7.18-7.15 (2H, m), 6.91 (1H, br t, J=6.6 Hz), 6.67 (1H, br t, J=6.5 Hz), 4.45 (2H, d, J=6.6 Hz), 4.41 (2H, d, J=6.6 Hz), 4.33 (2H, m), 1.79 (1H, m), 1.56 (2H, m), 1.01 (6H, d, J=6.6 Hz).

### EXAMPLE 155

3-(7-Amino-1,1dioxo-1,4-dihydro-1-benzo[1,2,4]thiadiazin-3-yl)-1-(2-cyclopropyl-ethyl)-6-fluoro-4-hydroxy-1H-quinolin-2-one

a) 1-(2-Cyclopropyl-ethyl)-6-fluoro-4-hydroxy-3-(7-nitro-1,1-dioxo-1,4-dihydro-1-benzo[1,2,4]thiadiazin-3-yl)-1H-quinolin-2-one

To the product obtained in Example 74c, 1-(2-Cyclopropylethyl)-3-(1,1-dioxo-1,4-dihydrobenzo[1,2,4]thiadiazin-3-yl)-6-fluoro-4-hydroxy-1-quinolin-2-one, (25 mg, 0.058 mmol) in 1 ml of sulfolane was added 1M nitronium tetrafluoroborate in sulfolane (58 μl, 0.058 mmol). The mixture was stirred for one hour at 80° C. The mixture was cooled to room temperature and 5 ml of water were added. A yellow precipitate formed and was collected by filtration, washed with ethyl acetate and ether to yield 11.9 mg (0.025 mmol, 43%) of desired product. MS(ES+) m/e 473 [M+H]⁺.

b) 3-(7-Amino-1,1-dioxo-1,4-dihydro-1-benzo[1,2,4]thiadiazin-3-yl)-1-(2-cyclopropyl-ethyl)-6-fluoro-4-hydroxy-1H-quinolin-2-one

A solution of the compound from Example 155a (11.9 mg, 0.025 mMol) in ethanol (10.0 ml) and 2M NaOH (1.0 ml) with 10% palladium on charcoal was shaken under an atmosphere of hydrogen at 50 psi for 30 min. The mixture was filtered through Celite®, washed through with ethanol and ethyl acetate then concentrated. Purification by chromatography (silica gel, chloroform/methanol gradient) yielded 2.9 mg (0.0065 mmol, 26%) of the desired product. MS(ES+) m/e 443 [M+H]⁺.

### EXAMPLE 156

{3-[1-(2-Cyclopropylethyl)-6-fluoro-4-hydroxy-2-oxo-1,2-dihydroquinolin-3-yl]-1,1-dioxo-1,4-dihydrobenzo[1,2,4]thiadiazin-7-yloxy}acetic Acid

To a solution of the compound from Example 136(b) (20 mg, 0.045 mmol) in DMF (3 mL) was added sodium hydride (7.2 mg of a 60% suspension in mineral oil, 0.18 mmol). The mixture was stirred under N₂ for fifteen minutes, followed by the addition of chloroacetic acid (6.4 mg, 0.068 mmol). The mixture was heated at 60° C. for four hours. After cooling to ambient temperature, the reaction was quenched by the addition of ice water and acetic acid. The precipitate was collected by centrifuge, washed with water and diethyl ether, and dried in vacuo to give the title compound as a yellow solid (15 mg, 67%). MS(ES+) m/e 502 [M+H]⁺. ¹H NMR (400 MHz, d₆-DMSO) δ 15.2 (s, 1H), 14.2 (s, 1H), 13.1 (s, 1H), 7.78 (dd, J=3, 9 Hz, 1H), 7.72 (dd, J=4, 9 Hz, 1H), 7.67-7.61 (m, 1H), 7.58 (d, J=9 Hz, 1H), 7.26 (dd, J=3, 9 Hz, 1H), 7.23 (d, J=3 Hz, 1H), 4.78 (s, 2H), 4.32 (t, J=7 Hz, 2H), 1.50 (m, 2H), 0.74 (m, 1H), 0.33 (m, 2H), 0.0 (m, 2H).

### EXAMPLE 157

2-[3-(7-Carbamoylmethoxy-1,1-dioxo-1,4dihydro-1-benzo[1,2,4]thiadiazin-3-yl)-4-hydroxy-1-(3-methylbutyl)-2-oxo-1,2-dihydroquinolin-6-ylamino]acetamide

Water (0.5 mL) was added slowly to a stirred solution of [3-(7-cyanomethoxy-1,1-dioxo-1,4dihydro-1-benzo[1,2,4]thiadiazin-3-yl)-4-hydroxy-1-(3-methyl-butyl)-2-oxo-1,2-dihydro-quinolin-6-ylamino]-acetonitrile (example 150, 0.056 g, 0.108 mmol) in sulfuric acid (3 mL) at room temperature. After stirring for 64 h, iced water (80 mL) was added and the solid filtered and washed with water and ether. The crude product was boiled in a solution of sodium acetate (0.05 g, 0.61 mmol) in methanol/water (2:1, 30 mL) before cooling and filtering. The residue was heated in dichloromethane/methanol (5:1, 20 mL) and the solid filtered after cooling, then washed with dichloromethane and dried to leave the tide compound (0.031 g, 52%) as a powder. ¹H NMR (400 MHz, D₆-DMSO) δ 15.15 (1H, br s), 14.76 (1H, br s), 7.70 (1H, d, J=7.9 Hz), 7.64 (1H, s), 7.51 (1H, d, J=8.9 Hz), 7.45-7.39 (4H, m), 7.33 (1H, d, J=8.4 Hz), 7.13-7.11 (2H, m), 6.32 (1H, br s), 4.60 (2H, s), 4.31 (2H, m), 3.71 (2H, d, J=4.3 Hz), 1.78 (1H, m), 1.54 (2H, m), 1.01 (6H, d, J=6.6 Hz).

### EXAMPLE 158

6-Fluoro-4-hydroxy-3-7-hydroxy-1,1-dioxo-1,4-dihydro-1-benzo[1,2,4]thiadiazin-3-yl)-1-pyridin-4-ylmethyl-1H-quinolin-2-one

a) 6-Fluoro-1-pyridinylmethyl-1H-benzo[d][1,3]oxazine-2,4-dione

Diisopropyl azodicarboxylate (0.326 mL, 1.66 mmol) was added dropwise to a cooled suspension (0° C.) of the compound from Example 14a) (300 mg, 1.66 mmol), PhP₃ (434 mg, 1.66 mmol) and 4-pyridinemethanol (181 mg, 1.66 mmol) in CH₂Cl₂ (15 mL). Stirring was continued for 20 h at room temperature, after which time the solvent was evaporated and the residue was triturated in Et₂O. The solid was collected by filtration, washed with Et₂O to afford a mixture of the desired product and residual starting material. The solid mixture was suspended in CHCl₃ (75 mL), warmed, cooled and filtered. The filtrate was evaporated to afford the title compound (230 mg, 51%) as a white solid. ¹H-NMR (d₆-DMSO) δ 8.59-8.57 (m, 2H); 7.90 (dd, J=7.9, 3.0 Hz, 1H); 7.70 (ddd, J=9.1, 8.3, 3.1 Hz, 1H); 7.51-7.49 (m, 2H); 7.26 (dd, J=9.2, 4.0 Hz, 1H); 5.39 (s, 2H).

b) 6-Fluoro-4-hydroxy-3-(7-hydroxy-1,1-dioxo-1,4-dihydro-1-benzo[1,2,4]thiadiazin-3-yl)-1-pyridin ylmethyl-1H-quinolin-2-one

DBU (0.209 mL, 1.39 mmol) was added to a solution of the compound from Example 158a) (152 mg, 0.558 mmol) and the compound from Example 136a) (158.7 mg, 0.558 mmol) in DMF (4.5 mL) and the mixture was stirred at room temperature for 22 h. Acetic acid (2 mL) was then added and stirring was continued for 40 min at 65° C. The mixture was cooled to room temperature and poured into 1 N HCl. The fine precipitate obtained was collected, after reducing the aqueous volume, and washed with H₂O. The filtrate was concentrated again and some more solid was collected for a total yield of 75 mg (29%) of the title compound as a pale yellow powder as the hydrochloride salt. ¹H-NMR (d₆-DMSO) δ 14.00 (br s, 1H); 10.63 (br s, 1H); 8.78-8.76 (m, 2H); 8.08 (dd, J=8.7, 2.9 Hz, 1H); 7.81 (td, J=9.1, 2.9 Hz, 1H); 7.69-7.49 (m, 4H); 7.31-7.28 (m, 2H); 5.89 (s, 2H). MS(ES+) m/e 467 [M+H]+.

### EXAMPLE 159

1-(2-Cyclopropylethyl)-6-fluoro-4-hydroxy-3-[7-(5-methyl-[1,3,4]oxadiazol-2-ylmethoxy)-1,1-dioxo-1,4-dihydro-1-benzo[1,2,4]thiadiazin-3-yl]-1H-quinolin-2-one

A solution of the compound from Example 146 (62 mg, 0.118 mmol) in acetic anhydride (5 mL) was refluxed under N₂ for two hours. After the mixture was cooled to ambient temperature, the organic solvent was removed under vacuum. The residue was purified by chromatography (silica gel, gradient 0-10% methanol in chloroform) to give the title compound as a white solid (22 mg, 35%). MS(ES+) m/e 540 [M+H]⁺. ¹H NMR (400 MHz, d₆-DMSO) δ 15.1 (s, 1H), 14.3 (s, 1H), 7.80 (dd, J=3, 9 Hz, 1H), 7.75-7.60 (m, 3H), 7.49 (d, J=3 Hz, 1H), 7.38 (dd, J=3, 9 Hz, 1H), 5.47 (s, 2H), 4.33 (m, 2H), 1.50 (m, 2H), 1.15 (s, 3H), 0.75 (m, 1H), 0.33 (m, 2H), 0.0 (m, 2H).

### EXAMPLE 160

6-Chloro-4-hydroxy-3-(7-hydroxy-1,1-dioxo-1,4-dihydro-1-benzo[1,2,4]thiadiazin-3-yl)-1-(3-methylbutyl)-1H-quinolin-2-one

A mixture of the compound from example 6a (700 mg, 2.62 mmol) and the compound from example 136a (780 mg, 2.62 mmol) was stirred in dimethylformamide (20 ml) and treated with 1,8-diazabicyclo[5.4.0]undec-7-ene (0.98 ml, 6.55 mmol). The mixture was stirred for 18 hours then acetic acid (5.0 ml) was added. Stirring continued for a further 3 hours, by which time a thick yellow precipitate had formed. The mixture was diluted with water and the solid collected. The solid was washed successively with water, ether and hexane to give the title compound (460 mg, 38%). ¹H NMR (400 MHz, D₆-DMSO) δ 15.10 (br.s, 1H), 13.90 (br.s, 1H), 10.27 (s, 1H), 7.78 (s, 1H), 7.73 (m, 1H), 7.54 (m, 1H), 7.42 (m, 1H), 7.00 (m, 2H), 4.14 (m, 2H), 1.61 (m, 1H), 0.92 (m, 2H), 0.82 (d, 6H).

### EXAMPLE 161

{3-[6-Chloro-4-hydroxy-2-oxo-1-(3-methylbutyl)-2-dihydroquinolin-3-yl]-1,1-dioxo-1,4-dihydrobenzo[1,2,4]thiadiazin-7-yloxy}acetonitrile

Sodium hydride (120 mg of a 60% suspension in mineral oil, 3.00 mmol) was added to a suspension of the compound from example 160 (336 mg, 0.73 mmol) in anhydrous dimethylformamide at 20° C. The mixture was warmed to 50° C. over 15 min. then bromoacetonitrile (0.15 ml, 6.0 mmol) was added dropwise. The mixture was stirred at 50° C. for 2 h, cooled, acetic acid (1.0 ml) added, followed by water. The solid was collected, washed with water, then ether and dried to give the title compound as a pale yellow solid (250 mg, 68%). ¹H NMR (400 MHz, D₆-DMSO) δ 14.80 (br.s, 1H), 13.98 (s, 1H), 7.97 (d, 1H), 7.75 (dd, 1H), 7.63 (d, 1H), 7.55 (d, 1H), 7.47 (d, 1H), 7.35 (dd, 1H), 5.23 (s, 2H), 4.17 (m, 2H), 1.68 (m, 1H), 1.42 (m, 2H), 0.86 (d, 6H).

### EXAMPLE 162

{3-[6-Chloro-hydroxy-2-oxo-1-(3-methylbutyl)-2-dihydroquinolin-3-yl]-1,1-dioxo-1,4-dihydrobenzo[1,2,4]thiadiazin-7-yloxy}acetamide

The compound from example 161 (200 mg, 0.40 mmol) was dissolved in sulfuric acid (5 ml) then water (0.5 ml) was added, ensuring the compound remained dissolved. The mixture was stirred for 72 h and poured onto ice. The solid was collected, washed with water, then ether and dried to give the title compound as a tan solid (190 mg, 91%). ¹H NMR (400 MHz, D₆-DMSO) δ 15.20 (Br. s, 1H), 14.02 (s, 1H), 8.05 (d, 1H), 7.83 (dd, 1H), 7.64 (m, 3H), 7.32 (m, 3H), 4.53 (s, 2H), 4.24 (m, 2H), 1.73 (m, 1H), 1.45 (m, 2H), 0.93 (d, 6H).

### EXAMPLE 163

3-(7-Amino-1,1-dioxo-1,4-dihydro-1-benzo[1,2,4]thiadiazin-3-yl)-6-fluoro-4-hydroxy-1-(3-methyl-butyl)-1H-quinolin-2-one

a) 6-Fluoro-hydroxy-1-(3-methyl-butyl)-3-(7-nitro-1,1-dioxo-1,4-dihydro-1l⁶-benzo[1,2,4]thiadiazin-3-yl)-1H-quinolin-2-one

To the product obtained in example 14c, 3-(1,1-dioxo-1,2-dihydrobenzo[1,2,4]thiadiazin-3-yl)-6-fluoro-4-hydroxy-1-(3-methylbutyl)-1H-quinolin-2-one, (138 mg, 0.322 mmol) in 3 ml of sulfolane was added 0.5M nitronium tetrafluoroborate in sulfolane (644 μl, 0.322 mmol). The mixture was stirred for 1 h at 80° C. The reaction was cooled to room temperature and 10 ml of water was added. A yellow precipitate formed and was collected by filtration, washed with ethyl acetate and ether to yield 81 mg (0.170 mmol, 53%) of desired product. MS(ES+) m/e 475 [M+H]⁺.

b) 3-(7-Amino-1,1-dioxo-1,4-dihydro-1-benzo[1,2,4]thiadiazin-3-yl)-6-fluoro-4-hydroxy-1-(3-methyl-butyl)-1H-quinolin-2-one

A solution of the compound from Example 163a (81 mg, 0.170 mmol) in methanol (10.0 ml) and 2M NaOH (1.0 ml) with catalytic 5% palladium on charcoal was shaken under an atmosphere of hydrogen at 50 psi for 3 h. The mixture was filtered through Celite®, washed through with methanol and ethyl acetate. The solvents were then evaporated and the residue purified by chromatography (silica gel, 9:1 chloroform/acetone) to give the title compound (30 mg; 0.0675 mmol, 40%). ¹H NMR (d₆-DMSO) 15.1 (s, 1H), 14.4 (s, 1H), 7.93 (s, 1H), 7.78 (d, J=7.57 Hz, 1H), 7.71 (d, J=6.83 Hz, 1H), 7.57 (s, 1H), 7.20 (d, 1H), 7.08 (d, 1H) 5.75 (s, 2H), 4.64 (m, 2H), 1.78 (m, 1H), 1.41 (m, 2H), 0.84 (s, 6H). MS(ES+) m/e 445 [M+H]⁺.

### EXAMPLE 164

{3-[6-Amino-4-hydroxy-1-(3-methylbutyl)-2-oxo-1,2-dihydroquinolin-3-yl]-1,1-dioxo-1,4-dihydro-16-benzo[1,2,4]thiadiazin-7-yloxy}acetonitrile

Sodium hydride (0.016 g of a 60% oil suspension, 0.409 mmol) was added to a stirred solution of 6-amino-4-hydroxy-3-(7-hydroxy-1,1-dioxo-1,2-dihydrobenzo[1,2,4]thiadiazin-3-yl)-1-(3-methylbutyl)-1H-quinolin-2-one (example 150c, 0.071 g, 0.136 mmol) in dimethylformamide (1 mL). After gas evolution had finished, bromoacetonitrile (0.020 g, 0.163 mmol) was added and the mixture stirred at 60° C. (microwave synthesizer) for 15 min, then cooled. Acetic acid (0.3 mL) was added dropwise with stirring followed by water (10 mL). The precipitate was filtered, washed with water and ether and dried to give the title compound (0.057 g, 88%) as a green powder. ¹H NMR (400 MHz, D₆-DMSO) δ 14.87 (1H, br s), 7.77 (1H, d, J=9.0 Hz), 7.60 (1H, s), 7.49 (1H, dd, J=9.0, 2.7 Hz), 7.43 (1H, d, J=9.1 Hz), 7.31 (1H, d, J=2.4 Hz), 7.23 (1H, d, J=7.3 Hz), 5.55 (2H, br s), 5.37 (2H, s), 4.28 (2H, m), 1.78 (1H, m), 1.53 (2H, m), 1.00 (6H, d, J=6.6 Hz).

### EXAMPLE 165

2-{3-[1-(2-Cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yloxy}-N,N-dimethylacetamide

To a solution of the compound from Example 136(b) (50 mg, 0.1 mmol) in dichloromethane (5 mL) was added dimethylamine hydrochloride (8.9 mg, 0.1 mmol), diisopropylethylamine (19 μL, 0.1 mmol), 1-hydroxy-7-azabenzotriazole (13.6 mg, 0.1 mmol) and polystyrene-bound ethyl diispropylcarbodiimide (161 mg, 0.2 mmol). The mixture was stirred under N₂ overnight. The polymer was filtered and washed with dichloromethane (3×10 mL). The filtrate was concentrated and purified by chromatography (silica gel, gradient 0-10% methanol in chloroform) to give the title compound as a white solid (21 mg, 40%). MS(ES+) m/e 529 [M+H]⁺. ¹H NMR (400 MHz, d6-DMSO) δ 15.17 (s, 1H), 14.18 (s, 1H), 8.23 (s, 1H), 7.83 (dd, J=3, 9 Hz, 1H), 7.70-7.56 (m, 3H), 7.28 (m, 2H), 4.94 (s, 2H), 4.34 (m, 2H), 2.92(s, 3H), 2.77 (s, 3H), 1.51 (m, 2H), 0.69 (m, 1H), 0.33 (m, 2H), 0.0 (m, 2H).

### EXAMPLE 166

2-{3-[-(2-Cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yloxy}-N-methylacetamide

To a solution of the compound from Example 136(b) (120 mg, 0.24 mmol) in dichloromethane (15 mL) was added methylamine (0.12 mL of 2 M solution in THF, 0.24 mmol), 1-hydroxy-7-azabenzotriazole (32.6 mg, 0.24 mmol) and polystyrene-bound ethyl diispropylcarbodiimide (387 mg, 0.48 mmol). The mixture was stirred under N₂ overnight. The polymer was filtered and washed with dichloromethane (3×20 mL). The filtrate was concentrated and purified by chromatography (silica gel, gradient 0-10% methanol in chloroform) to give the title compound as a white solid (32.6 mg, 27%). MS(ES+) m/e 515 [M+H]⁺. ¹H NMR (400 MHz, d6-DMSO) δ 15.2 (s, 1H), 14.2 (s, 1H), 8.08 (m, 1H), 7.81 (dd, J=2, 6 Hz, 1H), 7.70-7.56 (m, 3H), 7.33 (s, 2H), 4.55 (s, 2H), 4.32 (d, J=4.0 Hz, 2H1), 2.58 (d, J=4 Hz, 3H), 1.51 (m, 2H), 0.70 (m, 1H), 0.33 (m, 2H), 0.0 (m, 2H).

### EXAMPLE 167

{3-[4-Hydroxy-6-(2-hydroxyethylamino)-1-(3-methylbutyl)-2-oxo-1,2-dihydroquinolin-3-yl]-1,1-dioxo-1,4-dihydro-1-benzo[1,2,4]thiadiazin-7-yloxy}acetonitrile

A mixture of {3-[6-amino-4-hydroxy-1-(3-methylbutyl)-2-oxo-1,2-dihydroquinolin-3-yl]-1,1-dioxo-1,4-dihydro-16-benzo[1,2,4]thiadiazin-7-yloxy}acetonitrile (example 164, 0.045 g, 0.093 mmol), bromoethanol (0.023 g, 0.187 mmol), potassium iodide (0.031 g, 0.187 mmol), potassium carbonate (0.026 g, 0.187 mmol) and dimethylformamide (1 mL) was stirred in a microwave synthesizer at 100° C. for 0.5 h. Additional bromoethanol (0.032 g, 0.256 mmol) and potassium carbonate (0.026 g, 0.188 mmol) were added and heating continued another 0.5 h. Acetic acid (0.5 mL) and water (30 mL) were added and the mixture extracted with ethyl acetate. The extracts were washed with water, dried (sodium sulfate) and evaporated under reduced pressure. The residue was purified by chromatography (silica gel, 3% methanol/dichloromethane) to give a solid which was triturated with ether to give the title compound (0.020 g, 41%) as a yellow powder. ¹H NMR (400 MHz, D₆-DMSO) δ 15.06 (1H, br s), 14.85 (1H, br s), 7.75 (1H, br m), 7.59 (1H, s), 7.47 (2H, m), 7.32 (1H, br m), 7.18 (1H, s), 6.06 (1H, br s), 5.37 (2H, s), 4.78 (1H, br s), 4.29 (2H, m), 3.62 (2H, m), 3.18 (2H, m), 1.77 (1H, m), 1.53 (2H, m), 1.00 (6H, d, J=6.6 Hz).

### EXAMPLE 168

1-(2-Cyclopropylethyl)-3-[1,1-dioxo-7-(2-oxo-2-pyrrolidin-1-yl-ethoxy)-1,4-dihydro-1-benzo[1,2,4]thiadiazin-3-yl]-6-fluoro-4-hydroxy-1H-quinolin-2-one

To a solution of the compound from Example 136(b) (50 mg, 0.1 mmol) in dichloromethane (10 nL) was added pyrrolidine (8.3 μL, 0.1 mmol), 1-hydroxy-7-azabenzotriazole (13.6 mg, 0.1 mmol) and polystyrene-bound ethyl diispropylcarbodiimide (161 mg, 0.2 mmol). The mixture was stirred under N₂ overnight. The polymer was filtered and washed with dichloromethane (3×20 mL). The filtrate was concentrated and purified by chromatography (silica gel, gradient 0-10% methanol in chloroform) to give the tide compound as a white solid (22 mg, 40%). MS(ES+) m/e 555 [M+H]⁺. ¹H NMR (400 MHz, d₆-DMSO) δ 15.2 (s, 1H), 14.2 (s, 1H), 7.81 (dd, J=2, 6 Hz, 1H), 7.70-7.56 (m, 3H), 7.27 (d, J=7 Hz, 2H), 4.84 (s, 2H), 4.33 (t, J=4.0 Hz, 2H), 3.39 (t, J=6 Hz, 2H), 3.30 (m, 2H), 1.82 (t, J=7 Hz, 2H), 1.70 (t, J=7 Hz, 2H), 1.51 (m, 2H), 0.70 (m, 1H), 0.33 (m, 2H), 0.0 (m, 2H).

### EXAMPLE 169

1-(2-Cyclopropylethyl)-6-fluoro-4-hydroxy-3-[7-(2-morpholin-4-yl-2-oxo-ethoxy)-1,1dioxo-1,4-dihydro-1-benzo[1,2,4]thiadiazin-3-yl]-1H-quinolin-2-one

To a solution of the compound from Example 136(b) (50 mg, 0.1 mmol) in dichlorometane (10 mL) was added morphine (8.8 μL, 0.1 mmol), 1-hydroxy-7azabenzitriazole (13.6 mg, 0.1 mmol) and polystyrene-bound ethyl diispropylcarbodiimide imide (161 mg, 0.2 mmol). The mixture was stirred under N₂ overnight. The polymer was filtered and washed with dichloromethane (3×10 mL). The filtrate was concentrated and purified by chromatography (silica gel, gradient 0-10% methanol in chloroform) to give the title compound as a white solid (9 mg, 16%). MS(ES+) m/e 571 [M+H]⁺. ¹H NMR (400 MHz, d₆-DMSO) δ 15.2 (s, 1H), 14.2 (s, 1H), 7.81 (dd, J=3, 9 Hz, 1H), 7.73-7.56 (m, 3H), 7.25-7.29 (m, 2H), 4.94 (s, 2H), 4.33 (t, J=4.0 Hz, 2H), 3.54 (t, J=4 Hz, 3H), 3.31 (t, J=4 Hz, 2H), 3.47 (t, J=4 Hz, 2H), 3.37 (t, J=4 Hz, 2H), 1.51 (m, 2H), 0.70 (m, 1H), 0.33 (m, 2H), 0.0 (m, 2H).

### EXAMPLES 170-227

The title compounds were prepared according to the methods of the Examples above. AU products were subjected to purification and provided satisfactory LCMS analysis, as follows:

| Example # | Name | [M + H]⁺ |
|---|---|---|
| 170 | 4-[3-(1,1-Dioxo-1,2-dihydrobenzo[1,2,4]thiadiazin-3-yl)-4-hydroxy-2- | 427 |
| | oxoquinolin-1-yl]butyramide | |
| 171 | 3-(1,1-Dioxo-1,2-dihydrobenzo[1,2,4]thiadiazin-3-yl)-4-hydroxy-1-(3- | 456 |
| | methylbutyl)-2-oxo-1,2-dihydro-quinoline-7-carboxylic acid | |
| 172 | 4-Hydroxy-1-(3-methylbutyl)-6-nitro-3-(7-nitro-1,1-dioxo-1,2- | 502 |
| | dihydrobenzo[1,2,4]thiadiazin-3-yl)-1H-quinolin-2-one | |
| 173 | 7-bromo-3-(1,1-dioxo-1,2-dihydrobenzo[1,2,4]thiadiazin-3-yl)-4- | 491 |
| | hydroxy-1-(3-methylbutyl)-1H-quinolin-2-one | |
| 174 | 3-(1,1-Dioxo-7-phenyl-1,4-dihydro-1-benzo[1,2,4]thiadiazin-3-yl)-4- | 489 |
| | hydroxy-1-(3-methylbutyl)-1H-quinolin-2-one | |
| 175 | N-[3-(1,1-Dioxo-1,4-dihydro-1-benzo[1,2,4]thiadiazin-3-yl)-4- | 550 |
| | hydroxy-1-(3-methylbutyl)-2-oxo-1,2-dihydro-quinolin-6-yl]-4- | |
| | fluorobenzamide | |
| 176 | Thiophene-2-carboxylic acid [3-(1,1-dioxo-1,4-dihydro-1- | 538 |
| | benzo[1,2,4]thiadiazin-3-yl)-4-hydroxy-1-(3-methylbutyl)-2-oxo-1,2- | |
| | dihydro-quinolin-6-yl]amide | |
| 177 | N-[3-(1,1-Dioxo-1,4-dihydro-1-benzo[1,2,4]thiadiazin-3-yl)-4- | 600 |
| | hydroxy-1-(3-methylbutyl)-2-oxo-1,2-dihydro-quinolin-6-yl]-3- | |
| | trifluorobenzamide | |
| 178 | N-[3-(1,1-Dioxo-1,4-dihydro-1-benzo[1,2,4]thiadiazin-3-yl)-4- | 550 |
| | hydroxy-1-(3-methylbutyl)-2-oxo-1,2-dihydro-quinolin-6-yl]-3- | |
| | fluorobenzamide | |
| 179 | N-[3-(1,1-Dioxo-1,4-dihydro-1-benzo[1,2,4]thiadiazin-3-yl)-4- | 533 |
| | hydroxy-1-(3-methylbutyl)-2-oxo-1,2-dihydro-quinolin-6-yl]- | |
| | nicotinamide | |
| 180 | N-[3-(1,1-Dioxo-1,4-dihydro-1-benzo[1,2,4]thiadiazin-3-yl)-4- | 470 |
| | hydroxy-1-(3-methylbutyl)-2-oxo-1,2-dihydro-quinolin-6- | |
| | yl]acetamide | |
| 181 | 4-Hydroxy-3-(7-iodo-1,1-dioxo-1,4-dihydro-1-benzo[1,2,4]thiadiazin- | 538 |
| | 3-yl)-1-(3-methylbutyl)-1H-quinolin-2-one | |
| 182 | 4-[3-(1,1-Dioxo-1,4-dihydro-1-benzo[1,2,4]thiadiazin-3-yl)-4- | 456 |
| | hydroxy-2-oxo-2H-quinolin-1-yl]-butyric acid ethyl ester | |
| 183 | 4-[3-(1,1-Dioxo-1,4-dihydro-1-benzo[1,2,4]thiadiazin-3-yl)-4- | 428 |
| | hydroxy-2-oxo-2H-quinolin-1-yl]-butyric acid | |
| 184 | [3-(1,1-Dioxo-1,4-dihydro-1-benzo[1,2,4]thiadiazin-3-yl)-4-hydroxy- | 487 |
| | 6-(3-methyl-butoxy)-2-oxo-2H-quinolin-1-yl]acetic acid | |
| 185 | 3-(1,1-Dioxo-1,4-dihydro-1-benzo[1,2,4]thiadiazin-3-yl)-4-hydroxy-6- | 538 |
| | iodo-1-(3-methylbutyl)-1H-quinolin-2-one | |
| 186 | 3-(1,1-Dioxo-1,4-dihydro-1-benzo[1,2,4]thiadiazin-3-yl)-4-hydroxy-8- | 412 |
| | (3-methylbutyl)-1H-quinolin-2-one | |
| 187 | 3-(1,1-Dioxo-1,4-dihydro-1-benzo[1,2,4]thiadiazin-3-yl)-4-hydroxy-1- | 484 |
| | (3-methylbutyl)-2-oxo-1,2-dihydro-quinoline-5-carboxylic acid | |
| | dimethylamide | |
| 188 | 3-(1,1-Dioxo-1,4-dihydro-1-benzo[1,2,4]thiadiazin-3-yl)-4-hydroxy-1- | 456 |
| | (3-methylbutyl)-2-oxo-1,2-dihydro-quinoline-5-carboxylic acid | |
| 189 | 3-(1,1-Dioxo-1,4-dihydro-1-benzo[1,2,4]thiadiazin-3-yl)-4-hydroxy-6- | 549 |
| | (4-methoxy-benzyloxy)-1-(3-methylbutyl)-1H-quinolin-2-one | |
| 190 | 1-(2-Dimethylamino-ethyl)-3-(1,1-dioxo-1,4-dihydro-1- | 413 |
| | benzo[1,2,4]thiadiazin-3-yl)-4-hydroxy-1H-quinolin-2-one | |
| 191 | 4-Hydroxy-3-(6-methoxy-1,1-dioxo-1,4-dihydro-1- | 443 |
| | benzo[1,2,4]thiadiazin-3-yl)-1-(3-methylbutyl)-1H-quinolin-2-one | |
| 192 | 6-(6-Bromo-pyridin-2-yloxy)-3-(1,1-dioxo-1,4-dihydro-1- | 584 |
| | benzo[1,2,4]thiadiazin-3-yl)4-hydroxy-1-(3-methylbutyl)-1H- | |
| | quinolin-2-one | |
| 193 | 3-(1,1-Dioxo-1,4-dihydro-1-benzo[1,2,4]thiadiazin-3-yl)-4-hydroxy-1- | 386 |
| | (2-hydroxyethyl)-1H-quinolin-2-one | |
| 194 | 3-(1,1-Dioxo-1,4-dihydro-1-benzo[1,2,4]thiadiazin-3-yl)-4-hydroxy-6- | 536 |
| | (6-methoxy-pyridin-2-yloxy)-1-(3-methylbutyl)-1H-quinolin-2-one | |
| 195 | 3-(1,1-Dioxo-1,4-dihydro-1-benzo[1,2,4]thiadiazin-3-yl)-1-(5-fluoro- | 464 |
| | 2-methyl-benzyl)4-hydroxy-1H-quinolin-2-one | |
| 196 | 3-(1,1-Dioxo-1,4-dihydro-1-benzo[1,2,4]thiadiazin-3-yl)-4-hydroxy-1- | 477 |
| | (2-nitro-benzyl)-1H-quinolin-2-one | |
| 197 | 1-(2-Cyano3,5-dichlorobenzyl)-3-(1,1-dioxo-1,2- | 526 |
| | dihydrobenzo[1,2,4]thiadiazin-3-yl)-4-hydroxy-1H-quinolin-2-one | |
| 198 | 3-(1,1-Dioxo-1,2-dihydrobenzo[1,2,4]thiadiazin-3-yl)-4-hydroxy-1-(3- | 457 |
| | methylbutyl)-7-nitro-1H-quinolin-2-one | |
| 199 | 7-Chloro-3-(1,1-dioxo-1,2-dihydrobenzo[1,2,4]thiadiazin-3-yl)-4- | 447 |
| | hydroxy-1-(3-methylbutyl)-H-quinolin-2-one | |
| 200 | 1-(4-Aminobutyl)-3-(1,1-dioxo-1,2-dihydrobenzo[1,2,4]thiadiazin-3- | 413 |
| | yl)-4-hydroxy-1-1H-quinolin-2-one | |
| 201 | 3-(1,1-Dioxo-1,2-dihydrobenzo[1,2,4]thiadiazin-3-yl)-4-hydroxy-1- | 418 |
| | phenyl-1H-quinolin-2-one | |
| 202 | 6-Benzyloxy-3-(1,1-dioxo-1,2-dihydrobenzo[1,2,4]thiadiazin-3-yl)-4- | 519 |
| | hydroxy-1-(3-methylbutyl)-1H-quinolin-2-one | |
| 203 | 3-(8-Bromo-1,1-dioxo-1,4-dihydro-1-benzo[1,2,4]thiadiazin-3-yl)-4- | 491 |
| | hydroxy-1-(3-methylbutyl)-1H-quinolin-2-one | |
| 204 | (E)-3-[3-(1,1-Dioxo-1,4-dihydro-1-benzo[1,2,4]thiadiazin-3-yl)-4- | 482 |
| | hydroxy-1-(3-methylbutyl)-2-oxo-1,2-dihydro-quinolin-7- | |
| | yl]acrylamide | |
| 205 | [3-(1,1-Dioxo-1,4-dihydro-1-benzo[1,2,4]thiadiazin-3-yl)-4-hydroxy- | 543 |
| | 6-(3-methyl-butoxy)-2-oxo-2H-quinolin-1-yl]acetic acid tert-butyl | |
| | ester | |
| 206 | 3-(1,1-Dioxo-1,4-dihydro-1-benzo[1,2,4]thiadiazin-3-yl)-4-hydroxy-1- | 484 |
| | (3-methylbutyl)-2-oxo-1,2-dihydro-quinoline-6-carboxylic acid | |
| | dimethylamide | |
| 207 | 3-(1,1-Dioxo-1,4-dihydro-1-benzo[1,2,4]thiadiazin-3-yl)-4-hydroxy-1- | 440 |
| | (2-pyrrolidin-1-yl-ethyl)-1H-quinolin-2-one | |
| 208 | 1-(4-tert-Butylbenzyl)-3-(1,1-dioxo-1,4-dihydro-1- | 489 |
| | benzo[1,2,4]thiadiazin-3-yl)-4-hydroxy-1H-quinolin-2-one | |
| 209 | 2-{3-[6-Amino-4-hydroxy-1-(3-methylbutyl)-2-oxo-1,2- | 500 |
| | dihydroquinolin-3-yl]-1,1-dioxo-1,4-dihydro-1-benzo[1,2,4]thiadiazin- | |
| | 7-yloxy}acetamide | |
| 210 | 3-(1,1-Dioxo-1,4-dihydro-1-benzo[1,2,4]thiadiazin-3-yl)-4-hydroxy-1- | 453 |
| | (2-methyl-thiazol-4-ylmethyl)-1H-quinolin-2-one | |
| 211 | 2-{3-[6-Chloro-1-(2-cyclopropylethyl)-4-hydroxy-2-oxo-1,2- | 517, 519 |
| | dihydroquinolin-3-yl]-1,1-dioxo-1,4-dihydro-1-benzo[1,2,4]thiadiazin- | |
| | 7-yloxy}acetamide | |
| 212 | 1-(2-Cyclopropylethyl)-3-[1,1-dioxo-7-(2-oxo-2-piperazin-1-yl- | 570 |
| | ethoxy)-1,4-dihydro-1-benzo[1,2,4]thiadiazin-3-yl]-6-fluoro-4- | |
| | hydroxy-1H-quinolin-2-one | |
| 213 | 1-Cyclobutylmethyl-6-fluoro-4-hydroxy-3-(7-hydroxy-1,1-dioxo-1,4- | 444 |
| | dihydro-1-benzo[1,2,4]thiadiazin-3-yl)-1H-quinolin-2-one | |
| 214 | [3-(1-Cyclobutylmethyl-6-fluoro-4-hydroxy-2-oxo-1,2- | 483 |
| | dihydroquinolin-3-yl)-1,1-dioxo-1,4-dihydro-1-benzo[1,2,4]thiadiazin- | |
| | 7-yloxy]acetonitrile | |
| 215 | 2-[3-(1-Cyclobutylmethyl-6-fluoro-4-hydroxy-2-oxo-1,2- | 501 |
| | dihydroquinolin-3-yl)-1,1-dioxo-1,4-dihydro-1-benzo[1,2,4]thiadiazin- | |
| | 7-yloxy]acetamide | |
| 216 | {3-[1-(3,3-Dimethylbutyl)-6-fluoro-4-hydroxy-2-oxo-1,2- | 499 |
| | dihydroquinolin-3-yl]-1,1-dioxo-1,4-dihydro-benzo[1,2,4]thiadiazin-7- | |
| | yloxy}acetonitrile | |
| 217 | 1-Cyclobutylmethyl-6-fluoro-4-hydroxy-3-(7-methoxy-1,1-dioxo-1,4- | 458 |
| | dihydro-1-benzo[1,2,4]thiadiazin-3-yl)-1H-quinolin-2-one | |
| 218 | 1-(3,3-Dimethylbutyl)-6-fluoro-4-hydroxy-3-(7-methoxy-1,1-dioxo- | 474 |
| | 1,4-dihydro-1-benzo[1,2,4]thiadiazin-3-yl)-1H-quinolin-2-one | |
| 219 | 3-(7-Acetyl-1,1-dioxo-1,4-dihydro-1-benzo[1,2,4]thiadiazin-3-yl)-4- | 454 |
| | hydroxy-1-(3-methylbutyl)-1H-quinolin-2-one | |
| 220 | (S)-2-{3-[1-(2-Cyclopropylethyl)-6-fluoro-4-hydroxy-2-oxo-1,2- | 515 |
| | dihydroquinolin-3-yl]-1,1-dioxo-1,4-dihydro-1-benzo[1,2,4]thiadiazin- | |
| | 7-yloxy}propionamide | |
| 221 | (R)-2-{3-[1-(2-Cyclopropylethyl)-6-fluoro-4-hydroxy-2-oxo-1,2- | 515 |
| | dihydroquinolin-3-yl]-1,1-dioxo-1,4-dihydro-1-benzo[1,2,4]thiadiazin- | |
| | 7-yloxy}propionamide | |
| 222 | {3-[1-(3,3-Dimethylbutyl)-6-fluoro-4-hydroxy-2-oxo-1,2- | 517 |
| | dihydroquinolin-3-yl]-1,1-dioxo-1,4-dihydro-benzo[1,2,4]thiadiazin-7- | |
| | yloxy}acetamide | |
| 223 | 3-{3-[1-(2-Cyclopropylethyl)-6-fluoro-4-hydroxy-2-oxo-1,2-dihydro- | 513 |
| | quinolin-3-yl]-1,1-dioxo-1,4-dihydro-1-benzo[1,2,4]thiadiazin-7-yl}- | |
| | 3-oxopropionamide | |
| 224 | 2-{3-[4-Hydroxy-6-(2-hydroxyethylamino)-1-(3-methylbutyl)-2-oxo- | 544 |
| | 1,2-dihydro-quinolin-3-yl]-1,1-dioxo-1,4-dihydro-1- | |
| | benzo[1,2,4]thiadiazin-7-yloxy}acetamide | |
| 225 | 2-{3-[1-(2-Cyclopropylethyl)-4-hydroxy-6-(2-hydroxyethylamino)-2- | 542 |
| | oxo-1,2-dihydro-quinolin-3-yl]-1,1-dioxo-1,4-dihydro-1- | |
| | benzo[1,2,4]thiadiazin-7-yloxy}acetamide | |
| 226 | 2-{3-[1-(2-Cyclopropylethyl)-6-fluoro-4-hydroxy-2-oxo-1,2-dihydro- | 500 |
| | quinolin-3-yl]-1,1-dioxo-1,4-dihydro-1-benzo[1,2,4]thiadiazin-7- | |
| | ylamino}acetamide | |
| 227 | 2-{3-[1-(2-Cyclopropylethyl)-6-fluoro-4-hydroxy-2-oxo-1,2-dihydro- | 530 |
| | quinolin-3-yl]-1,1-dioxo-1,4-dihydro-1-benzo[1,2,4]thiadiazin-7- | |
| | yloxy}-2-methylpropionic acid | |

The HCV NS5B inhibitory activity of the compounds of Formulas I, II m and IV was determined using standard procedures well known to those skilled in the art and described in, for example Behrens et al., EMBO J. 15:12-22 (1996), Lohmann et al., Virology 249:108-118 (1998) and Ranjith-Kumar et al., J. Virology 75:8615-8623 (2001).

All publications, including, but not limited to, patents and patent applications cited in this specification, are herein incorporated by reference as if each individual publication were specifically and individually indicated to be incorporated by reference herein as though fully set forth.

The above description fully discloses how to make and use the present invention. However, this invention is not limited to the particular embodiments described hereinabove, but includes all modification thereof within the scope of the appended claims and their equivalents. Those skilled in the art will recognise through routine experimentation that various changes and modifications can be made without departing from the scope of this invention.

What is claimed is:

1. A compound according to Formula I: 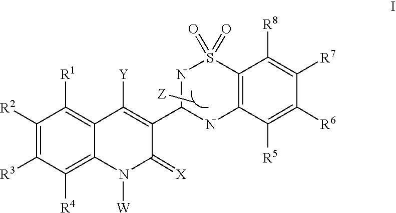
wherein:
R¹ is hydrogen, halogen, C₁-C₄ alkyl, —OR¹¹, —SR¹¹, —NR¹⁰R¹¹, aryl, —C(O)OH, —C(O)NHR¹¹, cyano or nitro;
R² is hydrogen, C₁-C₈ alkyl, C₂-C₈ alkenyl, C₂-C₈ alkynyl, C₃-C₆ cycloalkyl, heterocycloalkyl, aryl, heteroaryl, nitro, cyano, halogen, —C(O)OR⁹, —C(O)R⁹, —C(O)NR⁹R¹⁰, —OR⁹, —SR⁹, —S(O)R¹², —S(O)₂R¹², —NR⁹R¹⁰, protected —OH, —N(R¹⁰C(O)R⁹, —OC(O)NR⁹R¹⁰, —N(R¹⁰)C(O)NR⁹R¹⁰, —P(O)(OR⁹)₂, —SO₂NR⁹R¹⁰, —SO₃H, or —N(R¹⁰)SO₂R¹²,
where said C₁-C₈ alkyl, C₂-C₈ alkenyl or C₂-C₈ alkynyl is unsubstituted or substituted with one or more substituents independently selected from halogen, —OH, —SH, —OC₁-C₄ alkyl, —SC₁-C₄ alkyl, —NR¹⁰R¹¹, cyano, nitro, —CO₂R¹⁰, —C(O)OC₁-C₄ alkyl, —CONR¹⁰R¹¹, —CONH₂, aryl, and heteroaryl,
and where said cycloalkyl, heterocycloalkyl, aryl or heteroaryl is unsubstituted or substituted with one or more substituents independently selected from C₁-C₆ alkyl, C₁-C₆ haloalkyl, halogen, —OH, —SH, —NH₂, —OC₁-C₄ alkyl, —SC₁-C₄ alkyl, —N(C₁-C₄ alkyl)(C₁-C₄ alkyl), —NH(C₁-C₄ alkyl), cyano, nitro, —CO₂H, —C(O)OC₁-C₄ alkyl, —CON(C₁-C₄ alkyl)(C₁-C₄ alkyl), —CONH(C₁-C₄ alkyl) and —CONH₂;
R³ is hydrogen, halogen, cyano, C₁-C₆ alkyl, —OH, or —CO₂H;
R⁴, R⁵ and R⁶ are each independently selected from the group consisting of hydrogen, halogen, cyano, C₁-C₆ alkyl, —OH, and —OC₁-C₄ alkyl;
R⁷ is hydrogen, C₁-C₈ alkyl, C₂-C₈ alkenyl, C₂-C₈ alkynyl, C₃-C₆ cycloalkyl, heterocycloalkyl, aryl, heteroaryl, nitro, cyano, halogen, —C(O)OR⁹, —C(O)R⁹, —C(O)NR⁹R¹⁰, —OR⁹, —SR⁹, (O)R¹², —S(O)₂R¹², NR⁹R¹⁰, protected —OH, —N(R¹⁰)C(O)R⁹, —OC(O)NR⁹R¹⁰, —N(R¹¹C(O)NR⁹R¹⁰, —P(O)(OR⁹)₂, —SO₂NR⁹R¹⁰, —SO₃H, or —N(R¹⁰)SO₂R¹²,
where said C₁-C₈ alkyl, C₂-C₈ alkenyl or C₂-C₈ alkynyl is unsubstituted or substituted with one or more substituents independently selected from halogen, —OH, —SH, —OC₁-C₄ alkyl, —SC₁-C₄ alkyl, —NR¹⁰R¹¹, cyano, nitro, —CO₂H, —C(O)OC₁-C₄ alkyl, —CONR¹⁰R¹¹, —CONH₂, aryl, heteroaryl, heterocycloalkyl, —C(O)aryl, —C(O)heterocycloalkyl, and —C(O)heteroaryl, where said aryl, heteroaryl, heterocycloalkyl, aryl, —C(O)aryl, —C(O)heterocycloalkyl, or —C(O)heteroaryl is unsubstituted or substituted with one or more substituents independently selected from C₁-C₄ alkyl, C₁-C₄ haloalkyl, halogen, —OH, —SH, —NH₂, —OC₁-C₄ alkyl, —SC₁-C₄ alkyl, —N(C₁-C₄ alkyl)(C₁-C₄ alkyl), —NH(C₁-C₄ alkyl), cyano and nitro,
and where said cycloalkyl, heterocycloalkyl, aryl or heteroaryl is unsubstituted or substituted with one or more substituents independently selected from C₁-C₆ alkyl, C₁-C₆ haloalkyl, halogen, —OH, —SH, —NH₂, —OC₁-C₄ alkyl, —SC₁-C₄ alkyl, —N(C₁-C₄ alkyl)(C₁-C₄ alkyl), —NH(C₁-C₄ alkyl), cyano, nitro, —CO₂H, —C(O)OC₁-C₄ alkyl, —CON(C₁-C₄ alkyl)(C₁-C₄ alkyl), —CONH(C₁-C₄ alkyl) and —CONH₂;
R⁸ is hydrogen, halogen, hydroxyl or C₁-C₄ alkyl;
or R¹ and R² or R⁵ and R⁶ or R⁶ and R⁷ or R⁷ and R⁸ taken together are alkylenedioxy;
W is hydrogen, —C(O)OR¹¹, C₁-C₁₀ alkyl, C₂-C₁₀ alkenyl, C₂-C₁₀ alkynyl, C₃-C₆ cycloalkyl, —(C₁-C₆ alkyl)-(C₃-C₆ cycloalkyl), —(C₂-C₆ alkenyl)-(C₃-C₆ cycloalkyl), —(C₂-C₆ alkynyl)-(C₃-C₆ cycloalkyl), —(C₁-C₆ alkyl)-heterocycloalkyl, —(C₂-C₆ alkenyl)-heterocycloalkyl, —(C₂-C₆ alkynyl)-heterocycloalkyl, —(C₁-C₆ alkyl)-aryl, (C₂-C₆ alkenyl)-aryl, —(C₂-C₆ alkynyl)-aryl, —(C₁-C₆ alkyl)-heteroaryl, —(C₂-C₆ alkenyl)-heteroaryl, or —(C₂-C₆ alkynyl)-heteroaryl,
where said C₁-C₁₀ alkyl, C₂-C₁₀ alkenyl, C₂-C₁₀ alkynyl is unsubstituted or substituted with one or more substituents independently selected from halogen, cyano, —OH, —OC₁-C₄ alkyl, —SH, —SC₁-C₄ alkyl, —S(O)(C₁-C₄ alkyl), —SO₃H, and —S(O)₂(C₁-C₄ alkyl),
said C₃-C₆ cycloalkyl is unsubstituted or substituted with one or more substituents independently selected from halogen, cyano, C₁-C₄ alkyl, —OH, —OC₁-C₄ alkyl, —SH, —SC₁-C₄ alkyl, —S(O)(C₁-C₄ alkyl), —SO₃H, and —S(O)₂(C₁-C₄ alkyl),
and where the cycloalkyl, heterocycloalkyl, aryl or heteroaryl moiety of said —(C₁-C₆ alkyl)-(C₃-C₆ cycloalkyl), —(C₂-C₆ alkenyl)-(C₃-C₆ cycloalkyl), —(C₂-C₆ alkynyl)-(C₃-C₆ cycloalkyl), —(C₁-C₆ alkyl)-heterocycloalkyl, —(C₂-C₆ alkenyl)-heterocycloalkyl, —(C₂-C₆ alkynyl)-heterocycloalkyl, —(C₁-C₆ alkyl)-aryl, (C₂-C₆ alkenyl)-aryl, —(C₂-C₆ alkynyl)-aryl, —(C₁-C₆ alkyl)-heteroaryl, —(C₂-C₆ alkenyl)-heteroaryl, or —(C₂-C₆ alkynyl)-heteroaryl is unsubstituted or substituted with one or more substituents independently selected from C₁-C₆ alkyl, C₁-C₆ haloalkyl, halogen, cyano, nitro, —OH, —NH₂, —OC₁-C₄ alkyl, —N(C₁-C₄ alkyl)(C₁-C₄ alkyl), and —NH(C₁-C₄ alkyl);
X is O or S;
Y is —OH or —SH;
Z is hydrogen or C₁-C₄ alkyl;
wherein each R⁹ is independently selected from the group consisting of hydrogen, C₁-C₈ alkyl, C₂-C₈ alkenyl, C₂-C₈ alkynyl, C₃-C₈ cycloalkyl, heterocycloalkyl, aryl, heteroaryl, —C₁-C₆ alkyl-C₃-C₈ cycloalkyl, —C₁-C₆ alkyl-heterocycloalkyl, —C₁-C₆ alkyl-aryl, and —C₁-C₆ alkyl-heteroaryl, —C₂-C₆ alkenyl-C₃-C₈ cycloalkyl, —C₂-C₆ alkenyl-heterocycloalkyl, —C₂-C₆ alkenyl-aryl, —C₂-C₆ alkenyl-heteroaryl, —C₂-C₆ alkynyl-C₃-C₈ cycloalkyl, —C₂-C₆ alkynyl-heterocycloalkyl, —C₂-C₆ alkynyl-aryl, and —C₂-C₆ alkynyl-heteroaryl,
where said C₁-C₈ alkyl, C₂-C₈ alkenyl, or C₂-C₈ alkynyl is unsubstituted or substituted with one or more substituents independently selected from halogen, —OR¹¹, —NR¹⁰R¹¹, cyano, nitro, —CO₂R¹¹, —CONR¹⁰R¹¹, —NR¹⁰CONR¹⁰R¹¹, —OCONR¹⁰R¹¹, —SO₂NR¹⁰R¹¹, and —COR¹¹,
and where any of said cycloalkyl, heterocycloalkyl, aryl or heteroaryl (including the cycloalkyl, heterocycloalkyl, aryl or heteroaryl moieties of said —C₁-C₆ alkyl-C₃-C₈ cycloalkyl, —C₁-C₆ alkyl-heterocycloalkyl, —C₁-C₆ alkyl-aryl, or —C₁-C₆ alkyl-heteroaryl) is unsubstituted or substituted with one or more substituents independently selected from C₁-C₄ alkyl, C₁-C₄ haloalkyl, halogen, —OR¹¹, —NR¹⁰R¹¹, cyano, nitro, —CO₂R¹¹, —CONR¹⁰R¹¹, —NR¹⁰CONR¹⁰R¹¹, —OCONR¹⁰R¹¹, —SO₂NR¹⁰R¹¹, and —COR¹¹;
each R¹⁰ is independently selected from hydrogen and C₁-C₆ alkyl;
each R¹¹ is independently selected from the group consisting of hydrogen, C₁-C₆ alkyl, C₃-C₆ cycloalkyl, heterocycloalkyl, aryl, heteroaryl, —C₁-C₄ alkyl-C₃-C₈ cycloalkyl, —C₁-C₄ alkyl-heterocycloalkyl, —C₁-C₄ alkyl-aryl, or —C₁-C₄ alkyl-heteroaryl
where said cycloalkyl, heterocycloalkyl, aryl, heteroaryl, -alkylcycloalkyl, -alkylheterocycloalkyl, -alkylaryl or -alkylheteroaryl is unsubstituted or substituted with one or more substituents independently selected from C₁-C₆ alkyl, C₁-C₆ haloalkyl, halogen —OC₁-C₆ alkyl, —OC₁-C₆ haloalkyl, cyano, —N(C₁-C₆ allyl)(C₁-C₆ alkyl), —NH(C₁-C₆ alkyl), —NH₂, —CO₂C₁-C₆ alkyl, —CO₂H, —CON(C₁-C₆ alkyl)(C₁-C₆ alkyl), —CONH(C₁-C₆ allyl), and —CONH₂;
or, when present in any NR⁹R¹⁰ or NR¹⁰R¹¹, each R⁹ and R¹⁰ or each R¹⁰ and R¹¹, independently, taken together with the nitrogen to which they are attached represent a 3-6-membered saturated ring optionally containing one other heteroatom selected from oxygen and nitrogen, where said 3-6-membered ring is unsubstituted or substituted with one or more substituents independently selected from hydrogen, C₁-C₆ alkyl, halogen, cyano, —OC₁-C₆ alkyl, —OH, —N(C₁-C₆ alkyl)(C₁-C₆ alkyl), —NH(C₁-C₆ alkyl), —NH₂, —CO₂H, —C(O)OC₁-C₆ alkyl, —C(O)C₁-C₆ alkyl, —CON(C₁-C₆ alkyl)(C₁-C₆ alkyl), —CONH(C₁-C₆ alkyl), —CONH₂, C₃-C₆ cycloalkyl, heterocycloalkyl, aryl, heteroaryl, C₃-C₆ cycloalkyl-C₁-C₆ alkyl-, heterocycloalkyl-C₁-C₆ alkyl-, aryl-C₁-C₆ alkyl- and heteroaryl-C₁-C₆ alkyl-, and where said cycloalkyl, heterocycloalkyl, aryl, heteroaryl, cycloalkylalkyl-, heterocycloalkylalkyl-, arylalkyl- or heteroarylalkyl- is unsubstituted or substituted with one or more substituents independently selected from C₁-C₆ alkyl, C₁-C₆ haloalkyl, halogen —OC₁-C₆ alkyl, —OC₁-C₆ haloalkyl, cyano, —N(C₁-C₆ alkyl)(C₁-C₆ alkyl), —NH(C₁-C₆ alkyl), —NH₂, —CO₂C₁-C₆ alkyl, —CO₂H, —CON(C₁-C₆ alkyl)(C₁-C₆ alkyl), —CONH(C₁-C₆ alkyl), and —CONH₂;
each R¹² is independently selected from the group consisting of C₁-C₈ alkyl, C₂-C₈ alkenyl, C₂-C₈ alkynyl, C₃-C₈ cycloalkyl, heterocycloalkyl, aryl, heteroaryl, —C₁-C₆ alkyl-C₃-C₈ cycloalkyl, —C₁-C₆ alkyl-heterocycloalkyl, —C₁-C₆ alkyl-aryl, and —C₁-C₆ alkyl-heteroaryl, —C₂-C₆ alkenyl-C₃-Cg cycloalkyl, —C₂-C₆ alkenyl-heterocycloalkyl, —C₂-C₆ alkenyl-aryl, —C₂-C₆ alkenyl-heteroaryl, —C₂-C₆ alkynyl-C₃-C₈ cycloalkyl, —C₂-C₆ alkynyl-heterocycloalkyl, —C₂-C₆ alkynyl-aryl, and —C₂-C₆ alkynyl-heteroaryl,
where said C₁-C₈ alkyl, C₂-C₈ alkenyl, or C₂-C₈ alkynyl is unsubstituted or substituted with one or more substituents independently selected from halogen, —OR¹³, —NR¹⁰R¹³, cyano, nitro, —O₂R¹⁰R¹³, —CONR¹⁰R¹³, —NR¹⁰CONR¹⁰R¹³, —OCONR¹⁰OR¹³, —SO₂NR¹⁰R¹³, and —COR¹³,
and where any of said cycloalkyl, heterocycloalkyl, aryl or heteroaryl is unsubstituted or substituted with one or more substituents independently selected from C₁-C₄ alkyl, C₁-C₄ haloalkyl, halogen, —OR¹³, —NR¹⁰R¹³, cyano, nitro, —CO₂R¹³, —CONR¹⁰R¹³, —NR¹⁰CONR¹⁰R¹³, —OCONR¹⁰R¹³, —SO₂NR¹⁰R¹³, and —COR¹³;
each R¹³ is independently selected from the group consisting of hydrogen, C₁-C₈ alkyl, C₂-C₈ alkenyl, C₂-C₈ alkynyl, C₃-C₈ cycloalkyl, heterocycloalkyl, aryl, heteroaryl, —C₁-C₆ alkyl-C₃-C₈ cycloalkyl, —C₁-C₆ alkyl-heterocycloalkyl, —C₁-C₆ alkyl-aryl, and —C₁-C₆ alkyl-heteroaryl;
provided that when X is 0, Y is OH and Z, R¹, R², R³, R⁴, R⁵, R⁶, R⁷ and R⁸ are hydrogen: W is not hydrogen, —CH₃, —C₂H₅, -nC₃H₇, -nC₄H₉, -nC₅H₁₁, -nC₆H₁₃, -nC₇H₁₅, —(CH₂)CH(CH₃)₂, —(CH₂)₂CH(CH₃)₂, —CH₂CH═CH₂, —CH₂CH═CH(CH₃), —(CH₂)₃CN, —(CH₂)₄CN, —(CH₂)phenyl, —(CH₂)pyridin-2-yl, or —(CH₂)₂OCH₃;
or a tautomer thereof, or a pharmaceutically acceptable salt or solvate thereof.

2. The compound according to claim 1, wherein:
R¹ is hydrogen, halogen, C₁-C₄ alkyl, aryl, —OR^{a}, —C(O)OR⁶, —C(O)NR^{a}R^{a} or cyano;
R² is hydrogen, C₁-C₆ alkyl, C₁-C₆ haloalkyl, aryl, heteroaryl, nitro, cyano, halogen, —C(O)OR^{a}, —C(O)C₁-C₆ alkyl, —C(O)NR^{a}R^{a}, —OR^{b}, protected —OH, —SR^{b}, —S(O)R^{c}, —S(O)₂R^{b}, —NR^{a}R^{c}, —NR^{a}C(O)C₁-C₆ alkyl, —NR^{a}COaryl, —NR^{a}CO(C₁-C₄ alkyl)aryl, —NR^{a}C(O)heteroaryl, —NR^{a}C(O)(C₁-C₄ alkyl)heteroaryl, —NR^{a}C(O)cycloalkyl, —NR^{a}C(O)(C₁-C₄ alkyl)cycloalkyl, —NR^{a}C(O)heterocycloalkyl, —NR^{a}C(O)(C₁-C₄ alkyl)heterocycloalkyl, where each of said C₁-C₆ alkyl is optionally unsubstituted or substituted by one or more substituents independently selected from the group consisting of cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ aklyl, —CON(C₁-C₄ alkyl)(C₁-C₄ alkyl), —CONH(C₁-C₄ alkyl), and —CONH₂, and where each of said aryl, heteroaryl, cycloalkyl, or heterocycloalkyl is optionally unsubstituted or substituted with one or more substituents independently selected from C₁-C₄ alkyl, C₁-C₄ haloalkyl, halogen, —OR^{a}, —SR^{a}, —NR^{a}R^{a}, —CON(C₁-C₄ alkyl)(C₁-C₄ alkyl), —CONH(C₁-C₄ alkyl), —CONH₂, nitro and cyano;
R³ is H, halogen or —C(O)OH;
R⁴ is H, halogen, or C₁-C₄ alkyl;
R⁵ is H, halogen, C₁-C₄ alkyl, or —OR^{a};
R⁶ is H, halogen, or —OR^{a};
R⁷ is hydrogen, C₁-C₆ alkyl, C₂-C₆ alkenyl, C₂-C₆ alkynyl, aryl, heteroaryl, nitro, cyano, halogen, —C(O)OR^{a}, —C(O)C₁-C₆ alkyl, —C(O)NR^{a}R^{d}, —OR^{b}, —NR^{a}R^{d}, —N(R^{a})C(O)R^{d}, OC(O)NR^{a}R^{d}, or —N(R^{a})C(O)NR^{a}R^{d}, where said alkyl, alkenyl or alkynyl is unsubstituted or substituted with one or more substituents independently selected from halogen, —OR^{a}, —SR^{a}, —NR^{a}R^{a}, cyano, nitro, —CO₂H, —C(O)OC₁-C₄ alkyl, —CON(C₁-C₄ alkyl)(C₁-C₄ alkyl), —CONH(C₁-C₄ alkyl), —CONH₂, aryl, and heteroaryl, and where said aryl or heteroaryl is unsubstituted or substituted with one or more substituents independently selected from C₁-C₆ alkyl, C₁-C₆ haloalkyl, halogen, —OR^{a}, —SR^{a}, —NR^{a}R^{a}, cyano and nitro;
R⁸ is hydrogen or halogen;
or R¹ and R² or R⁵ and R⁶ or R⁶ and R⁷ or R⁷ and R⁸ taken together are alkylenedioxy;
W is hydrogen, —C(O)OR^{a}, C₃-C₈ alkyl, C₃-C₆ alkenyl, C₃-C₆ alkynyl, —(C₁-C₄ alkyl)-(C₃-C₆ cycloalkyl), —(C₁-C₄ alkyl)-heterocycloalkyl, —(C₁-C₄ alkyl)-aryl, or —(C₁-C₄ alkyl)-heteroaryl, where the C₁-C₈ alkyl, C₂-C₆ alkenyl or C₂-C₆ alkynyl is unsubstituted or substituted with one or more substituents independently selected from halogen, cyano, —OR^{a}, —SR^{a}, —S(O)C₁-C₄ alkyl, —S(O)₂C₁-C₄ alkyl, and where the cycloalkyl, heterocycloalkyl, aryl or heteroaryl moiety of the —(C I—C₄ alkyl)-(C₃-C₆ cycloalkyl), —(C₁-C₄ alkyl)-heterocycloalkyl, —(C₁-C₄ alkyl)-aryl, or —(C₁-C₄ alkyl)-heteroaryl is unsubstituted or substituted with one or more substituents independently selected from C₁-C₄ alkyl, C₁-C₄ haloalkyl, halogen, nitro, cyano, —OR^{a}, —NR^{a}R^{a};
X is O; Y is OH; and Z is hydrogen or methyl;
each R^{a} is independently H or C₁-C₄ alkyl;
each R^{b} is independently H or C₁-C₄ alkyl, where the alkyl is optionally unsubstituted or substituted by one or more substituents independently selected from the group consisting of halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —CON(C₁-C₄ alkyl)(C₁-C₄ alkyl), —CONH(C₁-C₄ alkyl), —CONH₂, aryl, heteroaryl, heterocycloalkyl, —C(O)aryl, —C(O)heterocycloalkyl, and —C(O)heteroaryl, where said aryl, heteroaryl, heterocycloalkyl, —C(O)aryl, —C(O)heterocycloalkyl, or —C(O)heteroaryl is unsubstituted or substituted with one or more substituents independently selected from C₁-C₄ alkyl, C₁-C₄ haloalkyl, halogen, —OR^{a}, —SR^{a}, —NR^{a}R^{a}, cyano and nitro;
each R^{c} is independently C₁-C₄ alkyl, optionally unsubstituted or substituted by one or more substituents independently selected from the group consisting of halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —CON(C₁-C₄ alkyl)(C₁-C₄ alkyl), —CONH(C₁-C₄ alkyl), —CONH₂, aryl and heteroaryl, and where said aryl or heteroaryl is unsubstituted or substituted with one or more substituents independently selected from C₁-C₄ alkyl, C₁-C₄ haloalkyl, halogen, —OR^{a}, —SR¹, —NR^{a}R^{a}, cyano and nitro;
each R^{d} is independently H or C₁-C₄ alkyl, where the alkyl is optionally substituted by one or more substituents independently selected from the group consisting of halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —CON(C₁-C₄ alkyl)(C₁-C₄ alkyl), —CONH(C₁-C₄ alkyl), —CONH₂, —C(O)C₁-C₄ alkyl, —C(O)aryl, —C(O)heteroaryl, cycloalkyl, heterocycloalkyl, aryl and heteroaryl, and where said aryl or heteroaryl is unsubstituted or substituted with one or more substituents independently selected from C₁-C₄ alkyl, C₁-C₄ haloalkyl, halogen, —ORB, —SR^{a}, —NR^{a}R^{a}, cyano and nitro;
or, when present in any NR^{a}R^{b} or NR^{a}R^{d}, each R^{a} and R^{b} or each ^{a} and R^{d}, independently, taken together with the nitrogen atom to which they are attached form a 5- or 6-membered heterocycloalkyl ring, which optionally contains one or more heteroatoms selected from oxygen or nitrogen and which is unsubstituted or substituted with one or more substituents selected from the group halogen, cyano, —OC₁-C₄ alkyl, —OH, —N(C₁-C₄ alkyl)(C₁-C₄ alkyl), —NH(C₁-C₄ alkyl), —NH₂, —CO₂H, —C(O)OC₁-C₄ alkyl, —C(O)C₁-C₄ alkyl, —CON(C₁-C₄ alkyl)(C₁-C₄ alkyl), —CONH(C₁-C₄ alkyl), —CONH₂, —C(O)C₁-C₄ alkyl;
provided that when Z, R¹, R², R³, R⁴, R⁵, R⁶, R⁷ and R⁸ are hydrogen: W is not hydrogen, —CH₃, —C₂HS, —IC₃H₇, -nC₄H₉, -nC₅H₁₁, -nC₆H₁₃, -nC₇H₁₅, —(CH₂)CH(CH₃)₂, —(CH₂)₂CH(CH₃)₂, —CH₂CH═CH₂, —CH₂CH═CH(CH₃), —(CH₂)₃CN, —(CH₂)₄CN, —(CH₂)phenyl, —(CH₂)pyridin-2-yl, or —(CH₂)₂OH₃;
or a tautomer thereof, or a pharmaceutically acceptable salt or solvate thereof.

3. The compound according to claim 1, wherein:
R¹ is H, phenyl, —CH₃, F, Cl, Br, —OH, —C(O)OH, or —C(O)NHCH₃;
R² is H, F, Cl, Br, 1, —OH, —OCH₃, —CH₃, —CH₂(4-OCH₃-phenyl), —CH═CHC(O)NH₂, —NO₂, —NH₂, —NHCH₃, —N(CH₃)₂, —CONHCH₃, —CON(CH₃)₂, —CO₂H, —CO₂CH₂CH₃, —O(CH₂)₂CH(CH₃)₂, —O(CH₂)₃CN, —OCH₂CN, —O(CH₂)₂OCH₃, —O(CH₂)₂OH, —OCH₂CH(OH)CH₂CH₃, —O(CH₂)₂N(CH₃)₂, —OCH₂phenyl, —OCH₂CONH₂, —O(6-Br-pyridin-2-yl), —O(60CH₃-pyridin-2-yl), —OSi(CH₃)₂(tBu), —NHCH₂CO₂H, —NHCH₂CO₂CH₂CH₃, —NHCH₂-2-furyl, —NH(CH₂)₂OH, —NHCH₂CN, —NHCH₂C(O)NH₂, —NHC(O)CH₃, —NHC(O)CH₂CH(CH₃)₂, —NHC(O)CH₂N(CH₃)₂, —NHC(O)phenyl, —NHC(O)(3-CH₃O-phenyl), —NHC(O)(4-NO₂-phenyl), —NHC(O)(3-CN-phenyl), —NHC(O)(3-CF₃-phenyl), —NHC(O)(3-F-phenyl), —NHC(O)(3-pyridyl), —NHC(O)(2-furyl), —NHC(O)(2-thienyl), —NHC(O)(4-OCH₃-phenyl), —NHC(O)(cyclopentyl);
R³ is H, F, Cl, Br, or —CO₂H;
R⁴ is H, Br or —(CH₂)₂CH(CH₃)₂; R⁵ is H, —CH₃, —OCH₃ or —OH;
R⁶ is H, Br, —OH, or —OCH₃;
R⁷ is H, —CH₃, —OH, —OCH₃, phenyl, F, Cl, Br, I, NO₂, —NH₂, —N(CH₃)₂, —NHCH₂CN, —CN, —CH₂NH₂, —CH₂CH₂C(O)NH₂, —CH═CHC(O)NH₂, —(CH₂)₂CH(CH₃)OCH₃, —CHO, —C(O)CH₃, —CO₂CH₃, —CO₂H, —C(O)NH₂, —C(O)NHCH₃, —C(O)N(CH₃)₂, —OCH₂CO₂CH₃, —OCH₂CO₂H, —OCH₂CH(NH₂)CH₂CH₃, —O(CH₂)₂N(CH₃)₂, —OCH₂CN, —O(CH₂)₂NH₂, —OCH₂C(O)NH₂, —OCH₂CONHCH₃, —OCH₂CON(CH₃)₂, —OCH(CH₃)C(O)NH₂, —OCH₂-tetrazol-5-yl, —OCH₂C(O)(3-pyridyl), —OCH₂C(O)(N-pyrrolidinyl), —OCH₂C(O)(N-piperazinyl), —OCH₂C(O)(N-morpholinyl), —OCH₂(5-methyl-1,3,4-oxadiazol-2-yl), —C(O)NH(CH₂)₃(N-imidazoyl), —C(O)NHCH₂CH(OCH₃)₂, —C(O)(4-acetylpiperizin-1-yl), —C(O)NHCH₂(2-tetrahydrofuryl), —C(O)NHCH₂phenyl, —C(O)NH(CH₂)₃N(CH₂CH₃)₂, —C(O)(N-pyrrolidinyl), —C(O)NH(CH₂)₂(4-OCH₃phenyl), or —NHCH₂phenyl;
R⁸ is H;
or R¹ and R² taken together are methylenedioxy;
W is selected from the group consisting of —(CH₂)₁₋₃-phenyl, —CH₂-(2-CN-phenyl), —(CH₂)₁-2-cyclopropyl, —CH₂(2-CH₃-cycloprop-1-yl), —CH₂)-cyclobutyl, —(CH₂)-cyclopentyl, —(CH₂)-cyclohexyl, —CH₂-(2-tetrahydrofuryl), —CH₂-(3-tetrahydrofuryl), —CH₂-3-pyridyl), —CH₂-(6-NH₂-3-pyridyl), —CH₂-(4-pyridyl), —CH₂-(2-NH₂-4-pyridyl), —CH₂-(2-CH₃₄ pyridyl), —CH₂-(4-bromophenyl), —CH₂-(3-bromophenyl), —CH₂-(3-NO₂-phenyl), —CH₂-(3-furyl), —(CH₂)₂-(2-thienyl), —CH₂)-(3-thienyl), —(CH₂)₂CH(CH₃)₂, —(CH₂)₂C(CH₃)₃, —CH₂)₂CH(CH₃)CH₂CH₃, —(CH₂)₂CH(CH₃)(CF₃), —(CH₂)₂CH_{2,} —CH₂CH═CH_{2,}—(CH₂)₂CHBr(CH₃), —(CH₂)CH═C(CH₃)₂, —(CH₂)₃CF₃, —(CH₂)₃CN, —(CH₂)₃₋₄OH, —(CH₂)₂CH(CH₃)OCH₃, —CH₂)₂C≡CH, —(CH₂)₃C═CH, —CO₂CH₂CH₃, —(CH₂)₂CH(CH₃)CH₂CH₃, —(CH₂)₂SCH₃, (CH₂)₃SCH₃, —(CH₂)₂S(O)CH₃, —CH₂)₂S(O)₂CH₃;
X is O; Y is OH; and Z is hydrogen or methyl;
provided that when Z, R¹, R², R³, R⁴, R⁵, R⁶, R⁷ and R⁸ are hydrogen: W is not —(CH₂)₂CH(CH₃)₂, —CH₂CH═CH₂, —CH₂CH═CH(CH₃), —(CH₂)₃CN, or —(CH₂)phenyl;
or a tautomer thereof, or a pharmaceutically acceptable salt or solvate thereof.

4. The compound according to claim 1, wherein:
R¹ and R³ are each independently H or F;
R² is hydrogen halogen, —OR^{b′}, —NHR^{b′}, NO₂, where R^{b′} is H or C₁-C₂ alkyl, where the C₁-C₂ alkyl is optionally unsubstituted or substituted by a substituent selected from the group consisting of cyano, —OH, —CO₂H, —CONH₂, —C(O)OC₁-C₂ alkyl, —CONH(C₁-C₂ alkyl), and unsubstituted monocyclic heteroaryl;
R⁴, R⁶ and R⁸ are each H;
R⁵ is H or —OH;
R⁷ is hydrogen, halogen, C₁-C₂ alkyl, C₂ alkenyl, —C(O)OR^{a′}; —C(O)R^{a′}; —OR^{b″}, NR^{a′}R^{d′}; —C(O)NR^{a′}R^{d′}, where said alkyl or alkenyl is unsubstituted or substituted a substituent selected from —NH₂ and —CONH₂, R^{4′} is H or methyl, R^{b″} is H or C₁-C₄ alkyl, where the C₁-C₄ alkyl is optionally unsubstituted or substituted by a substituent selected from the group consisting of cyano, —NH₂, —CO₂H, —CONH₂, —C(O)OC₁-C₂ alkyl, —CON(C₁-C₄ alkyl)(C₁-C₄ alkyl), —CONH(C₁-C₄ alkyl), monocyclic heteroaryl, —C(O)monocyclic heterocycloalkyl, and —C(O)-monocyclic heteroaryl, where said heteroaryl, —C(O)heterocycloalkyl, or —C(O)heteroaryl are unsubstituted or substituted one or more of C₁-C₄ alkyl, halogen, cyano, —OH, —NH₂, and —CONH₂, R^{d′} is H or C₁-C₂ alkyl, where the C₁-C₂ alkyl is unsubstituted or substituted by a substituent selected from the group consisting of cyano and unsubstituted aryl, or R^{a′} and R^{d′} taken together with the nitrogen atom to which they are attached form a 5- or 6membered heterocycloalkyl ring, which optionally contains an additional nitrogen heteroatom and which is unsubstituted or substituted with —C(O)C₁-C₂ alkyl;
or R¹ and R² taken together are alkylenedioxy;
W is C₄-C₆ alkyl, C₄alkenyl, C₄ alkynyl, —(C₁-C₂ alkyl)-(C₃-C₆ cycloalkyl), —(C₁-C₄ alkyl)-heterocycloalkyl, —(C₁-C₄ alkyl)-aryl, or —(C₁ alkyl)-heteroaryl, where the C₄-C₆ alkyl, C₄ alkenyl or C₄ alkynyl is unsubstituted or substituted with one or more substituents independently selected from halogen, —OH, —OCH₃, —SCH₃, and where the cycloalkyl, heterocycloalkyl, aryl or heteroaryl moiety of the —(C₁-C₄ alkyl)-(C₃-C₆ cycloalkyl), —(C₁-C₄ alkyl)-heterocycloalkyl, —(C₁-C₄ alkyl)-aryl, or —(C₁-C₄ alkyl)-heteroaryl is unsubstituted or substituted with one or more substituents independently selected from —CH₃, halogen, nitro, cyano, —OR^{a}, —NR^{a}R^{a};
X is O; Y is OH; and Z is hydrogen;
provided that when R¹, R², R³, R⁴, R⁵, R⁶, R⁷ and R⁸ are hydrogen: W is not -nC₄H₉, -nC₅H₁₁, -nC₆H₁₃, -nC₇H₁₅, —(CH₂)CH(CH₃)₂, —(CH₂)₂CH(CH₃)₂, —CH₂CH═CH(CH₃), —(CH₂)phenyl, or —(CH₂)pyridin-2-yl;
or a tautomer thereof, or a pharmaceutically acceptable salt or solvate thereof.

5. A compound according to any one of claims 1-4, having the formula: 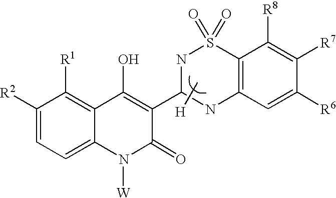
or a tautomer thereof, or a pharmaceutically acceptable salt or solvate thereof.

6. A compound according to any one of claims 14, having the formula: 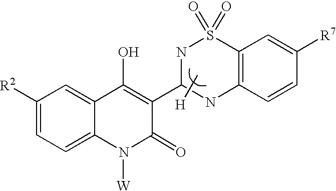
or a tautomer thereof, or a pharmaceutically aceptable salt or solvate thereof.

7. A compound selected from the group consisting of:
1-cyclopropylmethyl-3-(1,1-dioxo-1,4-dihydrobenzo[1,2,4]thiadiazin-3-yl)-4-hydroxy-1H-quinolin-2-one,
3-(1,1-dioxo-1,4dihydrobenzo[1,2,4]thiadiazin-3-yl)-4-hydroxy-1-(3-methylbutyl)-6-nitro-1H-quinolin-2-one,
6-chloro-3-(1,1-dioxo-1,4-dihydrobenzo[1,2,4]thiadiazin-3-yl)-4-hydroxy-1-(3-methylbutyl)-1H-quinolin-2-one,
6-bromo-3-(1,1-dioxo-1,4-dihydrobenzo[1,2,4]thiadiazin-3-yl)-4-hydroxy-1-3-methylbutyl)-1H-quinolin-2-one,
3-(1,1-dioxo-1,4-dihydrobenzo[1,2,4]thiadiazin-3-yl)-4-hydroxy-6-methoxy-1-(3-methylbutyl)-H-quinolin-2-one,
1-but-3-enyl-3-(1,1-dioxo-1,4-dihydrobenzo[1,2,4]thiadiazin-3-yl)-4-hydroxy-1H-quinolin-2-one,
1-(3-bromobutyl)-3-(1,1-dioxo-1,4-dihydrobenzo[1,2,4]thiadiazin-3-yl)-4-hydroxy-1-1H-quinolin-2-one,
N-[3-(1,1-dioxo-1,4dihydro-1-benzo[1,2,4]thiadiazin-3-yl)-4-hydroxy-1-(3-methylbutyl)-2-oxo-1,2-dihydro-quinolin-6-yl]A4-fluorobenzamide,
1-cyclohexylmethyl-3-(1,1-dioxo-1,4-dihydrobenzo[1,2,4]thiadiazin-3-yl)₄-hydroxy-1H-quinolin-2-one,
3-(1,1-dioxo-1,4-dihydrobenzo[1,2,4]thiadiazin-3-yl)-4-hydroxy-1-(3-methylbut-2-eneyl)-1H-quinolin-2-one,
3-(1,1-dioxo-1,4-dihydrobenzo[1,2,4]thiadiazin-3-yl)-4-hydroxy-6-methyl-1-(3-methylbutyl)-1H-quinolin-2-one,
3-(1,1-dioxo-1,4-dihydrobenzo[1,2,4]thiadiazin-3-yl)-6-fluoro-4-hydroxy-1-(3-methylbutyl)-1H-quinolin-2-one,
3-(1,1-dioxo-1,4-dihydrobenzo[1,2,4]thiadiazin-3-yl)-5-fluoro-4-hydroxy-1-(3-methylbutyl)-1H-quinolin-2-one,
6,7-difluoro-3-(1,1-dioxo-1,4-dihydrobenzo[1,2,4]thiadiazin-3-yl)-4-hydroxy-1-(3-methylbutyl)-1H-quinolin-2-one,
3-(1,1-dioxo-1,4-dihydrobenzo[1,2,4]thiadiazin-3-yl)-7-fluoro-4-hydroxy-1-(3-methylbutyl)-1H-quinolin-2-one,
3-(1,1-dioxo-1,4-dihydrobenzo[1,2,4]thiadiazin-3-yl)-4-hydroxy-1-(4,4,4-trifluorobutyl)-1H-quinolin-2-one,
6-amino-3-(1,1-dioxo-1,4-dihydrobenzo[1,2,4]thiadiazin-3-yl) hydroxy-1-(3-methylbutyl)-1H-quinolin-2-one,
3-(1,1-dioxo-1,4-dihydrobenzo[1,2,4]thiadiazin-3-yl)-4-hydroxy-1-(4-hydroxybutyl)-1H-quinolin-2-one,
3-(1, 1-dioxo-1,2-dihydro-7-nitrobenzo[1,2,4]thiadiazin-3-yl)-4-hydroxy-1-(3-methylbutyl)-1H-quinolin-2-one,
3-(1,1-dioxo-1,4-dihydrobenzo[1,2,4]thiadiazin-3-yl)-4-hydroxy-1-pent-4ynyl-1H-quinolin-2-one,
3-(7-bromo-1,1-dioxo-1,4-dihydrobenzo[1,2,4]thiadiazin-3-yl)-4-hydroxy-1-(3-methylbutyl)-1H-quinolin-2-one,
3-(7-amino-1,1-dioxo-1,4-dihydrobenzo[1,2,4]thiadiazin-3-yl)-4-hydroxy-1-(3-methylbutyl)-1H-quinolin-2-one,
3-(7-cyano-1,1-dioxo-1,4-dihydrobenzo[1,2,4]thiadiazin-3-yl)-4-hydroxy-1-(3-methylbutyl)-1H-quinolin-2-one,
8-bromo-3-(1,1-dioxo-1,4-dihydrobenzo[1,2,4]thiadiazin-3-yl)-4-hydroxy-1H-quinolin-2-one,
6-amino-3-(7-amino-1,1-dioxo-1,4-dihydrobenzo[1,2,4]thiadiazin-3-yl)-4-hydroxy-1-(3-methylbutyl)-1H-quinolin-2-one,
1-cyclopentylmethyl-3-(1,1-dioxo-1,4-dihydrobenzo[1,2,4]thiadiazin-3-yl)-4-hydroxy-1-1H-quinolin-2-one,
1-cyclobutylmethyl-3-(1,1-dioxo-1,4-dihydrobenzo[1,2,4]thiadiazin-3-yl)₄-hydroxy-1-1H-quinolin-2-one,
3-(1, 1-dioxo-1,4-dihydrobenzo[1,2,4]thiadiazin-3-yl)-4-hydroxy-5-methyl-1-(3-methylbutyl)-1H-quinolin-2-one,
5-chloro-3-(1,1-dioxo-1,4-dihydrobenzo[1,2,4]thiadiazin-3-yl)-4-hydroxy-1-(3-methylbutyl)-1H-quinolin-2-one,
5-bromo-3-(1,1-dioxo-1,4-dihydrobenzo[1,2,4]thiadiazin-3-yl)-4-hydroxy-1-(3-methylbutyl)-1H-quinolin-2-one,
4-[3-(1,1-dioxo-1,4-dihydrobenzo[1,2,4]thiadiazin-3-yl)-4-hydroxy-4-methoxy-2-oxo-2H-quinolin-1-yl]butyronitrile,
1-but-3-ynyl-3-(1,1-dioxo-1,4-dihydrobenzo[1,2,4]thiadiazin-3-yl)-4-hydroxy-1H-quinolin-2-one,
1-(3,3-dimethylbutyl)-3-(1,1-dioxo-1,4-dihydrobenzo[1,2,4]thiadiazin-3-yl)-4-hydroxy-1H-quinolin-2-one,
1-(2-cyclopropylethyl)-3-(1, 1-dioxo-1,4-dihydrobenzo[1,2,4]thiadiazin-3-yl)-4-hydroxy-1H-quinolin-2-one,
furan-2-carboxylic acid, [3-(1,1-dioxo-1,4-dihydrobenzo[1,2,4]thiadiazin-3-yl)-4-hydroxy-1-(3-methylbutyl)-2-oxo-1,2-dihydroquinolin-6-yl]amide,
3-cyano-N-[3-(1,1-dioxo-1,4-dihydrobenzo[1,2,4]thiadiazin-3-yl)-4-hydroxy-1-(3-methylbutyl)-2-oxo-1,2-dihydroquinolin-6-yl]benzamide,
cyclopentanecarboxylic acid [3-(1,1-dioxo-1,4-dihydrobenzo[1,2,4]thiadiazin-3-yl)-4-hydroxy-1-(3-methylbutyl)-2-oxo-1,2-dihydroquinolin-6-yl]amide,
N-[3-(1,1-dioxo-1,4-dihydrobenzo[1,2,4]thiadiazin-3-yl)-4-hydroxy-1-(3-methylbutyl)-2-oxo-1,2-dihydroquinolin-6-yl]-3-methoxybenzamide,
N-[3-(1,1-dioxo-1,4dihydrobenzo[1,2,4]thiadiazin-3-yl)-4-hydroxy-1-(3-methylbutyl)-2-oxo-1,2-dihydroquinolin-6-yl]-benzamide,
N-[3-(1,1-dioxo-1,4-dihydrobenzo[1,2,4]thiadiazin-3-yl)-4-hydroxy-1-(3-methylbutyl)-2-oxo-1,2-dihydroquinolin-6-yl]4nitrobenzamide,
3-[4-hydroxy-1-(3-methylbutyl)-2-oxo-1,2-dihydroquinolin-3-yl]-1,1-dioxo-1,4-dihydrobenzo[1,2,4]thiadiazine-7-carboxylic acid amide,
3-(6-bromo-1,1-dioxo-1,4-dihydrobenzo[1,2,4]thiadiazin-3-yl)-4-hydroxy-1-(3-methylbutyl)-1H-quinolin-2-one,
N-[3-(1,1-dioxo-1,4-dihydrobenzo[1,2,4]thiadiazin-3-yl)-4-hydroxy-1-(3-methylbutyl)-2-oxo-1,2-dihydroquinolin-6-yl]-3-methylbutyramide,
[3-(1,1-dioxo-1,4-dihydrobenzo[1,2,4]thiadiazin-3-yl)-4-hydroxy-1-(3-methylbutyl)-2-oxo-1,2-dihydroquinolin-6-ylamino]acetic acid ethyl ester,
4-hydroxy-1-(3-methylbutyl)-3-(5-methyl-1,1-dioxo-1,4-dihydrobenzo[1,2,4]thiadiazin-3-yl)-1H-quinolin-2-one,
2-dimethylamino-N-[3-(1,1-dioxo-1,4-dihydrobenzo[1,2,4]thiadiazin-3-yl)-4-hydroxy-1-(3-methylbutyl)-2-oxo-1,2-dihydroquinolin-6yl]acetamide,
3-(1,1-dioxo-1,4-dihydrobenzo[1,2,4]thiadiazin-3-yl)-4-hydroxy-1-(2-methylsulfanylethyl)-1H-quinolin-2-one,
3-(1, 1-dioxo-1,4-dihydrobenzo[1,2,4]thiadiazin-3-yl)-4-hydroxy-1-(3-methylsulfanylpropyl)-1H-quinolin-2-one,
3-(1,1-dioxo-1,4-dihydrobenzo[1,2,4]thiadiazin-3-yl)-1-furan-3-ylmethyl-4-hydroxy-1H-quinolin-2-one,
3-(1,1-dioxo-1,4-dihydrobenzo[1,2,4]thiadiazin-3-yl)-4-hydroxy-1-(2-thiophen-2-yl-ethyl)-1-quinolin-2-one,
3-(1,1-dioxo-1,4-dihydrobenzo[1,2,4]thiadiazin-3-yl)-4-hydroxy-1-(2-thiophen-3-yl-ethyl)-1H-quinolin-2-one,
3-(7-chloro-1,1-dioxo-1,4-dihydrobenzo[1,2,4]thiadiazin-3-yl)-4-hydroxy-1-(3-methylbutyl)-1H-quinolin-2-one,
3-[4-hydroxy-1-(3-methylbutyl)-2-oxo-1,2-dihydroquinolin-3-yl]-1,1-dioxo-1,4-dihydrobenzo[1,2,4]thiadiazine-7-carboxylic acid, methyl ester,
3-[4-hydroxy-1-(3-methylbutyl)-2-oxo-1,2-dihydroquinolin-3-yl]-1,1-dioxo-1,4-dihydrobenzo[1,2,4]thiadiazine-7carboxylic acid,
6-(tert-butyldimethylsilanyloxy)-3-(1,1-dioxo-1,4-dihydrobenzo[1,2,4]thiadiazin-3-yl)-4-hydroxy-1-(3-methylbutyl)-1H-quinolin-2-one,
3-(1,1-dioxo-1,4-dihydrobenzo[1,2,4]thiadiazin-3-yl)-4,6-dihydroxy-1-(3-methylbutyl)-1H-quinolin-2-one,
3-(1,1-dioxo-1,4-dihydrobenzo[1,2,4]thiadiazin-3-yl)-4-hydroxy-1-(4,4,4-trifluoro-3-methylbutyl)-1H-quinolin-2-one,
4hydroxy-3-(7-methoxy-1,1-dioxo-1,4-dihydrobenzo[1,2,4]thiadiazin-3-yl)-1-(3-methylbutyl)-1H-quinolin-2-one,
[3-(1,1-dioxo-1,4-dihydrobenzo[1,2,4]thiadiazin-3-yl)-4-hydroxy-1-(3-methylbutyl)-2-oxo-1,2-dihydroquinolin-6-ylamino]-acetic acid,
3-(1,1-dioxo-1,4-dihydrobenzo[1,2,4]thiadiazin-3-yl)-4-hydroxy-1-(3-methylbutyl)-5-phenyl-1H-quinolin-2-one,
4-hydroxy-3-(7-hydroxy-1,1-dioxo-1,4-dihydrobenzo[1,2,4]thiadiazin-3-yl)-1-(3-methylbutyl)-1H-quinolin-2-one,
{3-[4hydroxy-1-(3-methylbutyl)-2-oxo-1,2-dihydroquinolin-3-yl]-1,1-dioxo-1,4-dihydrobenzo[1,2,4]thiadiazin-7-yloxy)-acetic acid methyl ester,
{3-[4-hydroxy-1-(3-methylbutyl)-2-oxo-1,2-dihydroquinolin-3-yl]-1,1-dioxo-1,4-dihydrobenzo[1,2,4]thiadiazin-7-yloxy}-acetic acid,
1-(2-cyclopropylethyl)-3-(1,1-dioxo-1,4-dihydrobenzo[1,2,4]thiadiazin-3-yl)-4-hydroxy-6-nitro-1H-quinolin-2-one,
3-[4-hydroxy-1-(3-methylbutyl)-2-oxo-1,2-dihydroquinolin-3-yl]-1,1-dioxo-1,4-dihydrobenzo[1,2,4]thiadiazine-7-carboxylic acid dimethylamide,
6amino-1-(2-cyclopropylethyl)-3-(1, 1-dioxo-1,4-dihydrobenzo[1,2,4]thiadiazin-3-yl)-4-hydroxy-1H-quinolin-2-one,
3-(1,1-dioxo-1,4-dihydrobenzo[1,2,4]thiadiazin-3-yl)-4-hydroxy-1-(2-methanesulfinylethyl)-1H-quinolin-2-one,
3-(1,1-dioxo-1,4-dihydrobenzo[1,2,4]thiadiazin-3-yl)-4-hydroxy-1-(2-methanesulfonylethyl)-1H-quinolin-2-one,
(2-cyclopropylethyl)-6-dimethylamino-1,1-dioxo-1,4-dihydrobenzo[1,2,4]thiadiazin-3-yl)-4hydroxy-1H-quinolin-2-one,
1-(2-cyclopropylethyl)-3-(1,1-dioxo-1,4-dihydrobenzo[1,2,4]thiadiazin-3-yl)-4-hydroxy-6-methylamino-1H-quinolin-2-one,
1-(2-cyclopropylethyl)-3-(1,1-dioxo-1,4-dihydrobenzo[1,2,4]thiadiazin-3-yl)-6-fluoro-4-hydroxy-1H-quinolin-2-one,
1-(2-cyclopropylethyl)-6-(2-dimethylaminoethoxy)-3-(1,1-dioxo-1,4-dihydrobenzo[1,2,4]thiadiazin-3-yl)-4-hydroxy-1H-quinolin-2-one,
3-[7-(2-dimethylaminoethoxy)-1,1-dioxo-1,4-dihydrobenzo[1,2,4]thiadiazin-3-yl]-4-hydroxy-1-(3-methylbutyl)-1H-quinolin-2-one,
3-(1,1-dioxo-1,4-dihydrobenzo[1,2,4]thiadiazin-3-yl)-4-hydroxy-1-(3-methylbutyl)-2-oxo-1,2-dihydroquinoline-6-carboxylic acid methylamide,
1-(2-cyclopropylethyl)-4-hydroxy-3-(7-methoxy-1,1-dioxo-1,4-dihydrobenzo[1,2,4]thiadiazin-3-yl)-1H-quinolin-2-one,
1-(2-cyclopropylethyl)-3-(1,1-dioxo-1,4-dihydrobenzo[1,2,4]thiadiazin-3-yl)-6-[(furan-2-ylmethyl)amino]4-hydroxy-1H-quinolin-2-one,
3-[1-(2-cyclopropylethyl)-4-hydroxy-2-oxo-1,2-dihydroquinolin-3-yl]-1,1-dioxo-1,4-dihydro-1-benzo[1,2,4]thiadiazine-7-carboxylic acid dimethylamide,
1-(2-cyclopropylethyl)-4-hydroxy-3-(7-iodo-1,1-dioxo-1,4-dihydrobenzo[1,2,4]thiadiazin-3-yl)-1H-quinolin-2-one,
1-(2-cyclopropylethyl)-3-(1,1-dioxo-1,4-dihydrobenzo[1,2,4]thiadiazin-3-yl)-4-hydroxy-6-(2-hydroxyethoxy)-1H-quinolin-2-one,
3-[1-(2-cyclopropylethyl)-4-hydroxy-2-oxo-1,2-dihydroquinolin-3-yl]-1,1-dioxo-1,4-dihydrobenzo[1,2,4]thiadiazine-7-carboxylic acid methylamide,
3-(1,1-dioxo-1,4-dihydrobenzo[1,2,4]thiadiazin-3-yl)-4-hydroxy-1-(3-methylbutyl)-2-oxo-1,2-dihydroquinoline-5-carboxylic acid methylamide,
1-(3,3-dimethylbutyl)-3-(1,1-dioxo-1,4-dihydrobenzo[1,2,4]thiadiazin-3-yl)-4-hydroxy-6-nitro-1H-quinolin-2-one,
3-[6-amino-4-hydroxy-1-(3-methylbutyl)-2-oxo-1,2-dihydroquinolin-3-yl]-1,1-dioxo-1,4-dihydrobenzo[1,2,4]thiadiazine-7-carboxylic acid amide,
6-amino-1-(3,3-dimethylbutyl)-3-(1,1-dioxo-1,4-dihydrobenzo[1,2,4]thiadiazin-3-yl)-4-hydroxy-1H-quinolin-2-one,
3-(1,1-dioxo-1,4-dihydrobenzo[1,2,4]thiadiazin-3-yl)-4-hydroxy-2-oxo-2H-quinoline-1-carboxylic acid ethyl ester,
1-(3,3-dimethylbutyl)-3-(1,1-dioxo-1,4-dihydrobenzo[1,2,4]thiadiazin-3-yl)-4-hydroxy-6-(2-hydroxyethylamino)-1H-quinolin-2-one,
6-benzyloxy-3-(1,1-dioxo-1,4-dihydrobenzo[1,2,4]thiadiazin-3-yl)-4-hydroxy-1-(3-methylbutyl)-1H-quinolin-2-one,
{3-[4-hydroxy-1-(3-methylbutyl)-2-oxo-1,2-dihydroquinolin-3-yl]-1,1-dioxo-1,4-dihydrobenzo[1,2,4]thiadiazin-7-yloxy}acetonitrile,
3-[7-(2-aminoethoxy)-1,1-dioxo-1,4-dihydrobenzo[1,2,4]thiadiazin-3-yl]-4-hydroxy-1-(3-methylbutyl)-1H-quinolin-2-one,
2-{3-[4-hydroxy-1-(3-methylbutyl)-2-oxo-1,2-dihydroquinolin-3-yl)-1,1-dioxo-1,4-dihydrobenzo[1,2,4]thiadiazin-7-yloxy}acetamide,
3-(1,1-dioxo-1,4-dihydrobenzo[1,2,4]thiadiazin-3-yl)-4-hydroxy-1-(tetrahydrofuran-3-ylmethyl)-1H-quinolin-2-one,
3-(1, 1-dioxo-1,4-dihydrobenzo[1,2,4]thiadiazin-3-yl)-4-hydroxy-1-(tetrahydrofuran-2-ylmethyl)-1H-quinolin-2-one,
3-7-fluoro-1,1-dioxo-1,4-dihydrobenzo[1,2,4]thiadiazin-3-yl)+hydroxy-1-(3-methylbutyl)-1H-quinolin-2-one,
4-[3-(1,1-dioxo-1,4-dihydrobenzo[1,2,4]thiadiazin-3-yl)-4-hydroxy-1-(3-methylbutyl)-2-oxo-1,2-dihydroquinolin-6-yloxy]butyronitrile,
3-(1,1-dioxo-1,4-dihydrobenzo[1,2,4]thiadiazin-3-yl)-4-hydroxy-6-(2-methoxy-ethoxy)-1-(3-methylbutyl)-1H-quinolin-2-one,
3-(1,1-dioxo-1,4dihydrobenzo[1,2,4]thiadiazin-3-yl)-4-hydroxy-1-(3-methylbutyl)-2-oxo-1,2-dihydroquinoline-6-carboxylic acid,
3-[1-(2-cyclopropylethyl)-4-hydroxy-2-oxo-1,2-dihydroquinolin-3-yl]-1,1-dioxo-1,4-dihydroenzo[1,2,4]thiadiazine-7-carboxylic acid (3-diethylaminopropyl)amide,
3-[1-(2-cyclopropylethyl)-4-hydroxy-2-oxo-1,2-dihydroquinolin-3-yl]-1,1-dioxo-1,4dihydrobenzo[1,2,4]thiadiazine-7-carboxylic acid benzylamide,
1-2-cyclopropylethyl)-3-[1,1-dioxo-7-(1-pyrrolidin-1-yl-methanoyl)-1,4-dihydrobenzo[1,2,4]thiadiazin-3-yl]4-hydroxy-1H-quinolin-2-one,
3-{7-[1-(4-acetylpiperazin-1-yl)-methanoyl]-1,1-dioxo-1,4-dihydrobenzo[1,2,4]thiadiazin-3-yl}-1-(2-cyclopropylethyl)-4-hydroxy-1H-quinolin-2-one,
3-[1-(2-cyclopropylethyl)-4-hydroxy-2-oxo-1,2-dihydroquinolin-3-yl]-1,1-dioxo-1,4-dihydrobenzo[1,2,4]thiadiazine-7-carboxylic acid (tetrahydrofuran-2-ylmethyl)-amide,
3-[1-(2-cyclopropylethyl)-4-hydroxy-2-oxo-1,2-dihydroquinolin-3-yl]-1,1-dioxo-1,4-dihydrobenzo[1,2,4]thiadiazine-7-carboxylic acid (2,2-dimethoxyethyl)amide,
3-[1-(2-cyclopropylethyl)-4-hydroxy-2-oxo-1,2-dihydroquinolin-3-yl]-1,1-dioxo-1,4-dihydrobenzo[1,2,4]thiadiazine-7carboxylic acid (3-imidazol-1-ylpropyl)amide,
4-hydroxy-3-(5-methoxy-1,1-dioxo-1,4-dihydrobenzo[1,2,4]thiadiazin-3-yl)-1-(3-methylbutyl)-1H-quinolin-2-one,
4-hydroxy-3-(5-hydroxy-1,1-dioxo-1,4-dihydrobenzo[1,2,4]thiadiazin-3-yl)-1-(3-methylbutyl)-1H-quinolin-2-one,
3-(1,1-dioxo-1,4-dihydrobenzo[1,2,4]thiadiazin-3-yl)-4-hydroxy-1-(3-methyl-pentyl)-1H-quinolin-2-one,
3-[4-hydroxy-1-(3-methylbutyl)-2-oxo-1,2-dihydroquinolin-3-yl]-1,1-dioxo-1,4-dihydrobenzo[1,2,4]thiadiazine-7-carbaldehyde,
3-(7-aminomethyl-1,1-dioxo-1,4-dihydrobenzo[1,2,4]thiadiazin-3-yl)-1-(2-cyclopropylethyl-4-hydroxy-1H-quinolin-2-one,
4-hydroxy-3-(6-hydroxy-1,1-dioxo-1,4-dihydrobenzo[1,2,4]thiadiazin-3-yl)-1-(3-methylbutyl)-1H-quinolin-2-one,
(E)-3-{3-[4-hydroxy-1-(3-methylbutyl)-2-oxo-1,2-dihydroquinolin-3-yl]-1,1-dioxo-1,4-dihydrobenzo[1,2,4]thiadiazin-7-yl}acrylamide,
3-(1,1-dioxo-1,4-dihydrobenzo[1,2,4]thiadiazin-3-yl)-4,5,6-trihydroxy-1-(3-methylbutyl)-1H-quinolin-2-one,
3-1,1-dioxo-1,4-dihydrobenzo[1,2,4]thiadiazin-3-yl)-4-hydroxy-1-(3-methoxybutyl)-1H-quinolin-2-one,
3-(1,1-dioxo-1,4-dihydrobenzo[1,2,4]thiadiazin-3-yl)-4-hydroxy-1 pyridin-3-ylmethyl)-1H-quinolin-2-one,
3-(1,1-dioxo-1,4-dibydrobenzo[1,2,4]thiadiazin-3-yl)-4-hydroxy-1-(pyridin-4-ylmethyl)-H-quinolin-2-one,
3-(1,1-dioxo-1,4-dihydrobenzo[1,2,4]thiadiazin-3-yl)-4-hydroxy-1-(3-methylbutyl)-(5,6-methylenedioxy)-1H-quinolin-2-one,
3-(1,1-dioxo-7-(2-oxo-2-pyridin-3-ylethoxy)-1,4-dihydrobenzo[1,2,4]thiadiazin-3-yl)-4-hydroxy-1-(3-methylbutyl)-1H-quinolin-2-one,
3-(1,1-dioxo-4-methyl-1,4-dihydrobenzo[1,2,4]thiadiazin-3-yl)-4-hydroxy-1-(3-methylbutyl)-1H-quinolin-2-one,
3-(1,1-dioxo-7-methyl-1,4-dihydrobenzo[1,2,4]thiadiazin-3-yl)+hydroxy-1-(3-methylbutyl)-1H-quinolin-2-one,
3-(1, 1-dioxo-1,4-dihydrobenzo[1,2,4]thiadiazin-3-yl)-4-hydroxy-1-(3-hydroxypropyl)-1H-quinolin-2-one,
3-(1,1-dioxo-1,4-dihydrobenzo[1,2,4]thiadiazin-3-yl)-4hydroxy-6-(2-hydroxybutoxy)-1-(3-methylbutyl)-1H-quinolin-2-one,
3-(7-dimethylamino-1,1-dioxo-1,4-dihydrobenzo[1,2,4]thiadiazin-3-yl)-4-hydroxy-1-(3-methylbutyl)-1H-quinolin-2-one,
3-{7-[(2R)-aminobutoxy]-1,1-dioxo-1,4-dihydrobenzo[1,2,4]thiadiazin-3-yl)-4 hydroxy-1-(3-methylbutyl)-1H-quinolin-2-one,
3-(1,1-dioxo-1,4-dihydrobenzo[1,2,4]thiadiazin-3-yl)-4-hydroxy-1-(4-nitrobenzyl)-1H-quinolin-2-one,
1-(6-aminopyridin-3-ylmethyl)-3-(1,1-dioxo-1,4-dihydrobenzo[1,2,4]thiadiazin-3-yl)-4-hydroxy-1H-quinolin-2-one,
1-(4-bromobenzyl)-3-(1,1-dioxo-1,4-dihydro-1-benzo[1,2,4]thiadiazin-3-yl)-4 hydroxy-1H-quinolin-2-one,
1-(3-bromobenzyl)-3-(1,1-dioxo-1,4-dihydro-1-benzo[1,2,4]thiadiazin-3-yl)-4-hydroxy-1H-quinolin-2-one,
1-(2-cyclopropylethyl)-6-fluoro-4-hydroxy-3-(7-methoxy-1,1-dioxo-1,4-dihydro-1-benzo[1,2,4]thiadiazin-3-yl)-1H-quinolin-2-one,
3-(7-benzylamino-1,1-dioxo-1,4-dihydro-1-benzo[1,2,4]thiadiazin-3-yl)-4-hydroxy-1-(3-methylbutyl)-1H-quinolin-2-one,
3-{3-[1-(2-cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yl}propionamide,
1-(2-cyclopropylethyl)-6-fluoro-4-hydroxy-3-(7-hydroxy-1,1-dioxo-1,4-dihydro-1-benzo[1,2,4]thiadiazin-3-yl)-1H-quinolin-2-one,
{3-[1-(2-cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yloxy}acetonitrile,
2-(3-[1-(2-cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yloxy}acetamide,
3-(1,1-dioxo-1,4-dihydro-1-benzo[1,2,4]thiadiazin-3-yl)-4-hydroxy-1-(2-methylcyclopropylmethyl)-1H-quinolin-2-one,
1-(2-aminopyridin-4-ylmethyl)-3-(1,1-dioxo-1,4-dihydro-1-benzo[1,2,4]thiadiazin-3-yl)-4-hydroxy-1H-quinolin-2-one,
6-fluoro-4-hydroxy-3-(7-hydroxy-1,1-dioxo-1,4-dihydro-1-benzo[1,2,4]thiadiazin-3-yl)-1-(3-methylbutyl)-1H-quinolin-2-one,
{3-[6-fluoro-4-hydroxy-1-(3-methylbutyl)-2-oxo-1,2-dihydro-quinolin-3-yl]-1,1-dioxo-1,4-dihydro-1-benzo[1,2,4]thiadiazin-7-yloxy]acetonitrile,
4,6-dihydroxy-3-(7-hydroxy-1,1-dioxo-1,4-dihydro-1-benzo[1,2,4]thiadiazin-3-yl)-1-(3-methyl-butyl)-1H-quinolin-2-one,
[3-(7-cyanomethoxy-1,1-dioxo-1,4-dihydro-1-benzo[1,2,4]thiadiazin-3-yl)-4-hydroxy-1-(3-methyl-butyl)-2-oxo-1,2-dihydro-quinolin-6-yloxy]-acetonitrile,
2-{3-[1-(2-cyclopropyl-ethyl)-6-fluoro-4-hydroxy-2-oxo-1,2-dihydro-quinolin-3-yl]-1,1-dioxo-1,4-dihydro-1-benzo[1,2,4]thiadiazin-7-yloxy}-propionamide,
1-(2-cyclopropyl-ethyl)-3-[1,1-dioxo-7-(tetrazol-5-ylmethoxy)-1,4-dihydro-1-benzo[1,2,4]thiadiazin-3-yl]-6-fluoro-4-hydroxy-1H-quinolin-2-one,
2-{3-[6-fluoro-4-hydroxy-1-(3-methyl-butyl)-2-oxo-1,2-dihydro-quinolin-3-yl]-1,1-dioxo-1,4-dihydro-1-benzo[1,2,4]thiadiazin-7-yloxy}-acetamide,
2-[3-(7-carbamoylmethoxy-1,1-dioxo-1,4-dihydro-1-benzo[1,2,4]thiadiazin-3-yl)-4-hydroxy-1-(3-methyl-butyl)-2-oxo-1,2-dihydro-quinolin-6-yloxy]-acetamide,
2-[3-(7-carbamoylmethoxy-1,1-dioxo-1,4-dihydro-1-benzo[1,2,4]thiadiazin-3-yl)-1-(2-cyclopropyl-ethyl)-4-hydroxy-2-oxo-1,2-dihydro-quinolin-6-yloxy]-acetamide,
[3-(7-cyanomethoxy-1,1-dioxo-1,4-dihydro-1-benzo[1,2,4]thiadiazin-3-yl)-4-hydroxy-1-(3-methyl-butyl)-2-oxo-1,2-dihydro-quinolin-6-ylamino]-acetonitrile,
2-{3-[1-(2-cyclopropyl-ethyl)-6-fluoro-4-hydroxy-2-oxo-1,2dihydro-quinolin-3-yl]-1,1-dioxo-1,4-dihydro-1-benzo[1,2,4]thiadiazin-7-yloxy}-2-methyl-propionamide,
3-(1,1-dioxo-1,4-dihydro-1-benzo[1,2,4]thiadiazin-3-yl)-4-hydroxy-1-(3-nitrobenzyl)-1H-quinolin-2-one,
3-(1,1-dioxo-1,4-dihydro-1-benzo[1,2,4]thiadiazin-3-yl)-4-hydroxy-1-(2-methylpyridin-4-ylmethyl)-1H-quinolin-2-one,
[3-[7-(cyanomethyl-amino)-1,1-dioxo-1,4-dihydro-1-benzo[1,2,4]thiadiazin-3-yl]4 hydroxy-1-(3-methyl-butyl)-2-oxo-1,2-dihydro-quinolin-6-ylamino]-acetonitrile,
3-(7-amino-1,1-dioxo-1,4 dihydro-1-benzo[1,2,4]thiadiazin-3-yl)-1-(2-cyclopropylethyl)-6-fluoro-4-hydroxy-1H-quinolin-2-one,
{3-[1 12-cyclopropylethyl)-6-fluoro-4-hydroxy-2-oxo-1,2-dihydroquinolin-3-yl]-1,1-dioxo-1,4-dihydrobenzo[1,2,4]thiadiazin-7-yloxy}acetic acid,
2-[3-7-carbamoylmethoxy-1,1-dioxo-1,4-dihydro-1-benzo[1,2,4]thiadiazin-3-yl)-4-hydroxy-1-(3-methylbutyl)-2-oxo-1,2-dihydroquinolin-6-ylamino]acetamide,
6-fluoro-4-hydroxy-3-(7-hydroxy-1,1-dioxo-1,4-dihydro-1-benzo[1,2,4]thiadiazin-3-yl)-1-pyridin-4-ylmethyl-1H-quinolin-2-one,
1-(2-cyclopropylethyl)-6-fluoro-4-hydroxy-3-[7-(5-methyl-[1,3,4]oxadiazol-2-ylmethoxy)-1,1-dioxo-1,4-dihydro-1-benzo[1,2,4]thiadiazin-3-yl]-1H-quinolin-2-one,
6-chloro-4-hydroxy-3-(7-hydroxy-1,1-dioxo-1,4-dihydro-1-benzo[1,2,4]thiadiazin-3-yl)-1-(3-methylbutyl)-1H-quinolin-2-one,
{3-[6-chloro-4-hydroxy-1-(3-methylbutyl)-2-oxo-1,2-dihydroquinolin-3-yl]-1,1-dioxo-1,4-dihydro-1-benzo[1,2,4]thiadiazin-7-yloxy}acetonitrile,
2-{3-[6-chloro-4-hydroxy-1-(3-methyl-butyl)-2-oxo-1,2-dihydroquinolin-3-yl]-1,1-dioxo-1,4-dihydro-1-benzo[1,2,4]thiadiazin-7-yloxy}acetamide,
3-(7-amino-1,1-dioxo-1,4-dihydro-1-benzo[1,2,4]thiadiazin-3-yl)-6-fluoro-4-hydroxy-1-(3-methylbutyl)-1H-quinolin-2-one,
{3-[6-amino-4-hydroxy-1-(3-methylbutyl)-2-oxo-1,2-dihydroquinolin-3-yl]-1,1-dioxo-1,4dihydro-1 6-benzo[1,2,4]thiadiazin-7-yloxy}acetonitrile,
2-{3-[1-(2-cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yloxy}-N,N-dimethylacetamide,
2-{3-[1-(2-cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yloxy}-N-methylacetamide,
{3-[4-hydroxy-6-(2-hydroxyethylamino)-1-(3-methylbutyl)-2-oxo-1,2-dihydro-quinolin-3-yl]-1,1-dioxo-1,4-dihydro-1-benzo[1,2,4]thiadiazin-7-yloxy}acetonitrile,
1-(2-cyclopropylethyl)-3-[1,1-dioxo-7-(2-oxo-2-pyrrolidin-1-yl-ethoxy)-1,4-dihydro-1-benzo[1,2,4]thiadiazin-3-yl]-6-fluoro-4-hydroxy-1H-quinolin-2-one,
1-(2-cyclopropylethyl)-6-fluoro-4-hydroxy-3-[7-(2-morpholin-4-yl-2-oxo-ethoxy)-1,1-dioxo-1,4-dihydro-1-benzo[1,2,4]thiadiazin-3-yl]-1H-quinolin-2-one,
2-{3-[6-amino-4-hydroxy-1-(3-methylbutyl)-2-oxo-1,2-dihydroquinolin-3-yl]-1,1-dioxo-1,4-dihydro-1-benzo[1,2,4]thiadiazin-7-yloxy)acetamide,
3-(1,1-dioxo-1,4-dihydro-1-benzo[1,2,4]thiadiazin-3-yl)-4-hydroxy-1-(2-methyl-thiazol-4-ylmethyl)-1H-quinolin-2-one,
2-{3-[6-chloro-1-(2-cyclopropylethyl)-4-hydroxy-2-oxo-1,2-dihydroquinolin-3-yl]-1,1-dioxo-1,4-dihydro-1-benzo[1,2,4]thiadiazin-7-yloxy}acetamide,
1-(2-cyclopropylethyl)-3-[1,1-dioxo-7-(2-oxo-2-piperazin-1-yl-ethoxy)-1,4-dihydro-1-benzo[1,2,4]thiadiazin-3-yl]-6-fluoro-4-hydroxy-1H-quinolin-2-one,
1-cyclobutylmethyl-6-fluoro-4-hydroxy-3-(7-hydroxy-1,1-dioxo-1,4-dihydro-1-benzo[1,2,4]thiadiazin-3-yl)-1H-quinolin-2-one,
[3-(1-cyclobutylmethyl-6-fluoro-4-hydroxy-2-oxo-1,2-dihydroquinolin-3-yl)-1,1-dioxo-1,4-dihydro-1-benzo[1,2,4]thiadiazin-7-yloxy]acetonitrile,
2-[3-(1-cyclobutylmethyl-6-fluoro-4-hydroxy-2-oxo-1,2-dihydroquinolin-3-yl)-1,1-dioxo-1,4-dihydro-1-benzo[1,2,4]thiadiazin-7-yloxy]acetamide,
{3-[1-(3,3-dimethylbutyl)-6-fluoro-4-hydroxy-2-oxo-1,2-dihydroquinolin-3-yl]-1,1-dioxo-1,4-dihydro-benzo[1,2,4]thiadiazin-7-yloxy}acetonitrile,
1-cyclobutylmethyl-6-fluoro-4-hydroxy-3-(7-methoxy-1,1-dioxo-1,4-dihydro-1-benzo[1,2,4]thiadiazin-3-yl)-1H-quinolin-2-one,
1-(3,3dimethylbutyl)-6-fluoro-4-hydroxy-3-(7-methoxy-1,1-dioxo-1,4-dihydro-1-benzo[1,2,4]thiadiazin-3-yl)-1H-quinolin-2-one,
3-(7-acetyl-1,1-dioxo-1,4-dihydro-1-benzo[1,2,4]thiadiazin-3-yl)-4-hydroxy-1-(3-methylbutyl)-1H-quinolin-2-one,
(S)-2-(3-[1-(2-cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yloxy}propionamide,
(R)-2-{3-[1-(2-cyclopropylethyl)-6-fluoro-4-hydroxy-2-oxo-1,2-dihydroquinolin-3-yl]-1,1-dioxo-1,4-dihydro-1-benzo[1,2,4]thiadiazin-7-yloxy}propionamide,
{3-[1-(3,3-dimethylbutyl)-6-fluoro-4-hydroxy-2-oxo-1,2-dihydroquinolin-3-yl]-1,1-dioxo-1,4-dihydro-benzo[1,2,4]thiadiazin-7-yloxy}acetamide,
3-{3-[1-(2-cyclopropylethyl)-6-fluoro-4-hydroxy-2-oxo-1,2-dihydro-quinolin-3-yl]-1,1-dioxo-1,4-dihydro-1-benzo[1,2,4]thiadiazin-7-yl}-3-oxopropionamide,
2-{3-[4-hydroxy-6-(2-hydroxyethylamino)-1-(3-methylbutyl)-2-oxo-1,2-dihydroquinolin-3-yl]-1,1-dioxo-1,4-dihydro-1-benzo[1,2,4]thiadiazin-7-yloxy}acetamide,
2-(3-[1-(2-cyclopropylethyl)-4-hydroxy-6-(2-hydroxyethylamino)-2-oxo-1,2-dihydro-quinolin-3-yl]-1,1-dioxo-1,4-dihydro-1-benzo[1,2,4]thiadiazin-7-yloxy}acetamide,
2-{3-[1-(2-cyclopropylethyl)-6-fluoro-4-hydroxy-2-oxo-1,2-dihydroquinolin-3-yl]-1,1-dioxo-1,4dihydro-1-benzo[1,2,4]thiadiazin-7-ylamino}acetamide, and
2-{3-[1-(2-cyclopropylethyl)-6-fluoro-4-hydroxy-2-oxo-1,2-dihydro-quinolin-3-yl]-1,1-dioxo-1,4-dihydro-1-benzo[1,2,4]thiadiazin-7-yloxy}-2-methylpropionic acid,
or a tautomer thereof, or a pharmaceutically acceptable salt or solvate thereof.

8. A pharmaceutically acceptable salt of the compound according to claim 7, or tautomer thereof, wherein said pharmaceutically acceptable salt is a sodium salt or a potassium salt.

9. The compound according to claim 7, selected from the group consisting of:
1-(2-cyclopropylethyl)-6-fluoro-4-hydroxy-3-(7-hydroxy-1,1-dioxo-1,4-dihydro-1-benzo[1,2,4]thiadiazin-3-yl)-1H-quinolin-2-one,
{3-[1-(2-cyclopropylethyl)-6-fluoro-4-hydroxy-2-oxo-1,2-dihydroquinolin-3-yl]-1,1-dioxo-1,1-dihydro-1-benzo[1,2,4]thiadiazin-7-yloxy}acetonitrile,
3-[4-hydroxy-1-(3-methylbutyl)-2-oxo-1,2-dihydroquinolin-3-yl]-1,1-dioxo-1,4 dihydrobenzo[1,2,4]thiadiazine-7-carboxylic acid amide,
2-{3-[1-(2-cyclopropylethyl)-6-fluoro-4-hydroxy-2-oxo-1,2-dihydroquinolin-3-yl]-1,1-dioxo-1,1-dihydro-1-benzo[1,2,4]thiadiazin-7-yloxy}acetamide,
2-[3-(7-carbamoylmethoxy-1,1-dioxo-1,4-dihydro-1-benzo[1,2,4]thiadiazin-3-yl)-4-hydroxy-1-(3-methyl-butyl)-2-oxo-1,2-dihydro-quinolin-6-yloxy]-acetamide,
1-(2-cyclopropylethyl)-3-(1, 1-dioxo-1,4-dihydrobenzo[1,2,4]thiadiazin-3-yl)-6-fluoro-4-hydroxy-1H-quinolin-2-one,
2-{3-[4-hydroxy-1-(3-methylbutyl)-2-oxo-1,2-dihydroquinolin-3-yl]-1,1-dioxo-1,4-dihydrobenzo[1,2,4]thiadiazin-7-yloxy}acetamide,
3-{3-[1-(2-cyclopropylethyl)-6-fluoro-4-hydroxy-2-oxo-1,2-dihydroquinolin-3-yl]-1,1-dioxo-1,1dihydro-1-benzo[1,2,4]thiadiazin-7-yl}propionamide,
{3-[6-fluoro-4hydroxy-1-(3-methylbutyl)-2-oxo-1,2-dihydro-quinolin-3-yl]-1,1-dioxo-1,4-dihydro-1-benzo[1,2,4]thiadiazin-7-yloxy}acetonitrile,
2-[3-(7-carbamoylmethoxy-1,1-dioxo-1,4dihydro-1-benzo[1,2,4]thiadiazin-3-yl)-1-(2-cyclopropyl-ethyl)-4-hydroxy-2-oxo-1,2-dihydro-quinolin-6-yloxy]-acetamide,
1-(2-cyclopropyl-ethyl)-3-[1,1-dioxo-7-(tetrazol-5-ylmethoxy)-1,4-dihydro-1-benzo[1,2,4]thiadiazin-3-yl]-6-fluoro-4-hydroxy-1H-quinolin-2-one,
2-{3-[1-(2-cyclopropyl-ethyl)-6-fluoro-4-hydroxy-2-oxo-1,2-dihydro-quinolin-3-yl]-1,1-dioxo-1,1-dihydro-1-benzo[1,2,4]thiadiazin-7-yloxy}-propionamide,
6-amino-3-(7-amino-1,1-dioxo-1,4-dihydrobenzo[1,2,4]thiadiazin-3-yl)-4hydroxy-1-(3-methylbutyl)-1H-quinolin-2-one,
{3-[4hydroxy-1-(3-methylbutyl)-2-oxo-1,2-dihydroquinolin-3-yl]-1,1-dioxo-1,4-dihydrobenzo[1,2,4]thiadiazin-7-yloxy}acetonitrile,
(E)-3-{3-[4-hydroxy-1-(3-methylbutyl)-2-oxo-1,2-dihydroquinolin-3-yl]-1,1-dioxo-1,4-dihydrobenzo[1,2,4]thiadiazin-7-yl}acrylamide,
6-fluoro-4-hydroxy-3-(7-hydroxy-1,1-dioxo-1,4-dihydro-1-benzo[1,2,4]thiadiazin-3-yl)-1-(3-methylbutyl)-1H-quinolin-2-one,
2-{3-[1-(2-cyclopropyl-ethyl)₆-fluoro-4-hydroxy-2-oxo-1,2-dihydroquinolin-3-yl]-1,1-dioxo-1,4-dihydro-1-benzo[1,2,4]thiadiazin-7-yloxy)-2-methylpropionamide,
[3-(7-cyanomethoxy-1,1-dioxo-1,4-dihydro-1-benzo[1,2,4]thiadiazin-3-yl)-4-hydroxy-1-(3-methyl-butyl)-2-oxo-1,2-dihydro-quinolin-6-yloxy]acetonitrile,
{3-[1-(2-cyclopropylethyl)-6-fluoro-4-hydroxy-2-oxo-1,2-dihydroquinolin-3-yl]-1,1-dioxo-1,4-dihydrobenzo[1,2,4]thiadiazin-7-yloxy}acetic acid,
2-[3-(7-carbamoylmethoxy-1,1-dioxo-1,4-dihydro-1-benzo[1,2,4]thiadiazin-3-yl)-4-hydroxy-1-(3-methylbutyl)-2-oxo-1,2-dihydroquinolin-6-ylamino]acetamide,
6-fluoro-4-hydroxy-3-(7-hydroxy-1,1-dioxo-1,4-dihydro-1-benzo[1,2,4]thiadiazin-3-yl)-1-pyridin-4-ylmethyl-1H-quinolin-2-one,
1-(2-cyclopropylethyl)-6-fluoro-4-hydroxy-3-[7-(5-methyl-[1,3,4]oxadiazol-2-ylmethoxy)-1,1-dioxo-1,4-dihydro-1-benzo[1,2,4]thiadiazin-3-yl]-1H-quinolin-2-one,
6-chloro-4-hydroxy-3-(7-hydroxy-1,1-dioxo-1,4-dihydro-1-benzo[1,2,4]thiadiazin-3-yl)-1-(3-methylbutyl)-1H-quinolin-2-one,
{3-[6-chloro-4-hydroxy-1-(3-methylbutyl)-2-oxo-1,2-dihydroquinolin-3-yl]-1,1-dioxo-1,4-dihydro-1-benzo[1,2,4]thiadiazin-7-yloxy}acetonitrile,
2-(3-[6chloro-4-hydroxy-1-(3-methyl-butyl)-2-oxo-1,2dihydro-quinolin-3-yl]-1,1-dioxo-1,4-dihydro-1-benzo[1,2,4]thiadiazin-7-yloxy}acetamide,
3-(7-amino-1,1-dioxo-1,4dihydro-1-benzo[1,2,4]thiadiazin-3-yl)-6-fluoro-4-hydroxy-1-(3-methylbutyl)-1H-quinolin-2-one,
{3-[6-amino-4-hydroxy-1-(3-methylbutyl)-2-oxo-1,2-dihydroquinolin-3-yl]-1,1-dioxo-1,4-dihydro-16-benzo[1,2,4]thiadiazin-7-yloxy}acetonitrile,
3-[1-(2-cyclopropylethyl)-6-fluoro-4-hydroxy-2-oxo-1,2-dihydroquinolin-3-yl]-1,1-dioxo-1,4-dihydrobenzo[1,2,4]thiadiazin-7-yloxy}acetic acid,
2-[3-(7-carbamoylmethoxy-1,1-dioxo-1,4-dihydro-1-benzo[1,2,4]thiadiazin-3-yl)-4-hydroxy-1-(3-methylbutyl)-2-oxo-1,2-dihydroquinolin-6ylamino]acetamide,
6-fluoro-4-hydroxy-3-(7-hydroxy-1,1-dioxo-1,4-dihydro-1-benzo[1,2,4]thiadiazin-3-yl)-1-pyridin-4-ylmethyl-1H-quinolin-2-one,
1-(2-cyclopropylethyl)-6-fluoro-4-hydroxy-3-[7-(5-methyl-[1,3,4]oxadiazol-2-ylmethoxy)-1,1-dioxo-1,4-dihydro-1-benzo[1,2,4]thiadiazin-3-yl]-1H-quinolin-2-one,
6-chloro-4-hydroxy-3-(7-hydroxy-1,1-dioxo-1,4-dihydro-1-benzo[1,2,4]thiadiazin-3-yl)-1-(3-methylbutyl)-1H-quinolin-2-one,
{3-[6-chloro-4-hydroxy-2-oxo-1-3-methylbutyl)-2-dihydroquinolin-3-yl]-1,1-dioxo-1,4-dihydrobenzo[1,2,4]thiadiazin-7-yloxy}acetamide, and
{3-[6-amino-4-hydroxy-1-(3-methylbutyl)-2-oxo-1,2-dihydroquinolin-3-yl]-1,1-dioxo-1,4-dihydro-16-benzo[1,2,4]thiadiazin-7-yloxy}acetonitrile,
or a tautomer thereof, or a pharmaceutically acceptable salt or solvate thereof.

10. A pharmaceutically acceptable salt of the compound according to claim 9, or tautomer thereof, wherein said pharmaceutically acceptable salt is a sodium salt or a potassium salt.

11. A compound selected from the group consisting of:
3-(1,1-dioxo-1,4-dihydrobenzo[1,2,4]thiadiazin-3-yl)-4-hydroxy-1-(2-phenylethyl)-1H-quinolin-2-one,
1-(2-cyanobenzyl)-3-(1,1-dioxo-1,4-dihydrobenzo[1,2,4]thiadiazin-3-yl)-4-hydroxy-1H-quinolin-2-one,
3-(1,1-dioxo-1,4-dihydrobenzo[1,2,4]thiadiazin-3-yl)-4-hydroxy-1-(3-phenylpropyl)-1H-quinolin-2-one,
N-[3-(1,1-dioxo-1,4-dihydrobenzo[1,2,4]thiadiazin-3-yl)-4-hydroxy-1-(3-methylbutyl)-2-oxo-1,2-dihydroquinolin-6-yl]4-methoxybenzamide,
3-(1,1-dioxo-1,4-dihydrobenzo[1,2,4]thiadiazin-3-yl)-4-hydroxy-1-(3-methylbutyl)-2-oxo-1,2-dihydroquinoline-6-carboxylic acid ethyl ester,
3-[1-(2-cyclopropylethyl)-4-hydroxy-2-oxo-1,2-dihydroquinolin-3-yl]-1,1dioxo-1,4-dihydroenzo[1,2,4]thiadiazine-7-carboxylic acid [2-(4-methoxyphenyl)ethyl]amide,
4-[3-(1,1-dioxo-1,2dihydrobenzo[1,2,4]thiadiazin-3-yl)-4-hydroxy-2-oxoquinolin-1-yl]butyramide,
3-(1,1-dioxo-1,2-dihydrobenzo[1,2,4]thiadiazin-3-yl)-4-hydroxy-1-(3-methylbutyl)-2-oxo-1,2-dihydro-quinoline-7-carboxylic acid,
7-bromo-3-(1,1-dioxo-1,2-dihydrobenzo[1,2,4]thiadiazin-3-yl)-4-hydroxy-1-(3-methylbutyl)-1H-quinolin-2-one,
3-(1,1-dioxo-7-phenyl-1,4-dihydro-1-benzo[1,2,4]thiadiazin-3-yl)-4-hydroxy-1-(3-methylbutyl)-1H-quinolin-2-one,
thiophene-2-carboxylic acid [3-(1,1-dioxo-1,4-dihydro-1-benzo[1,2,4]thiadiazin-3-yl)-4-hydroxy-1-(3-methylbutyl)-2-oxo-1,2 dihydro-quinolin-6-yl]amide,
N-[3-(1,1-dioxo-1,4-dihydro-1-benzo[1,2,4]thiadiazin-3-yl)-4-hydroxy-1-(3-methylbutyl)-2-oxo-1,2-dihydro-quinolin-6-yl]-3-trifluorobenzamide,
N-[3-(1,1-dioxo-1,4-dihydro-1-benzo[1,2,4]thiadiazin-3-yl)-4-hydroxy-1-(3-methylbutyl)-2-oxo-1,2-dihydro-quinolin-6-yl]-3-fluorobenzamide,
N-[3-(1,1-dioxo-1,4-dihydro-1-benzo[1,2,4]thiadiazin-3-yl)-4-hydroxy-1-(3-methylbutyl)-2-oxo-1,2dihydro-quinolin-6-yl]-nicotinamide,
N-[3-(1,1-dioxo-1,4-dihydro-1-benzo[1,2,4]thiadiazin-3-yl)+hydroxy-1-(3-methylbutyl)-2-oxo-1,2-dihydro-quinolin-6-yl]acetamide,
4-hydroxy-3-(7-iodo-1,1-dioxo-1,4-dihydro-1-benzo[1,2,4]thiadiazin-3-yl)-1-(3-methylbutyl)-1H-quinolin-2-one,
4-[3-(1,1dioxo-1,4-dihydro-1-benzo[1,2,4]thiadiazin-3-yl)-4-hydroxy-2-oxo-2H-quinolin-1-yl]-butyric acid ethyl ester,
4-[3-(1,1-dioxo-1,4-dihydro-1-benzo[1,2,4]thiadiazin-3-yl)-4-hydroxy-2-oxo-2H-quinolin-1-yl]-butyric acid,
[3-(1,1-dioxo-1,4-dihydro-1-benzo[1,2,4]thiadiazin-3-yl)-4-hydroxy-6-(3-methyl-butoxy)-2-oxo-2H-quinolin-1-yl]acetic acid,
3-(1,1-dioxo-1,4-dihydro-1-benzo[1,2,4]thiadiazin-3-yl)-4-hydroxy-6-iodo-1-(3-methylbutyl)-1H-quinolin-2-one,
3-(1,1-dioxo-1,1-dihydro-1-benzo[1,2,4]thiadiazin-3-yl)-4-hydroxy-8-(3-methylbutyl)-1H-quinolin-2-one,
3-(1, 1-dioxo-1,4-dihydro-1-benzo[1,2,4]thiadiazin-3-yl)-4-hydroxy-1-(3-methylbutyl)-2-oxo-1,2-dihydro-quinoline-5-carboxylic acid dimethylamide,
3-(1,1-dioxo-1,4-dihydro-1-benzo[1,2,4]thiadiazin-3-yl)-4-hydroxy-1-(3-methylbutyl)-2-oxo-1,2-dihydro-quinoline-5-carboxylic acid,
3-(1, 1-dioxo-1,4-dihydro-1-benzo[1,2,4]thiadiazin-3-yl)-4-hydroxy-6-(4-methoxy-benzyloxy)-1-(3-methylbutyl)-1H-quinolin-2-one,
1-(2-dimethylamino-ethyl)-3-(1,1-dioxo-1,4-dihydro-1-benzo[1,2,4]thiadiazin-3-yl)-4-hydroxy-1H-quinolin-2-one,
4-hydroxy-3-(6-methoxy-1,1-dioxo-1,4-dihydro-1-benzo[1,2,4]thiadiazin-3-yl)-1-(3-methylbutyl)-1H-quinolin-2-one,
6-(6-bromo-pyridin-2-yloxy)-3-(1,1-dioxo-1,4-dihydro-1-benzo[1,2,4]thiadiazin-3-yl)-4-hydroxy-1-(3-methylbutyl)-1H-quinolin-2-one,
3-(1,1-dioxo-1,4-dihydro-1-benzo[1,2,4]thiadiazin-3-yl)-4-hydroxy-1-(2-hydroxyethyl)-1H-quinolin-2-one,
3-(1,1-dioxo-1,4-dihydro-1-benzo[1,2,4]thiadiazin-3-yl)-4-hydroxy-6-(6-methoxy-pyridin-2-yloxy)-1-(3-methylbutyl)-1H-quinolin-2-one,
3-(1,1-dioxo-1,4-dihydro-1-benzo[1,2,4]thiadiazin-3-yl)-1-(5-fluoro-2-methyl-benzyl)-4-hydroxy-1H-quinolin-2-one, and
3-(1,1-dioxo-1,4-dihydro-1-benzo[1,2,4]thiadiazin-3-yl)-4-hydroxy-1-(2-nitrobenzyl)-1H-quinolin-2-one,
or a tautomer thereof, or a pharmaceutically acceptable salt or solvate thereof.

12. A pharmaceutically acceptable salt of the compound according to claim 11, or tautomer thereof, wherein said pharmaceutically acceptable salt is a sodium salt or a potassium salt.

13. A method of inhibiting an RNA-containing virus which comprises contacting said virus with an effective amount of the compound according to any one of claims 1 to 12.

14. A method of treating infection caused by an RNA-containing virus which comprises administering to a subject in need thereof an effective amount of the compound according to any one of claims 1 to 10.

15. A method according to claim 14 comprising treating an HCV infection.

16. A method according to claim 13 or claim 14 comprising inhibiting hepatitis C virus.

17. A method according to claim 15, wherein said HCV infection is acute hepatitis infection, chronic hepatitis infection, hepatocellular carcinoma or liver fibrosis.

18. A method according to claim 14 comprising treating an infection caused by Dengue, HIV or a picornavirus.

19. A method according to claim 14 comprising administering said compound in combination with one or more agents selected from the group consisting of an immunomodulatory agent and an antiviral agent.

20. A method according to claim 19, wherein the immunomodulatory agent is selected from the group consisting of alpha interferon, beta interferon, gamma interferon, a cytokine, a vitamin, a nutritional supplement, an antioxidant compound, a vaccine and a vaccine comprising an antigen and an adjuvant.

21. A method according to claim 14 comprising administering said compound in combination with an interferon.

22. A method according to claim 14 comprising administering said compound in combination with an HCV antisense agent.

23. A method according to claim 14 comprising administering said compound in combination with an immunoglobulin, a peptide-nucleic acid conjugate, an oligonucleotide, a ribozyme, a polynucleotide, an anti-inflammatory agent, a pro-inflammatory agent, an antibiotic or a hepatoprotectant.

24. A method for inhibiting replication of hepatitis C virus comprising inhibiting replication of both positive and negative strand HCV-RNA, said method comprising contacting a cell infected with said virus with an effective amount of the compound according to any one of claims 1 to 12.

25. A method of treating infection caused by hepatitis C virus comprising inhibiting replication of both positive and negative strand HCV-RNA, said method comprising administering to a subject in need thereof an effective amount of the compound according to any one of claims 1 to 10.

26. The method according to claim 24, wherein said compound substantially equally inhibits positive strand HCV-RNA replication and negative strand HCV-RNA replication.

27. The method according to claim 25, wherein said compound substantially equally inhibits positive strand HCV-RNA replication and negative strand HCV-RNA replication.

28. A method of preparing the compound of Formula I according to claim 1 comprising the steps of:
a) treating a 2-aminobenzoic acid having the formula: 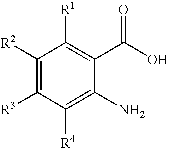
with phosgene or a phosgene equivalent to provide a benzo[d][1,3]oxazine having the formula: 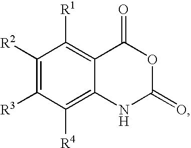
or treating an indole-2,3-dione having the formula: 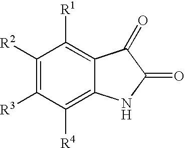
with a peracid to provide the benzo[d][1,3]oxazine having the formula: 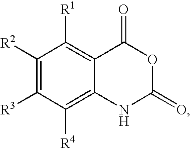
b) converting the benzo[d][1,3]oxazine to an N-alkylated benzo[d][1,3]oxazine-2,4-dione having the formula: 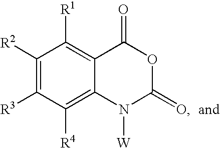
c) coupling the N-alkylated benzo[d][1,3]oxazine with a thiadiazine having the formula: 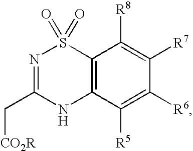
where R is C₁-C₄ alkyl, to provide the compound of Formula I.

29. A method of preparing the compound of Formula I according to claim 1 comprising the steps of:
a) treating a benzo[d][1,3]oxazine-2,4-dione having the formula: 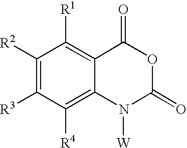
with a cyanoacetate to provide a 3-cyanoquinoline having the formula: 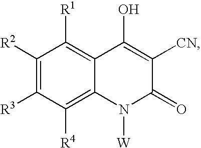
b) treating the 3-cyanoquinoline with a 2-aminobenzenesulfonamide to provide the compound of Formula I,
or converting the 3-cyanoquinoline to an amidine having the formula: 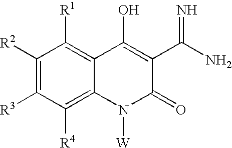
and treating the amidine with a 2-chlorobenzenesulfonyl chloride to provide the compound of Formula I.

30. A method of preparing the compound of Formula I according to claim 1 comprising the steps of:
a) treating an aniline with chlorosulfonylisocyanate then an acid to provide a thiadiazine compound having the formula: 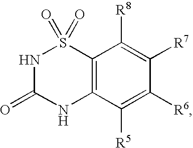
b) treating the thiadiazine compound with an aqueous acid to provide a 2-aminobenzenesulfonamide having the formula: 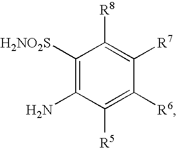
c) treating the 2-aminobenzenesulfonamide with ethyl chloromalonate in the presence of a base to provide an amide having the formula: 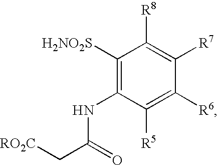
where R is C₁-C₄ alkyl,
d) treating the amide with a dehydrating agent to provide a thiadiazines having the formula: 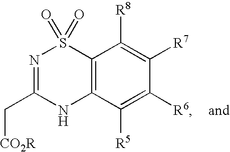
e) treating the thiadiazine with a benzo[d][1,3]oxazine having the formula: 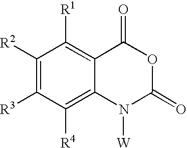
to provide the compound of Formula I.

31. A method of preparing the compound of Formula I according to claim 1 comprising the steps of:
a) converting a 7-methoxythiadiazine having the formula; 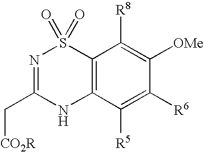
where R is C₁-C₄ alkyl, to a 7-hydroxy compound having the formula: 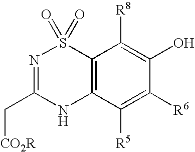
b) treating the 7-hydroxy compound with a benzo[d][1,3]oxazine having the formula: 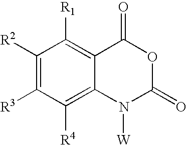
to provide a hydroxy-containing compound of Formula I having the formula: 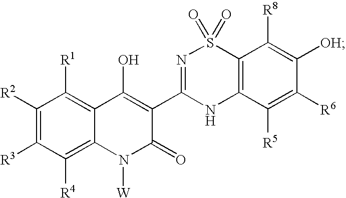
c) optionally treating the hydroxy-containing compound of Formula I with an agent to provide a compound of Formula I having the formula: 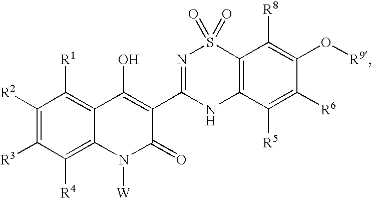
where R^{9′} is defined as R⁹, above, except that R^{9′} is not H.

32. A method of preparing the compound of Formula I according to claim 1 comprising the steps of:
a) treating a 2-aminobenzoic acid having the formula: 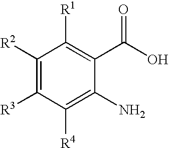
with an aldehyde, having the formula W-CHO, in the presence of a reducing agent to provide an N-akylated 2-aminobenzoic acid having the formula: 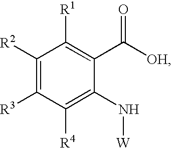
b) treating the N-akylated 2-aminobenzoic acid with phosgene or a phosgene equivalent to provide an N-alkylated benzo[d][1,3]oxazine-2,4-dione having the formula: 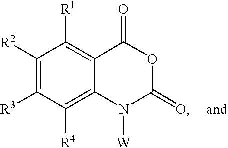
c) coupling the N-alkylated benzo[d][1,3]oxazine with a thiadiazine having the formula: 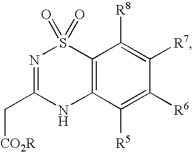
where R is C₁-C₄ alkyl, to provide the compound of Formula I.

33. A method of preparing the compound of Formula I according to claim 1 comprising the steps of:
a) treating a 2-halobenzoic acid having the formula: 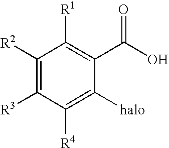
where the halogen is bromine or chlorine, with an N-substituted amine, having the formula W—NH, in the presence of a copper catalyst to form an N-akylated 2-aminobenzoic acid having the formula: 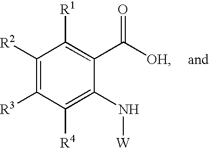
b) converting the N-akylated 2-aminobenzoic acid into the compound of Formula I.

34. The method I according to claim 33, further comprising the steps of:
a) treating the N-akylated 2-aminobenzoic acid with phosgene or a phosgene equivalent to provide an N-alkylated benzo[d][1,3]oxazine-2,4-dione having the formula: 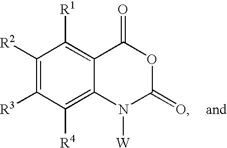
b) coupling the N-alkylated benzo[d][1,3]oxazine with a thiadiazine having the formula: 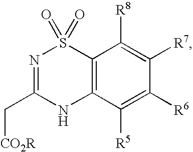
where R is C₁-C₄ alkyl, to provide the compound of Formula I.

35. A method of preparing the compound of Formula I according to claim 1 comprising the steps of:
a) treating a 2-halobenzoic acid having the formula: 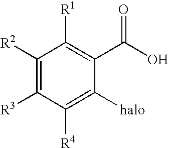
where the halogen is bromine or chlorine, with an N-substituted amine, having the formula W—NH, in the presence of a catalyst to form an N-akylated 2-aminobenzoic acid having the formula: 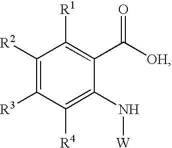
b) treating the N-akylated 2-aminobenzoic acid with phosgene or a phosgene equivalent to provide an N-alkylated benzo[d][1,3]oxazine-2,4-dione having the formula: 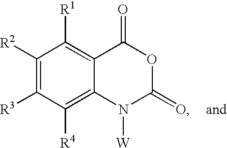
c) coupling the N-alkylated benzo[a][1,3]oxazine with a thiadiazine having the formula: 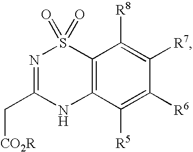
where R is C₁-C₄ alkyl, to provide the compound of Formula I.

36. The method according to any one of claims 28-35 further comprising the step of treating the compound of Formula I with a base to provide the pharmaceutically acceptable salt of the compound of Formula I.

37. The method according to claim 36 wherein said base is sodium hydroxide or potassium hydroxide.

38. The method according to claim 36 wherein said pharmaceutically acceptable salt is a sodium salt or a potassium salt.

39. A compound having the formula: 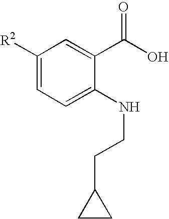
wherein R² is hydrogen halogen, —OR^{b}, —NHR^{b′}, NO₂, where R^{b′} is H or C₁-C₂ alkyl, where the C₁-C₂ alkyl is optionally unsubstituted or substituted by a substituent selected from the group consisting of cyano, —OH, —CO₂H, —CONH₂, —C(O)OC₁-C₂ alkyl, —CONH(C₁-C₂ alkyl), and unsubstituted monocyclic heteroaryl, or a pharmaceutically acceptable salt or solvate thereof.

40. A compound having the formula: 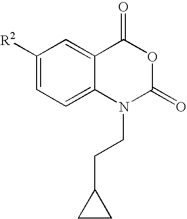
wherein R² is hydrogen halogen, —OR^{b′}, —NHR^{b′}, NO₂, where R^{b′} is H or C₁-C₂ alkyl, where the C₁-C₂ alkyl is optionally unsubstituted or substituted by a substituent selected from the group consisting of cyano, —OH, —CO₂H, —CONH₂, —C(O)OC₁-C₂ alkyl, —CONH(C₁-C₂ alkyl), and unsubstituted monocyclic heteroaryl, or a pharmaceutically acceptable salt or solvate thereof.

41. The compound according to claim 39 or claim 40, wherein R² is fluoro.

42. A compound selected from the group consisitng of: 1-phenethyl-1H-benzo[d][1,3]oxazine-2,4-dione, 1-(2-cyanobenzyl)-1H-benzo[d ][1,3]oxazine-2,4dione, 1-(3-phenylpropyl)-1H-benzo[d][1,3]oxazine-2,4-dione, 1-cyclopropylmethyl-1H-benzo[d][1,3]oxazine-2,4-dione, 1-(3-methylbutyl)-6-nitrobenzo[d][1,3]oxazine-2,4-dione, 6-chloro-1-(3-methylbutyl)benzo[d][1,3]oxazine-2,4-dione, 6-bromo-1-(3-methylbutyl)-benzo[d][1,3]oxazine-2,4-dione, 6-methoxy-3-methylbutyl)benzo[d][1,3]oxazine-2,4-dione, 1-but-3-enyl-1H-benzo[d][1,3]oxazine-2,4-dione, 1-cyclohexylmethyl-1H-benzo[d][1,3]oxazine-2,4-dione, 1-(3-methylbut-2-enyl)-1H-benzo[d][1,3]oxazine-2,4-dione, 1-(3-methylbutyl)-6-methylbenzo[d][1,3]oxazine-2,4-dione, 1-(3-methylbutyl)-6-fluorobenzo[d][1,3]oxazine-2,4-dione, 1-(3-methylbutyl)-5-fluorobenzo[d][1,3]oxazine-2,4-dione, 1-(3-methylbutyl)-6,7-difluorobenzo[d][1,3]oxazine-2,4-dione, 1-(3-methylbutyl)-7-fluorobenzo[d][1,3]oxazine-2,4-dione, 1-(4,4,4-trifluorobutyl)-1H-benzo[d][1,3]oxazine-2,4-dione, 3-cyano-4-hydroxy-1-(3-methylbutyl)-1H-quinolin-2-one, 4hydroxy-1-(3-methylbutyl)-2-oxo-1,2-dihydroquinoline-3-carboxamidine, 1-pent-4-ynyl-1H-benzo [d][1,3]oxazine-2,4-dione, methyl (7-bromo-1,1-dioxo-1,2-dihydrobenzo[1,2,4]thiadiazin-3-yl)acetate, 8-bromo-2-isobutoxybenzo[d][1,3]oxazin-4-one, 1-cyclopropylmethyl-1H-benzo[d][1,3]oxazine-2,4-dione, 1-(3-methylbutyl)-5-methylbenzo[d][1d,3]oxazine-2,4-dione, 1-(3-methylbutyl)-5-chlorobenzo[d][1,3]oxazine-2,4-dione, 5-bromo-2H-3,1-benzoxazine-1-(3-methylbutyl)-2,4-dione, 7-bromo-2H-3,1-benzoxazine-1-(3-methylbutyl)-2,4-dione, 4-(6-methoxybenzo[ ][1,3]oxazine-2,4-dione-1-yl)butyronitrile, 1-butyn-3-yl-1H-benzo[d ][1,3]oxazine-2,4-dione, 1-(3,3-dimethylbutyl)-1H-benzo[d][1,3]oxazine-2,4-dione, 1-2-cyclopropylethyl)-1H-benzo[d][1,3]oxazine-2,4-dione, 3-cyano-4-hydroxy-1-(3-methylbutyl)-1H-quinolin-2-one, 1-(2-methylthio)ethyl-1H-benzo[d][1,3]oxazine-2,4-dione, 1-(2-methylthio)propyl-1H-benzo[d][1,3]oxazine-2,4-dione, 1-(3-furanylmethyl)methyl-1H-benzo[d][1,3]oxazine-2,4-dione, 1-[2-(2-thienyl)]ethyl-1H-benzo[d][1,3]oxazine-2,4-dione, 1-[2-3-thienyl)]ethyl-1H-benzo[d][1,3]oxazine-2,4 dione, 1-(2-cyclopropylethyl)-6-fluorobenzo[d][1,3]oxazine-2,4-dione, 6-(tert-butyl-dimethylsilanyloxy)-1-(2-cyclopropylethyl)-1H-benzo[d]oxazine-2,4-dione, 3-cyano-1-(2-cyclopropylethyl)-4-hydroxy-1H-quinolin-2-one, (7-iodo-1,1-dioxo-1,4-dihydro-1-benzo[1,2,4]thiadiazin-3-yl)-acetic acid ethyl ester, 1-(3,3-dimethylbutyl)-6-nitrobenzo[d][1,3]oxazine-2,4-dione, 3-(1,1-dioxo-7-iodo-1,4-dihydrobenzo[1,2,4]thiadiazin-3-yl)-4-hydroxy-1-(3-methylbutyl)-6-nitro-1H-quinolin-2-one, 3-[4-hydroxy-1-(3-methylbutyl)-6-nitro-2-oxo-1,2-dihydroquinolin-3-yl]-1,1-dioxo-1,4-dihydrobenzo[1,2,4]thiadiazine-7-carboxylic acid amide, 1-(tetrahydrofuran-3-ylmethyl)-1H-benzo[ ][1,3]oxazine-2,4-dione, 6-iodo-1-(3-methylbutyl)-1H-benzo[d][1,3]oxazine-2,4-dione, 1-(tetrahydro-furan-2-ylmethyl)-1H-benzo[d][1,3]oxazine-2,4-dione, 1-(3-methylpentyl)-1H-benzo[d][1,3]oxazine-2,4-dione, 4-hydroxy-3-(7-iodo-1,1-dioxo-1,4-dihydrobenzo[1,2,4]thiadiazin-3-yl)-1-(3-methylbutyl)-1H-quinolin-2-one, 5,6dimethoxy-1-(3-methyl-butyl)-1H-benzo[d][1,3]oxazine-2,4-dione, 4-methyl-1,1-dioxo-1,4-dihydrobenzo[1,2,4]thiadiazin-3-one, N-methyl-N-(2-sulfamoylphenyl)malonamic acid ethyl ester, (4-methyl-1,1-dioxo-1,4-dihydrobenzo[1,2,4]thiadiazin-3-yl)acetic acid ethyl ester, 3-(1,1-dioxo-7-methyl-1,4-dihydrobenzo[1,2,4]thiadiazin-3-yl)-acetic acid ethyl ester, 1-(4-nitrobenzyl)-1H-benzo[d ][1,3]oxazine-2,4-dione, 1-(6-aminopyridin-3-ylmethyl)-1H-benzo[d][1,3]oxazine-2,4-dione, 1-(4-bromobenzyl)-1H-benzo[d][1,3]oxazine-2,4-dione, 1-(3-bromobenzyl)-1H-benzo[d][1,3]oxazine-2,4-dione, 7-methoxy-1,1-dioxo-1,4-dihydrobenzo[1,2,4]thiadiazin-3-one, N-(4-methoxy-2-sulfamoylphenyl)malonic acid ethyl ester, (7-methoxy-1,1-dioxo-1,4-dihydrobenzo[1,2,4]thiadiazin-3-yl)-acetic acid ethyl ester, 1-(2-cyclopropylethyl)-3-(1,1-Dioxo-1,4-dihydrobenzo[1,2,4]thiadiazin-7-iodo-3-yl)-6-fluoro-4-hydroxy-1H-quinolin-2-one, (7-hydroxy-1,1-dioxo-1,4-dihydrobenzo[1,2,4]thiadiazin-3-yl)acetic acid ethyl ether, 1-([(2-methylcyclopropyl)methyl]-1H-benzo[d][1,3]oxazine-2,4-dione, 1-([(2-methylcyclopropyl)methyl]-1H-benzo[d][1,3]oxazine-2,4-dione, 4,6-dihydroxy-3-(7-methoxy-1,1-dioxo-1,4-dihydrobenzo[1,2,4]thiadiazin-3-yl)-1-(3-methyl-butyl)-1H-quinolin-2-one, 1-(2-cyclopropyl-ethyl)-4,6-dihydroxy-3-(7-hydroxy-1,1-dioxo-1,4-dihydro-1-benzo[1,2,4]thiadiazin-3-yl)-1H-quinolin-2-one, 6-amino-1-(3-methylbutyl)-1H-benzo[d][1,3]oxazine-2,4-dione, 6-amino-4-hydroxy-3-(7-methoxy-1,1-dioxo-1,2-dihydrobenzo[1,2,4]thiadiazin-3-yl)-1-(3-methylbutyl)-1H-quinolin-2-one, 6-amino-4-hydroxy-3-(7-hydroxy-1,1-dioxo-1,2-dihydrobenzo[1,2,4]thiadiazin-3-yl)-1-(3-methylbutyl)-1H-quinolin-2-one, and 1-(3-nitrobenzyl)-1H-benzo[d][1,3]oxazine-2,4-dione, 1-[(2-methylpyridin-4yl)methyl]-1H-benzo[d][1,3]oxazine-2,4-dione, or a tautomer thereof, or a pharmaceutically acceptable salt or solvate thereof.
